(19) United States
(12) Patent Application Publication
(10) Pub. No.: US 2014/0243404 A1
(43) Pub. Date: Aug. 28, 2014

(54) UNIQUE HALOGEN-INDUCED CYCLIZATIONS, REAGENTS THEREFOR, AND COMPOUNDS PRODUCED THEREBY

(71) Applicants: Scott Alan Snyder, Dobbs Ferry, NY (US); Daniel S. Treitler, Jersey City, NJ (US); Alexandria P. Brucks, Barrington, IL (US); Andreas Gollner, Vienna (AT); Maria I. Chiriac, New York, NY (US); Nathan E. Wright, New York, NY (US); Jason J. Pflueger, Berkley, CA (US); Steven P. Breazzano, New York, NY (US)

(72) Inventors: Scott Alan Snyder, Dobbs Ferry, NY (US); Daniel S. Treitler, Jersey City, NJ (US); Alexandria P. Brucks, Barrington, IL (US); Andreas Gollner, Vienna (AT); Maria I. Chiriac, New York, NY (US); Nathan E. Wright, New York, NY (US); Jason J. Pflueger, Berkley, CA (US); Steven P. Breazzano, New York, NY (US)

(73) Assignee: The Trustees of Columbia University in the City of New York, New York, NY (US)

(21) Appl. No.: 13/823,565

(22) PCT Filed: Sep. 13, 2011
(86) PCT No.: PCT/US2011/051311

§ 371 Date: Nov. 1, 2013

Related U.S. Application Data
(60) Provisional application No. 61/403,406, filed on Sep. 14, 2010.

(51) Int. Cl. C07D 493/10; C07C 323/22; C07C 319/02; C07C 43/225; C07D 493/04; C07D 319/08; C07C 13/573; C07C 13/66; C07D 313/18; C07D 313/00; C07C 323/03; C07D 311/78

(52) U.S. Cl. CPC C07D 493/10; C07C 323/03; C07C 323/22; C07C 319/02; C07C 43/225; C07D 311/78; C07D 319/08; C07C 13/573; C07C 13/66; C07D 313/18; C07D 313/00; C07D 493/04
USPC 514/452; 568/56; 568/42; 568/632; 549/388; 549/228; 549/384; 585/26; 549/277; 549/278; 549/346; 549/230; 568/633; 514/717; 514/454; 514/753; 514/715; 514/675; 514/681; 514/765; 514/510; 514/719

(57) ABSTRACT

This disclosure is related to halonium compounds useful for cyclization of polyenes, alkenoic acids, and alkenyl alkyl ethers, and halogenation of aromatic compounds. The synthesis of such halonium compounds, compounds made using such halonium compounds, and synthesis of natural compounds, including decalins, using the halonium compounds is also disclosed. A representative halonium compound of the disclosure is:

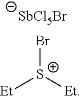

This application claims priority of U.S. Provisional Application No. 61/403,406, filed Sep. 14, 2010, the contents of which are hereby incorporated by reference.

The work disclosed herein was made with government support under Grant No. CHE0844593 from the National Science Foundation and Grant No. GM-084994 from the National Institutes of Health. Accordingly, the U.S. Government has certain rights in this invention.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

With little question, the ability to convert polyene starting materials into far more complex frameworks via stereoselective cation-π cyclizations constitutes one of the most important strategies currently available for C—C bond construction (1). Indeed, in the half century since Stork and Eschenmoser first advanced their hypothesis (2) for the existence of such processes, chemists have devised numerous sets of unique substrates, reaction conditions, and reagent combinations that enable such reactions to be conducted with very high levels of stereoselectivity. Specifically, numerous versions of non-metal- (3) and metal-induced (4) [especially Hg(II) (4a-4i), Pd(II) (4j-4i), Pt(II) (4l-4o), and Au(I) (40-4-q)]cyclizations have been developed and honed to the point where the efficient synthesis of dozens of molecules of natural and designed origin can readily be achieved (5).

What remains to be accomplished, however, is broadly initiating such processes with halogen electrophiles. Nature takes advantage of such reactivity with some frequency, as vanadium- and heme-based haloperoxidases (6) have been shown (or hypothesized) to convert simple polyene precursors into the highlighted rings of the natural products drawn in FIG. 1 (7) these molecules represent a select subset of the nearly 200 chlorine- and bromine-containing compounds which possess such ring systems that have been isolated to date from both marine and terrestrial sources (8). Yet, mirroring such reactivity in the laboratory flask has proven elusive unless haloperoxidases themselves have been utilized (9). Indeed, the use of simple halogen electrophiles to achieve such cyclizations, even in racemic form, typically has led to modest product yields and then only for a narrow range of substrates with certain halogens (10-13).

To the best of our knowledge, there have been no examples of any chemical reagents effecting a chloronium-induced polyene cyclization in any yield (10). Explorations with bromine-based systems, by contrast, have been much more extensive. Nevertheless, no reagent possesses the scope of reactivity needed to handle the diverse range of C═C double bond nucleophilicity possible in functionalized terpene precursors (11). Most reagents convert electron-rich systems into multiple, and often challenging to separate, products due to issues of olefin chemoselectivity, with electron-poor substrates typically leading to products where the cyclizations stall after forming a single ring (i6→7) (11e) or an exogenous species behaves as nucleophile or base prior to cation-π cyclization (8→9) (11g). In fact, yields of cyclized material from electron-deficient systems using electrophilic bromine initiators have always been less than 30%.

Iodonium-induced reactions (12) are the best developed, largely due to two recent advances. The first is Ishihara's use of a phosphorous-complexed form of N-iodosuccinimide (NIS) to cyclize three aryl-containing polyenes derived from geraniol (including 10); when certain chiral phosphoramidites were used in stoichiometric amounts, the cyclization could be achieved with high enantioselection (95% e.e.) (12a). Key was the use of 30 hours of controlled cryogenic conditions in the initial halonium-induced reaction followed by the addition of ClSO₃H in a separate step to convert partially-cyclized materials (such as 11) into the final tricycle (i.e. 12). Efforts to deploy such reactivity for enantioselective bromonium-induced cyclizations, however, were not as successful (14). The second advance is the recent disclosure of Barluenga's hypervalent iodonium-reagent Ipy₂BF₄. When coupled with an additional equivalent of HBF₄, this reagent was able to convert several terpenes into cyclized products (12b). However, neither of these two reagent combinations has been reported to successfully cyclize an electron-deficient polyene substrate.

Thus, given this global range of present capabilities for all direct halonium-based cyclizations, especially that for bromine and chlorine, most natural product structures of the types represented by 1-5 (cf. FIG. 1) have been targeted through strategies that feature indirect, multistep alternatives. These variants have included the formation and cyclization of halohydrin intermediates (15), stoichiometric Hg(II)-induced cyclizations followed by stereoselective replacement with chlorine, bromine, or iodine (16, 4a, 17) or two-step inversion and replacement sequences from oxygen-cyclized materials (18).

### SUMMARY OF THE INVENTION

The invention provides a process for cyclizing an alkene comprising contacting the alkene with a compound having the structure:

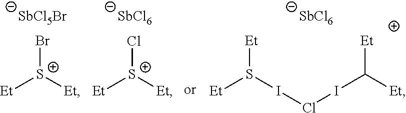

under conditions permitting cyclization of the alkene.

The invention provides a process for halogenating an aromatic ring comprising contacting the aromatic ring with a compound having the structure:

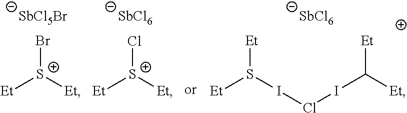

under conditions permitting halogenation of the aromatic ring.

The invention provides a compound having the structure:

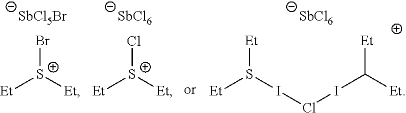

The invention provides a compound having the structure:

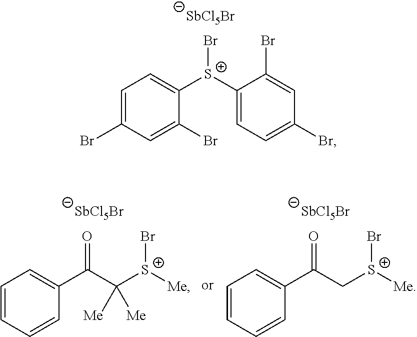

The invention provides a process for producing a compound having the structure:

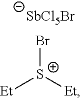

comprising contacting Br₂ with excess Et₂S and SbCl₅ in a suitable solvent at a suitable temperature so as to thereby produce the compound.

A process for producing a compound having the structure:

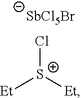

comprising contacting Cl₂ with Et₂S and SbCl₅ in a suitable solvent at a first suitable temperature, and subsequently contacting the resulting product with hexanes prior to cooling to a second suitable temperature so as to thereby produce the compound.

The invention provides a process for producing a compound having the structure:

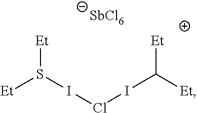

comprising contacting I₂ with excess Et₂S and SbCl₅ in a suitable solvent at a first suitable temperature, warming the resulting product, and subsequently contacting the resulting product with hexanes at a second suitable temperature so as to thereby produce the compound.

The invention provides a compound having the structure:

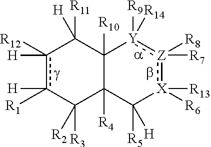

wherein
Y and X are, independently, a C atom or an O atom,
   wherein when X is O, R₆ and R₁₃ are absent and when Y is O, R₉ and R₁₄ are absent;
Z is a carbon atom;
α, β and γ are, independently, present or absent, and when present each is a bond;
R₁ is OH or a halogen, or is absent if bond γ is present;
R₂, R₃, R₅, R₁₀, R₁₁ and R₁₂ are, independently, H, OH or a C₁₋₄ alkyl;
R₄ is H, OH or a C₁₋₄ alkyl;
R₆ is H, OH or a C₁₋₄ alkyl, or R₆ with R₇ forms a substituted aryl;
R₇ is H, OH, a C₁ alkyl, or R₇ with R₈ forms a ═CH₂, or R₇ with R₈ forms a ═O, or R₇ is absent when (a) R₈ is joined to R₉ to form a substituted aryl or unsubstituted aryl, or (b) bond α is present; R₈ is H, OH or a C₁₋₄ alkyl, or R₈ with R₉ forms a substituted oxane, or R₈ with R₉ forms a substituted dioxane, or R₈ with R₉ forms a substituted aryl or an unsubstituted aryl, or R₈ with R₉ forms the structure:

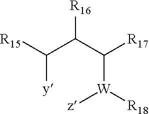

wherein W is a C atom or an O atom, and when W is an O atom, R₁₈ is absent;
wherein end y′ is bonded to atom Y and end z′ is bonded to atom Z and
   wherein when W is a C atom, R₁₅ and R₁₆ are each, independently, H or OH, and R₁₇ and R₁₈ are each, independently, H or OH, or R₁₇ and R₁₈ together form a substituted or unsubstituted aryl,
   and wherein when W is an O atom R₁₅ is H or OH, and wherein R₁₆ and R₁₇ are, independently, H or OH, or R₁₆ and R₁₇ together form a substituted aryl or unsubstituted aryl;
R₉ is H, —CHO, —CH₂OAc, —CH₂—R₁₉, —C(═O)(OEt) or —C(═O)(OMe),
   wherein R₁₉ is a substituted aryl or an unsubstituted aryl;
R₁₂ is H, OH or a C₁₋₄ alkyl, or is absent if bond γ is present;
R₁₃ is H or, is absent when (a) R₆ is joined to R₇ to form a substituted aryl or (b) bond 13 is present;
R₁₄ is H or, is absent when (a) R₉ is joined to R₈ to form a substituted aryl or unsubstituted aryl, or (b) Y is an O atom, or (c) bond a is present;
wherein bond α is only present if bond γ is present and R₉ is —C(═O)(OEt) or —C(═O)(OMe), and
Y and Z are each a C atom;
wherein bond β is only present if bonds α and γ are absent and R₉ is —C(═O)(OEt) or —C(═O)(OMe), and Z and X are carbon atoms, and R₇ together with R₈ is other than ═O;
wherein when Y and X are C atoms, R₁ is Br, R₂, R₃ and R₁₀ are CH₃, R₄, R₅, R₆, R₁₁, R₁₂, R₁₃ and
R₁₄ are H, R₇ and R₈ form a ═CH₂, and R₉ is —CH₂—R₁₉ with R₁₉ having the structure:

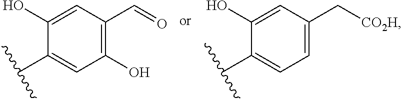 [SMILES: *c1cc(O)c(C=O)cc1O ; "or" ; *c1ccc(CC(=O)O)cc1O]

then R₉ and R₁₀ have the following stereochemistry:

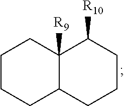

wherein when Y and X are C atoms, R₁ is Br, R₂, R₃, R₈ and R₁₀ are CH₃, R₄, R₅, R₆, R₁₁, R₁₂, R₁₃ and R₁₄ are H, and R₇ is OH, then R₉ is other than —C₂H₄C(CH₃)(CHCH₂OH);
wherein when R₉ is —C(═O)(OMe) and bonds α, β and γ are absent, and R₇ and R₈ together from ═O, then R₁ is other than I;
or a pharmaceutically acceptable salt thereof.

The invention provides a composition comprising a compound having the structure:

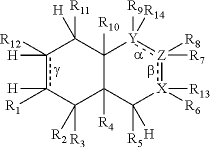

wherein
Y and X are, independently, a C atom or an O atom,
   wherein when X is O, R₆ and R₁₃ are absent and when Y is O, R₉ and R₁₄ are absent;
Z is a carbon atom;
α, β and γ are, independently, present or absent, and when present each is a bond;
R₁ is OH, CH₃ or a halogen, or is absent if bond γ is present;
R₂, R₃, R₅, R₁₀, R₁₁ and R₁₂ are, independently, H, OH or a C₁₋₄ alkyl;
R₄ is H, OH or a C₁₋₄ alkyl;
R₆ is H, OH or a C₁₋₄ alkyl, or R₆ with R₇ forms a substituted aryl;
R₇ is H, OH, a C₁₋₄ alkyl, or R₇ with R₈ forms a ═CH₂, or R₇ with R₈ forms a ═O, or R₇ is absent when (a) R₈ is joined to R₉ to form a substituted aryl or unsubstituted aryl or (b) bond α is present;
R₈ is H, OH or a C₁ alkyl, or R₈ with R₉ forms a substituted oxane, or R₈ with R₉ forms a substituted dioxane, or R₈ with R₉ forms a substituted aryl or an unsubstituted aryl, or R₈ with R₉ forms the structure:

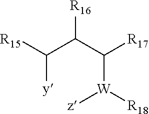

wherein W is a C atom or an O atom, and when W is an O atom, R₁₈ is absent;
wherein end y′ is bonded to atom Y and end z′ is bonded to atom Z and
   wherein when W is a C atom, R₁₅ and R₁₆ are each, independently, H, or OH, and R₁₇ and R₁₈ are each, independently, H, or OH, or R₁₇ and R₁₈ together form a substituted or unsubstituted aryl,
   and wherein when W is an O atom R₁₅ is H, or OH, and wherein R₁₆ and R₁₇ are, independently, H, or OH, or R₁₆ and R₁₇ together form a substituted aryl or unsubstituted aryl;
R₉ is H, —CHO, —CH₂OAc, —CH₂—R₁₉, —C(═O)(OEt) or —C(═O)(OMe),
   wherein R₁₉ is a substituted aryl or an unsubstituted aryl;
R₁₂ is H, OH or a C₁₋₄ alkyl, or is absent if bond γ is present;
R₁₃ is H or, is absent when (a) R₆ is joined to R₇ to form a substituted aryl or (b) bond is present;
R₁₄ is H or, is absent when (a) R₉ is joined to R₈ to form a substituted aryl or unsubstituted aryl, or (b) Y is an O atom, or (c) bond α is present;
wherein bond α is only present if bond γ is present and R₉ is —C(═O)(OEt) or —C(═O)(OMe), and Y and Z are carbon atoms;
wherein bond β is only present if bonds α γ is absent and R₉ is —C(═O)(OEt) or —C(═O)(OMe), and Z and X are carbon atoms, and R₇ together with R₈ is other than ═O;
wherein when Y and X are C atoms, R₁ is Br, R₂, R₃, R₈ and R₁₀ are CH₃, R₄, R₅, R₆, R₁₁, R₁₂, R₁₃ and R₁₄ are H, R₇ is OH then R₉ is other than —C₂H₄C(CH₃)(CHCH₂OH);
or a pharmaceutically acceptable salt thereof,
wherein the composition is free of plant extract.

The invention provides a polyene having the structure:

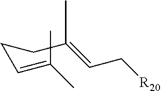

wherein R₂₀ is:

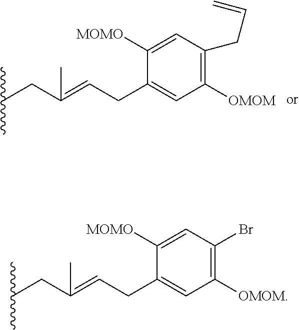

The invention provides a compound having the structure:

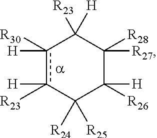

wherein
α is a bond which is absent or present, and when present R₂₃ and R₃₀ are absent;
R₂₃ is a halogen or is absent;
R₂₄ and R₂₅ are independently, a C₁₋₄ alkyl;
R₂₆ is —CH₂CN, —CHO, —CH₂(C═O)(CH₃);
R₂₇ is a C₁₋₄ alkyl, or OH;
or R₂₆ and R₂₇ together with R₂₇ forms a dihydrofuran-2-one,
R₂₈ is H, H or a C₁₋₄ alkyl;
R₂₉ is H or OH,
R₃₀ is H, or OH, or is absent.

The invention provides a compound having the structure:

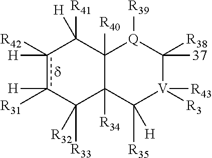

wherein
Q and V are, independently, a C atom or an O atom,
   wherein when Q is O, R₃₉ and R₄₄ are absent and when V is O, R₃₆ and R₄₃ are absent;
δ is absent or present, and when present is a bond;
R₃₁ is H, OH, or a halogen, or is absent if bond δ is present;
R₃₂, R₃₃, R₃₅, R₄₀, R₄₁ and R₄₂ are, independently, H, OH or a C₁₋₄ alkyl;
R₃₄ is H, OH or a C₁₋₄ alkyl, or is absent when X and Y are O atoms and R₃₇ with R₃₈ forms a ═O;
R₃₆ is absent, or is H, or with R₃₇ forms a substituted aryl;
R₃₇ with R₃₈ forms a ═O, or is absent;
R₃₈ with R₃₉ forms a substituted aryl or an unsubstituted aryl, or is absent;
R₄₂ is H, OH or a C₁₋₄ alkyl, or is absent if bond δ is present;
R₄₃ is H or, is absent when (a) R₃₆ is joined to R₃₇ to form a substituted aryl;
wherein when R₃₂, R₃₃ and R₄₀ are CH₃, R₃₄, R₃₆, R₄₀, R₄₁, R₄₂ and R₄₃ are H, R₃₇ is absent, and R₃₈ and R₃₉ are joined to form an unsubstituted aryl, then R₁ is I or Br,
or a pharmaceutically acceptable salt thereof.

The invention provides a compound having the structure:

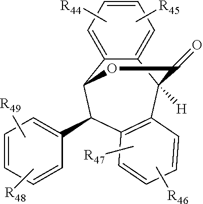

wherein
R₄₄, R₄₅, R₄₆, R₄₇, R₄₈, and R₄₉ are independently H, CN, acetate, OH, OR₅₀, a substituted or unsubstituted C₁₋₆ alkyl, a substituted or unsubstituted C₂₋₆ alkenyl, a ketone, an ester, a substituted or unsubstituted cycloalkyl, a substituted or unsubstituted cycloalkenyl, a substituted or unsubstituted aryl, a substituted or unsubstituted heteroaryl, a substituted or unsubstituted heterocyclyl,
   wherein each occurrence of R₅₀ is independently H, methyl, substituted or unsubstituted alkyl, substituted or unsubstituted heteroalkyl substituted or unsubstituted aryl, substituted or unsubstituted heteroaryl, phosphate, sulfate, sulfonic ester, or ester,
      or a pharmaceutically acceptable salt thereof.

The invention provides a composition comprising a compound having the structure:

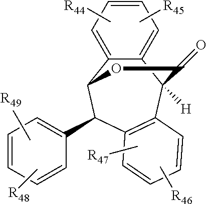

wherein
R₄₄, R₄₅, R₄₆, R₄₇, R₄₈, and R₄₉ are independently H, CN, acetate, OH, OR₅₀, a substituted or unsubstituted C₁₋₆ alkyl, a substituted or unsubstituted C₂₋₆ alkenyl, a ketone, an ester, a substituted or unsubstituted cycloalkyl, a substituted or unsubstituted cycloalkenyl, a substituted or unsubstituted aryl, a substituted or unsubstituted heteroaryl, a substituted or unsubstituted heterocyclyl,
   wherein each occurrence of R₅₀ is independently H, methyl, substituted or unsubstituted alkyl, substituted or unsubstituted heteroalkyl substituted or unsubstituted aryl, substituted or unsubstituted heteroaryl, phosphate, sulfate, sulfonic ester, or ester,
      or a pharmaceutically acceptable salt thereof,
      wherein the composition is free of plant extract.

The invention provides a compound having the structure:

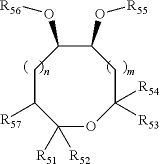

wherein
n=1 or 2;
m=1 or 2;
R₅₁, R₅₂, R₅₃ and R₅₄ are independently H, alkyl, or a haloalkyl;
R₅₅ and R₅₆ are both H or combine to form a carbonate; and
R₅₇ is H, Br, I or Cl,
   or a pharmaceutically acceptable salt, diastereomer, or enantiomer thereof.

The invention provides a composition comprising a compound having the structure:

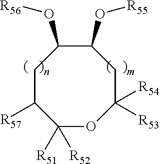

wherein
n=1 or 2;
m=1 or 2;
R₅₁, R₅₂, R₅₃ and R₅₄ are independently H, alkyl, or a haloalkyl;
R₅₅ and R₅₆ are both H or combine to form a carbonate; and
R₅₇ is H, Br, I or Cl,
   or a pharmaceutically acceptable salt, diastereomer, or enantiomer thereof.
   wherein the composition is free of plant extract.

The invention provides a compound having the structure:

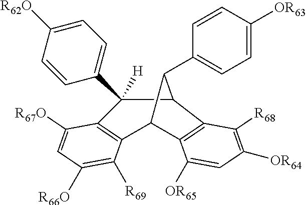

wherein
R₆₂, R₆₃, R₆₄, R₆₅, R₆₆, and R₆₇ are independently H, methyl, substituted or unsubstituted alkyl, substituted or unsubstituted heteroalkyl, substituted or unsubstituted aryl, substituted or unsubstituted heteroaryl, phosphate, sulfate, sulfonic ester, or ester; and
R₆₈ and R₆₉ are independently H, Cl, Br or I;
   or a pharmaceutically acceptable salt thereof.

The invention provides a composition comprising a compound having the structure:

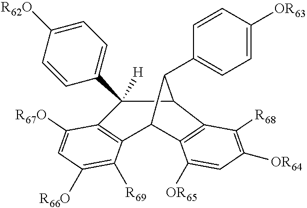

wherein
R₆₂, R₆₃, R₆₄, R₆₅, R₆₆, and R₆₇ are independently H, methyl, substituted or unsubstituted alkyl, substituted or unsubstituted heteroalkyl, substituted or unsubstituted aryl, substituted or unsubstituted heteroaryl, phosphate, sulfate, sulfonic ester, or ester; and
R₆₈ and R₆₉ are independently H, Cl, Br or I;
   or a pharmaceutically acceptable salt thereof,

### BRIEF DESCRIPTION OF THE FIGURES

FIG. 1: Selected natural products with rings that arise via halonium induced cation-π cyclizations.

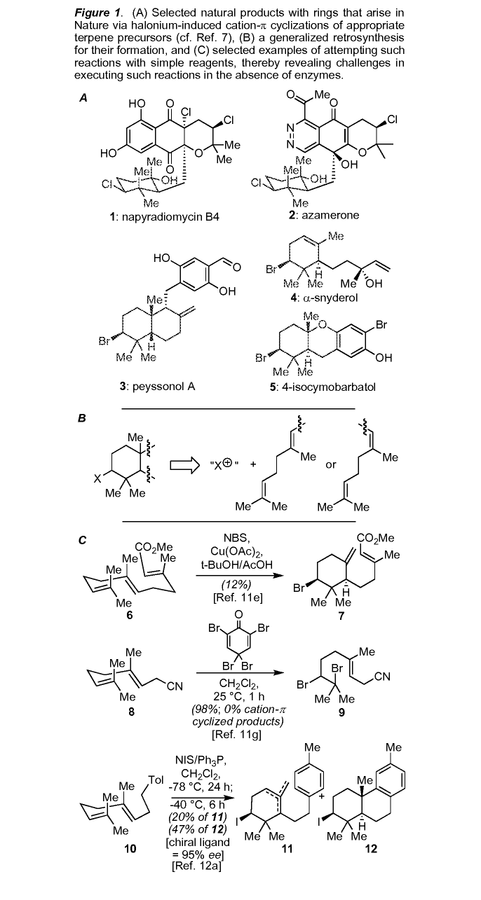

FIG. 2: Structures of previously synthesized materials incorporating molecular Br₂.

FIG. 3: X-ray structure of compound 40.

FIG. 4: X-ray structure of compound 194, 196, 198, 201, 203, 208 and 214.

FIG. 5: Antiviral activity of Peyssonol A and derivatives.

### DETAILED DESCRIPTION OF THE INVENTION

The invention provides a process for cyclizing an alkene comprising contacting the alkene with a compound having the structure:

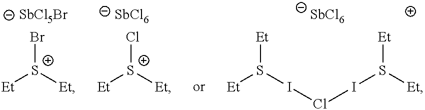

under conditions permitting cyclization of the alkene.

In some embodiments of the process, the alkene is a polyene.

In some embodiments of the process, the alkene is an alkenoic acid.

In some embodiments of the process, the alkene is an alkenyl alkyl ether.

In some embodiments of the process, the cyclization is a ring-forming halolactonization

In some embodiments of the process, the cyclization is a ring-expanding bromoetherification.

The invention provides a process for halogenating an aromatic ring comprising contacting the aromatic ring with a compound having the structure:

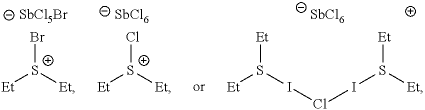

under conditions permitting halogenation of the aromatic ring.

In some embodiments of the process, the halogenation is a mono-halogenation.

In some embodiments of the process, the aromatic ring is a substituted phenyl.

The invention provides a compound having the structure:

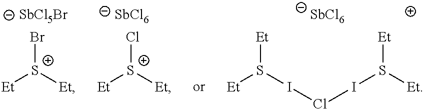

In some embodiments of the process, the compound has the structure:

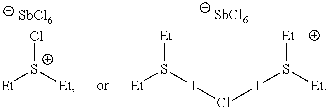

The invention provides a compound having the structure:

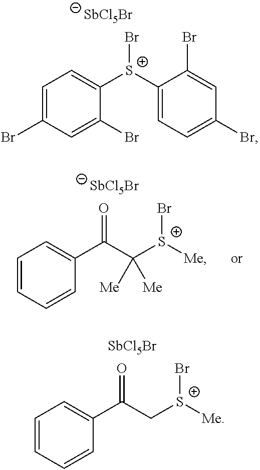

The invention provides a process for producing a compound having the structure:

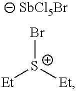

comprising contacting Br₂ with excess Et₂S and SbCl₅ in a suitable solvent at a suitable temperature so as to thereby produce the compound.

In some embodiments of the process, the suitable solvent is 1,2-dichloroethane.

In some embodiments of the process, the suitable temperature is about −30° C.

The invention provides a process for producing a compound having the structure:

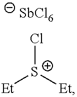

comprising contacting Cl₂ with Et₂S and SbCl₅ in a suitable solvent at a first suitable temperature, and subsequently contacting the resulting product with hexanes prior to cooling to a second suitable temperature so as to thereby produce the compound.

In some embodiments of the process, the first suitable temperature is about −25° C. and the resulting product is warmed to about 30° C.

In some embodiments of the process, the second suitable temperature is about −20° C.

In some embodiments of the process, the suitable solvent is 1,2-dichloroethane.

The invention provides a process for producing a compound having the structure:

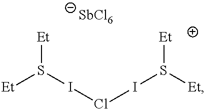

comprising contacting I₂ with excess Et₂S and SbCl₅ in a suitable solvent at a first suitable temperature, warming the resulting product, and subsequently contacting the resulting product with hexanes at a second suitable temperature so as to thereby produce the compound.

In some embodiments of the process, the first suitable temperature is about 0° C. and the resulting product is warmed to about 25° C.

In some embodiments of the process, the second suitable temperature is about −20° C.

In some embodiments of the process, the suitable solvent is 1,2-dichloroethane.

The invention provides a compound having the structure:

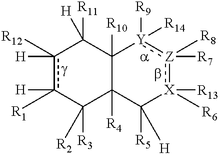

wherein
Y and X are, independently, a C atom or an O atom,
   wherein when X is O, R₆ and R₁₃ are absent and when Y is O, R₉ and R₁₄ are absent;
Z is a carbon atom;
α, β and γ are, independently, present or absent, and when present each is a bond;
R₁ is OH or a halogen, or is absent if bond γ is present;
R₂, R₃, R₅, R₁₀, R₁₁ and R₁₂ are, independently, H, OH or a C₁₋₄ alkyl;
R₄ is H, OH or a C₁₋₄ alkyl;
R₆ is H, OH or a C₁₋₄ alkyl, or R₆ with R₇ forms a substituted aryl;
R₇ is H, OH, a C₁ alkyl, or R₇ with R₈ forms a ═CH₂, or R₇ with R₈ forms a ═O, or R₇ is absent when (a) R₈ is joined to R₉ to form a substituted aryl or unsubstituted aryl, or (b) bond α is present;
R₈ is H, OH or a C₁₋₄ alkyl, or R₈ with R₉ forms a substituted oxane, or R₈ with R₉ forms a substituted dioxane, or R₈ with R₉ forms a substituted aryl or an unsubstituted aryl, or R₈ with R₉ forms the structure:

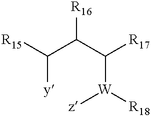

wherein W is a C atom or an O atom, and when W is an O atom, R₁₈ is absent;
wherein end y′ is bonded to atom Y and end z′ is bonded to atom Z and
   wherein when W is a C atom, R₁₅ and R₁₆ are each, independently, H or OH, and R₁₇ and R₁₈ are each, independently, H or OH, or R₁₇ and R₁₈ together form a substituted or unsubstituted aryl,
   and wherein when W is an O atom R₁₅ is H or OH, and wherein R₁₆ and R₁₇ are, independently, H or OH, or R₁₆ and R₁₇ together form a substituted aryl or unsubstituted aryl;
R₉ is H, —CHO, —CH₂OAc, —CH₂—R₁₉, —C(═O)(OEt) or —C(═O)(OMe),
   wherein R₁₉ is a substituted aryl or an unsubstituted aryl;
R₁₂ is H, OH or a C₁₋₄ alkyl, or is absent if bond γ is present;
R₁₃ is H or, is absent when (a) R₆ is joined to R₇ to form a substituted aryl or (b) bond 13 is present;
R₁₄ is H or, is absent when (a) R₉ is joined to R₈ to form a substituted aryl or unsubstituted aryl, or (b) Y is an O atom, or (c) bond α is present;
wherein bond α is only present if bond γ is present and R₉ is —C(═O)(OEt) or —C(═O)(OMe), and Y and Z are each a C atom;
wherein bond β is only present if bonds α and γ are absent and R₉ is —C(═O)(OEt) or —C(═O)(OMe), and Z and X are carbon atoms, and R₇ together with R₈ is other than ═O;
wherein when Y and X are C atoms, R₁ is Br, R₂, R₃ and R₁₀ are CH₃, R₄, Rₛ, R₆, R₁₁, R₁₂, R₁₃ and R₁₄ are H, R₇ and R₉ form a ═CH₂, and R₉ is —CH₂—R₁₉ with R₁₉ having the structure:

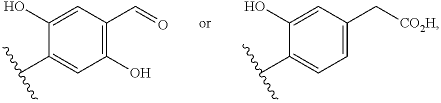 [SMILES: *c1cc(O)c(C=O)cc1O ; *c1ccc(CC(=O)O)cc1O]

then R₉ and R₁₀ have the following stereochemistry:

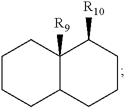

wherein when Y and X are C atoms, R₁ is Br, R₂, R₃, R₈ and R₁₀ are CH₃, R₄, R₅, R₆, R₁₁, R₁₂, R₁₃ and R₁₄ are H, and R₇ is OH, then R₉ is other than —C₂H₄C(CH₃)(CHCH₂OH);
wherein when R₉ is —C(═O)(OMe) and bonds α, β and γ are absent, and R₇ and R₈ together from ═O, then R₁ is other than I;
or a pharmaceutically acceptable salt thereof.

In some embodiments of the compound or pharmaceutically acceptable salt thereof, R₁ is Br, Cl or I.

In some embodiments of the compound or pharmaceutically acceptable salt thereof, R₁₀ is CH₃ and R₄ is H.

In some embodiments of the compound or pharmaceutically acceptable salt thereof, the bonds α, β and γ are absent.

In some embodiments of the compound or pharmaceutically acceptable salt thereof, R₂ and R₃ are CH₃.

In some embodiments of the compound or pharmaceutically acceptable salt thereof, R₁ is Br.

In some embodiments of the compound or pharmaceutically acceptable salt thereof, R₁ is Cl.

In some embodiments of the compound or pharmaceutically acceptable salt thereof, R₁ is I.

In some embodiments of the compound or pharmaceutically acceptable salt thereof, X and Z are C atoms.

In some embodiments of the compound or pharmaceutically acceptable salt thereof, R₇ is CH₃.

In some embodiments of the compound or pharmaceutically acceptable salt thereof, R₇ and R₈ together from a ═O.

In some embodiments of the compound or pharmaceutically acceptable salt thereof, R₇ and R₈ together from a substituted dioxane.

In some embodiments of the compound or pharmaceutically acceptable salt thereof, R₇ and R₈ together from a substituted aryl.

In some embodiments of the compound or pharmaceutically acceptable salt thereof, R₈ with R₉ forms the structure:

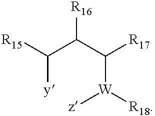

In some embodiments of the compound, Y
and X are C atoms;
R₁ is H, Br, Cl, or I;
R₂ and R₃ are CH₃;
R₄ is H;
R₅ is H;
R₆ is H, or together with R₇ forms a bromo-substituted, methoxymethoxy-substituted benzene attached to atoms Z and X;
R₇ is absent, is CH₃, together with R₈ forms a ═O, or together with R₈ forms a ═CH₂;
R₉ is H, —CHO, —CH₂OAc, —CH₂—R₁₉, —C(═O)(OEt) or —C(═O)(OMe), wherein R₁₉ has the structure:

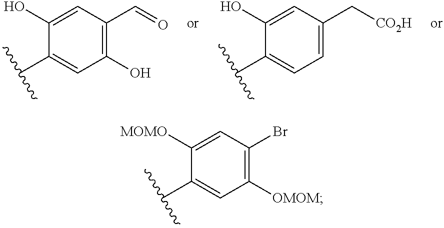

R₁₀ is CH₃;
R₁₁ is H;
R₁₂ is H;
R₁₃ is H or is absent; and
R₁₄ is H or is absent.

In some embodiments of the compound or pharmaceutically acceptable salt thereof, the compound has the structure:

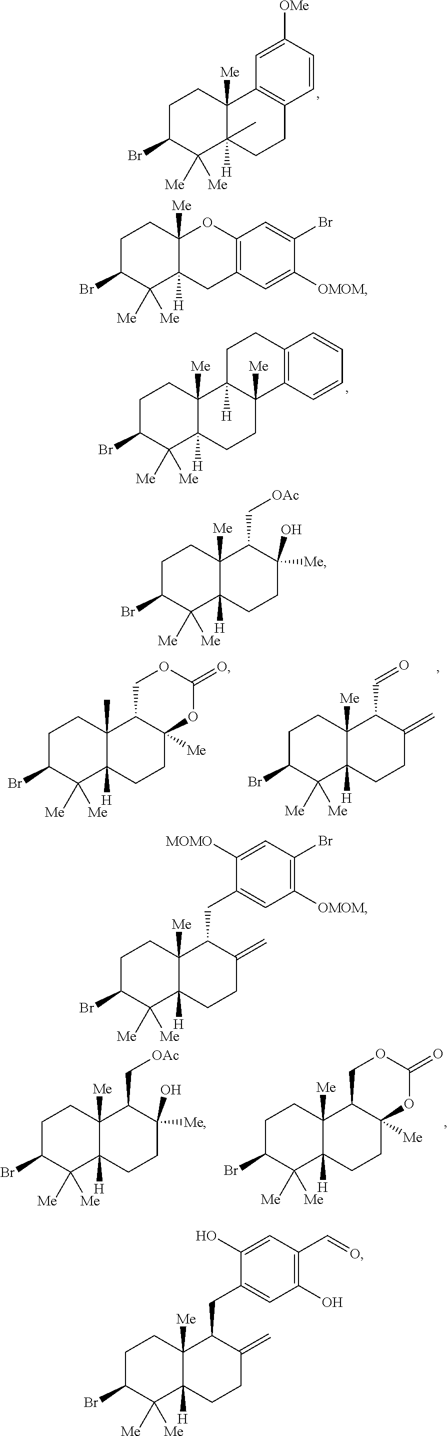

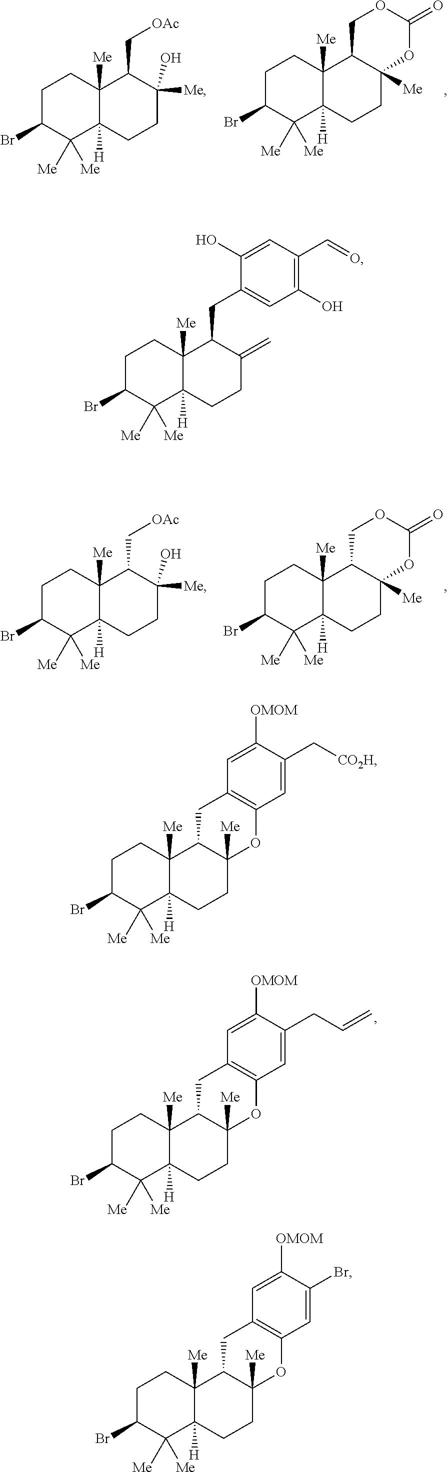

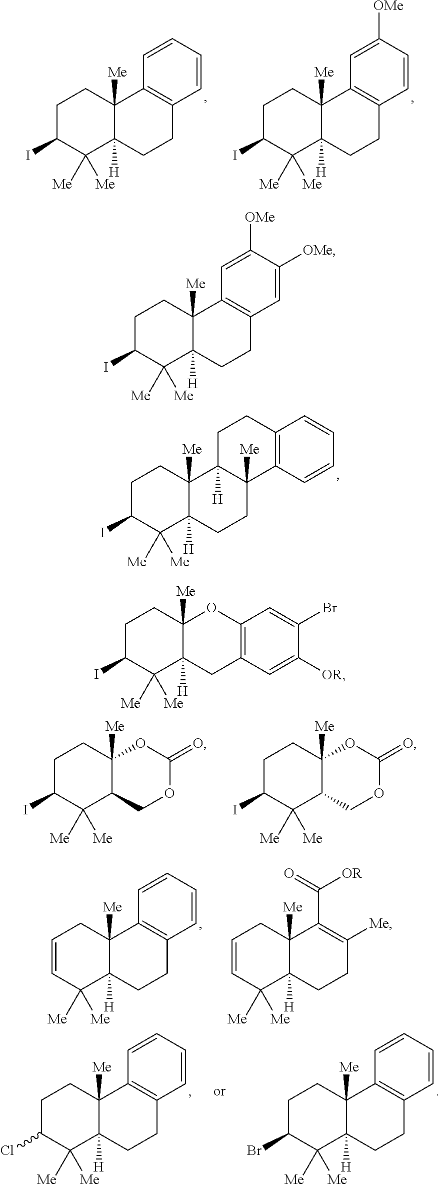

The invention provides a composition comprising a compound having the structure:

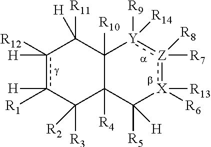

wherein
Y and X are, independently, a C atom or an O atom,
   wherein when X is O, R₆ and R₁₃ are absent and when Y is O, R₉ and R₁₄ are absent;
Z is a carbon atom;
α, β and γ are, independently, present or absent, and when present each is a bond;
R₁ is OH, CH₃ or a halogen, or is absent if bond γ is present;
R₂, R₃, R₅, R₁₀, R₁₁ and R₁₂ are, independently, H, OH or a C₁₋₄ alkyl;
R₄ is H, OH or a C₁₋₄ alkyl;
R₆ is H, OH or a C₁₋₄ alkyl, or R₆ with R₇ forms a substituted aryl;
R₇ is H, OH, a C₁ alkyl, or R₇ with R₈ forms a ═CH₂, or R₇ with R₈ forms a ═O, or R₇ is absent when (a) R₈ is joined to R₉ to form a substituted aryl or unsubstituted aryl or (b) bond α is present;
R₈ is H, OH or a C₁ alkyl, or R₈ with R₉ forms a substituted oxane, or R₈ with R₉ forms a substituted dioxane, or R₈ with R₉ forms a substituted aryl or an unsubstituted aryl, or R₈ with R₉ forms the structure:

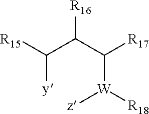

wherein W is a C atom or an O atom, and when W is an O atom, R₁₈ is absent;
wherein end y′ is bonded to atom Y and end z′ is bonded to atom Z and
   wherein when W is a C atom, R₁₅ and R₁₆ are each, independently, H, or OH, and R₁₇ and R₁₈ are each, independently, H, or OH, or R₁₇ and R₁₈ together form a substituted or unsubstituted aryl,
   and wherein when W is an O atom R₁₅ is H, or OH, and wherein R₁₆ and R₁₇ are, independently, H, or OH, or R₁₆ and R₁₇ together form a substituted aryl or unsubstituted aryl;
R₉ is H, —CHO, —CH₂OAc, —CH₂—R₁₉, —C(═O)(OEt) or —C(═O)(OMe),
   wherein R₁₉ is a substituted aryl or an unsubstituted aryl;
R₁₂ is H, OH or a C₁₋₄ alkyl, or is absent if bond γ is present;
R₁₃ is H or, is absent when (a) R₆ is joined to R₇ to form a substituted aryl or (b) bond β is present;
R₁₄ is H or, is absent when (a) R₉ is joined to R₈ to form a substituted aryl or unsubstituted aryl, or (b) Y is an O atom, or (c) bond α is present;
wherein bond α is only present if bond γ is present and R₉ is —C(═O)(OEt) or —C(═O)(OMe), and Y and Z are carbon atoms;
wherein bond β is only present if bonds αγ is absent and R₉ is —C(═O)(OEt) or —C(═O)(OMe), and Z and X are carbon atoms, and R₇ together with R₈ is other than ═O;
wherein when Y and X are C atoms, R₁ is Br, R₂, R₃, R₈ and R₁₀ are CH₃, R₄, R₅, R₆, R₁₁, R₁₂, R₁₃ and R₁₄ are H, R₇ is OH then R₉ is other than —C₂H₄C(CH₃)(CHCH₂OH);
or a pharmaceutically acceptable salt thereof,
wherein the composition is free of plant extract.

In some embodiments of the composition, the compound has the structure:

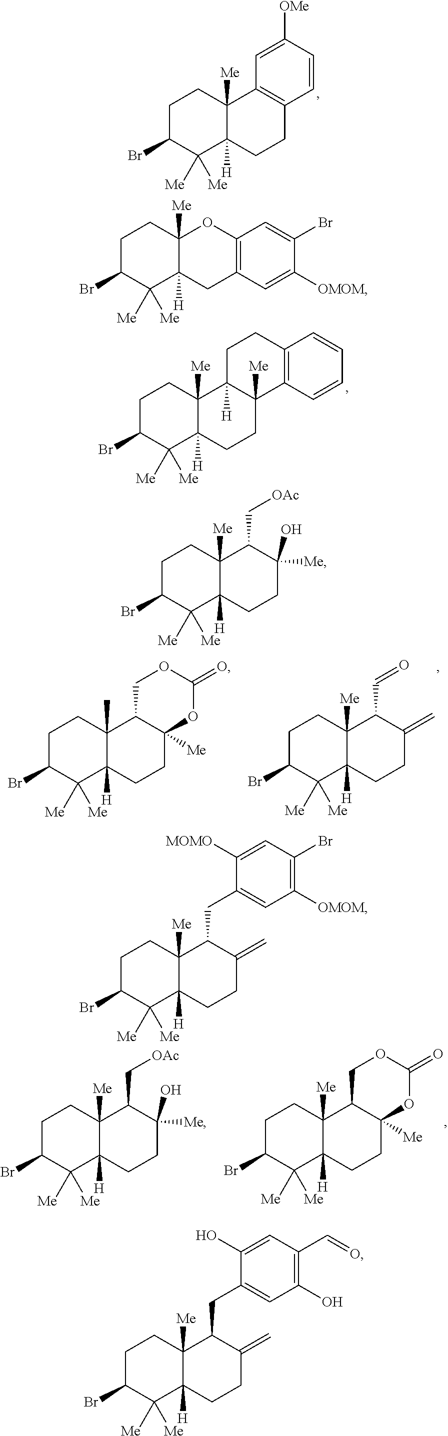

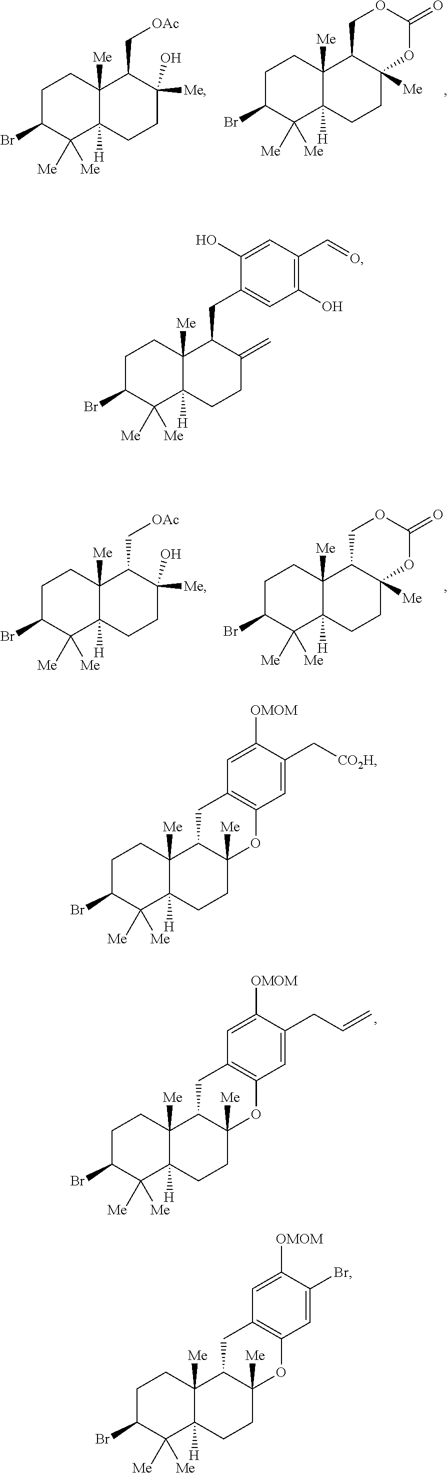

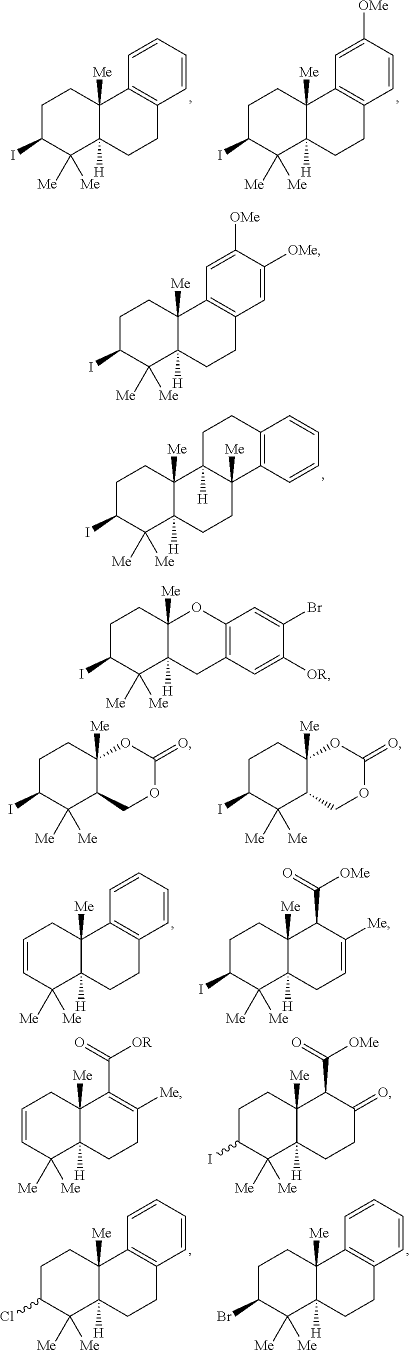

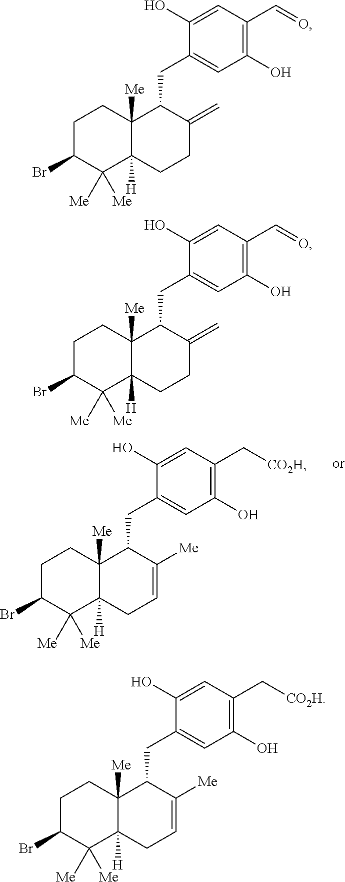

In some embodiments, a process for producing the instant compound comprising reacting a polyene having the structure:

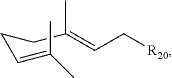

wherein R₂₀ is —CN, an ether, an ester, an acetate, OH, C₁₋₆ alkyl, C₂₋₆ alkenyl, a ketone, an ester, cycloalkyl, cycloalkenyl, a substituted or unsubstituted aryl, a substituted or unsubstituted heteroaryl, a substituted or unsubstituted heterocyclyl,
   wherein each occurrence of alkyl, alkenyl, cycloalkyl, and cycloalkenyl is substituted or unsubstituted,
with a second compound having the structure:

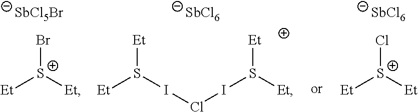

in a suitable solvent at a suitable temperature so as to thereby produce the compound.

In some embodiments of the process, R₂₀ is

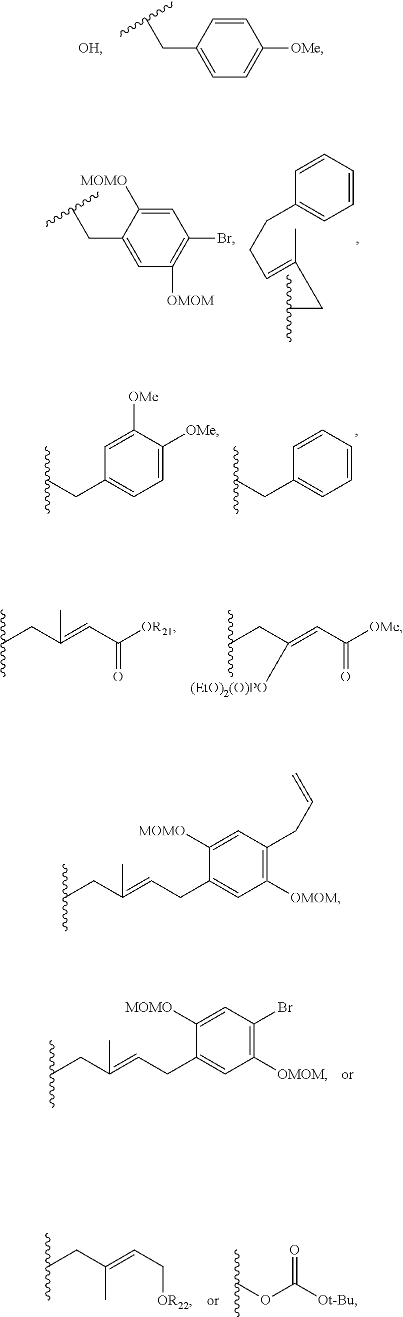

wherein R₂₁ is CH₃ or C₂H₃, and R₂₂ is H, Ac or Boc.

In some embodiments, a process for producing the instant compound comprising
a) reacting a polyene having the structure:

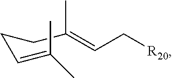

wherein R₂₀ is —CN, an ether, an ester, an acetate, OH, C₁₋₆ alkyl, C₂₋₆ alkenyl, a ketone, an ester, cycloalkyl, cycloalkenyl, a substituted or unsubstituted aryl, a substituted or unsubstituted heteroaryl, a substituted or unsubstituted heterocyclyl,
   wherein each occurrence of alkyl, alkenyl, cycloalkyl, and cycloalkenyl is substituted or unsubstituted,
with a second compound having the structure:

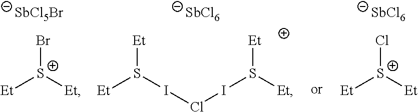

in a suitable solvent at a suitable temperature so as to thereby produce the compound of the composition; and
b) admixing the product of step a) with a carrier so as to thereby produce the composition.

In some embodiments of the process, R₂₀ is:

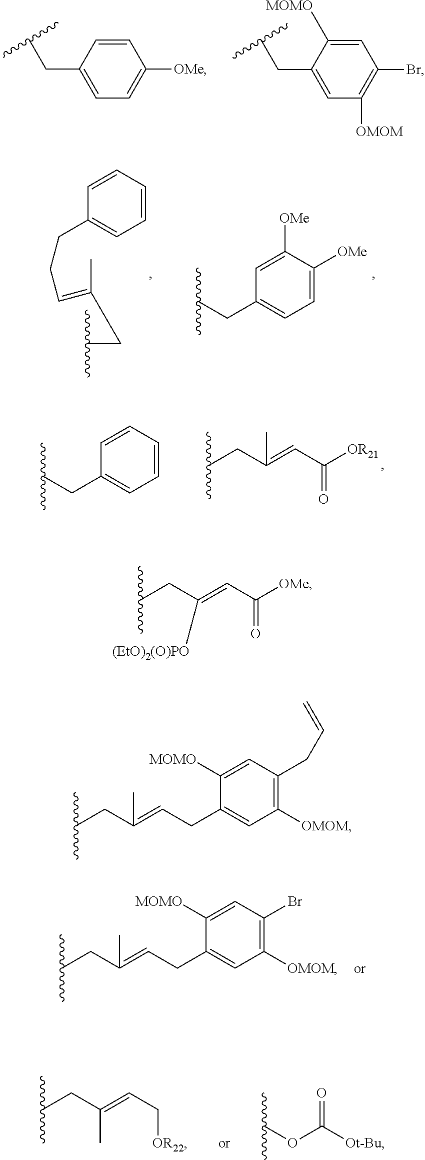

wherein R₂₁ is CH₃ or C₂H₃ and R₂₂ is H, Ac or Boc.

In some embodiments of the process, the compound has a Br at position R₁ and the second compound has the structure:

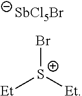

In some embodiments of the process, the compound has a I at position R₁ and the second compound has the structure:

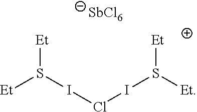

In some embodiments of the process, the compound has a Cl at position R₁ and the second compound has the structure:

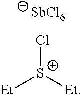

In some embodiments of the process, the compound Y and X are O atoms and wherein the polyene has the structure:

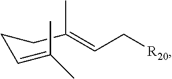

wherein

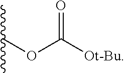 [SMILES: *OC(=O)OC(C)(C)C]

In some embodiments of the process, the suitable solvent is MeNO₂.

In some embodiments of the process, the suitable temperature is about −25° C. to 25° C.

In some embodiments of the process, the compound has the structure:

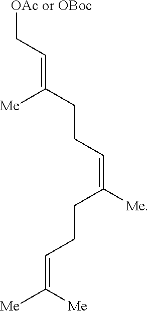

and the polyene has the structure:

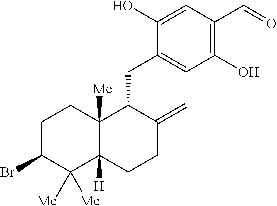 [SMILES: C=C1CC[C@@H]2C(C)(C)[C@@H](Br)CC[C@]2(C)[C@@H]1Cc1cc(O)c(C=O)cc1O]

In some embodiments of the process, the compound has the structure:

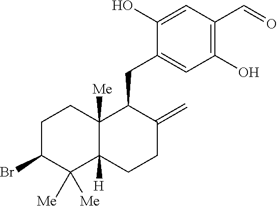 [SMILES: C=C1CC[C@@H]2C(C)(C)[C@@H](Br)CC[C@]2(C)[C@H]1Cc1cc(O)c(C=O)cc1O]

and the polyene has the structure:

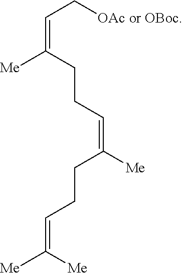

In some embodiments of the process, the compound has the structure:

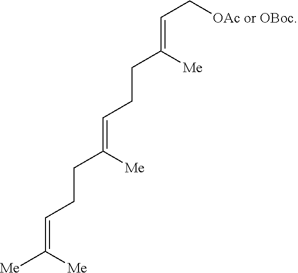

and the polyene has the structure

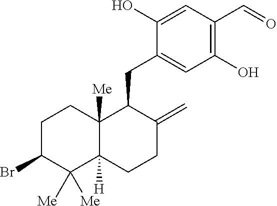 [SMILES: C=C1CC[C@H]2C(C)(C)[C@@H](Br)CC[C@]2(C)[C@H]1Cc1cc(O)c(C=O)cc1O]

In some embodiments of the process, the compound has the structure:

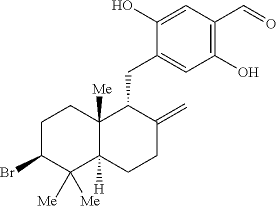 [SMILES: C=C1CC[C@H]2C(C)(C)[C@@H](Br)CC[C@]2(C)[C@@H]1Cc1cc(O)c(C=O)cc1O]

and the polyene has the structure:

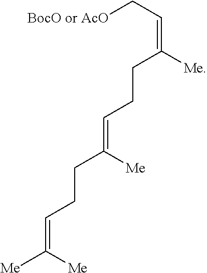

The invention provides a polyene having the structure:

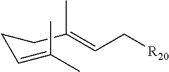

wherein R₂₀ is:

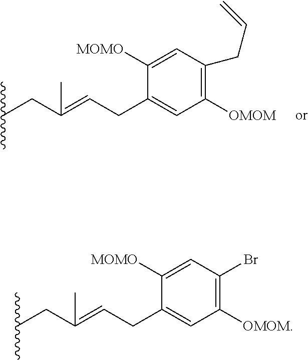

The invention provides a compound having the structure:

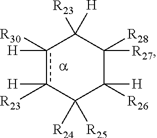

wherein
α is a bond which is absent or present, and when present R₂₃ and R₃₀ are absent;
R₂₃ is a halogen or is absent;
R₂₄ and R₂₅ are independently, a C₁ alkyl;
R₂₆ is —CH₂CN, —CHO, —CH₂(C═O)(CH₃);
R₂₇ is a C₁₋₄ alkyl, or OH;
or R₂₆ and R₂₇ together with R₂₇ forms a dihydrofuran-2-one,
R₂₈ is H, H or a C₁₋₄ alkyl;
R₂₉ is H or OH,
R₃₀ is H, or OH, or is absent.

In some embodiments of the process, R₂₃ is present and is Br, Cl or I.

In some embodiments of the process, R₂₄ and R₂₅ are CH₃.

In some embodiments, the compound has the structure:

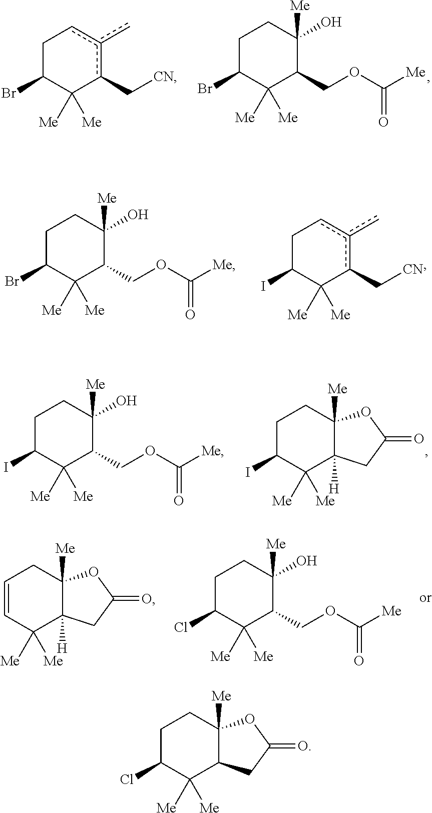

In some embodiments, a process for producing the instant compound comprising:

a) reacting a polyene having the structure:

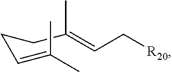

wherein R₂₀ is:

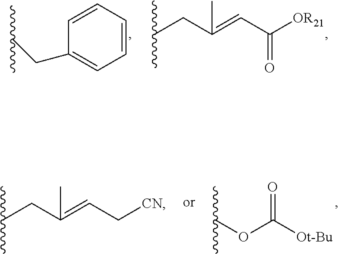

wherein R₂₁ is CH₃ or OH₃ and R₂₂ is H, Ac or Boc,
with a second compound having the structure

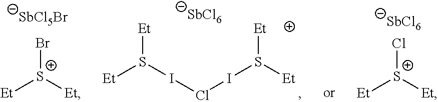

in a suitable solvent at a suitable temperature so as to thereby produce the compound.

The invention provides a compound having the structure:

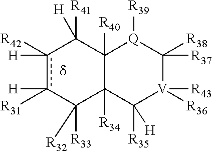

wherein
Q and V are, independently, a C atom or an O atom,
   wherein when Q is O, R₃₉ and R₄₄ are absent and when V is O, R₃₆ and R₄₃ are absent;
δ is absent or present, and when present is a bond;
R₃₁ is H, OH, or a halogen, or is absent if bond δ is present;
R₃₂, R₃₃, R₃₅, R₄₀, R₄₁ and R₄₂ are, independently, H, OH or a C₁₋₄ alkyl;
R₃₄ is H, OH or a C₁₋₄ alkyl, or is absent when X and Y are O atoms and R₃₇ with R₃₈ forms a ═O;
R₃₆ is absent, or is H, or with R₃₇ forms a substituted aryl;
R₃₇ with R₃₈ forms a ═O, or is absent;
R₃₈ with R₃₉ forms a substituted aryl or an unsubstituted aryl, or is absent;
R₄₂ is H, OH or a C₁₋₄ alkyl, or is absent if bond δ is present;
R₄₃ is H or, is absent when (a) R₃₆ is joined to R₃₇ to form a substituted aryl;
wherein when R₃₂, R₃₃ and R₄₀ are CH₃, R₃₄, R₃₆, R₄₀, R₄₁, R₄₂ and R₄₃ are H, R₃₇ is absent, and R₃₈ and R₃₉ are joined to form an unsubstituted aryl, then R₁ is I or Br,
or a pharmaceutically acceptable salt thereof.

In some embodiments, the compound wherein
R₃₁ is H, Br or I;
R₃₂, R₃₃, R₄₀, are CH₃;
R₃₄ and R₃₅ are H;
R₃₆ is absent, is H or is joined or R₃₆ with R₃₇ forms a bromo-substituted, methoxymethoxy-substituted benzene ring;
R₃₇ with R₃₈ forms a ═O, or is absent;
R₃₈ with R₃₉ forms a methoxy-substituted benzene ring or an unsubstituted benzene ring, or is absent;
R₄₂ is H, or is absent;
R₄₃ is H or, is absent.

In some embodiments, the compound has the structure:

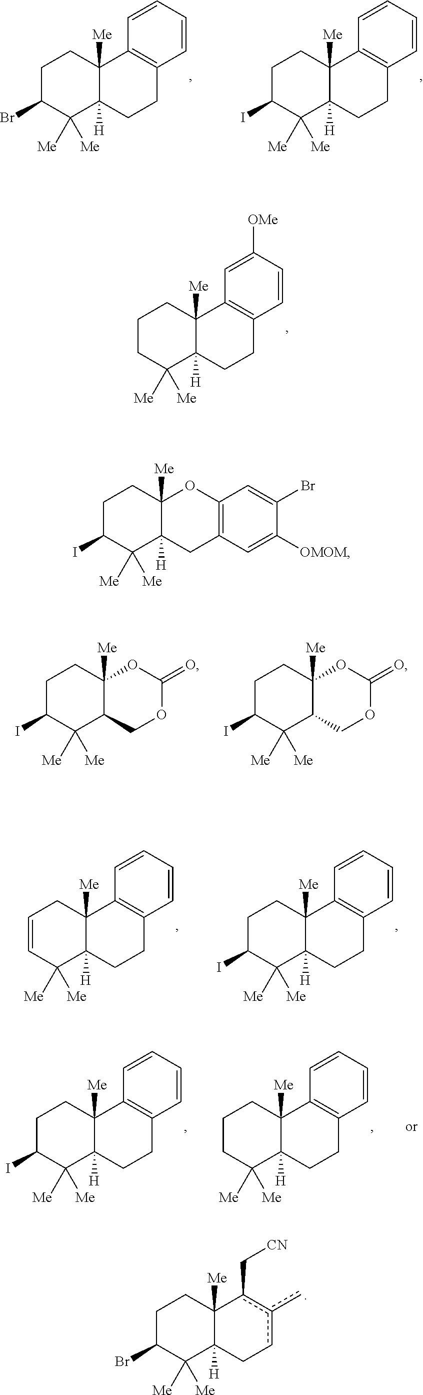

In some embodiments, a process for producing the instant compound comprising:

reacting a polyene having the structure:

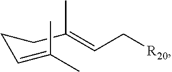

wherein R₂₀ is:

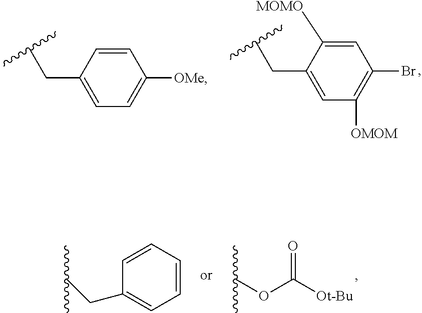

with a second compound having the structure:

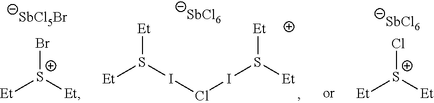

in a suitable solvent at a suitable temperature so as to thereby produce the compound.

In some embodiments of the process, the suitable solvent is MeNO₂.

In some embodiments of the process, the suitable temperature is about −25° C. to 25° C.

In some embodiments, a compound having the structure:

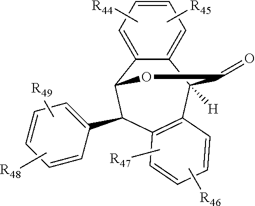

wherein
R₄₄, R₄₅, R₄₆, R₄₇, R₄₈, and R₄₉ are independently H, CN, acetate, OH, OR₅₀, a substituted or unsubstituted C₁₋₆ alkyl, a substituted or unsubstituted C₂₋₆ alkenyl, a ketone, an ester, a substituted or unsubstituted cycloalkyl, a substituted or unsubstituted cycloalkenyl, a substituted or unsubstituted aryl, a substituted or unsubstituted heteroaryl, a substituted or unsubstituted heterocyclyl,
   wherein each occurrence of R₅₀ is independently H, methyl, substituted or unsubstituted alkyl, substituted or unsubstituted heteroalkyl substituted or unsubstituted aryl, substituted or unsubstituted heteroaryl, phosphate, sulfate, sulfonic ester, or ester,
      or a pharmaceutically acceptable salt thereof.

In some embodiments of the compound, wherein
R₄₄, R₄₅, R₄₆, R₄₇, R₄₈, and R₄₉ are independently H, OCH₃ or OCH₂Ph,
   or a pharmaceutically acceptable salt thereof.

In some embodiments, a compound having the structure

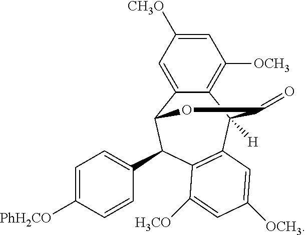

In some embodiments, a composition comprising a compound having the structure:

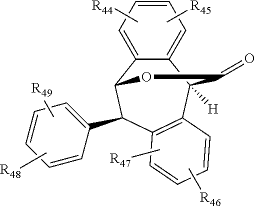

wherein
R₄₄, R₄₅, R₄₆, R₄₇, R₄₈, and R₄₉ are independently H, CN, acetate, OH, OR₅₀, a substituted or unsubstituted C₁₋₆ alkyl, a substituted or unsubstituted C₂₋₆ alkenyl, a ketone, an ester, a substituted or unsubstituted cycloalkyl, a substituted or unsubstituted cycloalkenyl, a substituted or unsubstituted aryl, a substituted or unsubstituted heteroaryl, a substituted or unsubstituted heterocyclyl,
   wherein each occurrence of R₅₀ is independently H, methyl, substituted or unsubstituted alkyl, substituted or unsubstituted heteroalkyl substituted or unsubstituted aryl, substituted or unsubstituted heteroaryl, phosphate, sulfate, sulfonic ester, or ester,
      or a pharmaceutically acceptable salt thereof,
      wherein the composition is free of plant extract.

In some embodiments of the composition, wherein in the compound,
R₄₄, R₄₅, R₄₆, R₄₇, R₄₈, and R₄₉ are independently H, OCH₃ or OCH₂Ph,
   or a pharmaceutically acceptable salt thereof.

In some embodiments of the composition, wherein the compound has structure is

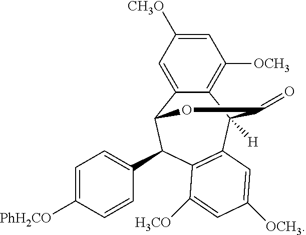

In some embodiments, a process for producing the compound of the instant invention comprising reacting an alkenoic acid having the structure:

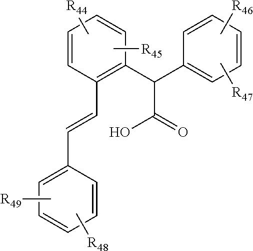

wherein
R₄₄, R₄₅, R₄₆, R₄₇, R₄₈, and R₄₉ are independently H, CN, acetate, OH, OR₅₀, a substituted or unsubstituted C₁₋₆ alkyl, a substituted or unsubstituted C₂₋₆ alkenyl, a ketone, an ester, a substituted or unsubstituted cycloalkyl, a substituted or unsubstituted cycloalkenyl, a substituted or unsubstituted aryl, a substituted or unsubstituted heteroaryl, a substituted or unsubstituted heterocyclyl,
   wherein each occurrence of R₅₀ is independently H, methyl, substituted or unsubstituted alkyl, substituted or unsubstituted heteroalkyl substituted or unsubstituted aryl, substituted or unsubstituted heteroaryl, phosphate, sulfate, sulfonic ester, or ester,
      with a second compound having the structure:

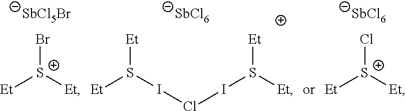

in a suitable solvent at a suitable temperature so as to thereby produce the compound.

In some embodiments, a process for producing the compound of the instant invention comprising

a) reacting an alkenoic acid having the structure:

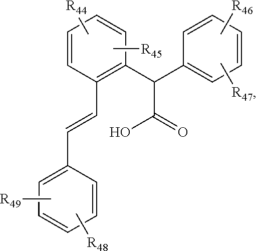

wherein
R₄₄, R₄₅, R₄₆, R₄₇, R₄₈, and R₄₉ are independently H, CN, acetate, OH, OR₅₀, a substituted or unsubstituted C₁₋₆ alkyl, a substituted or unsubstituted C₂₋₆ alkenyl, a ketone, an ester, a substituted or unsubstituted cycloalkyl, a substituted or unsubstituted cycloalkenyl, a substituted or unsubstituted aryl, a substituted or unsubstituted heteroaryl, a substituted or unsubstituted heterocyclyl,
   wherein each occurrence of R₅₀ is independently H, methyl, substituted or unsubstituted alkyl, substituted or unsubstituted heteroalkyl substituted or unsubstituted aryl, substituted or unsubstituted heteroaryl, phosphate, sulfate, sulfonic ester, or ester,
      with a second compound having the structure:

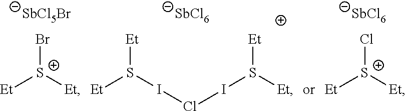

in a suitable solvent at a suitable temperature so as to thereby produce the compound of the composition; and
   b) admixing the product of the step a) with a carrier so as to thereby produce the composition.

In some embodiments of the process,
R₄₄, R₄₅, R₄₆, R₄₇, R₄₈, and R₄₉ are independently H, —OCH₃ or —OCH₂Ph.

In some embodiments of the process, the alkenoic acid has the structure

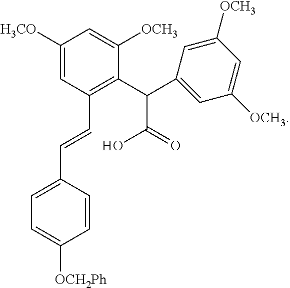

In some embodiments of the process, the compound produced or the compound of the composition produced has the structure

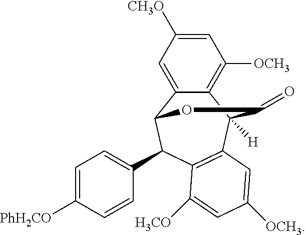

In some embodiments of the process, the second compound has the structure

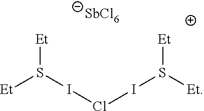

In some embodiments of the process, the suitable solvent is acetonitrile.

In some embodiments of the process, the suitable temperature is about −25° C. to 25° C.

In some embodiments, a compound having the structure:

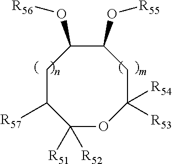

wherein
n=1 or 2; m=1 or 2; R₅₁, R₅₂, R₅₃ and R₅₄ are independently H, alkyl, or a haloalkyl; R₅₅ and R₅₆
are both H or combine to form a carbonate; and R₅₇ is H, Br, I or Cl,
   or a pharmaceutically acceptable salt, diastereomer, or enantiomer thereof.

In some embodiments of the compound,
n=1; m=1; one of R₅₁ or R₅₂ is H and the other is a haloalkyl; one of R₅₃ or R₅₄ is CH₃ and the other is H; R₅₅ and R₅₆ are both H or combine to form a carbonate; and R₅₇ is H,
   or a pharmaceutically acceptable salt, diastereomer, or enantiomer thereof.

In some embodiments of the compound,
n=1; m=1; one of R₅₁ or R₅₂ is H and the other is a alkyl; one of R₅₃ or R₅₄ is CH₃ and the other is H; R₅₅ and R₅₆ are both H or combine to form a carbonate; and R₅₇ is Br,
   or a pharmaceutically acceptable salt, diastereomer, or enantiomer thereof.

In some embodiments of the compound, one of R₅₁ or R₅₂ is

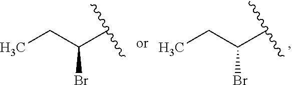

or a pharmaceutically acceptable salt, diastereomer, or enantiomer thereof.

In some embodiments of the compound, one of R₅₁ or R₅₂ is CH₂CH₃,

or a pharmaceutically acceptable salt, diastereomer, or enantiomer thereof.

In some embodiments of the compound,
n=2; m=1; one of R₅₁ or R₅₂ is H and the other is a haloalkyl; one of R₅₃ or R₅₄ is alkyl and the other is H; R₅₅ and R₅₆ are both H or combine to form a carbonate; and R₅₇ is H,
   or a pharmaceutically acceptable salt, diastereomer, or enantiomer thereof.

In some embodiments of the compound,
n=1; m=2; one of R₅₁ or R₅₂ is H and the other is a alkyl; one of R₅₃ or R₅₄ is CH₂(CH₂)₃CH₃ and the other is H; R₅₅ and R₅₆ are both H or combine to form a carbonate; and R₅₇ is Br,
   or a pharmaceutically acceptable salt, diastereomer, or enantiomer thereof.

In some embodiments of the compound, one of R₅₁ or R₅₂ is

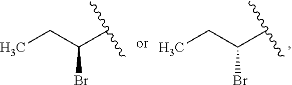

or a pharmaceutically acceptable salt thereof.

In some embodiments of the compound,
one of R₅₁ or R₅₂ is CH₂CH₃,
   or a pharmaceutically acceptable salt, diastereomer, or enantiomer thereof.

In some embodiments, a composition comprising a compound having the structure:

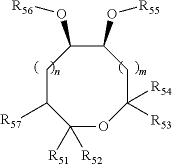

wherein
n=1 or 2; m=1 or 2; R₅₁, R₅₂, R₅₃ and R₅₄ are independently H, alkyl, or a haloalkyl; R₅₅ and R₅₆ are both H or combine to form a carbonate; and R₅₇ is H, Br, I or Cl,
   or a pharmaceutically acceptable salt, diastereomer, or enantiomer thereof.
   wherein the composition is free of plant extract.

In some embodiments, the compound or compound of the composition having the structure

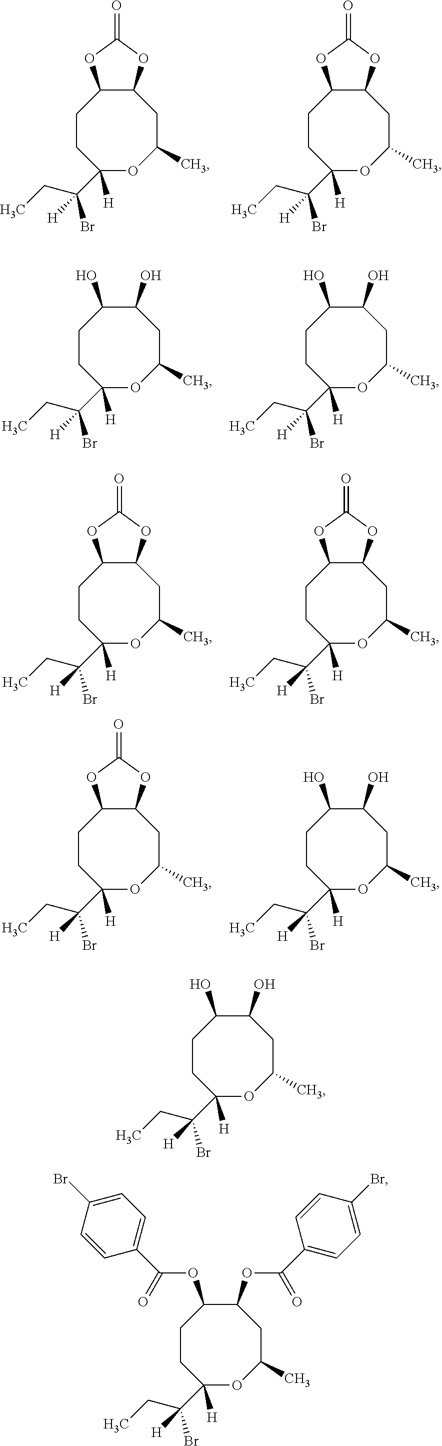

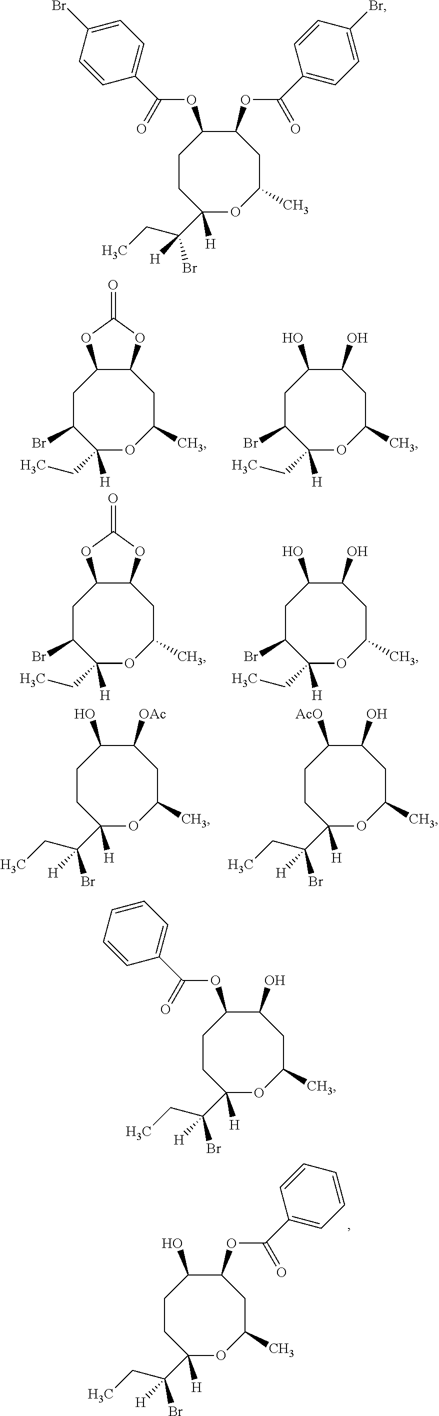

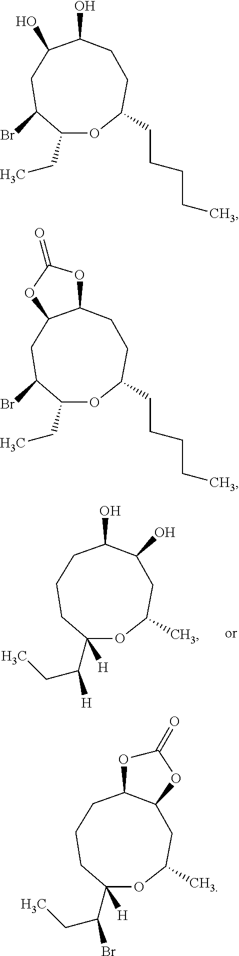

In some embodiments, a process for producing the compound of the instant invention comprising reacting the alkenyl alkyl ether having the structure:

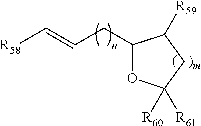

wherein
n=1, 2 or 3; m=1 or 2; R₅₈ is alkyl; R₅₉ is OAc, OBoc, or OBz; and R₆₀ and R₆₁ are independently H or alkyl;
   with a second compound having the structure:

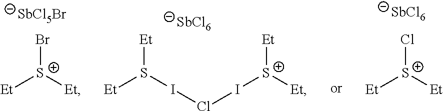

in a suitable solvent at a suitable temperature so as to thereby produce the compound.

In some embodiments, a process for producing the compound of the instant invention comprising a) reacting the alkenyl alkyl ether having the structure:

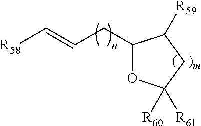

wherein n=1, 2 or 3; m=1 or 2; R₅₈ is alkyl; R₅₉ is OAc, OBoc, or OBz; and R₆₀ and R₆₁ are independently H or alkyl;
   with a second compound having the structure:

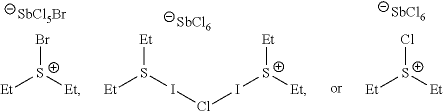

in a suitable solvent at a suitable temperature so as to thereby produce the compound of the composition; and
b) admixing the product of the step a) with a carrier so as to thereby produce the composition.

In some embodiments of the process, R₅₉ is OBoc.

In some embodiments, a process for producing the compound of the instant invention, wherein the compound produced has the structure:

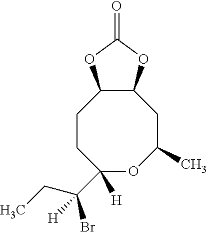 [SMILES: CC[C@H](Br)[C@@H]1CC[C@H]2OC(=O)O[C@H]2C[C@@H](C)O1]

and the alkenyl alkyl ether has the structure:

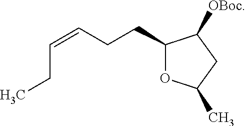

In some embodiments, a process for producing the compound of the instant invention, wherein the compound produced has the structure:

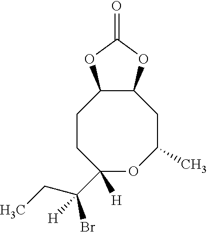 [SMILES: CC[C@H](Br)[C@@H]1CC[C@H]2OC(=O)O[C@H]2C[C@H](C)O1]

and the alkenyl alkyl ether has the structure:

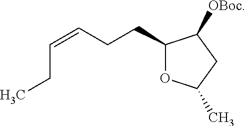

In some embodiments, a process for producing the compound of the instant invention, wherein the compound produced has the structure:

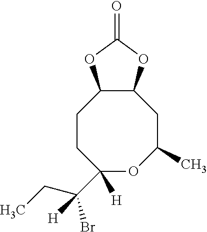 [SMILES: CC[C@@H](Br)[C@@H]1CC[C@H]2OC(=O)O[C@H]2C[C@@H](C)O1]

and the alkenyl alkyl ether has the structure:

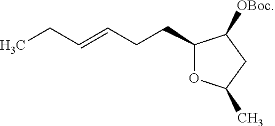

In some embodiments, a process for producing the compound of the instant invention, wherein the compound produced has the structure:

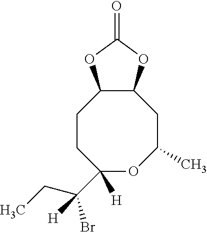 [SMILES: CC[C@@H](Br)[C@@H]1CC[C@H]2OC(=O)O[C@H]2C[C@H](C)O1]

and the alkenyl alkyl ether has the structure:

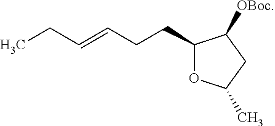

In some embodiments, a process for producing the compound of the instant invention, wherein the compound produced has the structure:

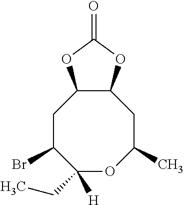 [SMILES: CC[C@H]1O[C@H](C)C[C@@H]2OC(=O)O[C@@H]2C[C@@H]1Br]

and the alkenyl alkyl ether has the structure:

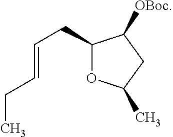

In some embodiments, a process for producing the compound of the instant invention, wherein the compound produced has the structure:

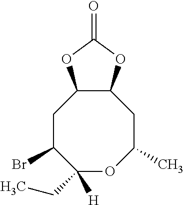 [SMILES: CC[C@H]1O[C@@H](C)C[C@@H]2OC(=O)O[C@@H]2C[C@@H]1Br]

and the alkenyl alkyl ether has the structure:

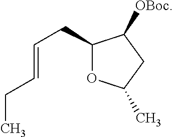

In some embodiments, a process for producing the compound of the instant invention, wherein the compound produced has the structure:

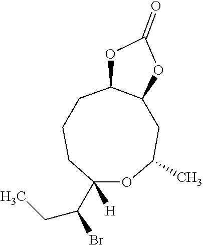 [SMILES: CC[C@H](Br)[C@@H]1CCC[C@H]2OC(=O)O[C@H]2C[C@H](C)O1]

and the alkenyl alkyl ether has the structure:

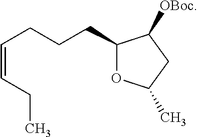

In some embodiments, a process for producing the compound of the instant invention, wherein the compound produced has the structure:

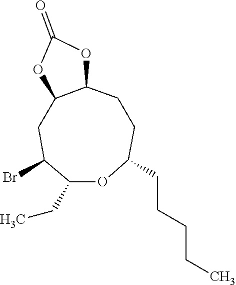 [SMILES: CCCCC[C@H]1CC[C@@H]2OC(=O)O[C@@H]2C[C@H](Br)[C@@H](CC)O1]

and the alkenyl alkyl ether has the structure:

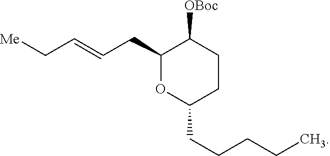

In some embodiments of the process, the second compound has the structure

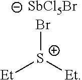

In some embodiments of the process, the suitable solvent is nitromethane.

In some embodiments of the process, the suitable temperature is about −25° C. to 25° C.

In some embodiments, a compound having the structure:

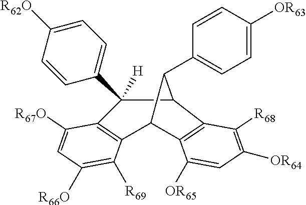

wherein
R₆₂, R₆₃, R₆₄, R₆₅, R₆₆, and R₆₇ are independently H, methyl, substituted or unsubstituted alkyl, substituted or unsubstituted heteroalkyl, substituted or unsubstituted aryl, substituted or unsubstituted heteroaryl, phosphate, sulfate, sulfonic ester, or ester; and
R₆₈ and R₆₉ are independently H, Cl, Br or I;
   or a pharmaceutically acceptable salt thereof.

In some embodiments, a compound wherein
R₆₂, R₆₃, R₆₄, R₆₅, R₆₆, and R₆₇ are each CH₃,
   or a pharmaceutically acceptable salt thereof.

In some embodiments, a compound having the structure

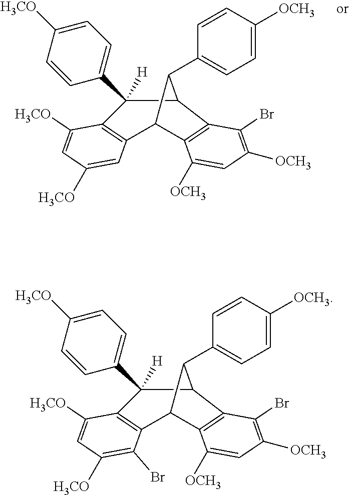 [SMILES: COc1ccc([C@H]2c3c(OC)cc(OC)cc3C3c4c(OC)cc(OC)c(Br)c4[C@@H]2[C@@H]3c2ccc(OC)cc2)cc1 ; COc1ccc([C@H]2c3c(OC)cc(OC)c(Br)c3C3c4c(OC)cc(OC)c(Br)c4[C@@H]2[C@@H]3c2ccc(OC)cc2)cc1]

In some embodiments, a composition comprising a compound having the structure:

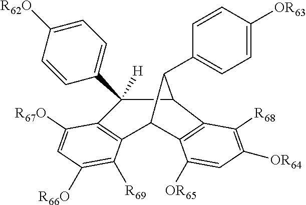

wherein
R₆₂, R₆₃, R₆₄, R₆₅, R₆₆, and R₆₇ are independently H, methyl, substituted or unsubstituted alkyl, substituted or unsubstituted heteroalkyl, substituted or unsubstituted aryl, substituted or unsubstituted heteroaryl, phosphate, sulfate, sulfonic ester, or ester; and
R₆₈ and R₆₉ are independently H, Cl, Br or I;
   or a pharmaceutically acceptable salt thereof,
   wherein the composition is free of plant extract.

In some embodiments, a composition comprising a compound wherein
R₆₂, R₆₃, R₆₄, R₆₅, R₆₆, and R₆₇ are each CH₃,
   or a pharmaceutically acceptable salt thereof.

In some embodiments, a composition comprising a compound having the structure

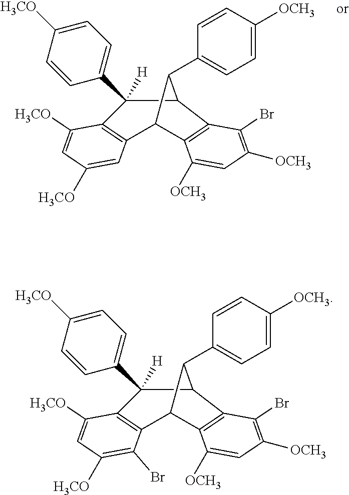 [SMILES: COc1ccc([C@H]2c3c(OC)cc(OC)cc3C3c4c(OC)cc(OC)c(Br)c4[C@@H]2[C@@H]3c2ccc(OC)cc2)cc1 ; COc1ccc([C@H]2c3c(OC)cc(OC)c(Br)c3C3c4c(OC)cc(OC)c(Br)c4[C@@H]2[C@@H]3c2ccc(OC)cc2)cc1]

In some embodiments, a process for producing the instant compound comprising reacting an aromatic ring-containing compound having the structure:

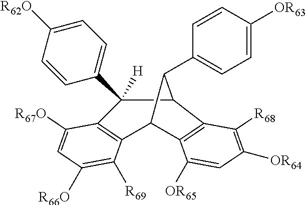

wherein
R₆₂, R₆₃, R₆₄, R₆₅, R₆₆, and R₆₇ are independently H, methyl, substituted or unsubstituted alkyl, substituted or unsubstituted heteroalkyl, substituted or unsubstituted aryl, substituted or unsubstituted heteroaryl, phosphate, sulfate, sulfonic ester, or ester;
R₆₈ is H, Cl, Br or I; and
R₆₉ is H,
   with a second compound having the structure:

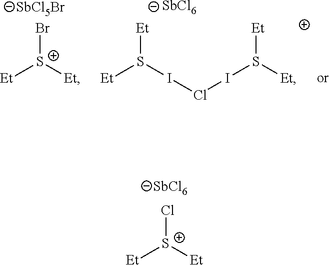

in a suitable solvent at a suitable temperature so as to thereby produce the compound.

In some embodiments, a process for producing the instant composition comprising a) reacting a aromatic ring-containing compound having the structure:

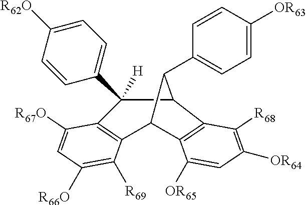

wherein
R₆₂, R₆₃, R₆₄, R₆₅, R₆₆, and R₆₇ are independently H, methyl, substituted or unsubstituted alkyl, substituted or unsubstituted heteroalkyl, substituted or unsubstituted aryl, substituted or unsubstituted heteroaryl, phosphate, sulfate, sulfonic ester, or ester;
R₆₈ is H, Cl, Br or I; and
R₆₉ is H,
   with a second compound having the structure:

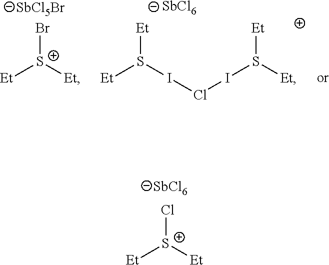

in a suitable solvent at a suitable temperature so as to thereby produce the compound of the composition; and
b) admixing the product of the step a) with a carrier so as to thereby produce the composition.

In some embodiments of the process, R₆₂, R₆₃, R₆₄, R₆₅, R₆₆, and R₆₇ are each CH₃.

In some embodiments of the process, the compound produced has the structure:

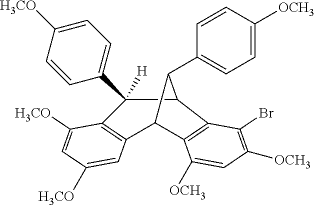 [SMILES: COc1ccc([C@H]2c3c(OC)cc(OC)cc3C3c4c(OC)cc(OC)c(Br)c4[C@@H]2[C@@H]3c2ccc(OC)cc2)cc1]

and the aromatic ring-containing compound has the structure:

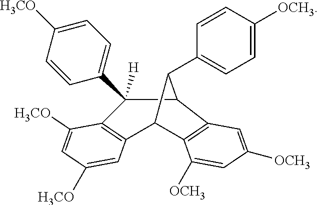 [SMILES: COc1ccc([C@H]2c3c(OC)cc(OC)cc3C3c4c(OC)cc(OC)cc4[C@@H]2[C@@H]3c2ccc(OC)cc2)cc1]

In some embodiments of the process, the compound produced has the structure:

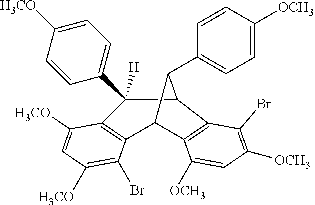 [SMILES: COc1ccc([C@H]2c3c(OC)cc(OC)c(Br)c3C3c4c(OC)cc(OC)c(Br)c4[C@@H]2[C@@H]3c2ccc(OC)cc2)cc1]

and the aromatic ring-containing compound has the structure:

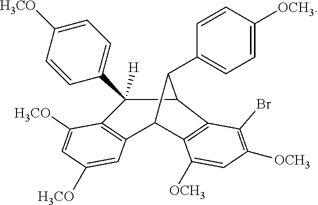 [SMILES: COc1ccc([C@H]2c3c(OC)cc(OC)cc3C3c4c(OC)cc(OC)c(Br)c4[C@@H]2[C@@H]3c2ccc(OC)cc2)cc1]

In some embodiments of the process, the second compound has the structure

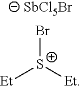

In some embodiments of the process, the suitable solvent is dichloromethane.

In some embodiments of the process, the suitable temperature is about −78° C. to 25° C.

This invention also provides isotopic variants of the compounds disclosed herein, including wherein the isotopic atom is ²H and/or wherein the isotopic atom ¹³C. Accordingly, in the compounds provided herein hydrogen can be enriched in the deuterium isotope. It is to be understood that the invention encompasses all such isotopic forms.

It is understood that the structures described in the embodiments of the methods hereinabove can be the same as the structures of the compounds described hereinabove.

It is understood that where radicals are respresented by structure, the point of attachment to the main structure is represented by a wavy line.

It is understood that where a numerical range is recited herein, the present invention contemplates each integer between, and including, the upper and lower limits, unless otherwise stated.

As used herein, the term “activity” refers to the activation, production, expression, synthesis, intercellular effect, and/or pathological or aberrant effect of the referenced molecule, either inside and/or outside of a cell. Such molecules include, but are not limited to, cytokines, enzymes, growth factors, pro-growth factors, active growth factors, and pro-enzymes. Molecules such as cytokines, enzymes, growth factors, pro-growth factors, active growth factors, and pro-enzymes may be produced, expressed, or synthesized within a cell where they may exert an effect. Such molecules may also be transported outside of the cell to the extracellular matrix where they may induce an effect on the extracellular matrix or on a neighboring cell. It is understood that activation of inactive cytokines, enzymes and pro-enzymes may occur inside and/or outside of a cell and that both inactive and active forms may be present at any point inside and/or outside of a cell. It is also understood that cells may possess basal levels of such molecules for normal function and that abnormally high or low levels of such active molecules may lead to pathological or aberrant effects that may be corrected by pharmacological intervention.

The compounds of the present invention include all hydrates, solvates, and complexes of the compounds used by this invention. If a chiral center or another form of an isomeric center is present in a compound of the present invention, all forms of such isomer or isomers, including enantiomers and diastereomers, are intended to be covered herein unless the structure shows otherwise. Compounds containing a chiral center may be used as a racemic mixture, an enantiomerically enriched mixture, or the racemic mixture may be separated using well-known techniques and an individual enantiomer may be used alone. The compounds described in the present invention are in racemic form or as individual enantiomers. The enantiomers can be separated using known techniques, such as those described in Pure and Applied Chemistry 69, 1469-1474, (1997) IUPAC. In cases in which compounds have unsaturated carbon-carbon double bonds, both the cis (Z) and trans (E) isomers are within the scope of this invention.

The compounds of the subject invention may have spontaneous tautomeric forms. In cases wherein compounds may exist in tautomeric forms, such as keto-enol tautomers, each tautomeric form is contemplated as being included within this invention whether existing in equilibrium or predominantly in one form.

In the compound structures depicted herein, hydrogen atoms are not shown for carbon atoms having less than four bonds to non-hydrogen atoms. However, it is understood that enough hydrogen atoms exist on said carbon atoms to satisfy the octet rule.

As used herein, “alkyl” includes both branched and straight-chain saturated aliphatic hydrocarbon groups having the specified number of carbon atoms and may be unsubstituted or substituted. Thus, C₁₋ₙ as in “C₁₋ₙ alkyl” is defined to include groups having 1, 2, . . . , n−1 or n carbons in a linear or branched arrangement. For example, C₁₋₆, as in “C₁₋₆ alkyl” is defined to include groups having 1, 2, 3, 4, 5, or 6 carbons in a linear or branched arrangement, and specifically includes methyl, ethyl, n-propyl, isopropyl, n-butyl, t-butyl, pentyl, hexyl, and octyl.

As used herein, “alkenyl” refers to a non-aromatic hydrocarbon radical, straight or branched, containing at least 1 carbon to carbon double bond, and up to the maximum possible number of non-aromatic carbon-carbon double bonds may be present, and may be unsubstituted or substituted. For example, “C₂₋₆ alkenyl” means an alkenyl radical having 2, 3, 4, 5, or 6 carbon atoms, and up to 1, 2, 3, 4, or 5 carbon-carbon double bonds respectively. Alkenyl groups include ethenyl, propenyl, butenyl and cyclohexenyl.

The term “alkynyl” refers to a hydrocarbon radical straight or branched, containing at least 1 carbon to carbon triple bond, and up to the maximum possible number of non-aromatic carbon-carbon triple bonds may be present, and may be unsubstituted or substituted. Thus, “C₂-C₆ alkynyl” means an alkynyl radical having 2 or 3 carbon atoms and 1 carbon-carbon triple bond, or having 4 or 5 carbon atoms and up to 2 carbon-carbon triple bonds, or having 6 carbon atoms and up to 3 carbon-carbon triple bonds. Alkynyl groups include ethynyl, propynyl and butynyl.

“Alkylene”, “alkenylene” and “alkynylene” shall mean, respectively, a divalent alkane, alkene and alkyne radical, respectively. It is understood that an alkylene, alkenylene, and alkynylene may be straight or branched. An alkylene, alkenylene, and alkynylene may be unsubstituted or substituted.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include phenyl, p-toluenyl (4-methylphenyl), naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring.

As used herein, the term “polycyclic” refers to unsaturated or partially unsaturated multiple fused ring structures, which may be unsubstituted or substituted.

The term “arylalkyl” refers to alkyl groups as described above wherein one or more bonds to hydrogen contained therein are replaced by a bond to an aryl group as described above. It is understood that an “arylalkyl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “heterocycle”, “heterocyclyl” or “heterocyclic” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The alkyl, alkenyl, alkynyl, aryl, heteroaryl and heterocyclyl substituents may be substituted or unsubstituted, unless specifically defined otherwise.

In the compounds of the present invention, alkyl, alkenyl, alkynyl, aryl, heterocyclyl and heteroaryl groups can be further substituted by replacing one or more hydrogen atoms with alternative non-hydrogen groups. These include, but are not limited to, halo, hydroxy, mercapto, amino, carboxy, cyano and carbamoyl.

As used herein, the term “halogen” refers to F, Cl, Br, and I.

The term “substituted” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituents include the functional groups described above, and, in particular, halogens (i.e., F, Cl, Br, and I); alkyl groups, such as methyl, ethyl, n-propyl, isopropryl, n-butyl, tert-butyl, neopentyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing the compounds of the present invention, one of ordinary skill in the art will recognize that the various substituents, i.e. R₁, R₂, etc. are to be chosen in conformity with well-known principles of chemical structure connectivity.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5th Edition (1996).

The term “ester” is intended to a mean an organic compound containing the R—O—CO—R′ group.

The term “phosphate” is intended to mean an organic compound containing the R—O—P(O)(OR′)₂ group. In a non-limiting example, each occurrence of R′ may be identical or different. In a non-limiting example, R′ may be an H, alkyl or negative charge.

The term “sulfate” is intended to mean an organic compound containing the RO—SO₂—OR′ group. In a non-limiting example, R′ may be an H or a negative charge.

The term “sulfonic esters” is intended to mean an organic compound containing the R—O—SO₂R′ group.

The alkyl, alkenyl, alkynyl, cycloalkyl, cycloalkenyl, aryl, heteroaryl and heterocyclyl substituents may be unsubstituted or unsubstituted, unless specifically defined otherwise. In a non-limiting example, a C₂-C₆ alkyl may be substituted with one or more substituents selected from OH, oxo, halogen, alkoxy, dialkylamino, or heterocyclyl, such as morpholinyl, piperidinyl, and so on.

In the compounds of the present invention, alkyl, alkenyl, alkynyl, cycloalkyl, cycloalkenyl, aryl, heterocyclyl and heteroaryl groups can be further substituted by replacing one or more hydrogen atoms be alternative non-hydrogen groups. These include, but are not limited to, halo, hydroxy, mercapto, amino, carboxy, cyano and carbamoyl.

In the compounds used in the method of the present invention, the substituents may be substituted or unsubstituted, unless specifically defined otherwise.

In the compounds used in the method of the present invention, alkyl, heteroalkyl, aryl, heteroaryl, phosphate, sulfate, sulfonic ester, or ester groups can be further substituted by replacing one or more hydrogen atoms with alternative non-hydrogen groups. These include, but are not limited to, halo, hydroxy, mercapto, amino, carboxy, cyano and carbamoyl.

The various R groups attached to the aromatic rings of the compounds disclosed herein may be added to the rings by standard proceudres, for example those set forth in Advanced Organic Chemistry: Part B: Reaction and Synthesis, Francis Carey and Richard Sundberg, (Springer) 5th ed. Edition. (2007), the content of which is hereby incorporated by reference.

The compounds described in the present invention are in racemic form or as individual enantiomers. The enantiomers can be separated using known techniques, such as those described in Pure and Applied Chemistry 69, 1469-1474, (1997) IUPAC.

The compounds of the instant invention may be in a salt form. As used herein, a “salt” is the salt of the instant compounds which has been modified by making acid or base salts of the compounds. Acidic substances can form salts with acceptable bases, including, but not limited to, lysine, arginine, and the like.

In the case of compounds administered to a subject, eg. a human, the salt is pharmaceutically acceptable. Examples of pharmaceutically acceptable salts include, but are not limited to, mineral or organic acid salts formed at basic residues such as amino groups; alkali or organic base salts formed at acidic residues such as phenols, carboxylic acids, and carbons having at least 1 acidic hydrogen atom adjacent to a carbonyl. Where acid salts are formed, such salts can be made using an organic or inorganic acid. Such acid salts include, but are not limited to, chlorides, bromides, sulfates, nitrates, phosphates, sulfonates, formates, tartrates, maleates, malates, citrates, benzoates, salicylates, ascorbates, and the like. Because the compounds of the subject invention also possess carbons having at least 1 acidic hydrogen atom adjacent to a carbonyl, enolate salts may be formed by reaction with a suitable base. Suitable bases include, but are not limited, to inorganic bases, such as alkali and alkaline earth metal hydroxides; and organic bases, including, but not limited to, ammonia, alkyl amines, amino alcohols, amino sugars, amino acids, such as glycine, histidine, and lysine, and alkali metal amides, such as lithium diisopropylamide. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The compounds and compositions of this invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind Liposomes are also a pharmaceutically acceptable carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

The compounds and compositions of the present invention can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by topical administration, injection or other methods, to the afflicted area, such as a wound, including ulcers of the skin, all using dosage forms well known to those of ordinary skill in the pharmaceutical arts.

The compounds can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. In one embodiment the carrier can be a monoclonal antibody. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Specific examples of pharmaceutical acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

The term “prodrug” as used herein refers to any compound that when administered to a biological system generates the compound of the invention, as a result of spontaneous chemical reaction(s), enzyme catalyzed chemical reaction(s), photolysis, and/or metabolic chemical reaction(s). A prodrug is thus a covalently modified analog or latent form of a compound of the invention.

The active ingredient can be administered orally in solid dosage forms, such as capsules, tablets, powders, and chewing gum; or in liquid dosage forms, such as elixirs, syrups, and suspensions, including, but not limited to, mouthwash and toothpaste. It can also be administered parentally, in sterile liquid dosage forms.

Solid dosage forms, such as capsules and tablets, may be enteric coated to prevent release of the active ingredient compounds before they reach the small intestine. Materials that may be used as enteric coatings include, but are not limited to, sugars, fatty acids, waxes, shellac, cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), and methyl methacrylate-methacrylic acid copolymers.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Sustained release liquid dosage forms suitable for parenteral administration, including, but not limited to, water-in-oil and oil-in-water microemulsions and biodegradable microsphere polymers, may be used according to methods well-known to those having ordinary skill in the art. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field. Solubilizing agents may be used to enhance solubility of the compounds of the subject invention in the liquid dosage form. Suitable solubilizing agents include, but are not limited to, amines, amino alcohols, amino sugars, and amino acids, such as glycine, histidine, and lysine.

The compounds of the instant invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

The compounds and compositions of the invention can be coated onto stents for temporary or permanent implantation into the cardiovascular system of a subject.

Variations on the synthetic methods disclolsed herein will be readily apparent to those skilled in the art and are deemed to be within the scope of the present invention.

Of the starting compounds contemplated in the present invention, the non-novel ones may be purchased from commercial sources or may be synthesized using conventional functional group transformations well-known in the chemical arts, for example, those set forth in Organic Synthesis, Michael B. Smith, (McGraw-Hill) Second ed. (2001) and March's Advanced Organic Chemistry: Reactions, Mechanisms, and Structure, Michael B. Smith and Jerry March, (Wiley) Sixth ed. (2007).

Further, where substituents are contemplated, such substituents may be incorporated in the compounds of the present invention using conventional functional group transformations well-known in the chemical arts.

In some embodiments, the natural product analogs and the compositions of the present invention are useful in the inhibition of viral infection.

In some embodiments, the natural product analogs and the compositions of the present invention are useful as reverse transcriptase inhibitors of HIV-1.

In some embodiments, halogen-containing small molecules of the present invention are useful in the cyclization of polyenes.

It will be noted that any notation of a carbon in structures throughout this application, when used without further notation, are intended to represent all isotopes of carbon, such as 12C, 13C, or 14C. Furthermore, any compounds containing 13C or 14C may specifically have the structure of any of the compounds disclosed herein.

It will also be noted that any notation of a hydrogen in structures throughout this application, when used without further notation, are intended to represent all isotopes of hydrogen, such as 1H, 2H, or 3H. Furthermore, any compounds containing 2H or 3H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

An additional aspect of the invention provides a reagent useful for initiating a process which requires an electrophilic halogen source.

The compounds used in the method of the present invention may be prepared by techniques well know in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5th Edition (1996), March's Advanced Organic Chemistry: Reactions, Mechanisms, and Structure, Michael B. Smith, Jerry March, (Wiley-Interscience) 5th Edition (2007), and references therein, which are incorporated by reference herein. However, these may not be the only means by which to synthesize or obtain the desired compounds.

The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

The term “about” with regard to a temperature of X ° C. encompasses temperatures up to 5° C. greater than X and 5° C. less than X.

“Free of plant extract” with regard to a composition as used here means that the composition is absent any amount of plant material, including, but not limited to, Peyssonnelia sp. plant material, Neobalanacarpus heimii plant material, Laurencia plant material, or Resveratrol oligomer-based plant material. Thus, only synthetically produced compounds and compositions are free of plant extract. Any compound or compositions isolated from a plant would always contain at least some trace amount of plant material.

Each embodiment disclosed herein is contemplated as being applicable to each of the other disclosed embodiments. Thus, all combinations of the various elements described herein are within the scope of the invention.

Herein, where chemical substituents are disclosed in the alternative, it is intended that each such substituent can be used or combined with one or more other substituents disclosed in the alternative.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

Herein, we describe the development of the first class of reagents that can render possible the direct synthesis of a diverse range of chlorine-, bromine-, and iodine-containing polycycles via cation-π cyclizations. Each reagent is a readily prepared crystalline solid that reacts with olefins highly chemoselectively and rapidly, with reactions normally complete within 5 minutes at low temperature. Moreover, added acids are not typically required to drive cyclizations to completion. To date, these reagents have allowed us to accomplish racemic total and formal syntheses of 7 different natural products, 6 of which are disclosed for the first time in this article (including a substantial structural revision), as well as to cyclize nearly 20 additional substrates in yields that are often multifold improvements over previously available alternatives.

### Example 1

### The Development of BDSB

The initial research goal was to identify a novel reagent with higher alkene chemoselectivity and less proclivity for side-product formation. A preferred reagent would need to be a stable and easily-handled material rather than one that would have to be prepared in situ. In addition, its molecular structure would ideally prove applicable to generating the corresponding iodine- and chlorine-based variants and, eventually, chiral versions for asymmetric applications.

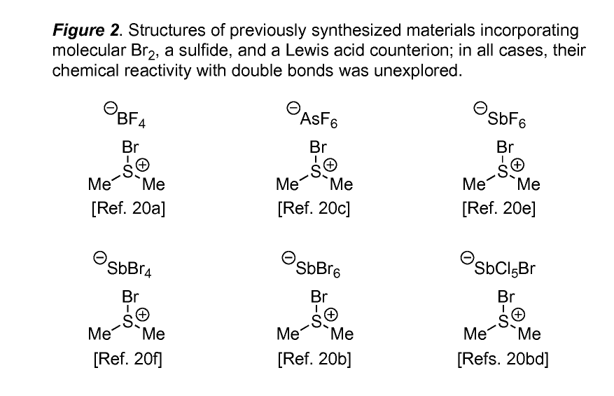

Initially the research was predicated on enhancing the electrophilicity of a typical bromine source (like Br₂ or NBS) while concurrently removing the potential for any other species (such as a counterion) to serve as either nucleophile or base (19). An extensive search of the literature revealed the existence of several reagents that formally met this criteria; 6 of these compounds are presented in FIG. 2, all of which are complexes of Br₂ with Me₂S and a Lewis acid (20).

Interestingly, although these materials have been known for some time (one was reported over 50 years ago), no report describes their chemical reactivity. Preliminary screens revealed that the use of SbCl₅ as the Lewis acid component most consistently afforded solid materials relative to boron or aluminum alternatives (21a). Additionally, of the various simple dialkyl sulfides that could be used (such as methyl, ethyl, isopropyl, or t-butyl), the ethyl variant was the most easily prepared (21b). As indicated in Scheme 1, addition of a slight excess of Et₂S and SbCl₅ to Br₂ in 1,2-dichloroethane at −30° C. immediately produced a yellow solid that could be recrystallized from the reaction solution to give the material shown in the inset photo in 87% yield. This odorless crystalline solid, which we have named BDSB (for bromodiethylsulfonium bromopentachloroantimonate, 13), can be prepared smoothly on hundred-gram scale (22) is stable at ambient temperature in an enclosed vial for at least 1 week (and for a year or more at −20° C.) and possesses good solubility in several organic solvents (22).

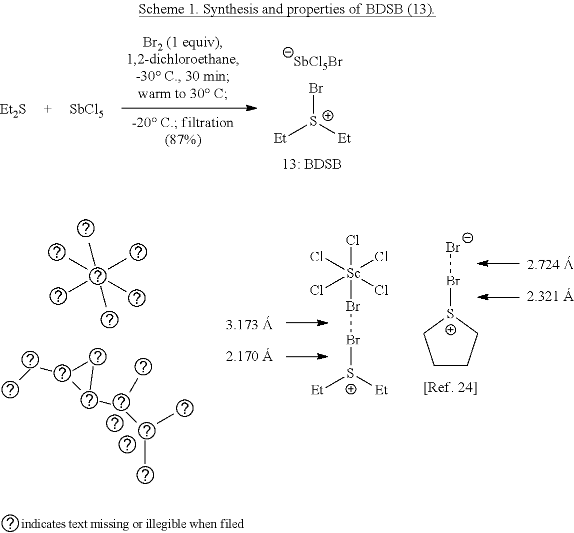

The X-ray crystal structure of BDASB revealed relatively short bromine-sulfur bond and effective sequestration of bromide to the antimonate counterion constitutes a significant departure from typical bromosulfonium complexes, an example of which is given in Scheme 1 (and which is ineffective for bromonium-induced cation-π cyclizations) (24). Indeed, as recently reported in a preliminary communication (25) BDSB is very effective at inducing cation-π cyclizations for a variety of substrates, including those that possess electron-deficient alkenes, as well as polyenes containing Z-alkenes (25).

Table 1 provides a subset of the examples that were previously disclosed. It is worth noting that some of these reactions have been conducted on scales as large as 5.0 mmol in equivalent yields, and that the nitromethane utilized can be recovered and reused in these large scale processes. Additionally, reactions are generally very fast (usually complete in less than 5 minutes), and in all cases product yields are superior to those obtained by other available methods reported in the literature. As with most cation-π cyclizations, reaction concentrations need to be kept dilute (0.05 M on small scale; 0.01 M on larger scale) for optimal yields. In terms of chemoselectivity, BDSB will typically react cleanly with olefins prior to aromatic systems, even those that are electron-rich (such as those in 17 and 19, Entries 3 and 4), though it can perform electrophilic aromatic bromination if no C═C bonds are present.

In its reactions with olefins, BDSB possesses typical electrophilic reactivity patterns: more substituted and more electronically activated double bonds will react faster, and usually selectively, over their less-substituted and/or electron-deficient counterparts (26). Fortunately, steric considerations appear to be more important than electronic considerations given that in polyenes such as 19, the more accessible, yet less electron-rich distal double bond consistently reacts preferentially to the hindered, more electron-rich central double bond. A minor side-product formed in many reactions is the proton-cyclized homologue; an acidic by-product, likely protonated Et₂S, is formed as the reaction progresses and is responsible for the observed yield (usually <5%) of this undesired compound (27). This acid cannot be neutralized in situ with added base, but is of value in that it may help to drive many cyclizations to completion by promoting formation of multiple rings, especially when synthesizing tricyclic or tetracyclic materials. We note as well that while catalytic versions of this reagent design are conceivable (in terms of the sulfide), we have not pursued such explorations since we anticipate that they would not be ideal in many cases. For instance, the conversion of 8 into 14 (Entry 1, Table 1) provides products with reactive olefins, which are able to obtain in good yield only because there is rapid consumption of the starting material prior to the formation of significant amounts of product (which would likely react with BDSB if it were formed via a slower, catalytic process).

TABLE 1
Exploration of the generality of direct, bromonium-induced cation-π
cyclizations using BDSB (1.1 equiv) and 0.1 mmol of substrate in nitromethane.

| | | | Temp. | Time | Yield |
|---|---|---|---|---|---|
| Entry | Starting Material | Product | (° C.) | (min) | (%) |
| 1 | 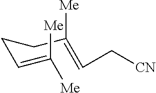 [SMILES: CC(C)=CCC/C(C)=C/CC#N] 8 | 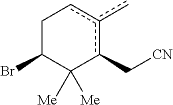 14 | 25 | 5 | 73^{a} |
| 2 | 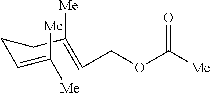 [SMILES: CC(=O)OC/C=C(\C)CCC=C(C)C] 15 | 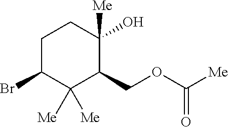 [SMILES: CC(=O)OC[C@H]1C(C)(C)[C@@H](Br)CC[C@]1(C)O] 16 | 0 | 1 | 80^{b} |
| 3 | 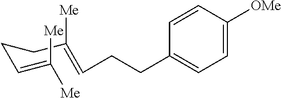 [SMILES: COc1ccc(CC/C=C(\C)CCC=C(C)C)cc1] 17 | 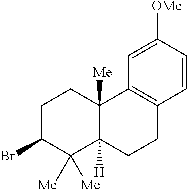 [SMILES: COc1ccc2c(c1)[C@@]1(C)CC[C@H](Br)C(C)(C)[C@@H]1CC2] 18 | −25 | 5 | 76 |
| 4 | 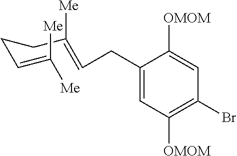 19 | 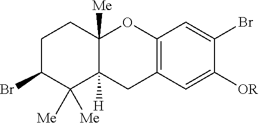 20: R = MOM | −25 | 5 | 74 |
| 5 | 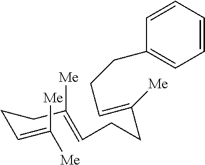 [SMILES: CC(C)=CCC/C(C)=C/CC/C(C)=C/CCc1ccccc1] 21 | 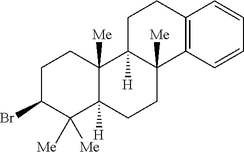 22 | −25 | 5 | 58^{c,d} |
| 6 | 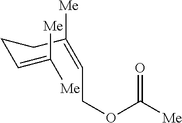 [SMILES: CC(=O)OC/C=C(/C)CCC=C(C)C] 23 | 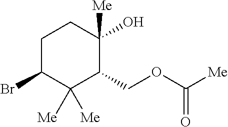 [SMILES: CC(=O)OC[C@@H]1C(C)(C)[C@@H](Br)CC[C@]1(C)O] 24 [X-ray obtained] | 0 | 1 | 71 |

^{a}Produced as a 6.5:2.5:1.0 mixture of tri:tetra:disubstituted alkene isomers;
^{b}Produced as a 3.8:1.0 mixture of separable diastereomers at the highlighted carbon favoring the drawn product;
^{c}Generated alongside some very minor diastereomers;
^{d}MeSO₃H (15 equiv) added with 1 h of additional stirring to promote the final cyclization.

### Example 2

### Further Explorations into the Power of BDSB: Total and Formal Syntheses of Peyssonol A, Peyssonoic Acid A, and Aplysin-20

Investigations with BDSB have centered on exploring its reactivity with progressively longer polyenes, especially trienes possessing unique (i.e. Z) stereochemistry in hopes of accessing the frameworks of several complex and structurally intriguing natural products. Attention was drawn to the structure of the secondary metabolite peyssonol A (3, Scheme 2), a material that was obtained from the Red Sea marine alga Peyssonnelia sp., that has been shown to act as an allosteric inhibitor of the reverse transcriptases of the Human Immunodeficiency Virus (7cd) To the best of our knowledge, this compound is the only known natural product possessing a cis-decalin framework likely arising from a halonium-induced cation-cyclization. As such, we felt it would be an ideal proving ground to evaluate the power of BDSB to effect a direct and highly challenging cation-π cyclization to access a framework distinct from those we had previously prepared (28).

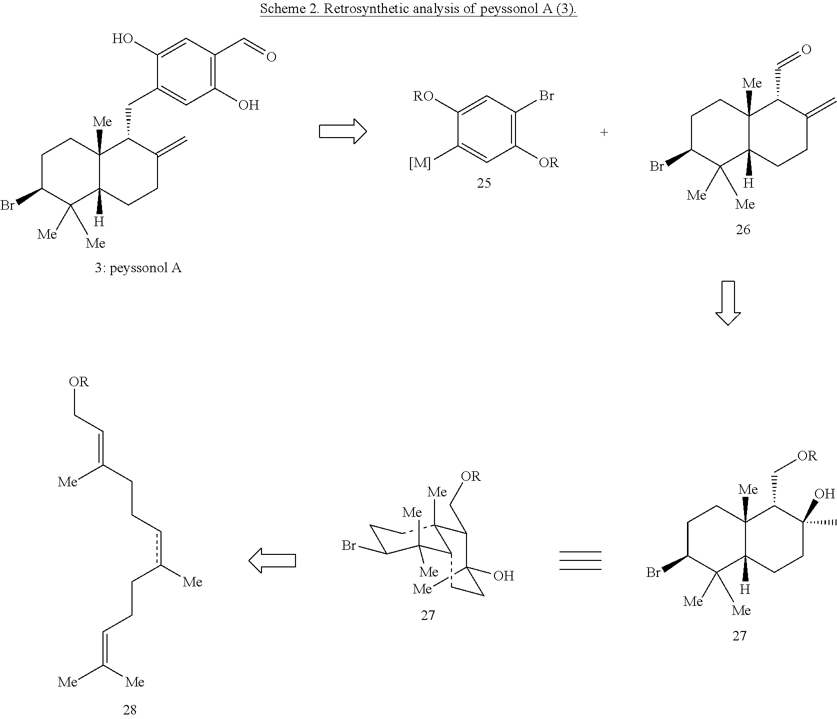

As indicated in Scheme 2, our retrosynthetic analysis suggested that a late-stage disconnection of the pendant aryl ring, projecting a nucleophilic addition onto the aldehyde within compound 26 to effect its incorporation, might afford the most efficient means to reach a suitable polyene cyclization precursor. Compound 26 could potentially arise from cis-decalin 27, which could in turn directly result from a bromonium-induced cation-π cyclization of the (2E,6Z)-farnesol derivative 28. Either an acetate or 30 carbonate as group R within 28 would hopefully give rise to the desired functionality within 27, assuming that the cation-cyclization could indeed be induced to proceed despite the higher degree of strain anticipated in the transition state to reach the requisite cis-fused ring system.

The translation of this general plan into a synthesis of the proposed structure for peyssonol A (3) proceeded largely without incident as shown in Scheme 3. Thus, commercially available nerol (29) was advanced into polyene cyclization precursors 30 and 31 in six steps each through a series of previously disclosed transformations (29) details of which can be found in the Supporting Information section. Subsequent exposure of these materials separately to 1.1 equiv of BDSB in nitromethane as solvent afforded access to cis-decalin 32 in 34% yield from 30 and its homologue 33 in 26% yield from 31. Although the efficiency of these transformations is not as high as it was for many of the substrates we explored previously, the strain within the cis-fused transition states leading to 32 and 33 is significantly higher than that for the corresponding trans-fused system.³⁰ In fact, to the best of our knowledge, these cyclizations constitute the first examples of halonium-induced cation-π cyclizations leading to cis-decalin frameworks, with an X-ray crystal structure of 33 (see Supporting Information section) confirming the stereochemical assignment.

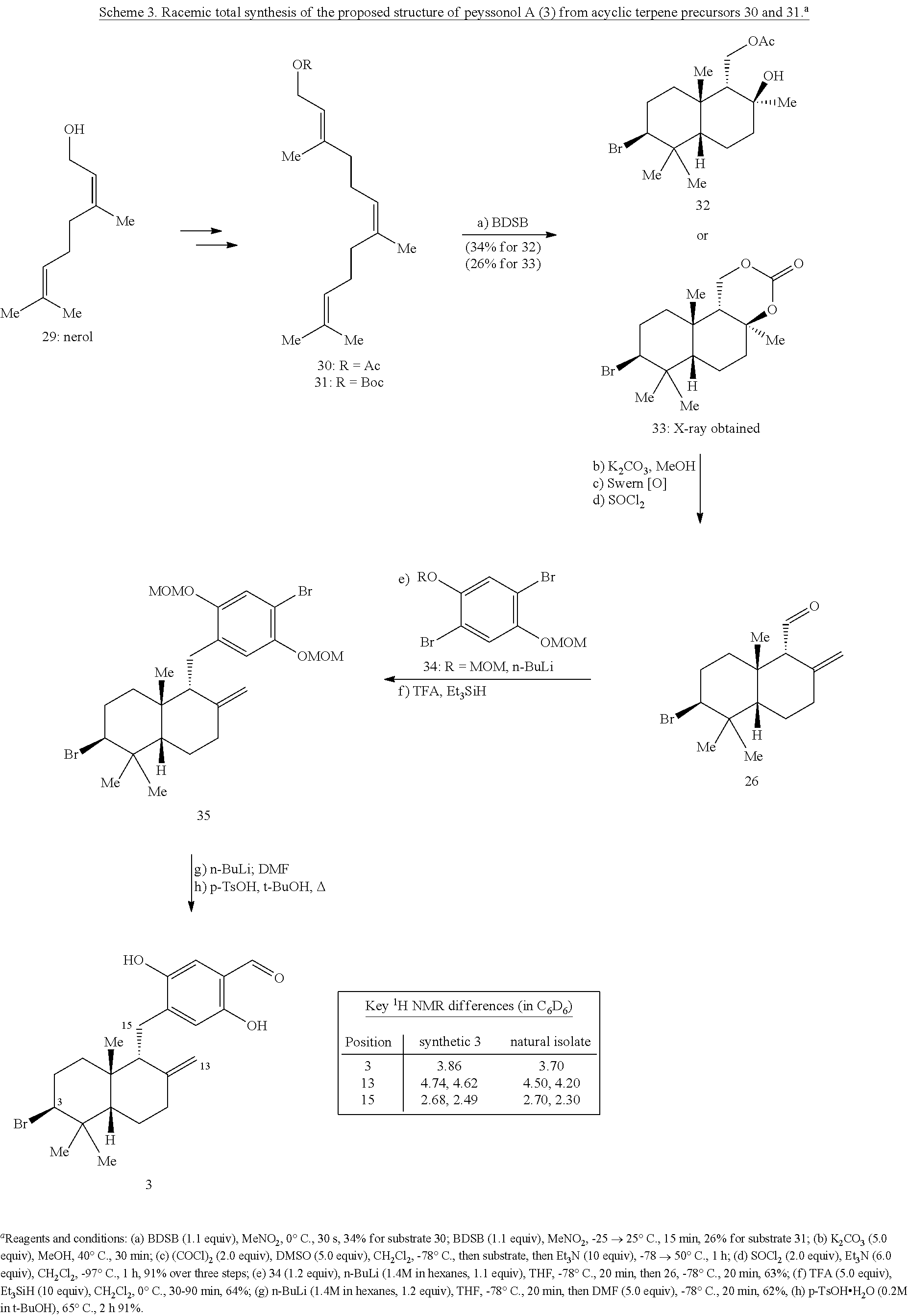

In any event, both 32 and 33 could be funneled into 26 through ester or carbonate hydrolysis as achieved with K₂CO₃ in MeOH, oxidation of the resultant primary alcohol, and regioselective elimination of the remaining tertiary alcohol as achieved with SOCl₂ and Et₃N in CH₂Cl₂ at −97° C. Use of warmer temperatures or a less hindered base (such as pyridine) in this final step led to the formation of significant amounts of the regioisomeric trisubstituted alkene (31). The remainder of the sequence proceeded smoothly as designed, with only 4 additional steps needed to complete a total synthesis of structure 3. To our surprise, however, comparison of the spectral properties of synthetic 3 to those reported for natural peyssonol A revealed stark differences; the inset table within Scheme 3 highlights several key, and readily identifiable, peaks from their respective ¹H NMR spectra. As such, assuming that our stereochemical assignment for synthetic 3 was accurate, the reported structure for peyssonol A (3) would have to be incorrect.

To confirm this hypothesis, especially given the potential for epimerization during the formation or subsequent arylation of aldehyde 26, cis-decalin 40 (see Scheme 4) was synthesized with altered stereochemistry at C-9 (the highlighted center). This compound was readily prepared utilizing the same 8 step sequence, with reduced cation-π cyclization efficiencies noted for conversion of (Z,Z)-isomers 36 and 37 into polycycles 38 and 39 (20% and 28% yield, respectively). More important, however, was that the homologue of aldehyde 26 (cf. Scheme 3) obtained through this sequence had a unique ¹H NMR spectrum, thus suggesting that neither material had been epimerized; all other intermediates were distinct as well. As a result, it was concluded that the ereochemical integrity of our assignments had not been compromised.

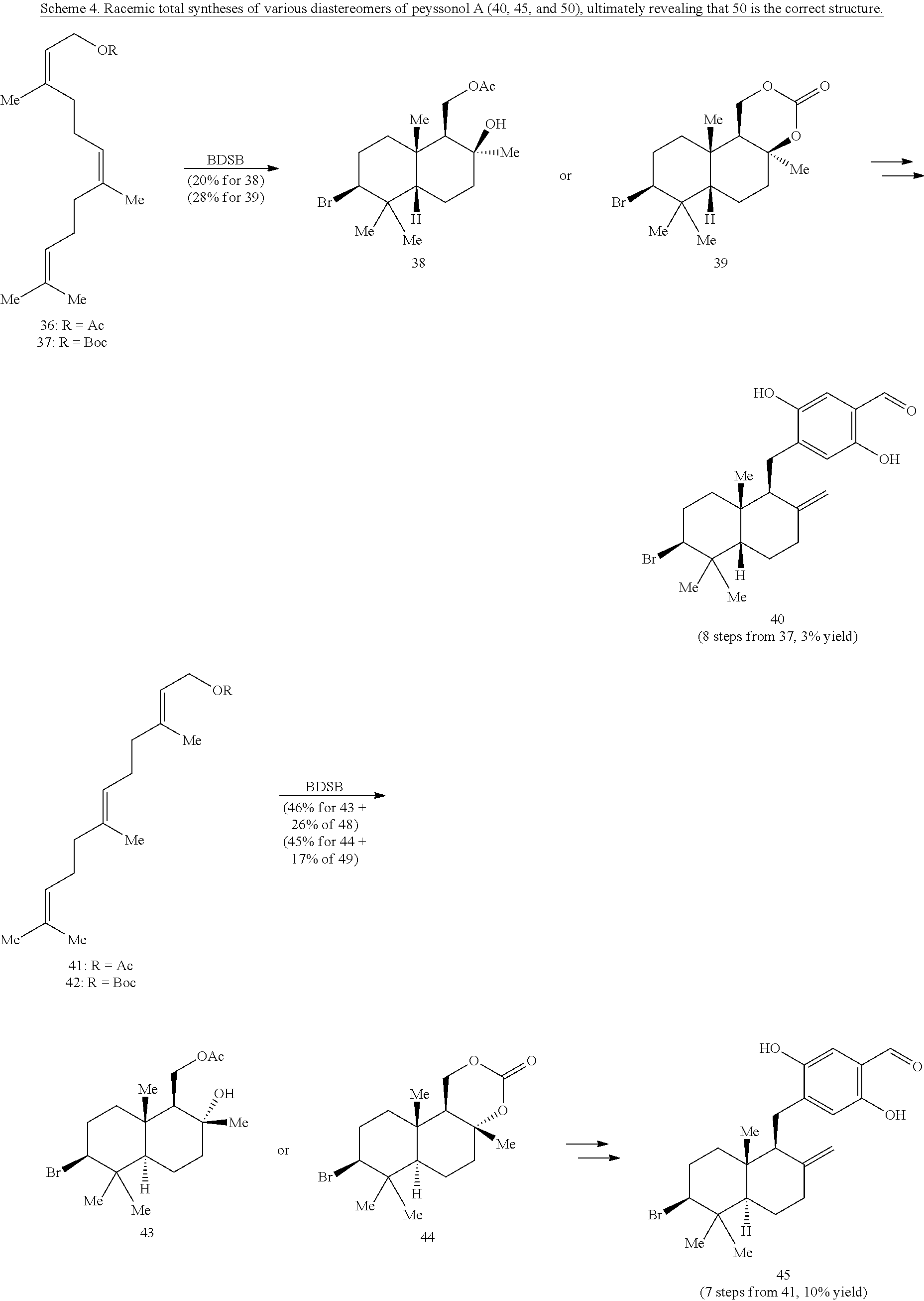

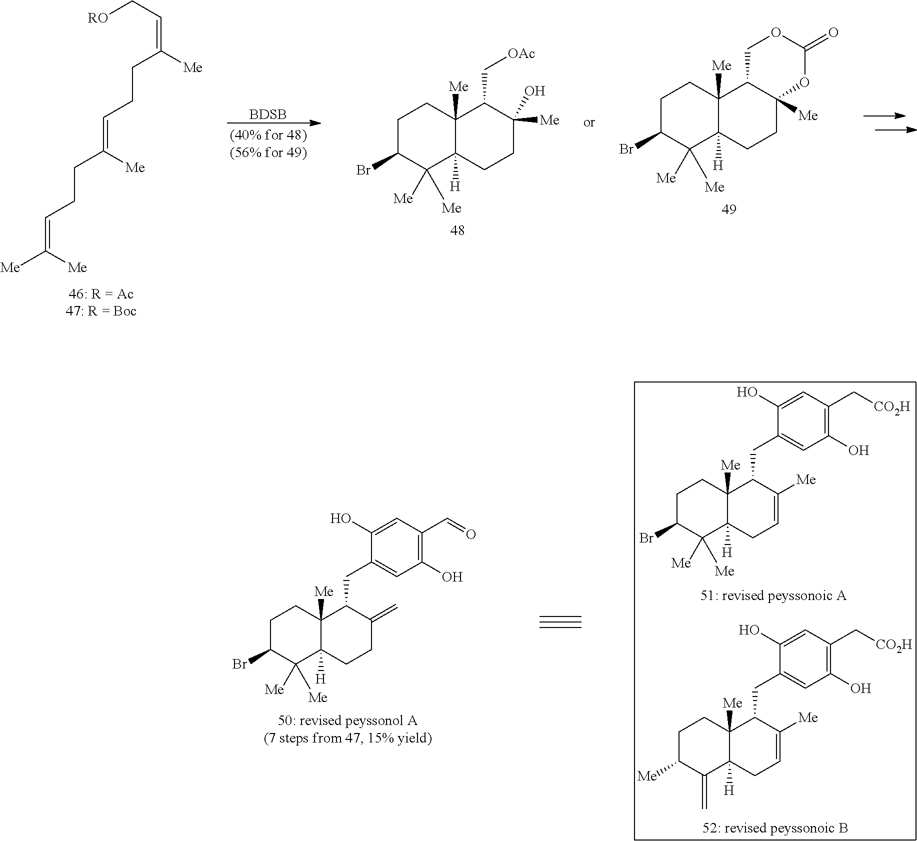

Unfortunately, the spectral data of 40 also did not match those reported for natural peyssonol A. Thus, based on these results, coupled with the fact that no other cis-decalin natural products of this type are known, it was hypothesized that the correct orientation for these rings must include a trans-decalin framework, despite the arguments counter to this analysis presented in the original isolation paper (7c).

Consequently, the two C-9 diastereomers of such a trans-ring fusion (i.e. 45 and 50) were prepared, and fit was ound that compound 50 had nearly identical ¹H and ¹³C spectral data to those published for the natural isolate (32) a crystal structure of this final product was obtained as well, thereby removing any potential ambiguity concerning the stereochemical integrity of our sequence (33). As such, we believe that 50 reflects the true configuration of peyssonol A, a reassignment strengthened by the fact that it matches the carbon framework of peyssonoic acid A (51), a compound which was recently obtained from a related marine alga along with the rearranged framework peyssonoic acid B (52) (34). These materials all possess an uncommon stereochemical configuration at C-9, one which places the large substituent axial; to the best of our knowledge, this synthesis of 50 constitutes a rare example of forming any such framework through an electrophilic-induced polyene cyclization (35). It is also worth noting that the BDSB-induced cation-π cyclization leading to this final structure was the highest yielding of all four diastereomers of t-butyl farnesyl carbonate, with an optimized yield of 56% obtained for tricycle 49. Intriguingly, the (E,E)-farnesol-derived substrates 41 and 42 also provided a fair amount (26% and 17% yield, respectively) of the cation-π cyclization products possessing the axial C-9 orientation of revised peyssonol A (i.e. 48 and 49) in addition to the expected materials (i.e. 43 and 44), thus reflecting a shift in reaction trajectory away from an all-chair conformation (36). A similar switch in selectivity was recently observed by Shenvi and Corey using a differentially protected oxygen-linked termination group in the same position along the carbon framework as 41 and 42 (3m).

Our next efforts sought to achieve additional refinement in the route to 50 to determine whether a sequence could be developed in which the aromatic ring was incorporated prior to cation-π cyclization, since much of the overall step count derived from the post-cyclization incorporation of this unit. It was hoped that such an approach would also enable a total synthesis of peyssonoic acid A (51) to be achieved, assuming that its alternate double bond location relative to peyssonol A could be formed readily and selectively. Scheme 5 presents those endeavors, efforts which were able ultimately to achieve the total synthesis of peyssonoic acid A (51), but not an enhanced preparation of peyssonol A (50).

Our sequence began by adding an allylated form of building block 34 (i.e. 54) onto a (2Z,6E)-farnesyl backbone to forge cation-π cyclization precursor 55. The allyl group was incorporated onto the aromatic ring to enable the eventual generation of the aryl acetic acid moiety of peyssonoic acid A (51) through oxidative cleavage. In addition, however, this monosubstituted double bond would provide a critical test for olefin chemoselectivity in the key BDSB-induced cyclization. Pleasingly, exposure of 55 to BDSB in nitromethane for 5 min at −25° C. afforded materials in which the allyl group remained intact; the isolated yield of 56 was 31%, thereby reflecting a cyclization efficiency of 68% per ring. From 56, the remainder of the sequence occurred smoothly, with the key operation being a terminating exposure to excess BCl₃ in CH₂Cl₂ at −78° C. for 1 h which served to remove the protecting group and cleave the C—O bond at C-8, regioselectively affording the trisubstituted alkene of the target molecule (51) (37, 38). Peyssonoic acid A (51) could also be accessed from polycycle 59 (prepared from 58 in 42% yield with BDSB) through a sequence involving initial lithiation and addition of CO₂ to afford a carboxylic acid that was then homologated via an Ardnt-Eistert sequence; this route, unfortunately, proceeded in significantly reduced yield relative to that of Scheme 5. In no case, however, were we ever able to convert tetracyclic materials like 56 or 59 into exocyclic alkenes, despite numerous attempts. As such, the route described earlier for peyssonol A (cf. Scheme 4) proved to be the only one capable of delivering the desired functionality chemoselectively.

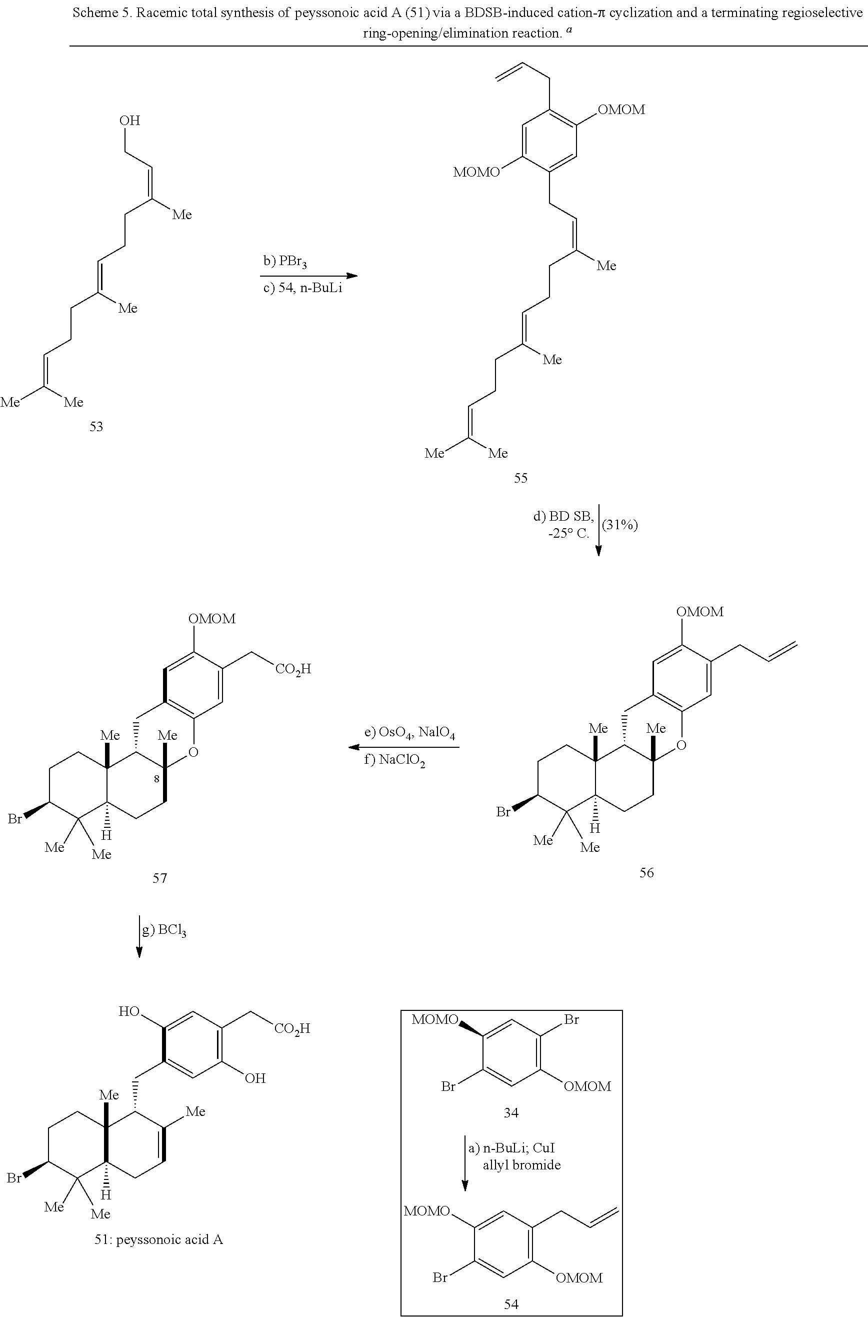

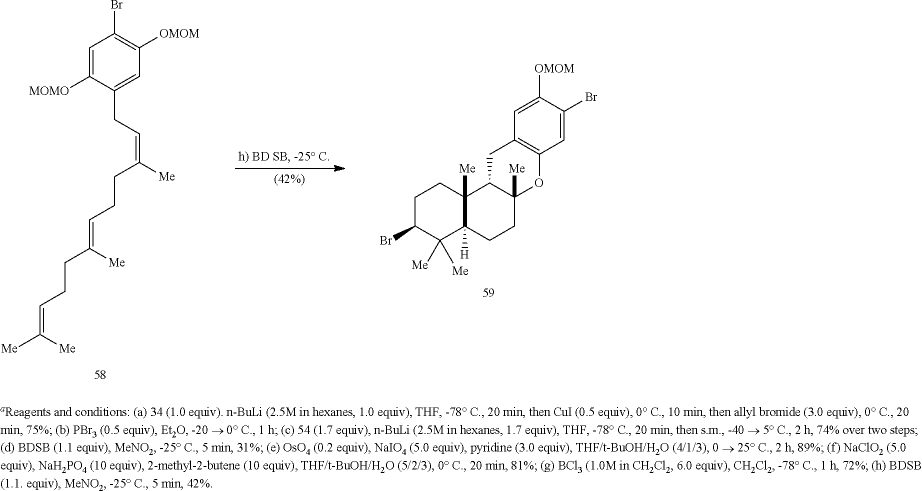

As a final investigation into the power of BDSB to cyclize trienes, we then targeted a formal total synthesis of the natural product aplysin-20 (64, Scheme 6) (39). This unique bicycle was synthesized by Murai and co-workers in 1984 (15b) through a route which employed a Lewis acid-catalyzed polyene cyclization of protected bromohydrin derivative 61, a compound formed in 2 steps from the known nitrile 60 (40). When the key cyclization reaction was conducted with BF₃.OEt₂ in CH₂Cl₂ at reflux for 40 min, polycycles 62 and 63 were obtained in 53% and 14% yield, respectively, from 61. Of these 4 cyclized diastereo- and regio-isomers, only 2 (39% combined yield) had the proper configuration (both —Br and —CH₂CN in equatorial, i.e. β-positions) for the natural product.

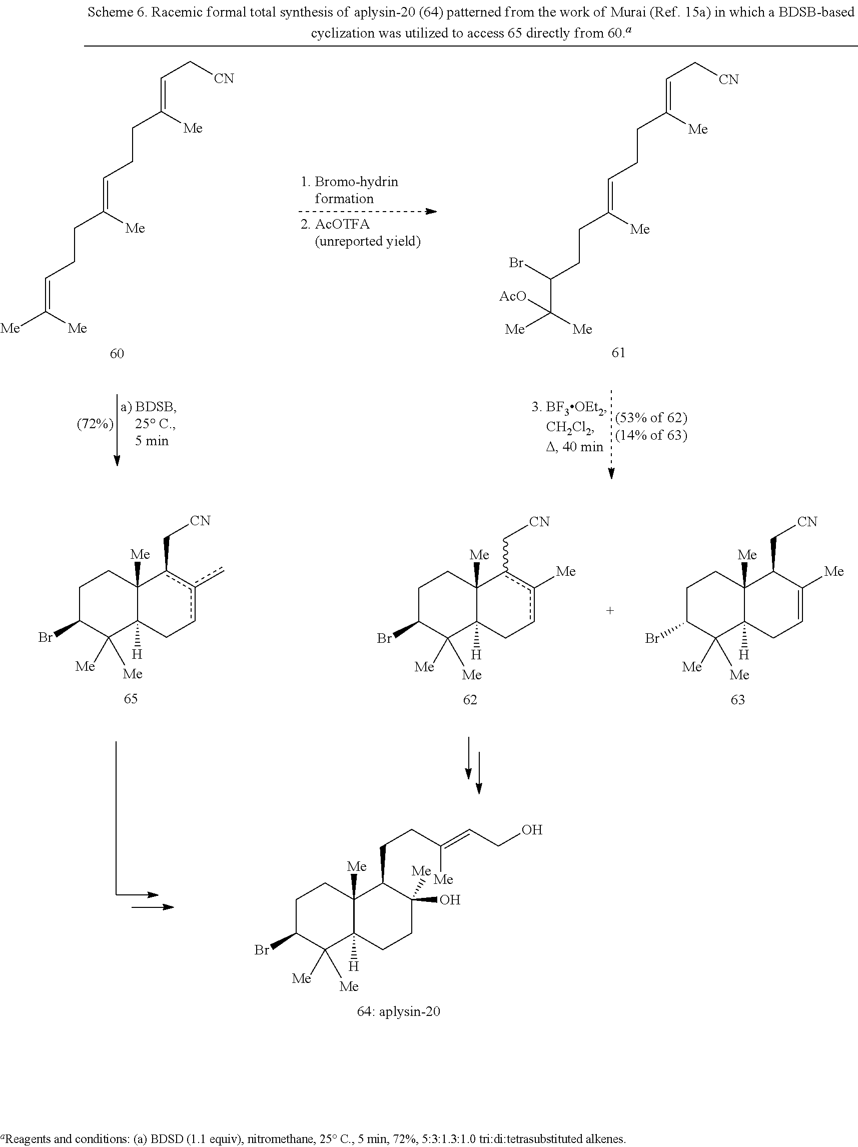

In an effort to render this sequence far more direct, we found that BDSB could convert 60 directly into 65 (as a 5.3/1.3/1.0 mixture of all alkene regioisomers) in 72% isolated yield. When using BDSB, in contrast to Murai's bromoacetate cyclization, stereochemical control was observed at the highlighted center for the di- and tri-substituted alkene forms of 65, indicative of the strong preference for a chair-chair transition state as well as the synchronous nature of this cyclization (2). Thus, all of the cyclized products (65) could formally be advanced to the natural product.

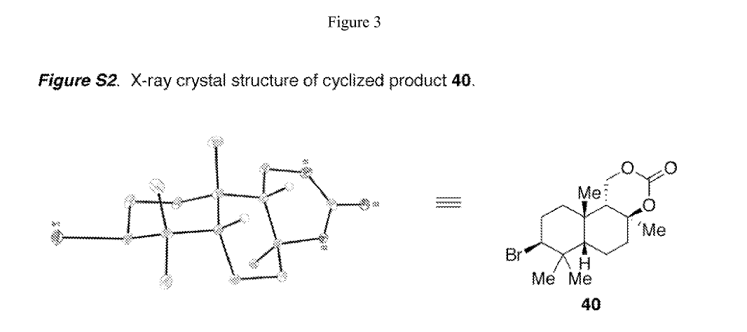

As a concluding comment on the uniqueness of BDSB as a reagent to effect polyene cyclizations, we note that many variants are not as effective overall, either due to challenges in their preparation or their global reactivity. For instance, attempts to prepare aryl variant 66 (FIG. 3) have failed, due entirely to the reagent brominating itself; this problem can be avoided by pre-halogenating the rings to form reagents such as 67, but these materials are not readily solidified or handled. By contrast, carbonyl variants such as 68 and 69 are easily prepared and crystallized but, interestingly, afford reduced stereocontrol in cation-π cyclizations, suggesting that they may react through a different mechanism.

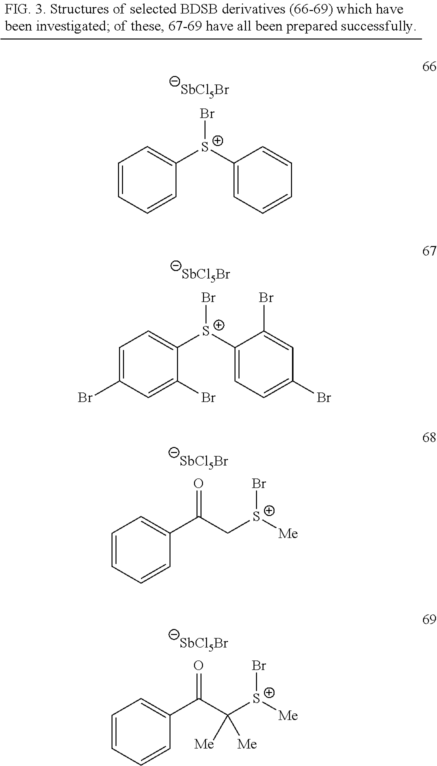

### Example 3

### The Synthesis and Reactivity of IDSI: Application to the Formal Total Syntheses of Loliolide, K-76, and Stemodin

We next sought to determine whether or not a related iodine variant of BDSB could be prepared. After several failed attempts, we were able to synthesize a crystalline form of such a material by combining molecular I₂, Et₂S, and SbCl₅ in 1,2-dichloroethane followed by the addition of hexanes to a saturated solution of the reagent prior to cooling (41). We have termed this material (70, Scheme 7) IDSI on the basis of what we hoped would be reactivity equivalent to BDSB in polyene cyclizations, given that the reagent itself does not possess a structure or level of stability commensurate to BDSB. Indeed, X-ray diffraction revealed that IDSI is actually a chlorine-linked dimer, one whose crystalline form requires a maximum of −20° C. for effective storage; in addition, though the reagent can be weighed normally in air, it will decompose relatively quickly (within 30 min at 25° C.) if not properly attended, losing ICI in the process (42). The inset picture of some needles within Scheme 7 shows this process through the discoloration of the paper on which the solid has been placed. Despite these differences, however, IDSI is quite effective and just as chemoselective as BDSB for initiating polyene cyclizations.

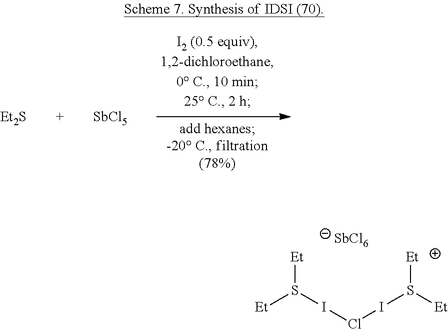

For instance, as shown in Scheme 8, exposure of polyene 71 to 1.2 equivalents of IDSI in nitromethane at −25° C. for 5 minutes at a reaction concentration of 0.05 M afforded polycycle 72 as a single diastereomer in 93% yield. By contrast, neither Ishihara's (12a) nor Barluenga's reagent combinations (12b) were nearly as effective. For instance, in the case of the latter species, we obtained (after multiple attempts using various solvents and differential amounts of added HBF₄) an optimized 41% yield of 72, with other major products being the partially-cyclized product 73, unidentified diastereomers of 72, and proton cyclized 74. Similar results were obtained with NIS/Ph₃P (43). Of course, materials like 73 can be converted into 72 in a subsequent step through the addition of acid; however, IDSI (like BDSB), typically avoids the need for this additional step as an acidic by-product is produced during the course of the cyclization which can complete the sequence effectively in most cases, thereby enabling a more direct and efficient synthetic protocol (44).

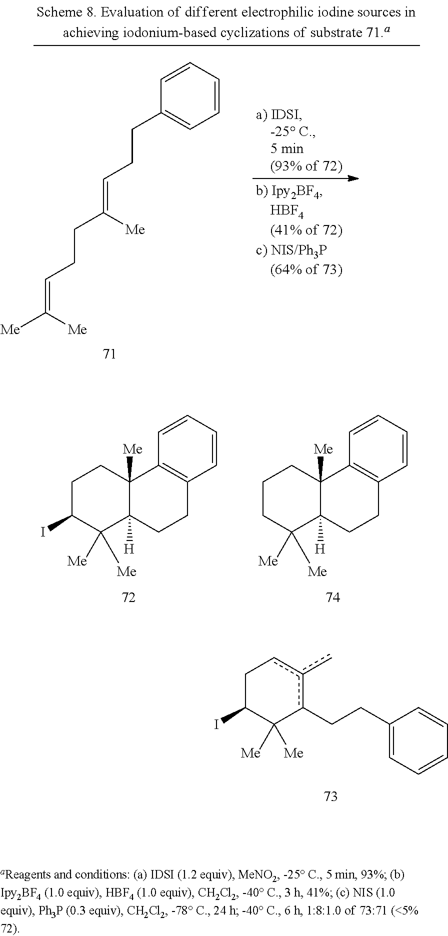

Table 2 provides our preliminary survey of IDSI reactivity with various electron-rich and electron-poor substrates derived from geraniol, farnesol, and nerol, each of which was performed with 0.1 mmol of substrate at a reaction concentration of 0.05 M. In the electron-rich cases (Entries 1-4), cyclization yields were commensurate with those observed previously with BDSB with equally fast reaction times, and only in the case of the conversion of 21 into 78 was an added acid needed at the end of the sequence to achieve complete cyclization. For electron-deficient systems, IDSI also worked well, though the use of various oxygen species to terminate those processes were not as efficient as BSDB (Entries 5-8; the final entry includes a nerol derivative). The main side-product in all of these cases was an uncyclized vicinal chloroiodide such as acetate 86 (formed from attempted IDSI cyclization of 15, Entry 6), revealing that IDSI may have potential as an effective ICI source outside of polyene cyclizations. In any event, it is important to note that Entries 5-8 represent, to the best of our knowledge, the first examples of successful iodonium-based cyclizations of electron-deficient polyenes. All product stereochemistries were established based on comparison to previously synthesized materials and/or literature data.

TABLE 2
Exploration of the generality of direct, iodonium-induced cation-π
cyclizations using IDSI (1.2 equiv) and 0.1 mmol of substrate in nitromethane.

| | | | Temp. | Time | Yield |
|---|---|---|---|---|---|
| Entry | Starting Material | Product | (° C.) | (min) | (%) |
| 1 | 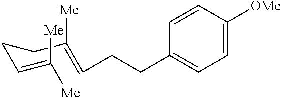 [SMILES: COc1ccc(CC/C=C(\C)CCC=C(C)C)cc1] 17 | 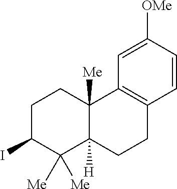 [SMILES: COc1ccc2c(c1)[C@@]1(C)CC[C@H](I)C(C)(C)[C@@H]1CC2] 75 | −25 | 5 | 90 |
| 2 | 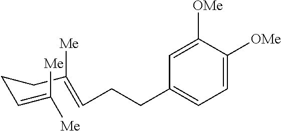 [SMILES: COc1ccc(CC/C=C(\C)CCC=C(C)C)cc1OC] 76 | 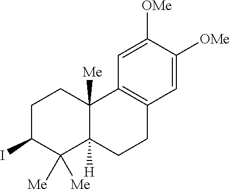 [SMILES: COc1cc2c(cc1OC)[C@@]1(C)CC[C@H](I)C(C)(C)[C@@H]1CC2] 77 | −25 | 5 | 73 |
| 3 | 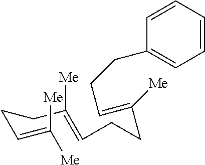 [SMILES: CC(C)=CCC/C(C)=C/CC/C(C)=C/CCc1ccccc1] 21 | 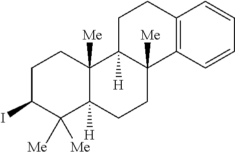 78 | −25 | 30 | 60^{a,b} |
| 4 | 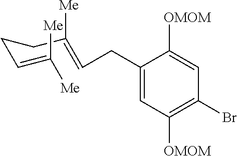 19 | 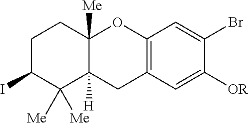 79: R = MOM | −25 | 5 | 85 |
| 5 | 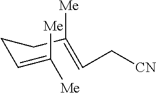 [SMILES: CC(C)=CCC/C(C)=C/CC#N] 8 | 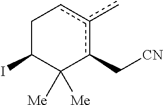 80 | 25 | 5 | 85^{c} |
| 6 | 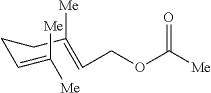 [SMILES: CC(=O)OC/C=C(\C)CCC=C(C)C] 15 | 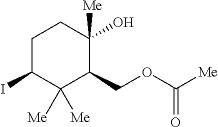 [SMILES: CC(=O)OC[C@H]1C(C)(C)[C@@H](I)CC[C@]1(C)O] 81 | 0 | 1 | 45 |
| 7 | 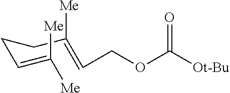 [SMILES: CC(C)=CCC/C(C)=C/COC(=O)OC(C)(C)C] 82 | 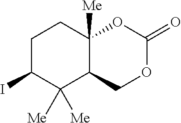 [SMILES: CC1(C)[C@@H](I)CC[C@]2(C)OC(=O)OC[C@@H]12] 83 | 0 → 25 | 30 | 57 |
| 8 | 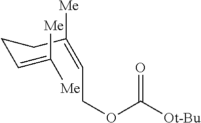 [SMILES: CC(C)=CCC/C(C)=C\COC(=O)OC(C)(C)C] 84 | 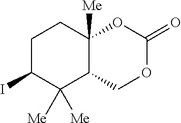 [SMILES: CC1(C)[C@H]2COC(=O)O[C@@]2(C)CC[C@@H]1I] 85 | 0 → 25 | 30 | 48 |

^{a}Isolated as a 2:1 mixture of inseparable stereoisomers about the highlighted carbon atom favoring the drawn diastereomer;
^{b}MeSO₃H (15 equiv) added with 1 h of additional stirring to promote the final cyclization;
^{c}Produced as a 8.5:1.4:1.0 mixture of tri:tetra:disubstituted alkene isomers.

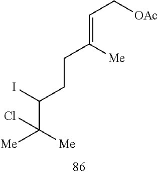 [SMILES: CC(=O)OC/C=C(\C)CCC(I)C(C)(C)Cl]

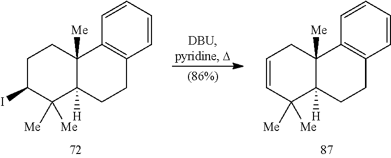

On a global level, however, the true value in a direct and high yielding iodine-based cyclization lies not in forming an iodinated material (as there are no natural products isolated to date resulting from iodonium-induced cation-π cyclizations), but rather the ability to couple, displace, or easily eliminate the alkyl iodide within the product. For instance, we were able to readily form an alkene (i.e. 87) in 86% yield from 72 with DBU in refluxing pyridine; the corresponding bromide is far more robust and does not participate in such chemistry.⁴⁵ As such, it seemed reasonable, given the established cyclization scope and capability for further iodine functionalization, to attempt to utilize IDSI to render more efficient and/or expeditious several previous total syntheses of various non-halogenated natural product polycycles, particularly those cases where stoichiometric amounts of metals were required for success.

For instance, in 1983, Rouessac and co-workers (46) synthesized the natural product loliolide (92, Scheme 9) 47) through a Hg(II)-based polyene cyclization of 88, which, following replacement of the organomercurial with iodine under radical conditions¹⁶ and subsequent elimination, afforded key alkene 91 in 25% overall yield. In our hands, exposure of 88 to 1.2 equivalents of IDSI afforded cation-cyclization product 93 in 79% yield with 19:1 diastereoselectivity at the bridgehead methyl position, while the use of the t-butyl ester-protected variant (89, formed in 68% yield from 88) enabled an IDSI-based synthesis of 93 as a single diastereomer in 88% yield. Subsequent LiCl-induced elimination afforded 91 in 97% yield, thereby accounting for an overall yield of 73% of alkene 91 (an ˜3 fold improvement in fewer steps) from 88, without the use of stoichiometric Hg(II). Similarly, IDSI proved quite effective in our efforts to prepare 96 (Scheme 10), a key intermediate in the McMurry and Erion total synthesis⁴⁸ of K-76 (97)⁴ (49) reported in 1985. In this case, bicycle 98 was prepared in 77% yield using IDSI, illustrating its utility as a powerful cation-π initiator as even the very electron-deficient olefin within 94 participated in this cyclization reaction. Typically, such non-nucleophilic olefins (in this case an α,β-unsaturated ester) do not participate in cation-π cyclizations unless Hg(II) is utilized (1,4). A subsequent elimination using DBU at elevated temperatures provided the requisite alkene 96 in 66% overall yield for the two-step sequence. This outcome compares favorably to the 53% overall yield obtained over the 4 steps of the McMurry and Erion route in which stoichiometric Hg(II) and Se were employed (50). It should be noted that in our hands neither NIS/Ph₃P nor Ipy₂BF₄/HBF₄ was able to fully cyclize the same substrate of Scheme 10 (i.e. 94) in any yield (43).

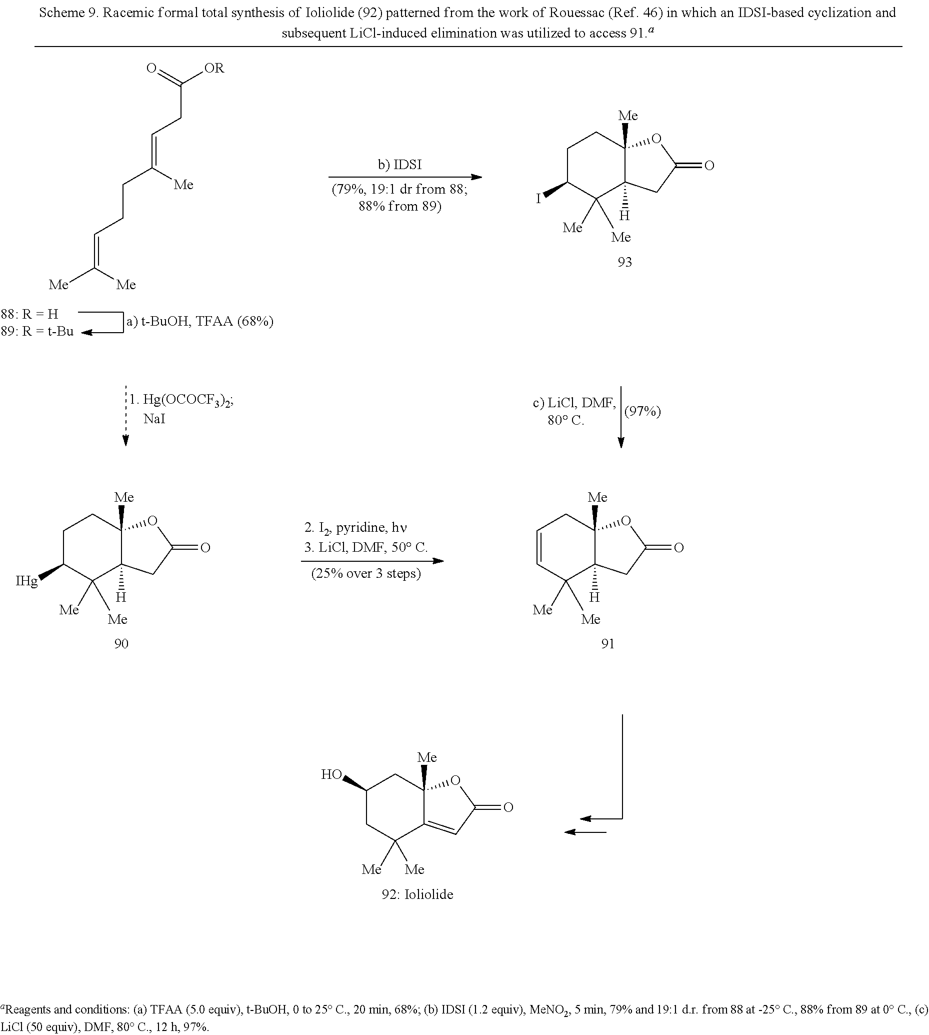

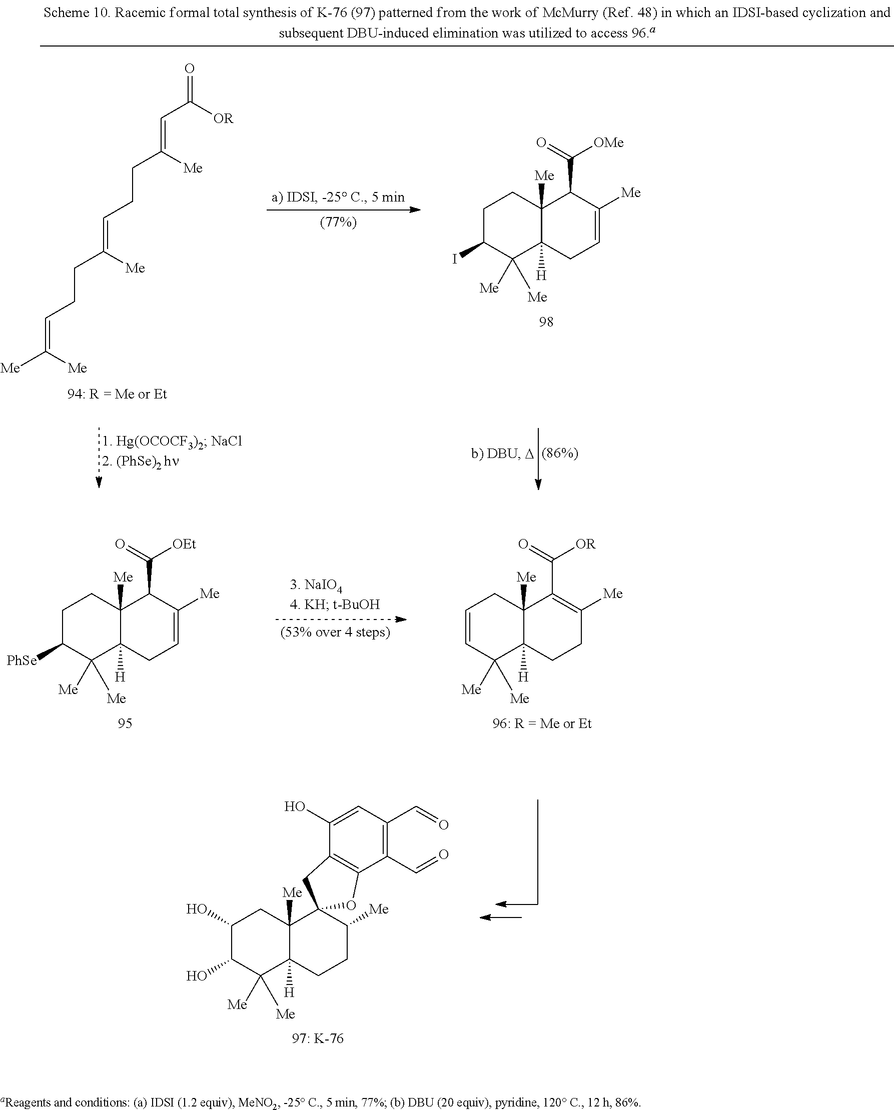

It must be mentioned, however, that Hg(II)-based cyclizations certainly do have merit. For instance, in the Corey total synthesis (51) of stemodin (101, Scheme 11)(52) polyene 99 was smoothly converted into 100 in 60% yield via treatment with Hg(OCOCF₃)₂ to effect the cyclization followed by replacement of the intermediate organomercurial with iodine (53). IDSI was able to form similar materials from 99, but in reduced yield as the predominant products obtained were partially-cyclized. In our hands, only a portion of these could only be successfully converted into 102 through the use of an acid-promoted cyclization (concentrated H₂SO₄ in toluene) in a separate step; extensive efforts to differentially functionalize the enol ether in 99 (including groups such as a methyl-, methoxymethyl-, and various silyl-enol ethers) afforded no improvement above the 40% yield indicated within Scheme 11. Thus, in this case, the overall yield of the polycycle was not superior through the use of IDSI, though the toxic metal species used for the polyene cyclization could still be avoided.

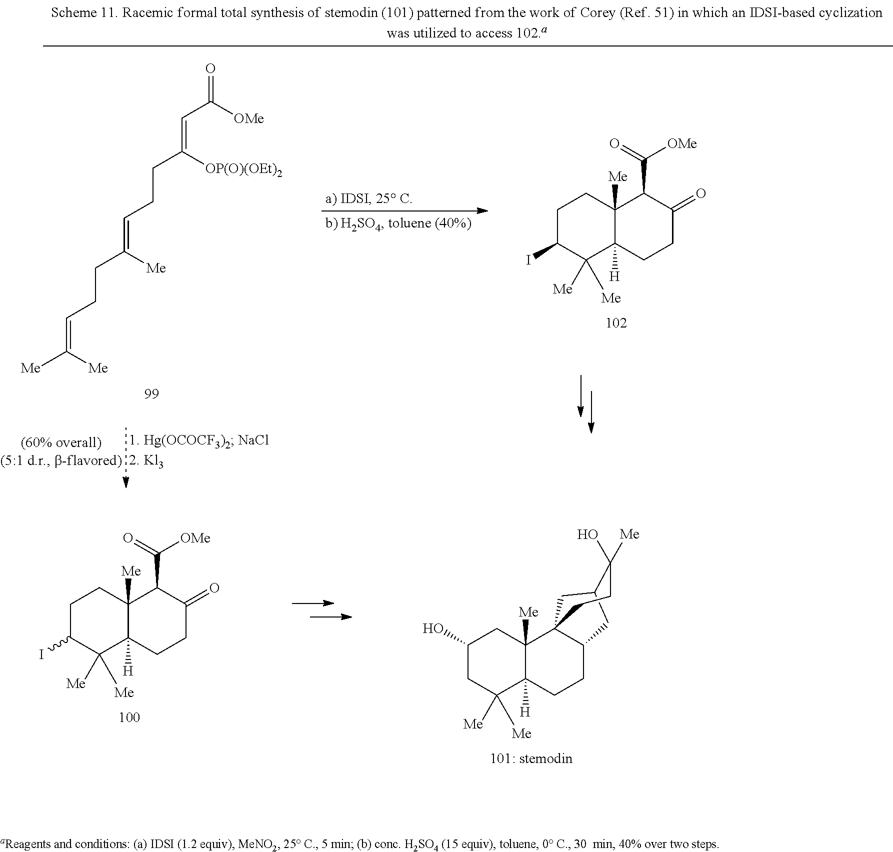

### Example 4

### The Synthesis and Reactivity of CDSC

We next sought to determine if direct, chloronium-induced cyclizations could be achieved with a reagent of the general design of BDSB and IDSI. The synthesis of our test reagent, a derivative of a previously reported Me₂S variant (54) which we name CDSC (chloro diethylsulfonium hexachloroantimonate, 103), is shown in Scheme 12.

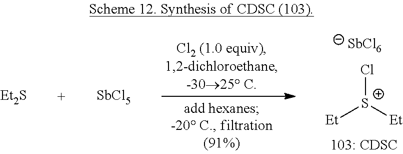

Similar to BDSB and IDSI, this compound is a crystalline solid that is stable at −20° C. for at least several months and can be handled and weighed in air. As indicated in Table 3, polyene cyclizations of various materials possessing differential electron wealth were undertaken with CDSC, all at a reaction concentration of 0.05 M. Though the resultant product yields are not nearly as high as those observed with BDSB and IDSI for the same substrates, these entries represent, to the best of our knowledge, the first examples of effecting chloronium-induced polyene cyclizations in any yield via an ionic pathway (10). Of note, these cyclizations do not, for the most part, possess diastereocontrol, perhaps reflecting a global challenge in reactivity due to greater tertiary carbocation rather than bridged chloronium-character in the initial reactive intermediate (as indicated by the structures at the bottom of Table 3) (55).

TABLE 3
Exploration of the generality of direct, chloronium-induced cation-π cyclizations
using CDSC (1.1 equiv) and 0.1 mmol of substrate in nitromethane.

| | | | Temp. | Time | Yield |
|---|---|---|---|---|---|
| Entry | Starting Material | Product | (° C.) | (min) | (%) |
| 1 | 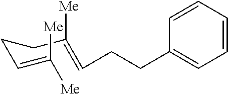 [SMILES: CC(C)=CCC/C(C)=C/CCc1ccccc1] 71 | 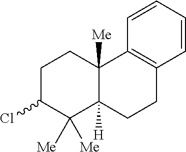 [SMILES: CC1(C)C(Cl)CC[C@]2(C)c3ccccc3CC[C@@H]12] 104 | −25 | 5 | 46^{a} |
| 2 | 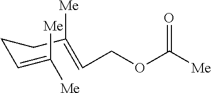 [SMILES: CC(=O)OC/C=C(\C)CCC=C(C)C] 15 | 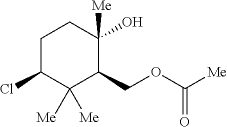 [SMILES: CC(=O)OC[C@H]1C(C)(C)[C@@H](Cl)CC[C@]1(C)O] 105 | 0 | 1 | 18^{b} |
| 3 | 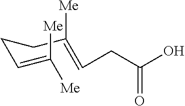 [SMILES: CC(C)=CCC/C(C)=C/CC(=O)O] 88 | 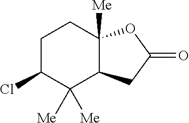 [SMILES: CC1(C)[C@@H](Cl)CC[C@]2(C)OC(=O)C[C@@H]12] 106 | −25 | 5 | 38^{c} |
| 4 | 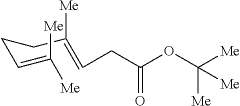 [SMILES: CC(C)=CCC/C(C)=C/CC(=O)OC(C)(C)C] 89 | 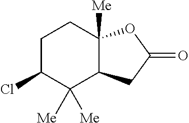 [SMILES: CC1(C)[C@@H](Cl)CC[C@]2(C)OC(=O)C[C@@H]12] 106 | 0 | 5 | 20^{c} |

^{a}Isolated as a 1.0:1.0 mixture of inseparable stereoisomers;
^{b}Isolated as a 2.2:1.0 mixture of separable diastereoisomers at the highlighted carbon favoring the drawn product;
^{c}Produced as a 4.0:1.0 mixture of separable diastereomers at the highlighted carbon favoring the drawn product.

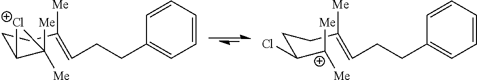 [SMILES: C/C(=C\CCc1ccccc1)CCC1[Cl+]C1(C)C ; C/C(=C\CCc1ccccc1)CCC(Cl)[C+](C)C]

### Example 5

### Efforts Towards Asymmetric Induction

Finally, we desired to prepare chiral versions of CDSC, BDSB, and IDSI in a preliminary investigation of their potential to achieve asymmetric versions of the reactions described above. Although several chiral sulfides are known, we focused our attention on materials with C₂-symmetry (56) using a sequence involving an enzymatically-controlled step to synthesize (2R,5R)-(+)-2,5-dimethylthiolane for reagents 107, 108, and 109 (Scheme 13, all putative structures) (57). Unfortunately, all endeavors with these, and related compounds, afforded no asymmetry in the cation-π cyclization of substrate 71, though they all led to the formation of the expected racemic products. Interestingly, however, with reagent 107 we were able to add Cl₂ across the double bond of 111 with some enantioselection (up to 14% e.e.) (58) initial screens have shown that solvent is a critical factor in the efficiency of this process, suggesting that further refinement may enable improvement on this preliminary finding. It is important to note that the reagent formed with the omission of SbCl₅ did not afford any 112, indicative of the importance of the normally inert SbCl₆ counterion. Explorations seeking to build upon these initial results are the subject of current endeavors.

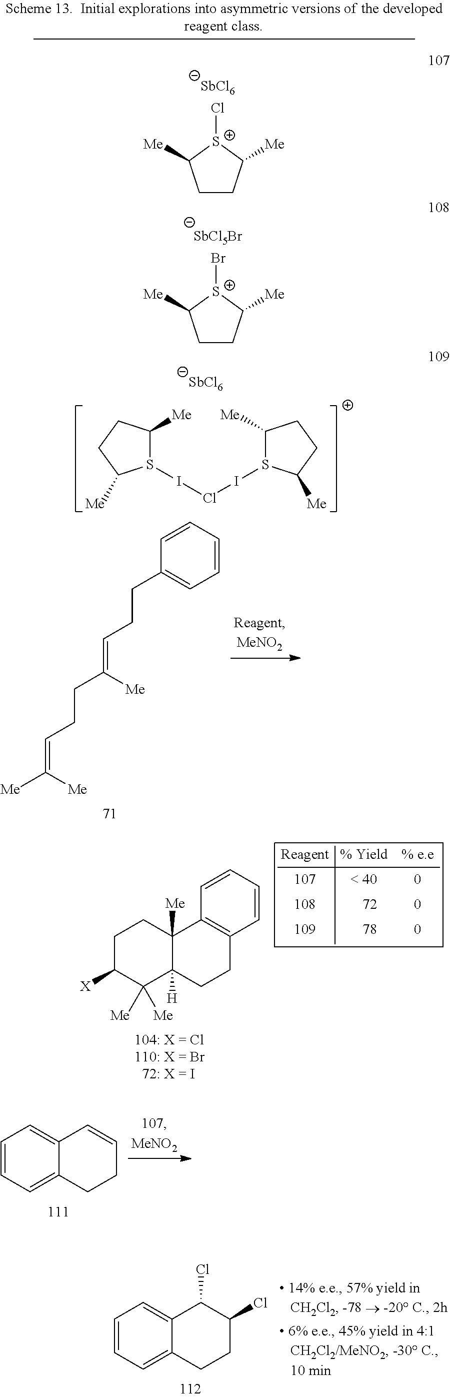

### Example 6

### Ring-Forming Halolactonization: Synthesis of Heimol a and Hopeahainol D

In 2001, Weber and co-workers reported their isolation and characterization of an architectural challenging natural product in the form of the oxidized resveratrol dimer 116 (59). This compound, which they named heimiol A, after its plant source (Neobalanacarpus heimii), merges one six-membered and two seven-membered ring systems into a [3.2.2] bicycle that displays four chiral centers, and it has since been shown to possess some antioxidant activity (60).

In addition to polyene cyclizations, these reagents (CDSC, BDSB, and IDSI) also appear to have potential to effect transformations that other electrophilic halogen sources cannot readily achieve. As shown in Scheme 14, a complex IDSI-promoted halolactonization cascade (113 to 114) was used to access the core of natural products heimiol A (116) and hopeahainol D (115); here, IDSI was the only stoichiometric reagent which accomplished this transformation. The yield was 36% following global phenol deprotection of the lactone product (i.e. 114). Conditions which generated iodonium in situ provided some yield of 114 (see inset box), but not as efficiently or easily as IDSI.

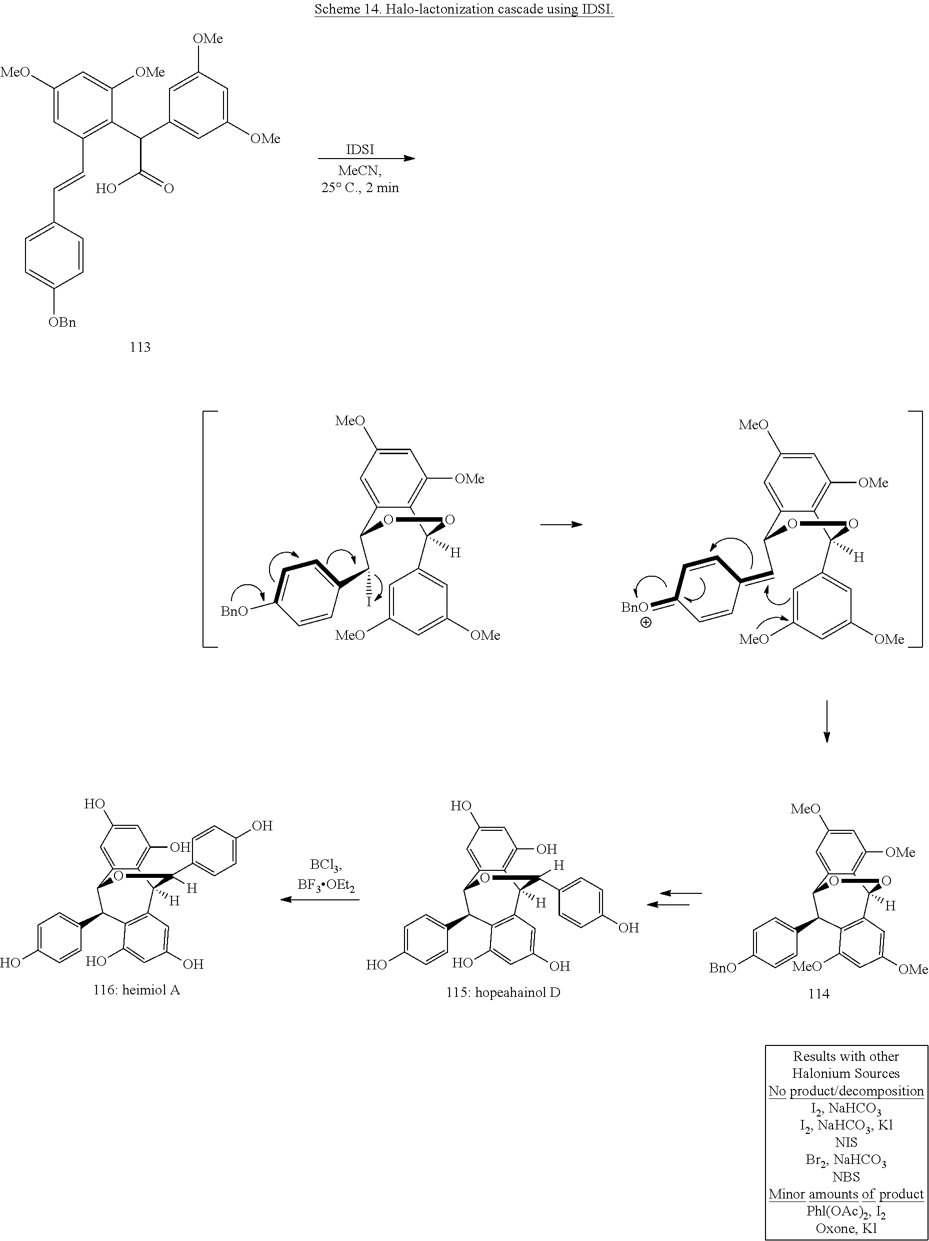

### Example 7

### Ring Expanding Bromoetherification: Preparation of 8- and 9-Membered Laurencia-Type Bromoethers

Some of the most fascinating halogenated natural products are the Laurencia C15 acetogenins, of which the inaugural member, laurencin (117, Scheme 15), was first reported by Irie and co-workers in 1965 (61, 62, 63). Since then, more than 140 members have been isolated from marine alga, most containing a cyclic bromoether core ranging in size from 4- to 12-membered (64). The lauroxocanes (including 117-120) possess an 8-membered ring system, and represent the largest subset of the family. These medium-ring bromoethers, encompassing more than 50 natural products, have elicited much attention not only for the synthetic challenges they provide, but also for the general question of their biogenesis.

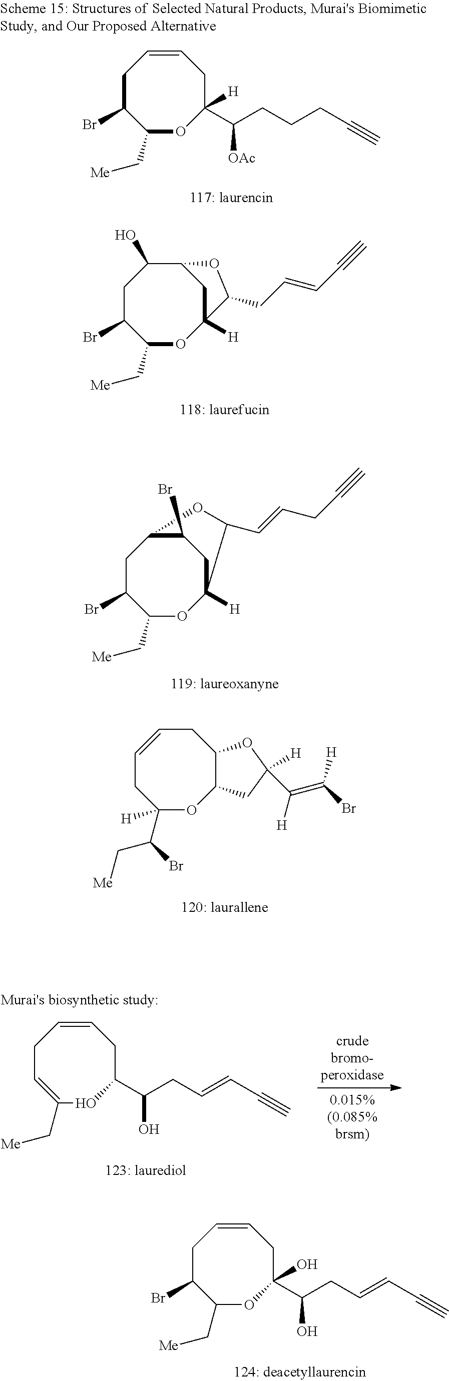

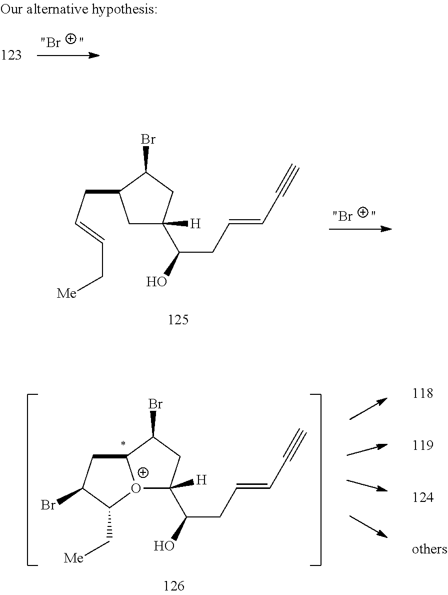

The Murai group first showed that these rings could arise via bromoperoxidase-catalyzed intramolecular bromoetherifications of linear precursors (as in 123→124) (65). The incredibly low yield of product observed, however, may imply that direct 8-endo cyclization of precursor 123 is an unfavorable event, even within the confines of an enzyme pocket which could preorganize the substrate (66). As such, we wondered these challenging domains could also arise via a series of two potentially more favorable 5-membered ring-forming steps. Specifically, if 5 underwent an initial 5-endo bromoetherification to form 125, a second bromoetherification using the tetrahydrofuran oxygen as nucleophile might then lead to a bicyclic oxonium intermediate (i.e. 126) (67). Such a material could then lead to lauroxocane natural products (118, 119, 124, and others) via reactions at the starred carbon, such as neighboring group participation, intramolecular cyclization, external nucleophile attack, and/or elimination (68, 69). Although this exact hypothesis has not, to the best of our knowledge, been published before, ring-expansions through oxonium formation have been demonstrated experimentally by Braddock for the formation of the 12-membered ring obtusallenes and related bicyclic marilzabicycloallenes in moderate yield (70, 71).

Additionally, Kim and co-workers (72) have published the opposite perspective on this idea: the tricyclic oxonium ion derived from a ring-contraction of the oxocane prelaurefucin could lead to two distinct tetrahydrofuran-containing natural products (73). The key challenge, however, is translating these ideas into practical and controlled laboratory syntheses of single members. Perhaps for this reason, none of the published total syntheses of lauroxocanes (63, 74) have forged their medium-sized cores through a direct bromonium-induced reaction (75). In this communication, we show that with the use of the proper brominating reagent and appropriately designed substrates, ring-expansion of oxonium species akin to 8 can, in fact, lead to selective and stereocontrolled laboratory syntheses of diverse 8- and 9-membered bromoethers (both exo and endo) resembling the Laurencia C15 acetogenins.

Our first insight that a ring-expansion process could afford 8-membered rings derived from the discovery that hydroxytetrahydrofuran 127 (Scheme 16) was converted into rearranged ketone 119 rather than bromoether 128 (a model compound resembling 118) upon exposure to BDSB (76). Although not an 8-membered ring product, its presumed formation through the indicated bicyclic oxonium formation-hydride shift process (77) suggested the key materials needed for a controlled ring-expanding bromoetherification. Specifically, if the alcohol of 9 was moved to the 4-position of the tetrahydrofuran ring and appropriately protected as an ester or carbonate (as in 130), then a similar rearrangement terminated by an internal ring-opening of the bicyclic oxonium ion (i.e. 131) (78) could yield an 8-exo (laurenan-like) (79) bromoether with differentiated oxygen functionalities on the ring (i.e. 132 or 133) (80). Application of the same idea to a substrate with one less methylene unit between the tetrahydrofuran ring and the alkene (i.e. 134) could afford the corresponding 8-endo (lauthisan-like)(79) materials (i.e. 136 and 137). Critically, if the process was fully stereocontrolled for all possible variants of these compounds, then all lauroxocane cores could be predictably accessed, one at a time.

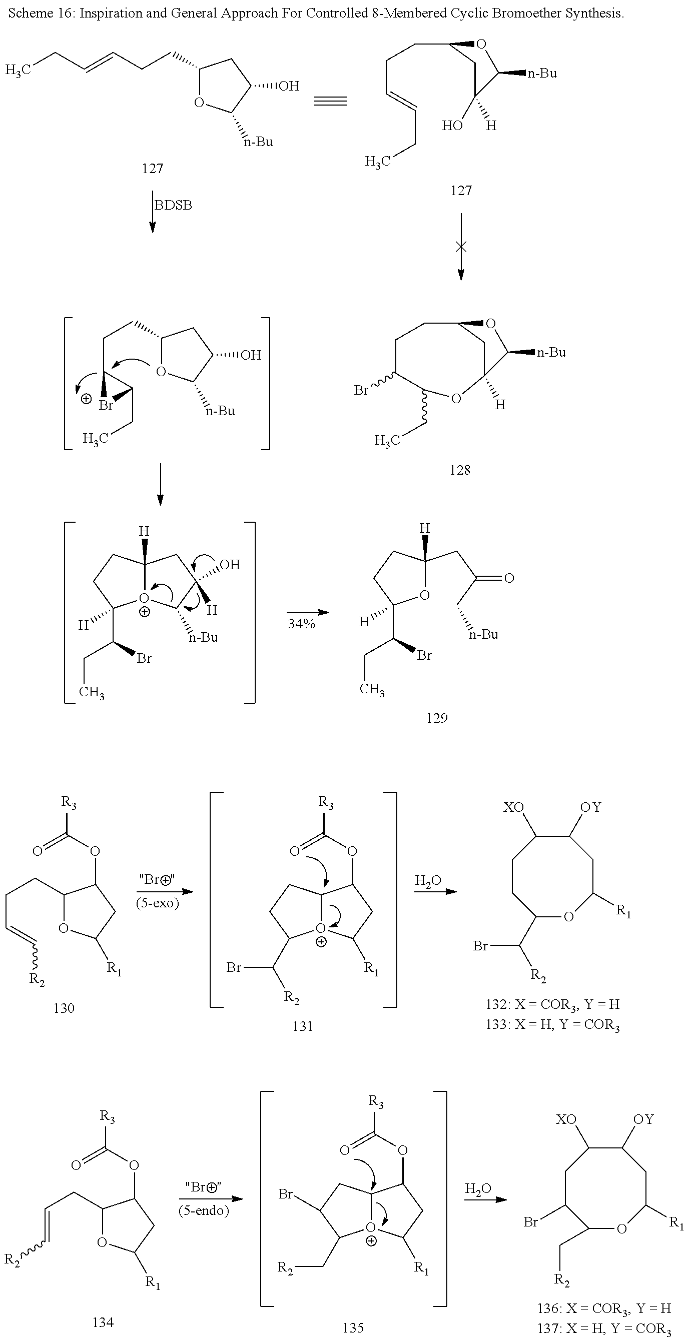

Our studies began with several variants of model compound 138 (Scheme 17), prepared readily through the approach delineated in a recent paper by Britton and co-workers (81). Although attempted cyclization of free alcohol variant (138a) failed to produce any ring-expanded ketone, exposure of the acetylated version (138b) to 1.2 equivalents of BDSB for 5 minutes at −25° C. yielded the desired 8-exo bromoether as a 3.6:1 mixture of acetate regioisomers (i.e. 139 and 140) in 74% yield. Significantly, this reaction process was both stereo- and regioselective, indicating that it proceeded through only one of two facially-distinct bromonium ions and exclusively with 5-exo attack by the tetrahydrofuran oxygen (not the 6-endo alternative). Since the alkene is significantly removed from the chirality of the tetrahydrofuran ring, a likely possibility is that both faces are accessible, but ultimately the more reactive bromonium ion is accessed by bromonium transfer processes to funnel to the observed single diastereomer (82). Molecular models accounting for the exclusive formation of a single diastereomer are drawn in Scheme 18; for steric reasons, it is likely that the brominated side chain of the oxonium species prefers an exo rather than endo orientation with respect to the concave oxonium ion intermediate.

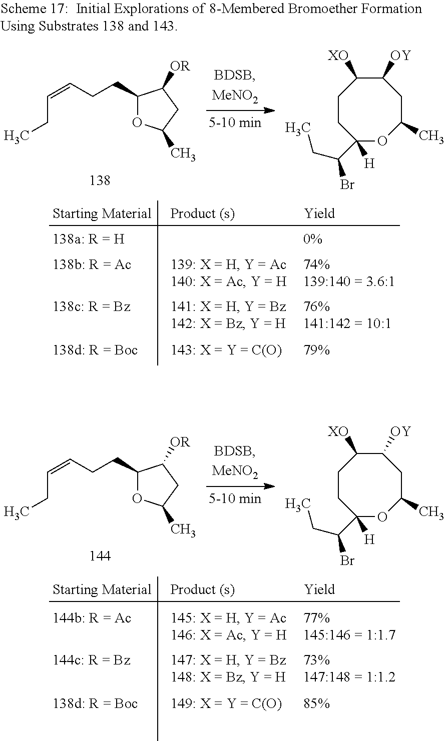

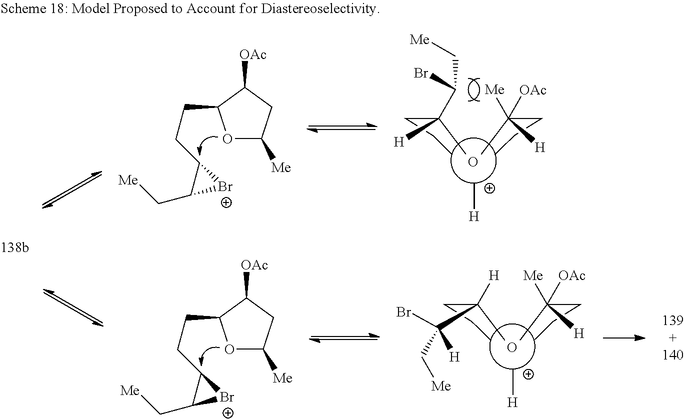

From a practical standpoint, however, the acetylated products proved difficult to handle due to facile migration of their acetate groups (i.e. 139 [symbol] 140). Pleasingly, the benzoate congener (138c) solved this problem (83) and led to higher regiochemical differentiation, affording a 10:1 mixture of separable 141 and 142 in 76% combined yield. Hydrolysis of these materials to the diol followed by rebenzoylation afforded predominantly 142 (6.6:1 ratio of 142:141 in 90% yield), allowing access to either monobenzoylated regioisomer in good yield. In the interest of affording only a single product, the t-butoxycarbonyl (Boc) variant 138d smoothly underwent ring-expansion to carbonate 143 in 79% yield. In addition to varying the identity of the ring-opening group, we also altered its stereochemistry with differentially protected substrate 144. We were delighted to find that all variants afforded diastereomeric 8-exo bromoethers with similarly good yields. The relative stereochemistry of 139-143 and 145-149 were confirmed by X-ray diffraction of their crystalline diol derivatives. Worth noting is that the efficiency of the cyclizations was dependent upon the bromonium source used. While BDSB provided the optimal yield for the synthesis of 143, two more conventional reagents proved less competent [TBCO (2,4,4,6-tetrabromo-2,5-cyclohexadienone) and (coll)₂BrOTf afforded 62% and 52% yield of 143, respectively], while NBS gave less than 10% of the desired product, even after 48 h (the use of N,N-dimethylacetamide as a nucleophilic promoter failed to enhance this yield) (84).

To explore the scope of the rearrangement and evaluate the diastereocontrol needed to access the entire range of lauroxocane natural products selectively, we next examined 7 analogues of 138d that systematically varied the relative stereochemistry of their C2- and C5-alkyl groups and the position and E/Z-stereochemistry of the alkene. All substrates possessed Boc groups for the convenience of affording a single product and were cyclized using BDSB. Although all bromoethers in this study were prepared without regard for absolute stereochemistry, each of these syntheses could be rendered asymmetric using the same protocol (81).

As shown in Table 4, three stereoisomers of 138d (150, 152, and 154) stereoselectively afforded the expected 8-exo bromoether products after only 10 min of reaction time. Of the analogous substrates shortened by one methylene unit (156, 158, 160, and 163), however, only E-alkenes 156 and 158 underwent ring-expansion to 8-endo bromoether products. The two cis-disposed starting materials (160 and 163) instead gave bicycles 162 and 165 as the predominant products (Scheme 19). It is well-documented that 5-endo haloetherifications are often significantly slower with Z-alkene substrates (85); here, that suggests substrates 160 and 163 failed due to side reactions achieving competitive reaction rates. For example, the Boc group may have been deprotected under the acidic conditions (BDSB is a Lewis acid at both sulfur and bromine, and could react with trace amounts of water to form HBr and HSbX₆; HSEt₂⁺ could also be generated), thereby enabling the resulting alcohol to attack the bromonium intermediate preferentially. This hypothesis is supported by the observation that BDSB cyclization of the unprotected alcohol precursors to 160 and 163 produced 162 and 165 in nearly quantitative yield. Despite the failure of these substrates, it is worth noting that the desired products from these events (i.e. 161 and 164) have the same stereochemical relationship as only one known Laurencia natural product. By contrast, the other 6 frameworks produced model at least 28 different isolates as well as one core (i.e. 143) that has not been observed in nature. As such, these collated results illustrate the potential power of the approach for controlled lauroxocane laboratory synthesis through a direct bromonium-induced process.

TABLE 4
Exploration of Ring-Expanding Bromoetherification

| | | | | Laurencia natural |
|---|---|---|---|---|
| Entry | Starting Material | Product | Yield (%) | product skeletons |
| 1^{a} | 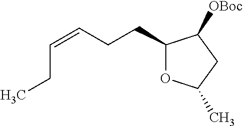 150 | 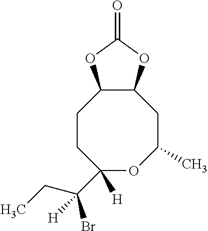 [SMILES: CC[C@H](Br)[C@@H]1CC[C@H]2OC(=O)O[C@H]2C[C@H](C)O1] 151 | 84 | 2 |
| 2^{a} | 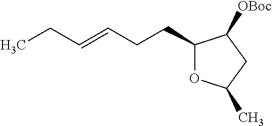 152 | 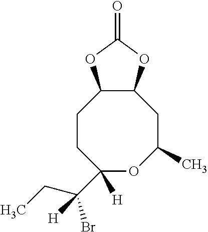 [SMILES: CC[C@@H](Br)[C@@H]1CC[C@H]2OC(=O)O[C@H]2C[C@@H](C)O1] 153 | 60 | 10 |
| 3^{a} | 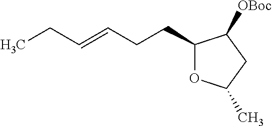 154 | 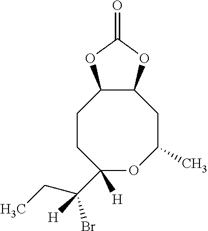 [SMILES: CC[C@@H](Br)[C@@H]1CC[C@H]2OC(=O)O[C@H]2C[C@H](C)O1] 155 | 83 | 1 |
| 4^{b} | 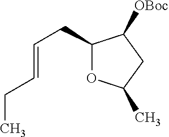 156 | 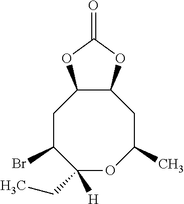 [SMILES: CC[C@H]1O[C@H](C)C[C@@H]2OC(=O)O[C@@H]2C[C@@H]1Br] 157 | 68 | 5 |
| 5^{b} | 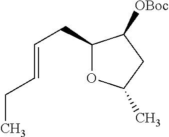 158 | 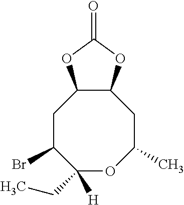 [SMILES: CC[C@H]1O[C@@H](C)C[C@@H]2OC(=O)O[C@@H]2C[C@@H]1Br] 159 | 67 | 10 |

Conditions:
^{a}0.1 mmol substrate, 1.2 eqiv BDSB, 0.02M in MeNO₂, 10 min (−25 to 25° C.)
^{b}1.5 equiv BDSB, 20 min (−25 to 25° C.)

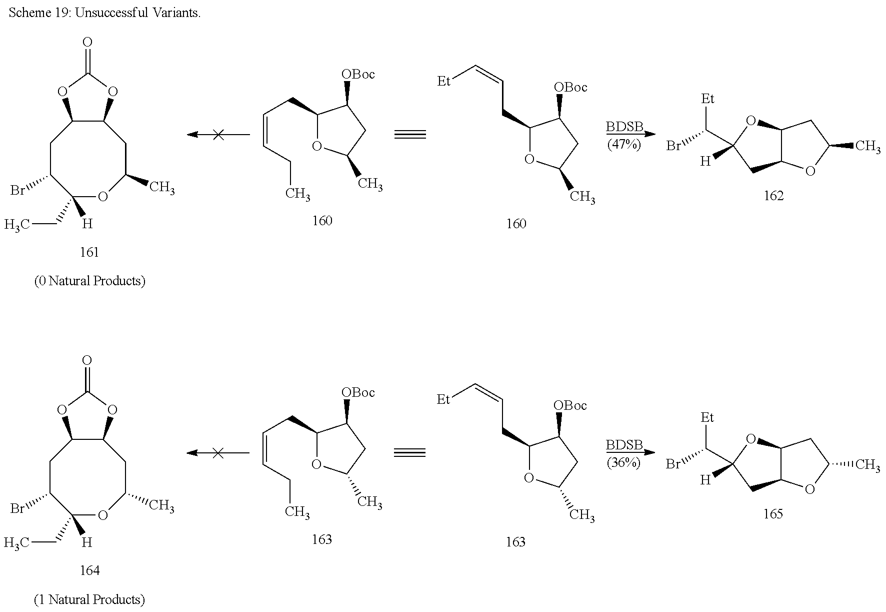

As a final exploration of reaction scope for this study, we evaluated its feasibility for 9-membered ring formation, as at least 10 naturally occurring lauroxonanes (9-membered bromoethers) have been isolated and characterized to date. Pleasingly, both substrates investigated thus far (i.e. tetrahydrofuran 116 and tetrahydropyran 168, Scheme 20) led to the expected products upon reaction with BDSB, ultimately yielding one example each of a 9-exo and 9-endo product (i.e. 167 and 169). We expect that other diastereomeric 9-membered products, and potentially even larger cyclic bromoethers, could arise from similar processes.

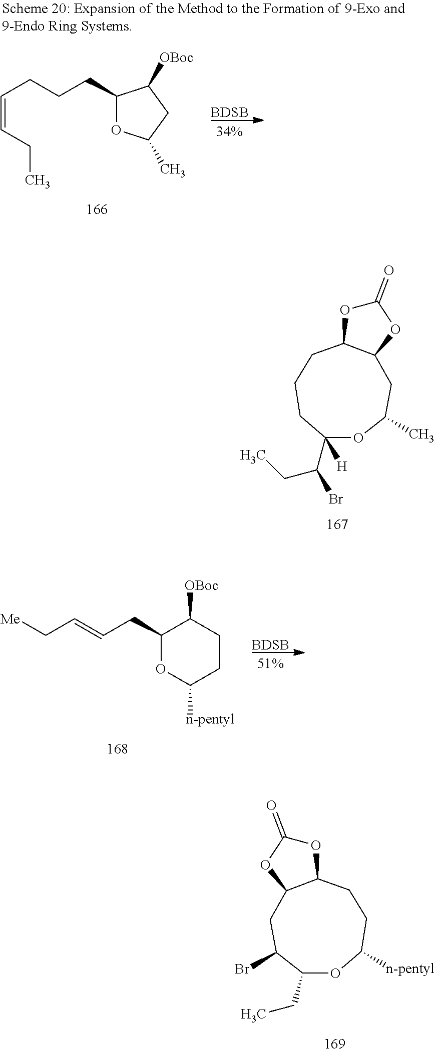

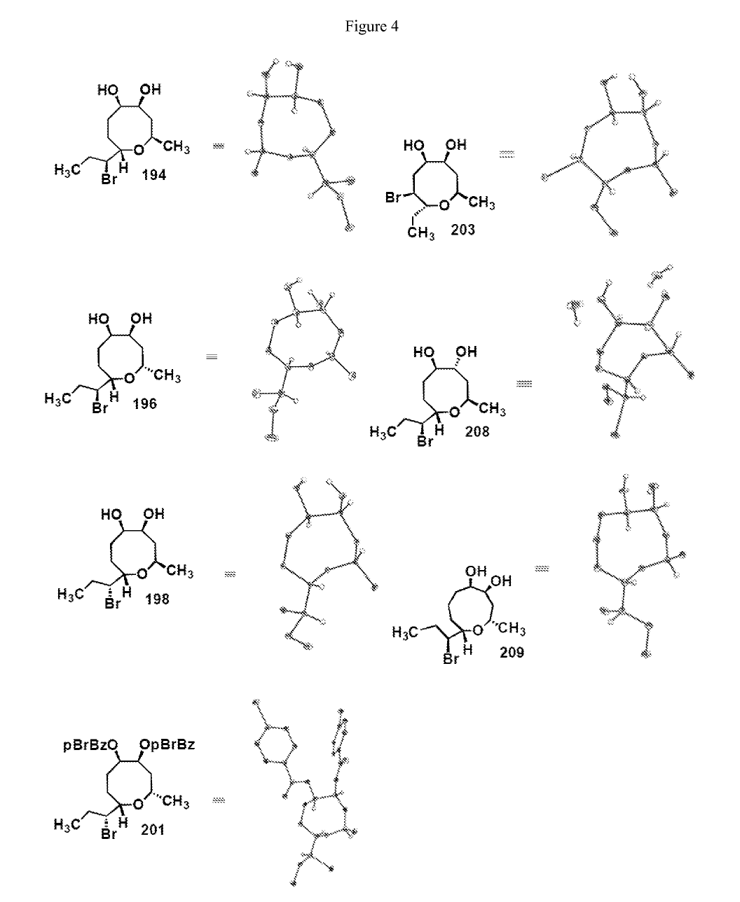

A novel procedure for bromonium-induced ring expansion effected by a unique bromonium source (BDSB) has afforded access to medium-sized cyclic bromothers resembling those of the Laurencia acetogenin family. The stereochemistry of the products was confirmed by X-ray crystal structure analysis (FIG. 4). This process is fast, regio- and stereoselective, and has been demonstrated to produce seven stereochemically and regiochemically distinct 8-membered bromoethers as well as two 9-membered derivatives. Additionally, its overall generality may shed new light on potential biosynthetic pathways that should be considered for the family. Current work is directed towards applying this approach to natural product syntheses as well as exploring the full range of ring sizes and stereochemistries accessible by this method.

### Example 8

### Halogenation of Aromatic Ring: Synthesis of Resveratrol Oligomers

In addition to polyene cyclizations and haloetherifications, these reagents (CDSC, BDSB, and IDSI) also appear to have potential to effect transformations that other electrophilic halogen sources cannot readily achieve. Scheme 21 provides two examples from work towards the resveratrol family of oligomers. The first is a case where BDSB halogenated substrate 170 at a site unique from other halogen sources in what we believe to be the most complex, positionally-selective bromination counter to standard reactivity yet known. This process afforded compound 173 instead of compound 171 as reached by every other reagent in 78% isolated yield, enabling eventual access to the natural product ampelopsin G. A second reaction with BDSB afforded dibrominated product 175.

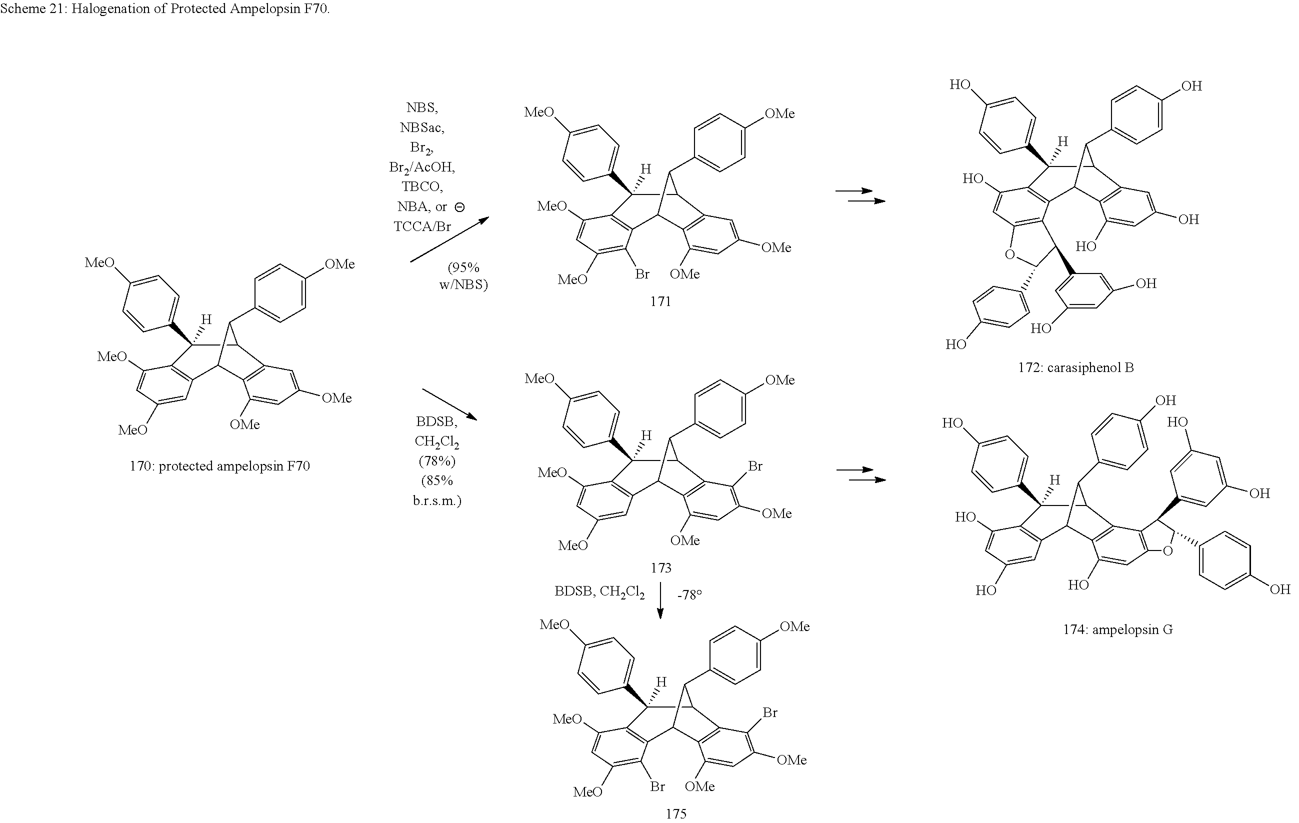

### Example 9

### Antiviral Activity of Peyssonol A and Derivatives

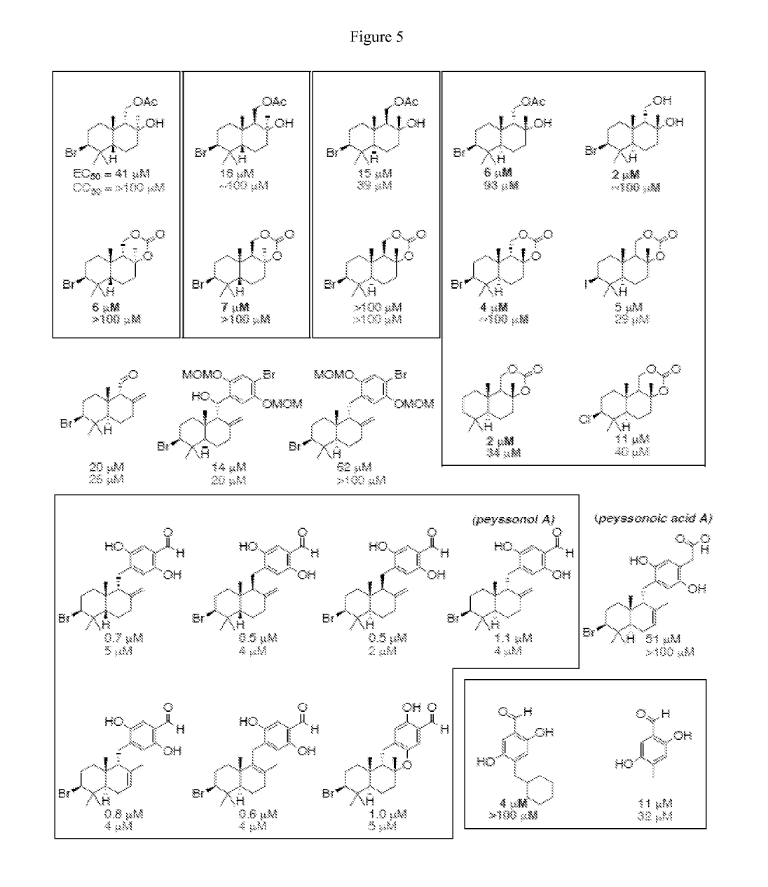

As an exploration into the biochemical potential of materials produced in these studies through the action of BDSB, CDSC, and IDSI, we analyzed the ability of synthetic materials related to, and including, peysonnol A and peysonnoic acid A, to serve as reverse transcriptase inhibitors of HIV-1 given published indications that the parent natural products possessed such activity as indicated earlier. In total, 25 congeners possessing different ring stereochemistries, the presence and/or absence of halogen atoms in the core ring system, and different terminal groups (aromatic or not) were screened. Several key structure-activity trends were identified as indicated by the data in FIG. 5. First, differential termination of the cation-π cyclization to afford either carbonates, diols, or protected alcohols in the form of acetates led to little activity differences. Second, among those materials possessing carbonates, activity as well as toxicity appear to be independent of the stereochemical disposition of the bicycle and the identity (or existence of a halogen atom). Third, for those compounds possessing aromatic rings, the major pharmacophore appears to be the aromatic ring with the stereochemistry and alkene location within the terpene-derived portion being irrelevant. Fourth, the presence of an aldehyde enhances both activity as well as toxicity. Finally, there are a few compounds, indicated by bold shading of their activities and toxicities, which possess useful therapeutic indexes worthy of further exploration as potential therapies. These compounds are also shown below.

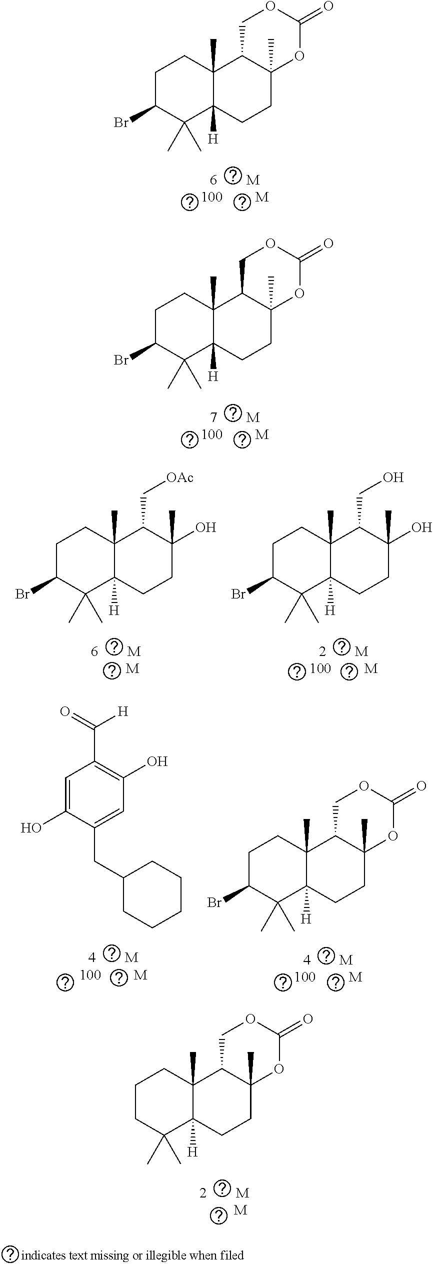

### Materials and Methods

### General Procedures.

All reactions were carried out under an argon atmosphere with dry solvents under anhydrous conditions, unless otherwise noted. Dry methylene chloride (CH₂Cl₂), benzene, toluene, diethyl ether (Et₂O) and tetrahydrofuran (THF) were obtained by passing commercially available pre-dried, oxygen-free formulations through activated alumina columns; nitromethane (MeNO₂) was stored over 3 Å molecular sieves; acetonitrile (MeCN) was dried over 3 Å molecular sieves, distilled, and stored over 3 Å molecular sieves; pyridine was distilled from CaH₂ and stored over 3 Å molecular sieves; triethylamine (Et₃N) was distilled from KOH; N,N-dimethylformamide (DMF) was stored over 3 Å molecular sieves; 1,2-dichloroethane, acetone, and methanol (MeOH) were purchased in anhydrous form from Sigma-Aldrich and used as received. Yields refer to chromatographically and spectroscopically (¹H and ¹³C NMR) homogeneous materials, unless otherwise stated. Reagents were purchased at the highest commercial quality and used without further purification, unless otherwise stated. Reactions were magnetically stirred and monitored by thin-layer chromatography (TLC) carried out on 0.25 mm E Merck silica gel plates (60E-254) using UV light as visualizing agent and an aqueous solution of phosphomolybdic acid and cerium sulfate, and heat as developing agents. Preparative thin-layer chromatography was carried out on 0.50 mm E Merck silica gel plates (60E-254). SiliCycle silica gel (60, academic grade, particle size 0.040-0.063 mm) was used for flash column chromatography. NMR spectra were recorded on Bruker DRX-300 and DRX-400 instruments and calibrated using residual undeuterated solvent as an internal reference. The following abbreviations were used to explain the multiplicities: s=singlet, d=doublet, t=triplet, q=quartet, m=multiplet, AB=AB quartet, br=broad, app=apparent. IR spectra were recorded on a Nicolet Avatar 370 DTGS series FT-IR spectrometer. High-resolution mass spectra (HRMS) were recorded in the Columbia University Mass Spectral Core facility on a JOEL HX110 mass spectrometer using FAB (Fast Atom Bombardment) and EI (Electron Ionization) techniques. All enantiomeric excess (e.e.) values were obtained by HPLC using a Daicel CHIRALCEL OD column. Abbreviations. Ac₂O=acetic anhydride, n-BuLi=n-butyllithium, t-BuLi=t-butyllithium, Boc₂O=di-t-butyl dicarbonate, DMSO=dimethylsulfoxide, 4-DMAP=4-dimethylaminopyridine, EtOAc=ethyl acetate, TFA=trifluoroacetic acid, Et₃SiH=triethylsilane, p-TsOH.H₂O=para-toluenesulfonic acid monohydrate, t-BuOH=tert-butanol, EtOH=ethanol, KOt-Bu=potassium tert-butoxide, Et₂S=diethyl sulfide, MeLi=methyllithium, MeMgBr=methylmagnesium bromide, DBU=1,8-diazabicyclo[5.4.0]undec-7-ene, IPA=2-propanol. AcOH=acetic acid, allylTMS=allyltrimethylsilane, BDSB=bromodiethylsulfonium bromopentachloroantimonate, (coll)₂BrOTf=bis-collidine bromonium trifluoromethanesulfonate, DIAD=diisopropyl azodicarboxylate, DIBAL-H=di-iso-butylaluminum hydride, Hg(TFA)₂=mercury(II) trifluoroacetate, KOt-Bu=potassium tert-butoxide, LDA=lithium diisopropylamide, NBS=N-bromosuccinimide, NCS=N-chlorosuccinimide, PhI(OAc)2=(diacetoxyiodo)benzene, TBAF=tetra-n-butyl ammonium fluoride, TBCO=2,4,4,6-tetrabromocyclohexa-2,5-dienone, TBSCI=tert-butylchlorodimethylsilane.

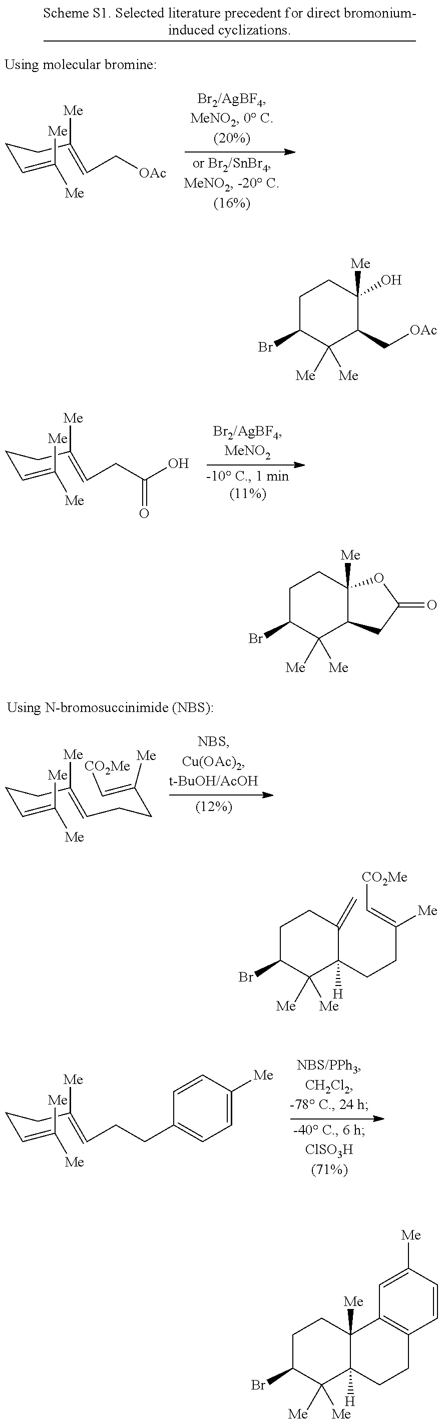

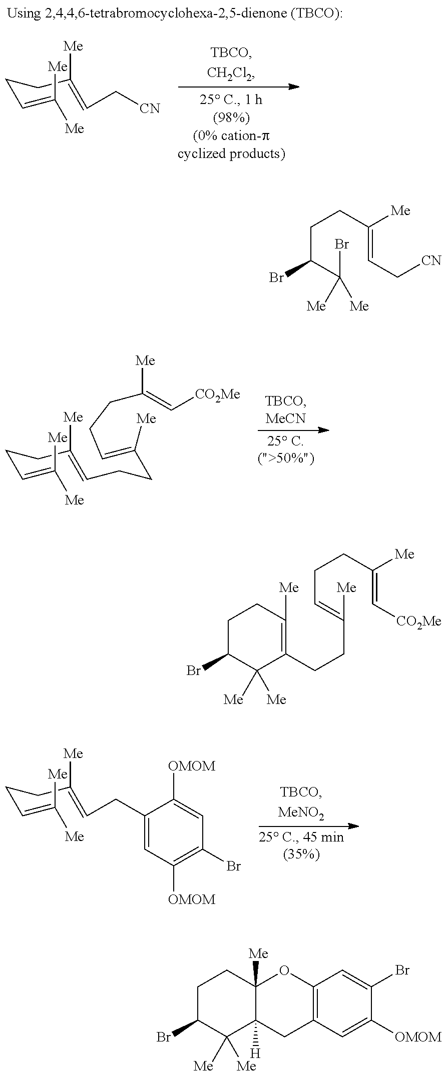

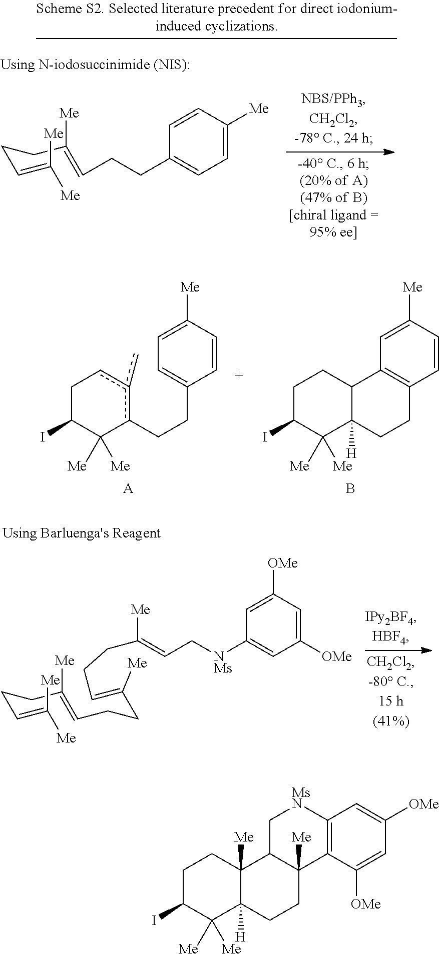

Synthetic procedures, complete characterization, and ¹H and ¹³C NMR spectra of 8, 14, 15, 16, 17, 18, 19, 20, 21, 22, 23, 24 (including X-ray analysis), 34, 71, 82, 88, 89, and 110 are available in (86).

### Investigations Using BDSB

### BDSB (13).

Et₂S (2.97 mL, 27.5 mmol, 1.1 equiv) and a solution of SbCl₅ (1.0 M in CH₂Cl₂, 30.0 mL, 30.0 mmol, 1.2 equiv) were added slowly and sequentially to a solution of Br₂ (1.28 mL, 25.0 mmol, 1.0 equiv) in 1,2-dichloroethane (60 mL) at −30° C. The dark red heterogeneous mixture was stirred for 20 min at −30° C., then warmed slowly using a water bath until the solution became homogeneous (˜30° C.). At this time, the reaction flask was allowed to cool slowly to 0° C. (4 h), then −20° C. (12 h) and large orange plates crystallized from the reaction solution. The solvent was decanted and the crystals were rinsed with cold CH₂Cl₂ (2×5 mL), then dried under vacuum to afford 11.9 g (87% yield) of BDSB.

### 1. Total Synthesis of Peyssonol A and Stereoisomers Thereof

### Total Synthesis of 3 (Purported Structure of Peyssonol A) (2E,6Z)-Farnesol (2)

Phosphorous tribromide (2.58 mL, 27.3 mmol, 0.5 equiv) was added dropwise to a solution of nerol (29, 8.42 g, 54.6 mmol, 1.0 equiv) in Et₂O (160 mL) at −20° C. The reaction mixture was stirred for 60 min, during which time the temperature was allowed to warm slowly to 0° C. Upon completion, the reaction mixture was quenched by the addition of ice-cold water (300 mL) and extracted with hexanes (4×100 mL). The combined organic layers were washed with saturated aqueous NaHCO₃ (200 mL) and brine (200 mL), dried (MgSO₄), filtered, and concentrated. The crude neryl bromide (11.7 g, 53.7 mmol, 1.0 equiv), K₂CO₃ (9.65 g, 69.8 mmol, 1.3 equiv), and ethyl acetoacetate (17.5 g, 134 mmol, 2.5 equiv) were combined in acetone (70 mL) and refluxed at 65° C. for 6 h. The reaction mixture was cooled to 25° C., quenched with saturated aqueous NH₄Cl (100 mL), poured into water (100 mL), and extracted with Et₂O (3×150 mL). The combined organic layers were washed with brine (200 mL), dried (MgSO₄), filtered, and concentrated. Excess ethyl acetoacetate was then removed by distillation (70° C. at 2 mmHg). The crude alkylation product was dissolved in MeOH (64 mL) and 5 M aqueous KOH (32.0 mL, 160 mmol, 3.0 equiv) was added at 25° C.

The mixture was refluxed at 80° C. for 2 h with stirring, then cooled to 0° C. and quenched by the slow addition of 1 M HCl (250 mL). The crude product was extracted into Et₂O (3×200 mL), and the combined organic layers were washed with saturated aqueous NaHCO₃ (200 mL) and brine (200 mL), dried (MgSO₄), filtered, and concentrated. Purification by flash column chromatography (silica gel, hexanes:EtOAc, 19:1) afforded nerylacetone (6.52 g, 61% yield over 3 steps) as a light yellow oil. Next, triethylphosphonoacetate (7.32 mL, 36.9 mmol, 1.1 equiv) was syringed dropwise (with a constant flow of argon) into a vigorously stirring suspension of NaH (60% dispersion in mineral oil, 1.54 g, 38.6 mmol, 1.15 equiv) in THF (70 mL) at −20° C. After 30 min of stirring at −20° C., a solution of nerylacetone (6.52 g, 33.6 mmol, 1.0 equiv) in THF (10 mL) was syringed slowly into the reaction mixture. The resultant reaction contents were allowed to warm slowly to 25° C. over the course of 4 h. After an additional 12 h of stirring at 25° C., the reaction mixture was quenched with saturated aqueous NH₄Cl (100 mL), poured into water (100 mL), and extracted with Et₂O (3×150 mL). The combined organic layers were washed with brine (200 mL), dried (MgSO₄), filtered, and concentrated. The crude product was found by ¹H NMR analysis to be a 4.2:1 mixture of E:Z isomers about the newly formed alkene. Careful purification by flash column chromatography (silica gel, hexanes:CH₂Cl₂, 4:1→1:2) afforded ethyl (2E,6Z)-farnesate (5.65 g, 64% yield) as a colorless oil. Finally, a portion of ethyl (2E,6Z)-farnesate (4.33 g, 16.38 mmol, 1.0 equiv) was syringed dropwise into a suspension of LiAlH₄ (0.373 g, 9.83 mmol, 0.6 equiv) in THF (66 mL) at −78° C. The reaction mixture was allowed to warm slowly to 25° C. over the course of 2 h and then was stirred at 25° C. for 2 h. At this time, the reaction mixture was quenched by careful dropwise addition of saturated aqueous NH₄Cl (2 mL). A 1 M aqueous solution of sodium/potassium tartrate (150 mL) was added, and the biphasic mixture was stirred vigorously for 2 h, at which time the crude product was extracted with Et₂O (4×100 mL). The combined organic layers were washed with brine (200 mL), dried (MgSO₄), filtered, and concentrated. Purification by flash column chromatography (silica gel, hexanes:Et₂O, 4:1) afforded (2E,6Z)-farnesol (2.98 g, 82% yield) as a colorless oil.

### (2E,6Z)-Farnesyl Acetate (30).

Ac₂O (0.111 mL, 1.17 mmol, 1.3 equiv) was added dropwise to a solution of (2E,6Z)-farnesol (0.200 g, 0.899 mmol, 1.0 equiv), 4-DMAP (0.002 g, 0.018 mmol, 0.02 equiv), and Et₃N (0.187 mL, 1.35 mmol, 1.5 equiv) in CH₂Cl₂ (3 mL) at 0° C. After stirring for 30 min at 0° C., the reaction contents were quenched by the addition of water (10 mL), and the crude product was extracted with CH₂Cl₂ (3×10 mL). The combined organic layers were washed with 1 M HCl (10 mL; back-extracted with 3 mL CH₂Cl₂), saturated aqueous NaHCO₃ (10 mL; back-extracted with 3 mL CH₂Cl₂), and brine (10 mL; back-extracted with 3 mL CH₂Cl₂). The combined organic layers were dried (MgSO₄), filtered, and concentrated. Filtration through a silica gel plug (20×50 mm) with hexanes:EtOAc (4:1, 50 mL) afforded 30 (0.233 g, 98% yield) as a light yellow viscous oil. 30: R_{f}=0.53 (silica gel, hexanes:EtOAc, 4:1); IR (film) νₘₐₓ 2965, 2926, 2856, 1742, 1447, 1378, 1365, 1232, 1023 cm⁻¹; ¹H NMR (400 MHz, CDCl₃) δ 5.34 (tt, J=7.2, 1.2 Hz, 1H), 5.14-5.07 (m, 2H), 4.59 (d, J=7.2 Hz, 2H), 2.15-1.98 (m, 8H), 2.05 (s, 3H), 1.70 (s, 3H), 1.69 (s, 6H), 1.61 (s, 3H); ¹³C NMR (100 MHz, CDCl₃) δ 171.2, 142.4, 135.7, 131.7, 124.6, 124.4, 118.4, 61.5, 39.9, 32.1, 26.7, 26.2, 25.8, 23.5, 21.2, 17.8, 16.6; HRMS (EI) calcd for C₁₇H₂₈O₂ [M]⁺ 264.2089. found 264.2083.

(2E,6Z)-Farnesyl t-Butyl Carbonate (31).

A solution of n-BuLi (1.5 M in hexanes, 0.733 mL, 1.10 mmol, 1.1 equiv) was added dropwise to a solution of (2E,6Z)-farnesol (0.222 g, 1.00 mmol, 1.0 equiv) in THF (4 mL) at −78° C. After stirring for 10 min at −78° C., a solution of Boc₂O (0.240 g, 1.10 mmol, 1.1 equiv) in THF (1 mL) was added via syringe. Upon completion of this addition, the reaction flask was immediately removed from the cold bath and the reaction contents were stirred for 30 min at 25° C. The reaction contents were then quenched by the slow addition of water (5 mL), poured into 1 M HCl (5 mL), and extracted with EtOAc (3×10 mL). The combined organic layers were washed with saturated aqueous NaHCO₃ (2×10 mL) and brine (10 mL), dried (MgSO₄), filtered, and concentrated. Purification by flash column chromatography (silica gel, hexanes:EtOAc, 19:1) afforded 31 (0.316 g, 93% yield) as a colorless viscous oil. 31: R_{f}=0.63 (silica gel, hexanes:EtOAc, 4:1); IR (film) νₘₐₓ 2967, 2930, 2857, 1740, 1277, 1254, 1166 cm⁻¹; ¹H NMR (400 MHz, CDCl₃) δ 5.36 (tq, J=7.2, 1.2 Hz, 1H), 5.13-5.06 (m, 2H), 4.58 (d, J=7.2 Hz, 2H), 2.13-1.98 (m, 8H), 1.70 (s, 3H), 1.68 (s, 6H), 1.60 (s, 3H), 1.48 (s, 9H); ¹³C NMR (75 MHz, CDCl₃) δ 153.8, 142.6, 135.7, 131.7, 124.6, 124.4, 118.2, 81.9, 63.9, 40.0, 32.1, 27.9 (3C), 26.7, 26.2, 25.8, 23.5, 17.8, 16.6; HRMS (FAB) calcd for C₂₀H₃₃O₃ [M−H]⁺ 321.2430. found 321.2418.

### Cis-Decalin Framework 32.

A solution of BDSB (13, 0.228 g, 0.42 mmol, 1.1 equiv) in nitromethane (1 mL) was syringed into a solution of 30 (0.100 g, 0.38 mmol, 1.0 equiv) in nitromethane (37 mL) at 0° C. After stirring for 30 s at 0° C., the reaction mixture was quenched by the sequential addition of 5% aqueous Na₂SO₃ (20 mL) and saturated aqueous NaHCO₃ (20 mL). The biphasic mixture was stirred vigorously for 1 h at 25° C., poured into brine (40 mL), and extracted with EtOAc (3×50 mL). The combined organic layers were then washed with brine (50 mL), dried (MgSO₄), filtered, and concentrated. Purification by flash column chromatography (silica gel, hexanes:EtOAc, 9:1→3:2) afforded a 4:1 mixture of 32 and 43 (0.058 g, 34% yield of 32 and 8% yield of 43) as a colorless solid that could not be further purified by chromatography or recrystallization. Analytically pure 32 was obtained by hydrolysis and monoacetylation of the cyclic carbonate product 33. 32: R_{f}=0.21 (silica gel, hexanes:EtOAc, 3:2); IR (film) νₘₐₓ 3431 (br), 2970, 2930, 2873, 1734, 1367, 1244, 1028 cm⁻¹; ¹H NMR (400 MHz, CDCl₃) δ 4.35 (dd, J=12.0, 4.0 Hz, 1H), 4.24 (dd, J=12.4, 6.4 Hz, 1H), 4.17 (dd, J=12.8, 4.4 Hz, 1H), 2.67 (s, 1H), 2.19 (dq, J=13.2, 4.0 Hz, 1H), 2.07 (m, 1H), 2.06 (s, 3H), 1.90-1.77 (m, 2H), 1.70-1.59 (m, 2H), 1.45-1.33 (m, 2H), 1.32-1.19 (m, 2H), 1.30 (s, 3H), 1.29 (s, 3H), 1.24 (s, 3H), 1.11 (s, 3H); ¹³C NMR (75 MHz, CDCl₃) δ 171.0, 72.0, 64.1, 63.5, 56.9, 56.0, 42.1, 40.1, 39.8, 32.6, 31.6, 29.8, 28.6, 28.0, 24.5, 22.9, 21.4; HRMS (FAB) calcd for C₁₇H₃₀BrO₃ [M+H]⁺ 361.1378. found 361.1396.

### Cis-Decalin Framework 33.

A solution of BDSB (13, 0.187 g, 0.34 mmol, 1.1 equiv) in nitromethane (1 mL) pre-cooled to −25° C. was syringed into a solution of 31 (0.100 g, 0.31 mmol, 1.0 equiv) in nitromethane (30 mL) at −25° C. Once the addition was complete, the reaction mixture was removed from the cold bath and stirred at 25° C. for 15 min. The reaction contents were then quenched by the sequential addition of 5% aqueous Na₂SO₃ (20 mL) and saturated aqueous NaHCO₃ (20 mL). The resultant biphasic mixture was stirred vigorously for 1 h at 25° C., then poured into brine (40 mL) and extracted with EtOAc (3×50 mL). The combined organic layers were washed with brine (50 mL), dried (MgSO₄), filtered, and concentrated. Purification by flash column chromatography (silica gel, hexanes:EtOAc, 9:1→1:1) afforded a sample of 33 contaminated with a small amount of 44 (0.036 g combined), the latter of which was removed by recrystallization from boiling Et₂O to provide 33 (0.028 g, 26% yield) as a white crystalline solid. 33: R_{f}=0.50 (silica gel, hexanes:EtOAc, 2:3); IR (film) νₘₐₓ 2973, 2938, 2873, 1747, 1223, 1120, 1079 cm⁻¹; ¹H NMR (400 MHz, CDCl₃) δ 4.53 (dd, J=10.8, 5.6 Hz, 1H), 4.32 (dd, J=12.8, 10.8 Hz, 1H), 4.13 (dd, J=12.4, 4.8 Hz, 1H), 2.25-1.95 (m, 5H), 1.69 (dt, J=4.4, 13.2 Hz, 1H), 1.63-1.47 (m, 2H), 1.49 (s, 3H), 1.38 (m, 1H), 1.31 (s, 3H), 1.29 (s, 3H), 1.15 (m, 1H), 1.11 (s, 3H); ¹³C NMR (100 MHz, CDCl₃) δ 149.0, 81.2, 67.4, 62.6, 54.7, 49.0, 39.9, 38.4, 38.1, 32.1, 31.2, 29.6, 28.3, 28.1, 22.5, 21.2; HRMS (FAB) calcd for C₁₆H₂₆BrO₃ [M+H]⁺ 345.1065. found 345.1073. [See FIG. 3]

### Aldehyde 26.

Solid K₂CO₃ (0.190 g, 1.38 mmol, 5.0 equiv) was added to a solution of 33 (0.095 g, 0.28 mmol, 1.0 equiv) in MeOH (14 mL) at 40° C. After stirring the resultant mixture for 30 min at 40° C., the reaction contents were quenched by the addition of ice-cold saturated aqueous NH₄Cl (10 mL). The crude product was extracted with EtOAc (4×20 mL), washed with brine (20 mL), dried (MgSO₄), filtered, and concentrated to afford the desired diol as a white solid (0.087 g, 99% yield) which was carried forward without any additional purification. [Note: the diol was co-evaporated with anhydrous toluene to remove any traces of water before being subjected to the subsequent oxidation procedure]. Next, a solution of DMSO (0.098 mL, 1.38 mmol, 5.0 equiv) in CH₂Cl₂ (1 mL) was added dropwise to a solution of oxalyl chloride (0.048 mL, 0.54 mmol, 2.0 equiv) in CH₂Cl₂ (4 mL) at −78° C. After stirring for 5 min at −78° C., a solution of the diol (0.087 g, 0.27 mmol, 1.0 equiv) in a mixture of CH₂Cl₂ (5 mL) and DMSO (0.5 mL, to enhance solubility) was added slowly. After stirring for an additional 5 min, Et₃N (0.38 mL, 2.7 mmol, 10 equiv) was added. The reaction contents were then allowed to warm slowly from −78° C. to −50° C. over the course of 1 h and quenched by the careful addition of saturated aqueous NaHCO₃ (20 mL). The crude product was extracted with CH₂Cl₂ (3×20 mL) and the combined organic layers were then washed with water (20 mL; back-extracted with 5 mL CH₂Cl₂) and brine (20 mL; back-extracted with 5 mL CH₂Cl₂), dried (MgSO₄), filtered, and concentrated to afford the desired aldehyde intermediate as a light yellow solid (0.084 g, 97% yield) which was carried forward without any additional purification. [Note: the aldehyde was co-evaporated with anhydrous toluene to remove any traces of water before being subjected to the subsequent dehydration procedure]. Finally, a solution of the aldehyde (0.084 g, 0.26 mmol, 1.0 equiv) and Et₃N (0.22 mL, 1.59 mmol, 6.0 equiv) in CH₂Cl₂ (5.5 mL) was cooled to −97° C. (liquid N₂/CH₂Cl₂ slurry). A solution of SOCl₂ (0.038 mL, 0.53 mmol, 2.0 equiv) in CH₂Cl₂ (0.5 mL) was added dropwise over approximately 3 min. The reaction mixture was stirred at −97° C. for 1 h, at which time it was removed from the cold bath and quenched by the addition of MeOH (0.5 mL). The crude reaction mixture was then filtered through a silica gel plug (20×50 mm) with CH₂Cl₂ (75 mL) to remove ammonium salts. Concentration yielded a crude yellow solid (which by ¹H NMR contained only exocyclic methylene signals; i.e. no trace of trisubstituted or tetrasubstituted alkenes) which was purified by flash column chromatography (silica gel, hexanes:CH₂Cl₂ 3:2) to afford 26 (0.075 g, 95%) as a colorless amorphous solid. 26: R_{f}=0.47 (silica gel, hexanes:EtOAc, 9:1); IR (film) νₘₐₓ 2966, 2934, 2869, 1720, 1454, 1393, 895 cm⁻¹; ¹H NMR (400 MHz, CDCl₃) δ 9.87 (d, J=4.8 Hz, 1H), 4.94 (s, 1H), 4.46 (s, 1H), 4.27 (dd, J=12.4, 4.8 Hz, 1H), 2.69 (m, 1H), 2.38-2.10 (m, 3H), 2.06-1.90 (m, 3H), 1.62 (m, 1H), 1.55-1.45 (m, 2H), 1.31 (s, 3H), 1.27 (s, 3H), 1.08 (s, 3H); ¹³C NMR (100 MHz, CDCl₃) δ 204.7, 144.5, 109.1, 65.4, 63.3, 54.3, 40.5, 40.4, 36.6, 31.9, 31.5, 29.3, 28.5, 28.0, 26.7; HRMS (EI) calcd for C₁₅H₂₃BrO [M]⁺ 298.0932. found 298.0930.

### Aryl Addition Product 35.

A solution n-BuLi (1.4 M in hexanes, 0.197 mL, 0.28 mmol, 1.1 equiv) was syringed dropwise into a solution of 34 (0.107 g, 0.30 mmol, 1.2 equiv) in THF (7 mL) at −78° C. After stirring for 15 min at −78° C., the resultant aryllithium solution was syringed quickly into a solution of aldehyde 26 (0.075 g, 0.25 mmol, 1.0 equiv) in THF (3 mL) at −78° C. After stirring for 10 min at −78° C., the reaction mixture was quenched by the addition of saturated aqueous NH₄Cl (5 mL) and water (5 mL). The reaction contents were then extracted with EtOAc (3×15 mL), washed with brine (20 mL), dried (MgSO₄), filtered, and concentrated. Careful purification of the resultant residue by flash column chromatography (silica gel, hexanes:EtOAc, 1:0→9:1) afforded a 2.2:1 ratio of separable benzylic alcohol diastereomers (0.029 g, 20% yield of the less polar diastereomer; 0.063 g, 43% yield of the more polar diastereomer), each as a colorless amorphous solid. [Note: originally these two diastereomers were reacted together in the next step, but it was found that one was significantly more reactive than the other, and since the product slowly decomposes in the acidic reaction media, the yield could be increased by reacting the two diastereomers separately]. Pressing forward, TFA (0.018 mL, 0.23 mmol, 5.0 equiv) was added dropwise to a solution of the less polar benzylic alcohol diastereomer (0.027 g, 0.047 mmol, 1.0 equiv) and Et₃SiH (0.075 mL, 0.47 mmol, 10 equiv) in CH₂Cl₂ (1 mL) under argon at 0° C. After stirring for 30 min at 0° C., the reaction mixture was quenched by the careful addition of saturated aqueous NaHCO₃ (5 mL) and extracted with CH₂Cl₂ (3×5 mL). The combined organic layers were then dried (MgSO₄), filtered, and concentrated. Separately, TFA (0.039 mL, 0.51 mmol, 5.0 equiv) was added dropwise to a solution of the more polar benzylic alcohol diastereomer (0.059 g, 0.102 mmol, 1.0 equiv) and Et₃SiH (0.163 mL, 1.02 mmol, 10 equiv) in CH₂Cl₂ (1 mL) under argon at 0° C. After stirring for 90 min at 0° C., the reaction mixture was quenched by the careful addition of saturated aqueous NaHCO₃ (5 mL) and extracted with CH₂Cl₂ (3×5 mL). The combined organic layers were then dried (MgSO₄), filtered, and concentrated. Combination of the two crude products and purification by flash column chromatography (silica gel, hexanes:CH₂Cl₂, 1:0→1:1) afforded aryl addition product 35 (0.053 g, 64% yield) as a colorless amorphous solid. 35: R_{f}=0.52 (silica gel, hexanes:EtOAc, 4:1); IR (film) νₘₐₓ 2953, 2932, 2855, 1488, 1151, 1081, 995 cm⁻¹; ¹H NMR (400 MHz, CDCl₃) δ 7.24 (s, 1H), 6.93 (s, 1H), 5.13 (s, 2H), 5.11 (d, J=1.6 Hz, 2H), 4.81 (s, 1H), 4.62 (s, 1H), 4.26 (dd, J=12.8, 4.8 Hz, 1H), 3.51 (s, 3H), 3.48 (s, 3H), 2.79 (m, 1H), 2.68 (m, 1H), 2.32-2.06 (m, 4H), 1.98 (dq, J=12.8, 4.0 Hz, 1H), 1.85 (dt, J=4.0, 12.8 Hz, 1H), 1.70-1.57 (m, 2H), 1.41 (s, 3H), 1.38 (m, 1H), 1.36 (s, 3H), 1.26 (m, 1H), 1.09 (s, 3H); ¹³C NMR (100 MHz, CDCl₃) δ 150.9, 148.5, 147.6, 131.8, 119.0, 118.9, 110.1, 107.9, 96.4, 95.2, 64.9, 56.5, 56.3, 55.5, 53.5, 41.8, 40.4, 37.9, 32.3, 31.9, 28.8 (2C), 28.6, 27.1, 25.1; HRMS (FAB) calcd for C₂₅H₃₆Br₂O₄ [M]⁺ 558.0980. found 558.0983.

### Originally Proposed Structure of Peyssonol A (3).

A solution of n-BuLi (1.4 M in hexanes, 0.043 mL, 0.060 mmol, 1.2 equiv) was syringed dropwise into a solution of 35 (0.028 g, 0.050 mmol, 1.0 equiv) in THF (1 mL) at −78° C. After stirring for 15 min at −78° C., DMF (0.019 mL, 0.25 mmol, 5.0 equiv) was syringed slowly into the reaction mixture. After stirring the resultant solution for an additional 20 min at −78° C., the reaction mixture was quenched by the addition of saturated aqueous NH₄Cl (1 mL), poured into water (4 mL), and extracted with EtOAc (3×5 mL). The combined organic layers were then washed with brine, dried (MgSO₄), filtered, and concentrated. Purification of the resultant residue by flash column chromatography (silica gel, hexanes:EtOAc, 1:0→2:1) afforded protected 3 (0.016 g, 62% yield) as a light yellow powder. Finally, a portion of the newly synthesized protected 3 (2.5 mg, 0.0049 mmol, 1.0 equiv) was dissolved in a solution of p-TsOH.H₂O (0.2 M in t-BuOH, 1 mL) and stirred at 65° C. for 2 h. Upon completion, the reaction contents were poured into water (5 mL) and extracted with EtOAc (3×5 mL). The combined organic layers were then washed with brine (5 mL), dried (MgSO₄), filtered, and concentrated. Purification of the resultant residue by flash column chromatography (silica gel, hexanes:EtOAc, 4:1) afforded the originally proposed structure for peyssonol A (3, 1.9 mg, 91% yield) as a light yellow amorphous solid. 3: R_{f}=0.38 (silica gel, hexanes:EtOAc, 2:1); IR (film) νₘₐₓ 3392 (br), 2927, 2855, 1648, 1440, 1348, 1172, 799 cm⁻¹; ¹H NMR (400 MHz, C₆D₆) δ 11.11 (s, 1H), 9.20 (s, 1H), 6.84 (s, 1H), 5.74 (s, 1H), 4.74 (s, 1H), 4.62 (s, 1H), 3.90 (br s, 1H), 3.86 (dd, J=12.8, 4.8 Hz, 1H), 2.68 (dd, J=16.4, 10.8 Hz, 1H), 2.49 (app d, J=16.0 Hz, 1H), 2.10-1.80 (m, 4H), 1.52-1.02 (m, 4H), 1.24 (s, 3H), 1.01-0.73 (m, 2H), 0.98 (s, 3H), 0.91 (s, 3H); ¹³C NMR (100 MHz, C₆D₆) δ 194.9, 156.6, 147.6, 146.8, 141.0, 128.6, 118.2, 117.0, 107.8, 64.5, 54.8, 53.0, 41.6, 40.1, 37.6, 32.3, 32.0, 28.6 (2C), 28.2, 26.8, 25.6; HRMS (FAB) calcd for C₂₂H₃₀BrO₃ [M+H]⁺ 421.1378. found 421.1362.

### 2. Total Synthesis of Potential Peyssonol a Structure 40.

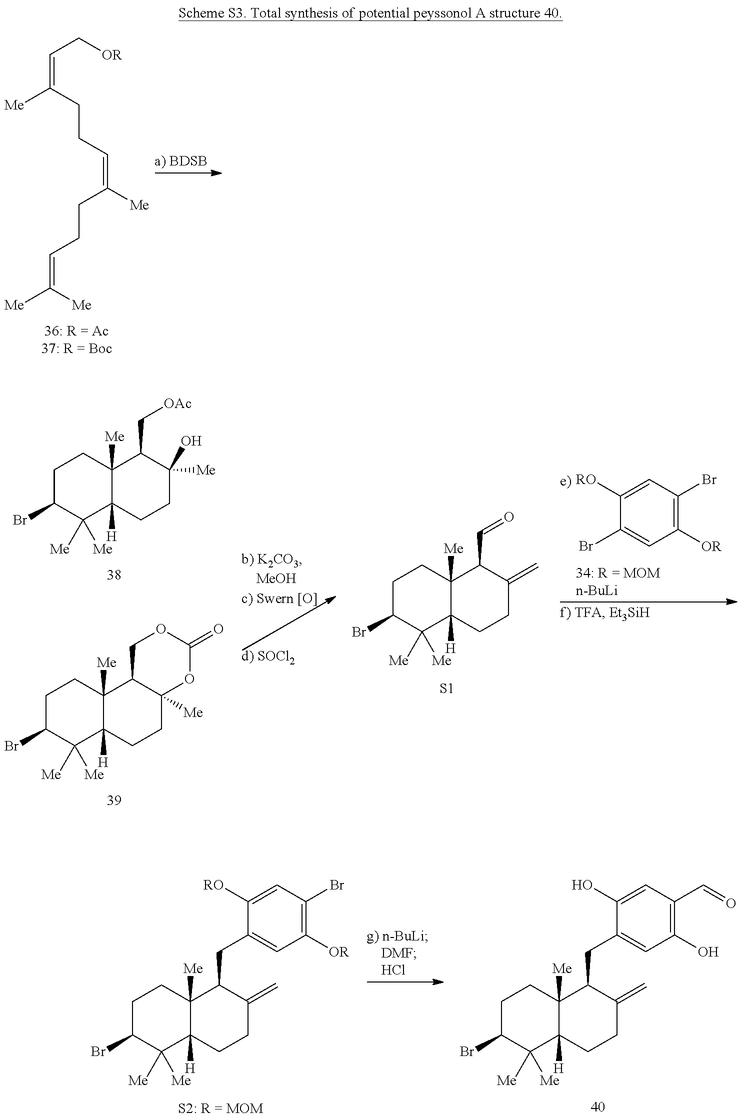

### (2Z,6Z)-Farnesol.

A solution of ethyl (2Z,6Z)-farnesate (obtained as the minor product from the Horner-Wadsworth-Emmons olefination of nerylacetone in the synthesis of ethyl (2E,6Z)-farnesate above, 0.470 g, 1.78 mmol, 1.0 equiv) in Et₂O (3 mL) was syringed slowly into a suspension of LiAlH₄ (0.068 g, 1.78 mmol, 1.0 equiv) in Et₂O (7 mL) at −78° C. The reaction mixture was allowed to warm to 0° C. over the course of 90 min, and then quenched by the dropwise addition of saturated aqueous NH₄Cl (1 mL). A 1 M aqueous solution of sodium potassium tartrate (20 mL) was added to the reaction contents and the resultant biphasic mixture was stirred vigorously for 12 h at 25° C., after which time the crude product was extracted with Et₂O (4×15 mL). The combined organic layers were then washed with brine (20 mL), dried (MgSO₄), filtered, and concentrated. Purification of the resultant residue by flash column chromatography (silica gel, hexanes:EtOAc, 9:1) afforded (2Z,6Z)-farnesol (0.320 g, 81% yield) as a light yellow oil.

### (2Z,6Z)-Farnesyl Acetate (36).

Prepared as in 30; 0.112 g (95% yield) as a light yellow viscous oil. 36: R_{f}=0.57 (silica gel, hexanes:EtOAc, 4:1); IR (film) νₘₐₓ 2966, 2930, 2858, 1741, 1447, 1377, 1233, 1023 cm⁻¹; ¹H NMR (400 MHz, CDCl₃) δ 5.35 (t, J=7.2 Hz, 1H), 5.13-5.06 (m, 2H), 4.54 (d, J=7.2 Hz, 2H), 2.14-1.98 (m, 11H), 1.75 (s, 3H), 1.67 (s, 6H), 1.59 (s, 3H); ¹³C NMR (75 MHz, CDCl₃) δ 171.1, 142.6, 136.0, 131.6, 124.4 (2C), 119.3, 61.2, 32.5, 32.1, 26.7, 26.5, 25.8, 23.6, 23.4, 21.1, 17.7; HRMS (FAB) calcd for C₁₇H₂₈O₂ [M]⁺ 264.2089. found 264.2092.

(2Z,6Z)-Farnesyl t-Butyl Carbonate (37).

Prepared as in 31; 0.139 g (91% yield) as a colorless viscous oil. 37: R_{f}=0.63 (silica gel, hexanes:EtOAc, 4:1); IR (film) νₘₐₓ 2968, 2932, 1740, 1277, 1254, 1168 cm⁻¹; ¹H NMR (400 MHz, CDCl₃) δ 5.37 (dt, J=1.2, 7.2 Hz, 1H), 5.13-5.06 (m, 2H), 4.55 (dd, J=7.2, 0.8 Hz, 2H), 2.12-1.98 (m, 8H), 1.75 (s, 3H), 1.68 (s, 6H), 1.60 (s, 3H), 1.47 (s, 9H); ¹³C NMR (75 MHz, CDCl₃) δ 153.8, 142.7, 136.0, 131.7, 124.4 (2C), 119.2, 81.9, 63.6, 32.6, 32.1, 27.9 (3C), 26.7, 26.5, 25.8, 23.6, 23.5, 17.8; HRMS (FAB) calcd for C₂₀H₃₅O₃ [M+H]⁺ 323.2586. found 323.2575.

### Cis-Decalin Framework 38.

A solution of BDSB (13, 0.220 g, 0.42 mmol, 1.1 equiv) in nitromethane (1 mL) was syringed into a solution of 36 (0.100 g, 0.38 mmol, 1.0 equiv) in nitromethane (37 mL) at 0° C. After stirring for 30 s at 0° C., the reaction mixture was quenched by the sequential addition of 5% aqueous Na₂SO₃ (20 mL) and saturated aqueous NaHCO₃ (20 mL). The biphasic mixture was stirred vigorously for 1 h at 25° C., then poured into brine (40 mL) and extracted with EtOAc (3×50 mL). The combined organic layers were washed with brine (50 mL), dried (MgSO₄), filtered, and concentrated. Purification of the resultant residue by flash column chromatography (silica gel, hexanes:EtOAc, 1:0→3:1) afforded cis-decalin framework 38 (0.028 g, 20% yield) as a white crystalline solid. 38: R_{f}=0.46 (silica gel, hexanes:EtOAc, 3:2); IR (film) νₘₐₓ 3490 (br), 2936, 1736, 1367, 1243, 1028 cm⁻¹; ¹H NMR (400 MHz, CDCl₃) δ 4.44 (dd, J=12.0, 4.0 Hz, 1H), 4.29 (app t, J=4.0 Hz, 1H), 4.25 (dd, J=12.4, 3.2 Hz, 1H), 2.12-2.00 (m, 2H), 2.04 (s, 3H), 1.90-1.75 (m, 5H), 1.67 (dd, J=5.2, 3.6 Hz, 1H), 1.63-1.48 (m, 2H), 1.29 (s, 1H), 1.27 (s, 3H), 1.23 (s, 3H), 1.19 (s, 3H), 1.16 (s, 3H); ¹³C NMR (75 MHz, CDCl₃) δ 171.0, 72.1, 70.8, 63.3, 47.0, 45.4, 39.5, 38.3, 36.9, 33.5, 33.0, 32.1, 28.1 (2C), 27.6, 21.4, 18.0; HRMS (FAB) calcd for C₁₇H₂₈BrO₃ [M−H]⁺ 359.1222. found 359.1206. [Note: although not germane to the final product of the synthesis, since it is eventually ablated, the orientation of the C-8 stereocenter was deduced from the outcome of the elimination reaction discussed below. The production of a significant amount of tetrasubstituted alkene product during this step, even at very low temperature, indicates that the C-8 hydroxyl group must be trans-diaxial to the C-9 hydrogen. Since the bromine in this structure is axial, as is apparent from the chemical shift and J-values of the geminal C-3 proton in the ¹H NMR, the likely chair-chair conformation of the cis-decalin structure would result in an axial C-9 hydrogen trans to an axial OH only if the OH group was on the β-face of C-8].

### Cis-Decalin Framework 39.

A solution of BDSB (13, 0.25 g, 0.46 mmol, 1.1 equiv) in nitromethane (1 mL) pre-cooled to −25° C. was syringed into a solution of 37 (0.135 g, 0.42 mmol, 1.0 equiv) in nitromethane (41 mL) at −25° C. Following this addition, the reaction mixture was removed from the cold bath and stirred for 15 min at 25° C. Upon completion, the reaction contents were then quenched by the sequential addition of 5% aqueous Na₂SO₃ (20 mL) and saturated aqueous NaHCO₃ (20 mL). The resultant biphasic mixture was stirred vigorously for 1 h at 25° C., then poured into brine (40 mL) and extracted with EtOAc (3×50 mL). The combined organic layers were washed with brine (50 mL), dried (MgSO₄), filtered, and concentrated. Purification of the resultant residue by flash column chromatography (silica gel, hexanes:EtOAc, 9:1→1:1) afforded cis-decalin framework 39 (0.040 g, 28% yield) as a white crystalline solid. 39: R_{f}=0.38 (silica gel, hexanes:EtOAc, 2:3); IR (film) νₘₐₓ 2976, 2939, 2884, 1736, 1231, 1218, 1128, 1109 cm⁻¹; ¹H NMR (400 MHz, CDCl₃) δ 4.62 (dd, J=12.4, 6.0 Hz, 1H), 4.50 (dd, J=12.0, 2.8 Hz, 1H), 4.24 (dd, J=4.8, 3.2 Hz, 1H), 2.14-1.81 (m, 7H), 1.75 (dt, J=14.4, 4.4 Hz, 1H), 1.72-1.62 (m, 2H), 1.51 (s, 3H), 1.19 (s, 3H), 1.18 (s, 3H), 1.17 (s, 3H); ¹³C NMR (75 MHz, CDCl₃) δ 149.4, 82.0, 68.4, 66.6, 44.2, 39.6, 39.2, 35.8, 34.4, 33.0, 32.2, 29.2, 28.1, 27.8, 27.6, 17.9; HRMS (FAB) calcd for C₁₆H₂₆BrO₃ [M+H]⁺ 345.1065. found 345.1077.

### Aldehyde S1.

Solid K₂CO₃ (0.084 g, 0.61 mmol, 5.0 equiv) was added to a solution of 39 (0.042 g, 0.122 mmol, 1.0 equiv) in MeOH (4 mL) at 50° C. The resultant reaction mixture was stirred for 2 h at 50° C., and then quenched by the addition of ice-cold saturated aqueous NH₄Cl (5 mL). The reaction contents were then extracted with EtOAc (4×10 mL), washed with brine (10 mL), dried (MgSO₄), filtered, and concentrated to afford the desired diol (0.039 g, quant.) as a white crystalline solid. [Note: the diol was co-evaporated with anhydrous toluene to remove any traces of water before being subjected to the subsequent oxidation procedure]. Next, a solution of DMSO (0.043 mL, 0.61 mmol, 5.0 equiv) in CH₂Cl₂ (0.5 mL) was added dropwise to a solution of oxalyl chloride (0.021 mL, 0.24 mmol, 2.0 equiv) in CH₂Cl₂ (2 mL) at −78° C. After stirring for 5 min at −78° C., a solution of the diol (0.039 g, 0.122 mmol, 1.0 equiv) in a mixture of CH₂Cl₂ (2 mL) and DMSO (0.2 mL, to enhance solubility) was added slowly. After stiffing for an additional 5 min, Et₃N (0.169 mL, 1.22 mmol, 10 equiv) was added. The reaction contents were then allowed to warm slowly from −78° C. to −45° C. over the course of 1 h and quenched by the careful addition of saturated aqueous NaHCO₃ (10 mL). The crude product was extracted with CH₂Cl₂ (3×10 mL) and the combined organic layers were washed with water (10 mL; back-extracted with 3 mL CH₂Cl₂) and brine (10 mL; back-extracted with 3 mL CH₂Cl₂), dried (MgSO₄), filtered, and concentrated to afford the aldehyde intermediate (0.036 g, 93% yield) as a light yellow amorphous solid which was carried forward without additional purification. [Note: the aldehyde was co-evaporated with anhydrous toluene to remove any traces of water before being subjected to the subsequent dehydration procedure]. Finally, a solution of the aldehyde (0.036 g, 0.113 mmol, 1.0 equiv) and Et₃N (0.094 mL, 0.68 mmol, 6.0 equiv) in CH₂Cl₂ (2 mL) was cooled to −97° C. (liquid N₂/CH₂Cl₂ slurry). A solution of SOCl₂ (0.016 mL, 0.23 mmol, 2.0 equiv) in CH₂Cl₂ (0.5 mL) was added dropwise over approximately 3 min. The reaction mixture was stirred at −97° C. for 1 h, at which time it was removed from the cold bath and quenched by the addition of MeOH (0.2 mL). The crude reaction mixture was then filtered through a silica gel plug (10×50 mm) with CH₂Cl₂ (50 mL) to remove ammonium salts. Concentration yielded a crude solid (an 85:12:3 mixture of exocyclic:tetrasubstituted: trisubstituted alkenes) that was purified by flash column chromatography (silica gel, hexanes:CH₂Cl₂, 3:2) to afford aldehyde S1 (0.027 g, 80% yield) as a colorless amorphous solid.

### Aryl Addition Product S2.

A solution of n-BuLi (1.6 M in hexanes, 0.060 mL, 0.096 mmol, 1.2 equiv) was syringed dropwise into a solution of 34 (0.043 g, 0.120 mmol, 1.5 equiv) in THF (3 mL) at −78° C. After stiffing for 20 min at −78° C., the resultant aryllithium solution was syringed quickly into a solution of aldehyde Si (0.024 g, 0.080 mmol, 1.0 equiv) in THF (2 mL) at −40° C. After stirring for 5 min at −40° C., the reaction contents were quenched by the addition of saturated aqueous NH₄Cl (5 mL) and water (5 mL). The resultant mixture was then extracted with EtOAc (3×10 mL), washed with brine (10 mL), dried (MgSO₄), filtered, and concentrated. Careful purification of the resultant residue by flash column chromatography (silica gel, hexanes:EtOAc, 1:0→8:2) afforded a 1.1:1.0 ratio of separable benzylic alcohol diastereomers (0.015 g, 32% yield of the less polar diastereomer; 0.013 g, 29% yield of the more polar diastereomer), each as a colorless amorphous solid. [Note: originally these two diastereomers were reacted together in the next step, but it was found that one was significantly more reactive than the other, and since the product slowly decomposes in the acidic reaction media, the yield could be increased by reacting the two diastereomers separately]. Pressing forward, TFA (0.010 mL, 0.13 mmol, 5.0 equiv) was added dropwise to a solution of the less polar benzylic alcohol diastereomer (0.015 g, 0.026 mmol, 1.0 equiv) and Et₃SiH (0.041 mL, 0.26 mmol, 10 equiv) in CH₂Cl₂ (0.5 mL) under argon at 0° C. After stirring for 60 min at 0° C., the reaction mixture was quenched by the careful addition of saturated aqueous NaHCO₃ (5 mL) and extracted with CH₂Cl₂ (3×5 mL). The combined organic layers were dried (MgSO-₄), filtered, and concentrated. Separately, TFA (0.018 mL, 0.23 mmol, 10 equiv) was added dropwise to a solution of the more polar benzylic alcohol diastereomer (0.013 g, 0.023 mmol, 1.0 equiv) and Et₃SiH (0.37 mL, 0.23 mmol, 10 equiv) in CH₂Cl₂ (0.5 mL) under argon at 0° C. After stirring for 90 min at 0° C., the reaction mixture was quenched by the careful addition of saturated aqueous NaHCO₃ (5 mL) and extracted into CH₂Cl₂ (3×5 mL). The combined organic layers were dried (MgSO₄), filtered, and concentrated. Combination of the two crude products and purification by flash column chromatography (silica gel, hexanes:CH₂Cl₂, 1:0→1:1) afforded aryl addition product S2 (0.012 g, 44% yield) as a colorless amorphous solid.

### Potential Peyssonol A Structure 40.

A solution of n-BuLi (1.6 M in hexanes, 0.016 mL, 0.026 mmol, 1.2 equiv) was syringed dropwise into a solution of S2 (0.012 g, 0.021 mmol, 1.0 equiv) in THF (1 mL) at −78° C. After stirring for 15 min at −78° C., a solution of DMF (0.008 mL, 0.11 mmol, 5.0 equiv) in THF (0.1 mL) was syringed slowly into the reaction mixture. After stirring for an additional 15 min at −78° C., concentrated aqueous HCl (12 M, 0.1 mL, final solution ˜1 M in HCl) was syringed into the reaction mixture, and the reaction contents were then warmed to 40° C. After stirring for 90 min at 40° C., the reaction mixture was quenched by the addition of water (5 mL), and extracted with EtOAc (3×5 mL). The combined organic layers were washed with brine (5 mL), dried (MgSO₄), filtered, and concentrated. Purification of the resultant residue by preparative TLC (silica gel, hexanes:EtOAc, 7:3) afforded potential peyssonol A structure 40 (5.2 mg, 58% yield) as a light yellow powder. 40: R_{f}=0.34 (silica gel, hexanes:EtOAc, 2:1); IR (film) νₘₐₓ 3381 (br), 2964, 2926, 2872, 1652, 1633, 1381, 1228, 866 cm⁻¹; ¹H NMR (400 MHz, C₆D₆) δ 11.14 (s, 1H), 9.24 (s, 1H), 6.70 (s, 1H), 5.74 (s, 1H), 4.51 (t, J=2.0 Hz, 1H), 4.24 (s, 1H), 3.97 (dd, J=12.4, 4.8 Hz, 1H), 3.76 (s, 1H), 2.80 (dd, J=13.2, 4.0 Hz, 1H), 2.39 (dd, J=13.2, 11.6 Hz, 1H), 2.16 (dq, J=4.0, 13.2 Hz, 1H), 2.05-1.85 (m, 3H), 1.78 (m, 1H), 1.59 (dt, J=4.0, 13.6 Hz, 1H), 1.48-1.32 (m, 2H), 1.21 (s, 3H), 1.06 (s, 3H), 0.97 (s, 3H), 0.91 (dd, J=12.8, 5.2 Hz, 1H), 0.64 (m, 1H); ¹³C NMR (100 MHz, C₆D₆) δ 195.0, 156.4, 147.2, 146.9, 140.3, 119.9, 118.9, 117.3, 110.0, 64.3, 57.7, 48.2, 39.7, 38.8, 34.4, 32.3, 31.5, 30.3, 29.3, 28.3, 27.8, 25.8; HRMS (FAB) calcd for C₂₂H₂₉BrO₃ [M]⁺ 420.1300. found 420.1315.

### 3. Total Synthesis of Potential Peyssonol A Structure 45.

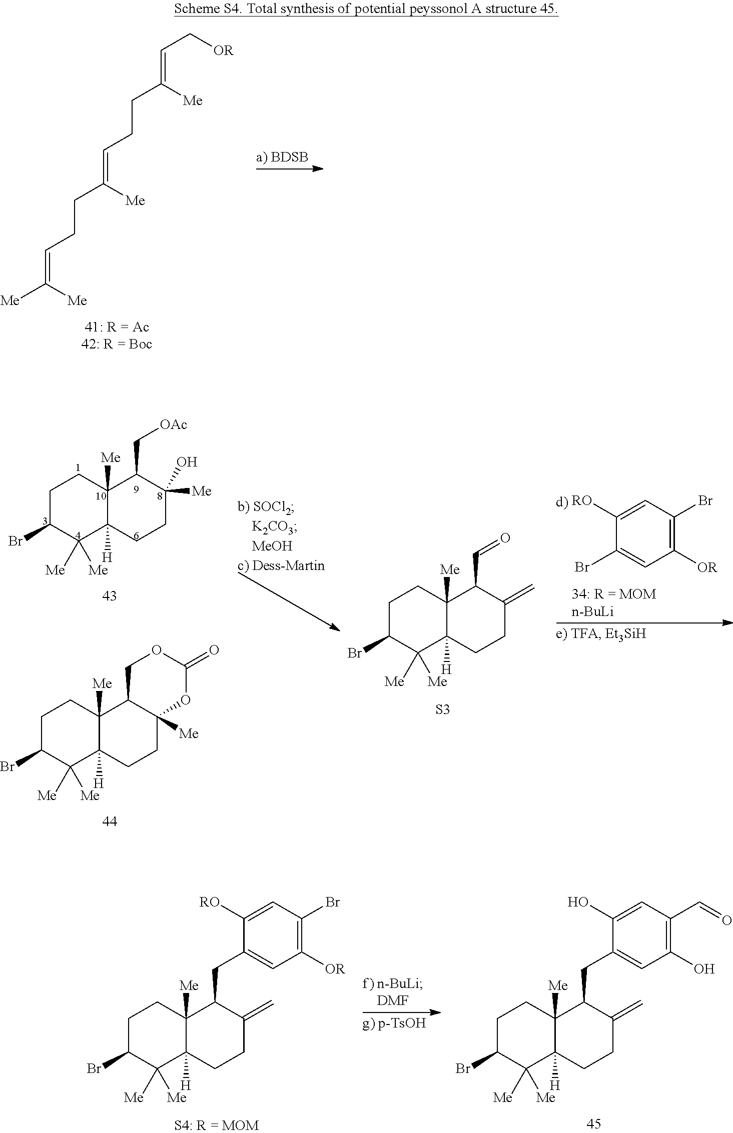

### (2E,6E)-Farnesyl Acetate (41).

Prepared as in 30 from commercially-available (2E,6E)-farnesol; 0.111 g (94% yield) as a colorless viscous oil. 41: R_{f}=0.52 (silica gel, hexanes:EtOAc, 4:1); IR (film) νₘₐₓ 2967, 2922, 2856, 1742, 1444, 1381, 1365, 1232, 1023 cm⁻¹; ¹H NMR (400 MHz, CDCl₃) δ 5.34 (tt, J=7.2, 1.2 Hz, 1H), 5.13-5.05 (m, 2H), 4.59 (d, J=7.2 Hz, 2H), 2.15-1.93 (m, 8H), 2.05 (s, 3H), 1.70 (s, 3H), 1.69 (s, 3H), 1.60 (s, 6H); ¹³C NMR (100 MHz, CDCl₃) δ 171.2, 142.4, 135.6, 131.4, 124.4, 123.7, 118.4, 61.5, 39.8, 39.6, 26.8, 26.3, 25.8, 21.2, 17.8, 16.6, 16.1; HRMS (EI) calcd for C₁₇H₂₈O₂ [M]⁺ 264.2089. found 264.2097.

(2E,6E)-Farnesyl t-Butyl Carbonate (42).

Prepared as in 31 from commercially-available (2E,6E)-farnesol; 0.139 g (91% yield) of a colorless viscous oil. 42: R_{f}=0.58 (silica gel, hexanes:EtOAc, 4:1); IR (film) νₘₐₓ 2979, 2927, 2856, 1740, 1276, 1254, 1165 cm⁻¹; ¹H NMR (400 MHz, CDCl₃) δ 5.37 (ft, J=7.2, 1.2 Hz, 1H), 5.12-5.06 (m, 2H), 4.59 (d, J=7.2 Hz, 2H), 2.15-1.92 (m, 8H), 1.71 (s, 3H), 1.68 (s, 3H), 1.60 (s, 3H), 1.59 (s, 3H), 1.48 (s, 9H); ¹³C NMR (100 MHz, CDCl₃) δ 153.8, 142.7, 135.6, 131.4, 124.4, 123.8, 118.2, 81.9, 63.9, 39.8, 39.6, 27.9 (3C), 26.8, 26.3, 25.8, 17.8, 16.6, 16.1; HRMS (EI) calcd for C₂₀H₃₃O₃ [M−H]⁺ 321.2430. found 321.2418.

### Trans-Decalin Framework 43.

A solution of BDSB (13, 0.228 g, 0.42 mmol, 1.1 equiv) in nitromethane (1 mL) was syringed into a solution of 41 (0.100 g, 0.38 mmol, 1.0 equiv) in nitromethane (37 mL) at 0° C. After stirring for 30 s at 0° C., the reaction mixture was quenched by the sequential addition of 5% aqueous Na₂SO₃ (20 mL) and saturated aqueous NaHCO₃ (20 mL). The resultant biphasic mixture was stirred vigorously for 1 h at 25° C., then poured into brine (40 mL) and extracted with EtOAc (3×50 mL). The combined organic layers were washed with brine (50 mL), dried (MgSO₄), filtered, and concentrated. Purification of the resultant residue by flash column chromatography (silica gel, hexanes:EtOAc, 1:0→6:4) afforded trans-decalin framework 43 (0.058 g, 43% yield) as a white crystalline solid in addition to the separable 48 (0.036 g, 26% yield). 43: R_{f}=0.27 (silica gel, hexanes:EtOAc, 3:2); IR (film) νₘₐₓ 3459 (br), 2971, 2946, 2875, 1735, 1391, 1369, 1244, 1031 cm⁻¹; ¹H NMR (400 MHz, CDCl₃) δ 4.35 (dd, J=12.0, 4.0 Hz, 1H), 4.17 (dd, J=11.6, 5.6 Hz, 1H), 3.98 (dd, J=12.4, 4.4 Hz, 1H), 2.36 (br s, 1H), 2.18 (dq, J=3.6, 12.8 Hz, 1H), 2.09 (m, 1H), 2.03 (s, 3H), 1.88 (dt, J=12.8, 3.2 Hz, 1H), 1.74 (m, 1H), 1.68 (dt, J=13.6, 3.6 Hz, 1H), 1.53-1.31 (m, 3H), 1.25 (dt, J=3.6, 13.2 Hz, 1H), 1.16 (s, 3H), 1.08 (s, 3H), 1.07 (m, 1H), 0.93 (s, 3H), 0.90 (s, 3H); ¹³C NMR (100 MHz, CDCl₃) δ 171.3, 72.2, 68.6, 62.2, 59.8, 56.1, 43.9, 41.0, 39.8, 38.2, 30.8, 30.7, 24.6, 21.9, 21.4, 18.3, 15.9; HRMS (FAB) calcd for C₁₇H₃₀BrO₃ [M+H]⁺361.1378. found 361.1376. [Note: the stereochemistry at C8 was originally ambiguous, although it is inconsequential since this stereocenter is later ablated. Nevertheless, NOE analysis of 43 showed cross peaks between the hydroxyl hydrogen and the α-H at C₁₋₉ as well as between the axial β-H at C₁₋₆ and the axial methyl groups at C-4, C-8, and C-10. This clearly indicated that the methyl group was in the axial β-position at C-8].

### Trans-Decalin Framework 44.

A solution of BDSB (13, 0.25 g, 0.46 mmol, 1.1 equiv) in nitromethane (1 mL) pre-cooled to −25° C. was syringed into a solution of 42 (0.135 g, 0.42 mmol, 1.0 equiv) in nitromethane (41 mL) at −25° C. Following this addition, the reaction mixture was removed from the cold bath and stirred at 25° C. for 15 min. Upon completion, the reaction contents were quenched by the sequential addition of 5% aqueous Na₂SO₃ (20 mL) and saturated aqueous NaHCO₃ (20 mL). The resultant biphasic mixture was then stirred vigorously for 1 h at 25° C., poured into brine (40 mL), and extracted with EtOAc (3×50 mL). The combined organic layers were washed with brine (50 mL), dried (MgSO₄), filtered, and concentrated. Purification of the resultant residue by flash column chromatography (silica gel, hexanes:EtOAc, 9:1→1:1) afforded a 72:28 mixture of 44 and 49 (0.064 g, 45% yield for 44 and 17% yield for 49) as an amorphous solid. Recrystallization of this mixture from boiling MeOH afforded pure 44 as a white crystalline solid. 44: R_{f}=0.43 (silica gel, hexanes:EtOAc, 2:3); IR (film) νₘₐₓ 2948, 1746, 1221, 1126, 1092 cm⁻¹; ¹H NMR (400 MHz, CDCl₃) δ 4.38-4.31 (m, 2H), 3.96 (dd, J=12.0, 5.6 Hz, 1H), 2.26-2.11 (m, 2H), 2.06 (dt, J=12.8, 3.2 Hz, 1H), 1.94-1.82 (m, 2H), 1.68 (dt, J=4.4, 13.6 Hz, 1H), 1.54 (dt, J=13.2, 3.6 Hz, 1H), 1.48 (s, 3H), 1.44 (m, 1H), 1.31 (dt, J=4.8, 12.8 Hz, 1H), 1.18 (dd, J=12.0, 2.0 Hz, 1H), 1.10 (s, 3H), 0.94 (s, 6H); ¹³C NMR (75 MHz, CDCl₃) δ 148.8, 81.6, 67.0, 66.5, 56.0, 51.1, 40.1, 39.9, 39.7, 36.5, 30.6, 30.3, 21.7, 21.1, 18.3, 15.6; HRMS (FAB) calcd for C₁₆H₂₆BrO₃ [M+H]⁺ 345.1065. found 345.1057.

### Aldehyde S3.

A solution of SOCl₂ (0.022 mL, 0.30 mmol, 2.0 equiv) in CH₂Cl₂ (0.2 mL) was added very slowly to a solution of 43 (0.054 g, 0.149 mmol, 1.0 equiv) in Et₃N (0.124 mL, 0.90 mmol, 6.0 equiv) and CH₂Cl₂ (1.3 mL) at −78° C. After stirring for 30 min at −78° C., the reaction was quenched by the addition of MeOH (0.5 mL). The reaction solvent was removed under reduced pressure and the reaction contents were redissolved in MeOH (4 mL). Solid K₂CO₃ (0.21 g, 1.49 mmol, 10 equiv) was then added and the resultant mixture was stirred for 60 min at 50° C. Upon completion, the reaction mixture was cooled to 25° C., quenched with ice cold 1 M HCl (10 mL), and extracted with CH₂Cl₂ (3×15 mL). The combined organic layers were then washed with saturated aqueous NaHCO₃ (10 mL; back-extracted with 3 mL CH₂Cl₂), and brine (10 mL; back-extracted with 3 mL CH₂Cl₂), dried (MgSO₄), filtered, and concentrated. Purification of the resultant residue by flash column chromatography (silica gel, hexanes:EtOAc, 3:1) afforded the desired alkene (0.037 g, 82% yield) as a colorless amorphous solid. Next, Dess-Martin periodinane (0.074 g, 0.174 mmol, 1.5 equiv) was added to a solution of the newly formed alkene (0.035 g, 0.116 mmol, 1.0 equiv) and solid NaHCO₃ (0.049 g, 0.58 mmol, 5.0 equiv) in CH₂Cl₂ (1.2 mL) at 0° C. After stirring for 60 min at 0° C., the reaction mixture was quenched by the addition of 5% aqueous Na₂SO₃ (5 mL). The reaction contents were then poured into saturated aqueous NaHCO₃ (5 mL), extracted with CH₂Cl₂ (3×5 mL), dried (MgSO₄), filtered, and concentrated. Purification of the resultant residue by flash column chromatography (silica gel, hexanes:EtOAc, 9:1) afforded aldehyde S3 (0.030 g, 86% yield) as a colorless amorphous solid.

### Aryl Addition Product S4.

A solution n-BuLi (1.5 M in hexanes, 0.080 mL, 0.120 mmol, 1.2 equiv) was syringed dropwise into a solution of 34 (0.046 g, 0.130 mmol, 1.3 equiv) in THF (3 mL) at −78° C. After stirring for 20 min at −78° C., the resultant aryllithium solution was syringed quickly into a solution of aldehyde S3 (0.030 g, 0.100 mmol, 1.0 equiv) in THF (2 mL) at −40° C. After stirring for 20 min at −40° C., the reaction mixture was quenched by the sequential addition of saturated aqueous NH₄Cl (5 mL) and water (5 mL). The reaction contents were then extracted with EtOAc (3×10 mL), washed with brine (10 mL), dried (MgSO₄), filtered, and concentrated. Purification of the resultant residue by flash column chromatography (silica gel, hexanes:EtOAc, 1:0→4:1) afforded an inseparable mixture of benzylic alcohol diastereomers (1.1:1.0, 0.053 g, 92% overall yield combined) as a colorless amorphous solid. Pressing forward, TFA (0.035 mL, 0.460 mmol, 5.0 equiv) was added dropwise to a solution of the mixture of benzylic alcohol diastereomers (0.053 g, 0.092 mmol, 1.0 equiv) and Et₃SiH (0.147 mL, 0.920 mmol, 10 equiv) in CH₂Cl₂ (1 mL) under argon at 0° C. After stirring for 2.5 h at 0° C., the reaction contents were quenched by the careful addition of saturated aqueous NaHCO₃ (5 mL) and extracted with CH₂Cl₂ (3×5 mL). The combined organic layers were then dried (MgSO₄), filtered, and concentrated. Purification of the resultant residue by flash column chromatography (silica gel, hexanes:CH₂Cl₂, 1:0→1:1) afforded aryl addition product S4 (0.030 g, 58% yield) as a colorless amorphous solid.

### Potential Peyssonol A Structure 45.

A solution of n-BuLi (1.5 M in hexanes, 0.046 mL, 0.070 mmol, 1.3 equiv) was syringed dropwise into a solution of aryl addition product S4 (0.030 g, 0.054 mmol, 1.0 equiv) in THF (1 mL) at −78° C. After stirring for 15 min at −78° C., DMF (0.041 mL, 0.54 mmol, 10 equiv) was added slowly into the reaction mixture via syringe. After stirring for an additional 30 min at −78° C., the reaction mixture was quenched by the addition of saturated aqueous NH₄Cl (2 mL), poured into water (3 mL), and extracted with EtOAc (3×5 mL). The combined organic layers were then washed with brine (5 mL), dried (MgSO₄), filtered, and concentrated. Purification of the resultant residue by flash column chromatography (silica gel, hexanes:EtOAc, 1:0→4:1) afforded protected 45 (0.019 g, 70% yield) as a light yellow amorphous solid. Finally, the newly-synthesized protected 45 (0.019 g, 0.038 mmol, 1.0 equiv) was dissolved in a solution of p-TsOH.H₂O (0.2 M in t-BuOH, 2 mL) and stirred at 70° C. for 2 h. Upon completion, the reaction mixture was poured into water (5 mL) and extracted with EtOAc (3×5 mL). The combined organic layers were then washed with brine (5 mL), dried (MgSO₄), filtered, and concentrated. Purification of the resultant residue by flash column chromatography (silica gel, hexanes:EtOAc, 4:1) afforded potential peyssonol A structure 45 (0.014 g, 88% yield) as a white crystalline solid. 45: R_{f}=0.40 (silica gel, hexanes:EtOAc, 2:1); IR (film) νₘₐₓ 3403 (br), 2971, 2945, 2849, 1643, 1348, 1173, 1156 cm⁻¹; ¹H NMR (400 MHz, C₆D₆) δ 11.13 (s, 1H), 9.24 (s, 1H), 6.85 (s, 1H), 5.78 (s, 1H), 4.76 (s, 1H), 4.65 (s, 1H), 3.85 (s, 1H), 3.83 (dd, J=12.4, 4.8 Hz, 1H), 2.73 (dd, J=16.0, 10.8 Hz, 1H), 2.42 (dd, J=16.0, 2.0 Hz, 1H), 2.15-1.87 (m, 4H), 1.67 (dt, J=4.8, 12.8 Hz, 1H), 1.49-1.32 (m, 2H), 1.15 (dq, J=4.0, 12.8 Hz, 1H), 1.01 (s, 3H), 0.90 (s, 3H), 0.90-0.80 (m, 2H), 0.62 (s, 3H); ¹³C NMR (75 MHz, C₆D₆) δ 195.0, 156.6, 147.2, 146.8, 140.7, 118.6, 118.5, 117.2, 108.6, 69.0, 55.6, 55.4, 40.2, 40.0, 39.9, 37.9, 31.9, 30.8, 25.7, 24.5, 18.5, 14.5; HRMS (FAB) calcd for C₂₂H₃₀BrO₃ [M+H]⁺421.1378. found 421.1398.

### 4. Total Synthesis of the Revised Structure of Peyssonol A (50).

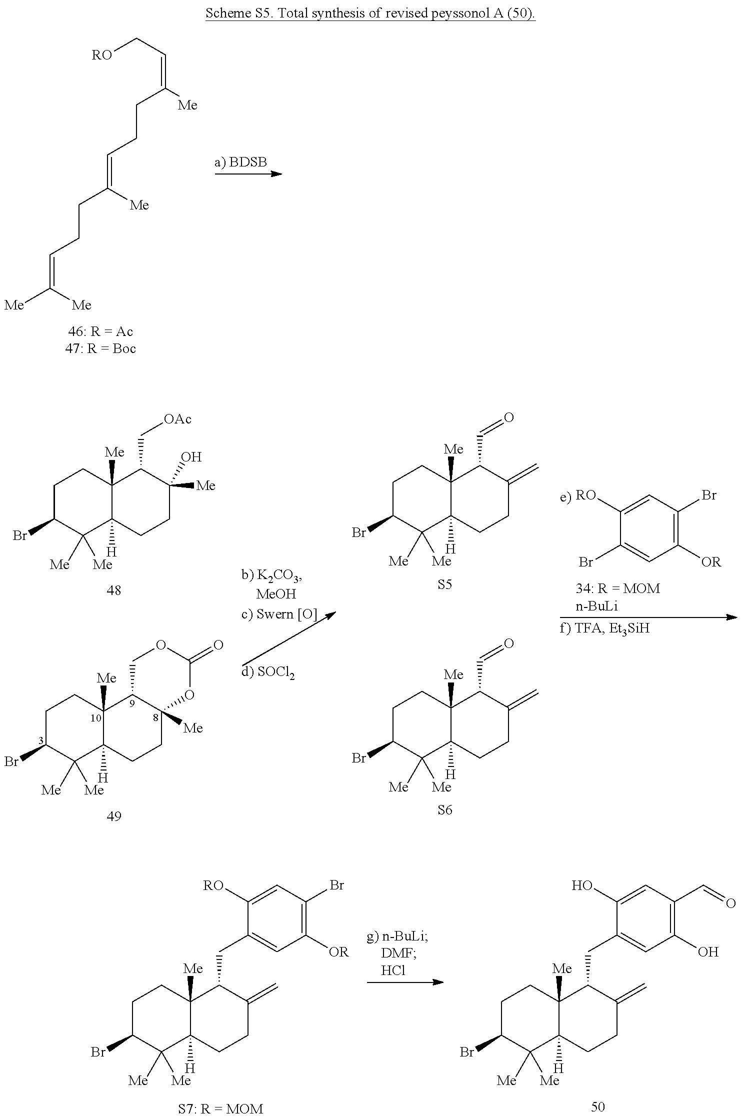

### (2Z,6E)-Farnesol (53)

[61] PBr₃ (4.7 mL, 50 mmol, 0.5 equiv) was added dropwise to a solution of geraniol (18.1 mL, 100 mmol, 1.0 equiv) in Et₂O (300 mL) at −20° C. The reaction mixture was stirred for 60 min, during which time the temperature was allowed to warm slowly to 0° C. Upon completion, the reaction contents were quenched by the addition of ice-cold water (500 mL) and extracted with hexanes (4×200 mL). The combined organic layers were washed with saturated aqueous NaHCO₃ (200 mL) and brine (200 mL), dried (MgSO₄), filtered, and concentrated. Any residual water was removed by co-evaporation with anhydrous benzene, and the crude geranyl bromide product was carried forward without additional purification. Next, ethyl acetoacetate (15.2 mL, 120 mmol, 1.2 equiv) was added dropwise under constant flow of argon to a suspension of NaH (60% dispersion in mineral oil, 5.00 g, 125 mmol, 1.25 equiv) in THF (260 mL) at 0° C. The reaction mixture was stirred for 30 min at 0° C., then a solution of n-BuLi (1.5 M in hexanes, 83.0 mL, 125 mmol, 1.25 equiv) was added slowly at 0° C. and the resultant light orange solution was stirred for an additional 10 min at 0° C. This dianion solution was then cannulated slowly into a solution of geranyl bromide (100 mmol presumed, 1.0 equiv) in THF (100 mL) at 0° C. After stirring for 15 min at 0° C., the reaction mixture was quenched by the careful addition of saturated aqueous NH₄Cl (250 mL) and water (250 mL). The crude product was extracted with hexanes:EtOAc (1:1, 3×300 mL), washed with brine (300 mL), dried (MgSO₄), filtered, and concentrated. The resultant yellow oil was purified by flash column chromatography (silica gel, hexanes:EtOAc, 9:1) to afford the desired intermediate (22.4 g, 84% over two steps) as a viscous yellow oil. Next, KOt-Bu (10.4 g, 92.5 mmol, 1.1 equiv) was sealed under argon, cooled to 0° C., and dissolved in DMF (250 mL). The newly synthesized material from above (22.4 g, 84.1 mmol, 1.0 equiv) was then added dropwise and the reaction mixture was stirred for 5 min at 0° C. Diethyl chlorophosphate (15.8 mL, 109 mmol, 1.3 equiv) was syringed into the reaction mixture, and the resultant contents were stirred for 15 min at 0° C. Upon completion, the reaction mixture was poured into 0.25 M HCl (500 mL) and extracted with EtOAc (3×200 mL). The combined organic layers were washed with saturated aqueous NaHCO₃ (200 mL) and brine (200 mL), dried (MgSO₄), filtered, and concentrated. The resultant crude orange oil, consisting of a 3.2:1 mixture of E:Z isomers based on ¹H NMR analysis, was purified by flash column chromatography (silica gel, hexanes:EtOAc, 9:1→7:3) to afford the pure E-isomer of the desired enol phosphate (23.5 g, 70% yield) as a light orange oil. Next, CuI (19.0 g, 100 mmol, 2.5 equiv) was sealed under argon and suspended in THF (375 mL) at 0° C. A solution of MeLi (1.6 M in Et₂O, 62.5 mL, 100 mmol, 2.5 equiv) was added slowly, and the resultant cloudy dark yellow solution was stirred for 15 min at 0° C., then cooled to −50° C. A solution of MeMgCl (3.0 M in THF, 53.3 mL, 160 mmol, 4.0 equiv) was added slowly, and the resultant cloudy pale yellow solution was stirred for 30 min at −50° C. A solution of previously synthesized E-alkene (16.1 g, 40.0 mmol, 1.0 equiv) in THF (25 mL) was then cannulated dropwise into the methyl cuprate solution, and the resultant mixture was stirred for 4 h at −50° C. and then allowed to warm slowly to −30° C. over the course of 1 h. Upon completion, the reaction mixture was quenched by the careful addition of saturated aqueous NH₄Cl (400 mL) and the resultant slurry was stirred vigorously for 60 min and then filtered to remove insoluble copper salts. The filtrate was extracted with hexanes:EtOAc (1:1, 3×300 mL), and the combined organic layers were washed with saturated aqueous NH₄Cl (2×200 mL) and brine (300 mL), dried (MgSO₄), filtered, and concentrated to afford crude ethyl (2Z,6E)-farnesate along with a small amount of its olefinic isomer (10:1). The minor isomer was removed by careful flash column chromatography (silica gel, hexanes:CH₂Cl₂, 1:0→1:1) to afford pure ethyl (2Z,6E)-farnesate (8.44 g, 80% yield) as a colorless viscous oil. Finally, reduction of ethyl (2Z,6E)-farnesate was completed using the same procedure elucidated above for the reduction of ethyl (2E,6Z)-farnesate to afford (2Z,6E)-farnesol (53, 6.24 g, 88% yield) as a colorless viscous oil. 53: R_{f}=0.30 (silica gel, hexanes:EtOAc, 4:1); IR (film) νₘₐₓ 3325 (br), 2966, 2916, 2857, 1446, 1376, 1001 cm⁻¹; ¹H NMR (400 MHz, CDCl₃) δ 5.44 (dt, J=1.6, 7.2 Hz, 1H), 5.14-5.05 (m, 2H), 4.10 (dd, J=7.2, 0.8 Hz, 2H), 2.14-1.96 (m, 8H), 1.75 (s, 3H), 1.68 (s, 3H), 1.60 (s, 6H), 1.27 (s, 1H); ¹³C NMR (75 MHz, CDCl₃) δ 140.1, 136.1, 131.5, 124.5, 124.4, 123.7, 59.2, 39.8, 32.1, 26.8, 26.6, 25.8, 23.6, 17.8, 16.1; HRMS (FAB) calcd for C₁₅H₂₇O [M+H]⁺ 223.2062. found 223.2060.

### (2Z,6E)-Farnesyl Acetate (46).

Prepared as in 30; 0.107 g (91% yield) of a colorless viscous oil. 46: R_{f}=0.55 (silica gel, hexanes:EtOAc, 4:1); IR (film) νₘₐₓ 2967, 2918, 2857, 1741, 1445, 1377, 1232, 1022 cm⁻¹; ¹H NMR (400 MHz, CDCl₃) δ 5.35 (t, J=7.2 Hz, 1H), 5.12-5.04 (m, 2H), 4.55 (d, J=7.2 Hz, 2H), 2.16-1.93 (m, 8H), 2.03 (s, 3H), 1.76 (s, 3H), 1.67 (s, 3H), 1.59 (s, 6H); ¹³C NMR (75 MHz, CDCl₃) δ 171.1, 142.7, 135.9, 131.4, 124.4, 123.5, 119.3, 61.2, 39.8, 32.3, 26.8, 26.7, 25.8, 23.6, 21.1, 17.8, 16.1; HRMS (FAB) calcd for C₁₇H₂₈O₂ [M]⁺ 264.2089. found 264.2084.

(2Z,6E)-Farnesyl t-Butyl Carbonate (47).

Prepared as in 31; 1.12 g (96% yield) of a colorless viscous oil. 47: R_{f}=0.67 (silica gel, hexanes:EtOAc, 4:1); IR (film) νₘₐₓ 2970, 2930, 1740, 1369, 1277, 1254, 1168 cm⁻¹; ¹H NMR (400 MHz, CDCl₃) δ 5.37 (dt, J=1.2, 7.2 Hz, 1H), 5.14-5.05 (m, 2H), 4.55 (dd, J=7.2, 0.8 Hz, 2H), 2.16-1.94 (m, 8H), 1.75 (s, 3H), 1.67 (s, 3H), 1.59 (s, 6H), 1.47 (s, 9H); ¹³C NMR (75 MHz, CDCl₃) δ 154.0, 142.6, 136.0, 131.3, 124.7, 123.9, 119.7, 81.7, 63.7, 39.9, 32.5, 28.1 (3C), 27.1, 26.9, 25.6, 23.4, 17.7, 16.1; HRMS (FAB) calcd for C₂₀H₃₅O₃ [M+H]⁺ 323.2586. found 323.2578.

### Trans-Decalin Framework 48.

A solution of BDSB (13, 0.228 g, 0.42 mmol, 1.1 equiv) in nitromethane (1 mL) was added via syringe into a solution of 46 (0.100 g, 0.38 mmol, 1.0 equiv) in nitromethane (37 mL) at 0° C. After stirring for 30 s at 0° C., the reaction mixture was quenched by the sequential addition of 5% aqueous Na₂SO₃ (20 mL) and saturated aqueous NaHCO₃ (20 mL). The resultant biphasic mixture was stirred vigorously for 1 h at 25° C., poured into brine (40 mL), and extracted with EtOAc (3×50 mL). The combined organic layers were then washed with brine (50 mL), dried (MgSO₄), filtered, and concentrated. Purification of the resultant residue by flash column chromatography (silica gel, hexanes:EtOAc, 1:0→3:2) afforded trans-decalin framework 48 (0.055 g, 40% yield) as a colorless amorphous solid. 48: R_{f}=0.34 (silica gel, hexanes:EtOAc, 3:2); IR (film) νₘₐₓ 3469 (br), 2964, 1734, 1385, 1366, 1245, 1029 cm⁻¹; ¹H NMR (400 MHz, CDCl₃) δ 4.61 (dd, J=12.0, 2.0 Hz, 1H), 4.27 (dd, J=12.0, 5.2 Hz, 1H), 3.97 (dd, J=12.0, 4.0 Hz, 1H), 2.27 (dq, J=3.6, 13.2 Hz, 1H) 2.17-2.08 (m, 2H), 2.04 (s, 3H), 1.75-1.67 (m, 3H), 1.46 (s, 3H), 1.45-1.38 (m, 3H), 1.35-1.27 (m, 2H), 1.14 (s, 3H), 1.07 (s, 3H), 0.94 (s, 3H); ¹³C NMR (75 MHz, CDCl₃) δ 171.5, 72.0, 68.8, 63.3, 59.2, 48.5, 39.9, 38.6, 38.2, 37.9, 32.2, 31.1, 30.8, 24.6, 21.7, 21.4, 18.1; HRMS (FAB) calcd for C₁₇H₂₈BrO₂ [M-OH]⁺ 343.1273. found 343.1256.

Trans-Decalin Framework 49. A solution of BDSB (13, 1.553 g, 2.83 mmol, 1.1 equiv) in nitromethane (8 mL) pre-cooled to −25° C. was added via syringe into a solution of 47 (0.831 g, 2.58 mmol, 1.0 equiv) in nitromethane (250 mL) at −25° C. Following this addition, the reaction mixture was removed from the cold bath and stirred for 60 min at 25° C., then quenched by the sequential addition of 5% aqueous Na₂SO₃ (100 mL) and saturated aqueous NaHCO₃ (100 mL). The resultant biphasic mixture was stirred vigorously for 1 h at 25° C. and then was poured into brine (200 mL) and extracted with EtOAc (3×200 mL). The combined organic layers were washed with brine (300 mL), dried (MgSO₄), filtered, and concentrated. Purification of the resultant residue by flash column chromatography (silica gel, hexanes:EtOAc, 9:1→1:1) afforded trans-decalin framework 49 (0.502 g, 56% yield) as a white crystalline solid. 49: R_{f}=0.46 (silica gel, hexanes:EtOAc, 2:3); IR (film) νₘₐₓ 2971, 2878, 1751, 1247, 1118, 732 cm⁻¹; ¹H NMR (400 MHz, CDCl₃) δ 4.39-4.23 (m, 2H), 3.87 (dd, J=12.4, 4.4 Hz, 1H), 2.26 (dq, J=4.4, 12.8 Hz, 1H), 2.11 (dq, J=13.6, 4.0 Hz, 1H), 2.06-1.78 (m, 4H), 1.62 (s, 3H), 1.51-1.37 (m, 3H), 1.20 (s, 3H), 1.17 (m, 1H), 1.06 (s, 3H), 0.92 (s, 3H); ¹³C NMR (75 MHz, CDCl₃) δ 149.5, 86.2, 66.9, 65.8, 50.6, 48.8, 39.8, 37.7, 37.3, 36.0, 30.6, 30.4 (2C), 23.9, 21.7, 18.5; HRMS (FAB) calcd for C₁₆H₂₆BrO₃ [M+H]⁺ 345.1065. found 345.1053. [Note: the stereochemistry at C-8 was originally ambiguous, although inconsequential since this stereocenter is later ablated. A chair-chair-chair conformation of the tricycle in which the C₁₋₃ hydrogen is axial (as apparent from J-values from the ¹H NMR) has the group at C-9 in the α-position and axial, implying that the neighboring carbon-oxygen bond at C-8 must also be in the α-orientation. This hypothesis was verified by NOE analysis, which clearly showed a cross peak between the methyl group at C-8 and the methyl group at C-10. This result indicates that the methyl group is in the axial β-position at C-8].

### Aldehyde S5.

Solid K₂CO₃ (0.742 g, 5.37 mmol, 3.0 equiv) was added to a solution of 49 (0.618 g, 1.790 mmol, 1.0 equiv) in MeOH (50 mL) at 40° C. The reaction mixture was stirred for 3 h at 40° C. and then was quenched by the addition of ice-cold saturated aqueous NH₄Cl (40 mL). The crude product was extracted into EtOAc (4×50 mL), washed with brine (50 mL), dried (MgSO₄), filtered, and concentrated under reduced pressure to afford the corresponding diol as a white crystalline solid which was carried forward without any additional purification. [Note: The diol was co-evaporated with anhydrous toluene to remove traces of water before being subjected to the subsequent oxidation procedure]. Next, a solution of DMSO (0.381 mL, 5.36 mmol, 3 equiv) in CH₂Cl₂ (3 mL) was added dropwise to a solution of oxalyl chloride (0.233 mL, 2.68 mmol, 1.5 equiv) in CH₂Cl₂ (17 mL) at −78° C. After stirring for 5 min at −78° C., a solution of the diol (1.790 mmol presumed, 1.0 equiv) in a mixture of CH₂Cl₂ (20 mL) and DMSO (1 mL, to enhance solubility) was added slowly. After stirring for an additional 5 min at −78° C., Et₃N (1.48 mL, 10.7 mmol, 6 equiv) was added. The reaction mixture was allowed to warm slowly from −78° C. to −40° C. over the course of 1 h and then was quenched by the careful addition of saturated aqueous NaHCO₃ (50 mL). The reaction contents were then extracted with CH₂Cl₂ (3×50 mL). The combined organic layers were washed with water (100 mL; back-extracted with 10 mL CH₂Cl₂) and brine (100 mL; back-extracted with 10 mL CH₂Cl₂), dried (MgSO₄), filtered, and concentrated to afford a light yellow solid. Purification of this residue by flash column chromatography (silica gel, hexanes:EtOAc, 7:3) afforded the desired aldehyde intermediate (0.516 g, 91% yield over 2 steps) as a white crystalline solid. R_{f}=0.49 (silica gel, hexanes:EtOAc, 2:3); IR (film) νₘₐₓ 3461 (br), 2952, 2873, 1716, 1464, 1389, 1158, 1102, 904, 733 cm⁻¹; ¹H NMR (400 MHz, CDCl₃) δ 9.86 (d, J=6.0 Hz, 1H), 3.90 (dd, J=12.4, 4.4 Hz, 1H), 2.23 (dq, J=4.0, 12.8 Hz, 1H), 2.12-1.88 (m, 6H), 1.60-1.50 (m, 2H), 1.45 (s, 3H), 1.40 (dt, J=14.0, 3.6 Hz, 1H), 1.31 (dt, J=4.0, 12.8 Hz, 1H), 1.13 (s, 3H), 1.12 (s, 3H), 0.94 (s, 3H); ¹³C NMR (75 MHz, CDCl₃) δ 205.2, 72.3, 71.9, 67.7, 50.6, 39.9, 39.2, 39.1, 38.0, 32.0, 30.7, 30.6, 23.3, 22.1, 18.5; HRMS (FAB) calcd for C₁₅H₂₄BrO₂ [M−H]⁺ 315.0960. found 315.0957. Finally, a solution of this newly prepared aldehyde (0.516 g, 1.63 mmol, 1.0 equiv) and Et₃N (1.13 mL, 8.13 mmol, 5.0 equiv) in CH₂Cl₂ (32 mL) under N₂ gas (do not use argon!) was frozen at −196° C. in a liquid N₂ bath. A solution of SOCl₂ (0.177 mL, 2.44 mmol, 1.5 equiv) in CH₂Cl₂ (1 mL) was added dropwise to this frozen mixture over approximately 3 min. The resultant reaction mixture was moved to a −97° C. bath (liquid N₂/CH₂Cl₂ slurry) and allowed to slowly melt/react for 1 h. The reaction flask was then removed from the cold bath and its contents were quenched by the addition of MeOH (1 mL). The crude reaction materials were filtered through a silica gel plug (25×100 mm) with CH₂Cl₂ (150 mL) to remove any ammonium salts. Concentration yielded a solid residue that was purified by careful flash column chromatography (silica gel, hexanes:CH₂Cl₂, 1:0→2:1) to afford aldehyde S5 (0.404 g, 83% yield) along with aldehyde S6 (0.052 g, 11% yield), both as white crystalline solids. S5: R_{f}=0.40 (silica gel, hexanes:CH₂Cl₂, 1:1); IR (film) νₘₐₓ 2973, 2948, 1715, 1459, 1385, 1370, 1157, 899 cm⁻¹; ¹H NMR (400 MHz, CDCl₃) δ 9.79 (d, J=3.2 Hz, 1H), 4.97 (s, 1H), 4.81 (s, 1H), 4.02 (dd, J=12.4, 4.0 Hz, 1H), 2.64 (d, J=3.2 Hz, 1H), 2.44 (m, 1H), 2.31-2.09 (m, 3H), 1.90-1.72 (m, 3H), 1.53 (m, 1H), 1.41 (dt, J=13.6, 3.6 Hz, 1H), 1.12 (s, 3H), 0.98 (s, 3H), 0.96 (s, 3H); ¹³C NMR (75 MHz, CDCl₃) 201.7, 141.3, 114.7, 70.6, 68.3, 48.5, 40.0, 39.1, 38.2, 33.2, 31.2, 30.8, 24.5, 21.5, 18.6; HRMS (EI) calcd for C₁₅H₂₃BrO [M]⁺ 298.0932. found 298.0923. S6: R_{f}=0.37 (silica gel, hexanes:CH₂Cl₂, 1:1); IR (film) νₘₐₓ 2974, 2917, 1714, 1451, 1439, 1388, 866, 818 cm⁻¹; ¹H NMR (400 MHz, CDCl₃) δ 9.56 (d, J=5.2 Hz, 1H), 5.75 (br s, 1H), 3.97 (dd, J=12.4, 3.6 Hz, 1H), 2.36-2.07 (m, 5H), 1.80 (dd, J=11.2, 5.2 Hz, 1H), 1.57 (s, 3H), 1.54 (m, 1H), 1.43 (dt, J=4.0, 13.2 Hz, 1H), 1.10 (s, 3H), 1.06 (s, 3H), 0.96 (s, 3H); ¹³C NMR (75 MHz, CDCl₃) δ 201.5, 126.4, 126.2, 68.0 (2C), 45.3, 39.7, 38.6, 36.1, 30.8, 30.4, 25.7, 22.4, 21.4, 18.3; HRMS (EI) calcd for C₁₅H₂₃BrO [M]⁺ 298.0932. found 298.0922.

### Aryl Addition Product S7.

A solution n-BuLi (1.4 M in hexanes, 1.06 mL, 1.48 mmol, 1.1 equiv) was added dropwise via syringe into a solution of 34 (0.577 g, 1.62 mmol, 1.2 equiv) in THF (50 mL) at −78° C. After stirring for 15 min at −78° C., the resultant aryllithium solution was cannulated slowly into a solution of aldehyde S5 (0.404 g, 1.35 mmol, 1.0 equiv) in THF (13 mL) at −40° C. After stirring for 5 min at −40° C., the reaction mixture was quenched by the addition of saturated aqueous NH₄Cl (20 mL) and water (30 mL). The crude product was extracted with EtOAc (3×40 mL), washed with brine (50 mL), dried (MgSO₄), filtered, and concentrated. Purification of the resultant residue by flash column chromatography (silica gel, hexanes:EtOAc, 1:0→9:1) afforded a 2.3:1 mixture of separable benzylic alcohol diastereomers (0.400 g, 51% yield of the less polar diastereomer; 0.222 g, 29% yield of the more polar diastereomer), each as a light yellow foam. [Note: originally these two diastereomers were reacted together in the next step, but it was found that one was significantly more reactive than the other, and since the product slowly decomposes in the acidic reaction media, the yield could be increased by reacting the two diastereomers separately]. Pressing forward, TFA (0.267 mL, 3.47 mmol, 5.0 equiv) was added dropwise to a solution of the less polar benzylic alcohol diastereomer (0.400 g, 0.694 mmol, 1.0 equiv) and Et₃SiH (1.106 mL, 6.94 mmol, 10 equiv) in CH₂Cl₂ (7 mL) under argon at 0° C. After stirring for 30 min at 0° C., the reaction mixture was quenched by the careful addition of saturated aqueous NaHCO₃ (20 mL) and extracted with CH₂Cl₂ (3×15 mL). The combined organic layers were then dried (MgSO₄), filtered, and concentrated. Separately, TFA (0.148 mL, 1.93 mmol, 5.0 equiv) was added dropwise to a solution of the more polar benzylic alcohol diastereomer (0.222 g, 0.385 mmol, 1.0 equiv) and Et₃SiH (0.613 mL, 3.85 mmol, 10 equiv) in CH₂Cl₂ (4 mL) under argon at 0° C. After stirring for 90 min at 0° C., the reaction mixture was quenched by the careful addition of saturated aqueous NaHCO₃ (20 mL) and extracted into CH₂Cl₂ (3×15 mL). The combined organic layers were then dried (MgSO₄), filtered, and concentrated. Combination of the two crude products and purification by flash column chromatography (silica gel, hexanes:CH₂Cl₂, 1:0→1:1) afforded aryl addition product S7 (0.351 g, 58% yield) as a colorless amorphous solid. S7: R_{f}=0.52 (silica gel, hexanes:EtOAc, 4:1); IR (film) νₘₐₓ 2948, 1489, 1380, 1217, 1151, 1081, 999 cm⁻¹; ¹H NMR (400 MHz, CDCl₃) δ 7.23 (s, 1H), 6.78 (s, 1H), 5.12 (d, J=1.6 Hz, 2H), 5.08 (s, 2H), 4.49 (t, J=2.0 Hz, 1H), 4.09 (t, J=2.0 Hz, 1H), 4.05 (dd, J=12.4, 4.0 Hz, 1H), 3.50 (s, 3H), 3.47 (s, 3H), 2.91 (dd, J=13.2, 4.0 Hz, 1H), 2.53 (dd, J=12.4, 11.2 Hz, 1H), 2.35-2.12 (m, 4H), 1.98-1.86 (m, 2H), 1.75 (m, 1H), 1.58-1.41 (m, 2H), 1.28 (dt, J=13.6, 3.6 Hz, 1H), 1.13 (s, 3H), 0.98 (s, 6H); ¹³C NMR (75 MHz, CDCl₃) δ 150.9, 148.3, 146.5, 131.2, 119.6, 119.0, 111.1, 110.2, 96.2, 95.4, 69.6, 58.2, 56.4, 56.2, 46.8, 39.9, 38.3, 37.9, 31.6, 31.4, 31.0, 27.9, 25.1, 22.6, 18.6; HRMS (FAB) calcd for C₂₅H₃₆Br₂O₄ [M]⁺ 558.0980. found 558.1001.

### Revised Peyssonol A (50).

A solution of n-BuLi (1.6 M in hexanes, 0.407 mL, 0.652 mmol, 1.1 equiv) was added dropwise via syringe to a solution of S7 (0.332 g, 0.592 mmol, 1.0 equiv) in THF (30 mL) at −78° C. After stirring for 15 min at −78° C., DMF (0.228 mL, 2.96 mmol, 5.0 equiv) was added via syringe slowly into the reaction mixture. After stirring for an additional 60 min at −78° C., concentrated aqueous HCl (2.8 mL; reaction solution now ˜1 M in HCl) was added dropwise and the reaction mixture was warmed slowly to 50° C. After stirring for 1 h at 50° C., the resultant green reaction mixture was allowed to cool to 25° C., poured into water (30 mL), and extracted with EtOAc (3×20 mL). The combined organic layers were washed with brine, dried (MgSO₄), filtered, and concentrated. Purification by flash column chromatography (silica gel, hexanes:EtOAc, 1:0→4:1) afforded revised peyssonol A (50, 0.192 g, 77% yield) as a light yellow powder. 50: R_{f}=0.41 (silica gel, hexanes:EtOAc, 2:1); IR (film) νₘₐₓ 3383 (br), 2972, 2947, 2859, 1651, 1369, 1197, 1167 cm⁻¹; ¹H NMR (400 MHz, C₆D₆) δ 11.13 (s, 1H), 9.25 (s, 1H), 6.68 (s, 1H), 5.81 (s, 1H), 4.50 (t, J=2.0 Hz, 1H), 4.23 (t, J=2.0 Hz, 1H), 3.82 (s, 1H), 3.74 (dd, J=12.4, 4.0 Hz, 1H), 2.69 (dd, J=12.8, 4.0 Hz, 1H), 2.33 (dd, J=12.8 Hz, 11.2 Hz, 1H), 2.14 (dq, J=3.2, 13.2 Hz, 1H), 2.08-1.90 (m, 3H), 1.88 (dd, J=11.2, 4.0 Hz, 1H), 1.50-1.36 (m, 2H), 1.21-1.12 (m, 2H), 1.04 (s, 3H), 0.93 (s, 3H), 0.92 (m, 1H), 0.80 (s, 3H); ¹³C NMR (100 MHz, C₆D₆) δ 195.0, 156.4, 147.0, 146.8, 140.4, 120.0, 119.0, 117.4, 111.2, 69.1, 57.6, 46.6, 39.8, 37.7, 31.7, 31.4, 30.9, 28.9, 25.0, 22.3, 18.6; HRMS (FAB) calcd for C₂₂H₃₀BrO₃ [M+H]⁺ 421.1378. found 421.1350.

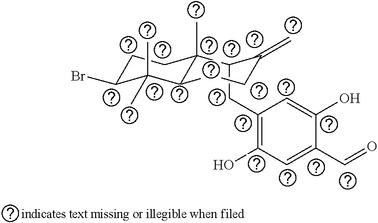 [SMILES: C=C1CCC2C(C)(C)C(Br)CCC2(C)C1Cc1cc(O)c(C=O)cc1O]

### Differences in the ¹³C Data Between Synthetic and Natural Peyssonol a (See Comparison Table on the Next Page)

| | | | | |
|---|---|---|---|---|
| Natural peyssonol A | 50 | 3 | 40 | 45 |

¹H

| | | | | |
|---|---|---|---|---|
| 0.80 (s, 3H) | 0.80 (s, 3H) | 0.91 (s, 3H) | 0.97 (s, 3H) | 0.62 (s, 3H) |
| 0.90 (s, 3H) | 0.93 (s, 3H) | 0.98 (s, 3H) | 1.06 (s, 3H) | 0.90 (s, 3H) |
| 0.92 (m) | 0.92 (m) | | | |
| 1.05 (s, 3H) | 1.04 (s, 3H) | 1.24 (s, 3H) | 1.21 (s, 3H) | 1.01 (s, 3H) |
| 1.15 (m, 2H) | 1.12-1.21 (m, 2H) | | | |
| 1.39 (m) | 1.36-1.50 (m 2H) | | | |
| 1.42 (m) | | | | |
| 1.85 (m) | 1.88 (dd, J = 11.2, | | | |
| | 4.0) | | | |
| 1.89 (m) | 1.95-2.08 (m, 3H) | | | |
| 1.96 (m) | | | | |
| 2.05 (m) | | | | |
| 2.14 (qd, J = 13, 3) | 2.14 (qd, J = 13.2, | | | |
| | 3.2) | | | |
| 2.30 (t, J = 12) | 2.33 (dd, J = 12.8, | 2.49 (app d, J = 16.0) | 2.39 (dd, J = 13.2, 11.6) | 2.42 (dd, J = 16.0, 2.0) |
| | 11.2) | | | |
| 2.70 (dd, J = 12, 4) | 2.69 (dd, J = 12.8, | 2.68 (dd, J = 16.4, | 2.80 (dd, J = 13.2, 4.0) | 2.73 (dd, J = 16.0, |
| | 4.0) | 10.8) | | 10.8) |
| 3.70 (dd, J = 10, 3) | 3.74 (dd, J = 12.4, | 3.86 (dd, J = 12.8, | 3.97 (dd, J = 12.4, 4.8) | 3.83 (dd, J = 12.4, 4.8) |
| | 4.0) | 4.8) | | |
| 4.20 (t, J = 2) | 4.23 (t, J = 2.0) | 4.62 (s) | 4.24 (s) | 4.65 (s) |
| 4.50 (t, J = 2) | 4.50 (t, J = 2.0) | 4.74 (s) | 4.51 (s) | 4.76 (s) |
| 5.80 (s) | 5.81 (s) | 5.74 (s) | 5.74 (s) | 5.78 (s) |
| 6.70 (s) | 6.68 (s) | 6.84 (s) | 6.70 (s) | 6.85 (s) |
| 9.40 (s) | 9.25 (s) | 9.20 (s) | 9.24 (s) | 9.24 (s) |
| 11.20 (s, OH) | 11.13 (s, OH) | 11.11 (s) | 11.14 (s) | 11.13 (s) |

¹³C

| | | | | |
|---|---|---|---|---|
| 19.2 | 18.6 | 25.6 | 25.8 | 14.5 |
| 22.5 | 22.3 | 26.8 | 27.8 | 18.5 |
| 25.6 | 25.0 | 28.2 | 28.3 | 24.5 |
| 29.0 | 28.9 | 28.6 | 29.3 | 25.7 |
| 31.3 | 30.9 | 28.6 | 30.3 | 30.8 |
| 32.1 | 31.4 | 32.0 | 31.5 | 31.9 |
| 32.2 | 31.7 | 32.3 | 32.3 | 37.9 |
| 37.2 | 37.7 | 37.6 | 34.4 | 39.9 |
| 39.0 | 38.4 | 40.1 | 38.8 | 40.0 |
| 40.1 | 39.8 | 41.6 | 39.7 | 40.2 |
| 46.0 | 46.6 | 53.0 | 48.2 | 55.4 |
| 57.3 | 57.6 | 54.8 | 57.7 | 55.6 |
| 69.5 | 69.1 | 64.5 | 64.3 | 69.0 |
| 111.4 | 111.2 | 107.8 | 110.0 | 108.6 |
| 117.6 | 117.4 | 117.0 | 117.3 | 117.2 |
| 118.2 | 119.0 | 118.2 | 118.9 | 118.5 |
| 120.2 | 120.0 | 128.6 | 119.9 | 118.6 |
| 140.0 | 140.4 | 141.0 | 140.3 | 140.7 |
| 146.0 | 146.8 | 146.8 | 146.9 | 146.8 |
| 146.1 | 147.0 | 147.6 | 147.2 | 147.2 |
| 156.0 | 156.4 | 156.6 | 156.4 | 156.6 |

### 5. Total Synthesis of Peyssonoic Acid A (51)

### Allylated Building Block 54.

A solution of n-BuLi (2.5 M in hexanes, 0.876 mL, 2.19 mmol, 1.0 equiv) was added via syringe dropwise into a solution of 34 (0.780 g, 2.19 mmol, 1.0 equiv) in THF (22 mL) at −78° C. After stirring for 20 min at −78° C., the resultant aryllithium solution was cannulated onto dry CuI (0.209 g, 1.10 mmol, 0.5 equiv) and stirred for 10 min at 0° C., during which time the solids dissolved to form a homogeneous yellow solution. Allyl bromide (0.568 mL, 6.57 mmol, 3.0 equiv) was then added via syringe into the aryl cuprate solution at 0° C. and the resultant mixture was stirred for 20 min at 0° C. Upon completion, the reaction contents were quenched by the careful addition of saturated aqueous NH₄Cl (15 mL). The reaction mixture was then poured into water (15 mL) and extracted with hexanes:EtOAc (1:1, 3×30 mL). The combined organic layers were washed with brine (30 mL), dried (MgSO₄), filtered, and concentrated. Purification of the resultant residue by flash column chromatography (silica gel, hexanes:EtOAc, 9:1) afforded 54 (0.525 g, 75% yield) as a colorless amorphous solid. 54: R_{f}=0.56 (silica gel, hexanes:CH₂Cl₂, 1:2); IR (film) νₘₐₓ 2955, 2903, 2827, 1489, 1151, 1081, 994, 921 cm⁻¹; ¹H NMR (400 MHz, CDCl₃) δ 7.28 (s, 1H), 6.95 (s, 1H), 5.92 (m, 1H), 5.16 (s, 2H), 5.12 (s, 2H), 5.07 (s, 1H), 5.03 (m, 1H), 3.53 (s, 3H), 3.47 (s, 3H), 3.34 (d, J=6.4 Hz, 2H); ¹³C NMR (100 MHz, CDCl₃) δ 150.6, 148.8, 136.3, 130.0, 119.5, 118.7, 116.1, 110.8, 96.1, 95.3, 56.6, 56.2, 34.4; HRMS (FAB) calcd for C₁₃H₁₇BrO₄ [M]⁺ 316.0310. found 316.0311.

Cation-π Cyclization Precursor 55. PBr₃ (0.050 mL, 0.53 mmol, 0.5 equiv) was added dropwise to a solution of (2Z,6E)-farnesol (53, 0.234 g, 1.052 mmol, 1.0 equiv) in Et₂O (4 mL) at −20° C. The reaction mixture was stirred for 60 min, during which time the temperature was allowed to warm slowly to 0° C. Upon completion, the reaction contents were quenched by the addition of ice-cold water (15 mL) and extracted with hexanes (4×10 mL). The combined organic layers were washed with saturated aqueous NaHCO₃ (15 mL) and brine (20 mL), dried (MgSO₄), filtered, and concentrated to afford (2Z,6E)-farnesyl bromide (0.264 g, 88% yield) which was carried forward without further purification. [Note: co-evaporation with anhydrous benzene was undertaken prior to arylation]. Next, a solution of n-BuLi (2.5 M in hexanes, 0.536 mL, 1.34 mmol, 1.7 equiv) was added dropwise via syringe into a solution of 54 (0.425 g, 1.34 mmol, 1.7 equiv) in THF (14 mL) at −78° C. After stirring 15 min at −78° C., the resultant aryllithium solution was added rapidly via syringe into a solution of (2Z,6E)-farnesyl bromide (0.204 g, 0.788 mmol, 1.0 equiv) in THF (2 mL) at −40° C. The reaction mixture was allowed to warm slowly to 5° C. over the course of 2 h and then was quenched by the careful addition of saturated aqueous NH₄Cl (10 mL). The reaction contents were then poured into water (10 mL) and extracted with hexanes:EtOAc (1:1, 3×20 mL). The combined organic layers were washed with brine (20 mL), dried (MgSO₄), filtered, and concentrated. Purification of the resultant residue by flash column chromatography (silica gel, hexanes:CH₂Cl₂, 4:1→2:3) afforded 55 (0.372 g, 84% yield) as a colorless viscous oil. 55: R_{f}=0.64 (silica gel, hexanes:CH₂Cl₂, 1:2); IR (film) νₘₐₓ 2927, 2855, 1503, 1149, 1080, 1011, 922 cm⁻¹; ¹H NMR (400 MHz, CDCl₃) δ 6.87 (s, 1H), 6.86 (s, 1H), 5.97 (m, 1H), 5.29 (t, J=7.2 Hz, 1H), 5.18 (t, J=6.4 Hz, 1H), 5.12 (s, 2H), 5.10 (s, 2H), 5.12-5.02 (m, 3H), 3.48 (s, 6H), 3.36 (d, J=6.8 Hz, 2H), 3.32 (d, J=7.6 Hz, 2H), 2.19-1.97 (m, 8H), 1.73 (s, 3H), 1.68 (s, 3H), 1.62 (s, 3H), 1.61 (s, 3H); ¹³C NMR (100 MHz, CDCl₃) δ 149.8 (2C), 137.0, 136.1, 135.2, 131.3, 129.9, 127.8, 124.4, 124.2, 123.2, 116.5 (2C), 115.4, 95.4, 95.2, 56.0 (2C), 39.7, 34.3, 32.0, 28.4, 26.7, 26.6, 25.7, 23.5, 17.7, 16.0; HRMS (FAB) calcd for C₂₈H₄₂O₄ [M]⁺ 442.3083. found 442.3073.

### Cation-Cyclization Product 56.

A solution of BDSB (13, 0.239 g, 0.435 mmol, 1.1 equiv) in nitromethane (2 mL) pre-cooled to −25° C. was syringed quickly into a solution of 55 (0.175 g, 0.40 mmol, 1.0 equiv) in nitromethane (38 mL) at −25° C. After stirring for 5 min at −25° C., the reaction mixture was quenched by the sequential addition of 5% aqueous Na₂SO₃ (20 mL) and saturated aqueous NaHCO₃ (20 mL). This heterogeneous mixture was then stirred vigorously for 1 h, poured into brine (40 mL), and extracted with hexanes:EtOAc (1:1, 3×60 mL). The combined organic layers were washed with brine (100 mL), dried (MgSO₄), filtered, and concentrated. Purification of the resultant residue by flash column chromatography (silica gel, hexanes:CH₂Cl₂, 9:1→1:1) afforded tetracycle 56 (0.073 g, contaminated with a small amount of an inseparable, unidentified diastereomer) as an off-white solid. This minor by-product could be completely removed by recrystallization from CH₂Cl₂:MeOH (1:1) with slow evaporation to afford pure 56 (0.058 g, 31%) as a white crystalline solid. 56: R_{f}=0.60 (silica gel, hexanes:CH₂Cl₂, 1:2); IR (film) νₘₐₓ 2946, 1500, 1222, 1150, 1066, 1012, 920 cm⁻¹; ¹H NMR (400 MHz, CDCl₃) δ 6.77 (s, 1H), 6.60 (s, 1H), 5.95 (m, 1H), 5.09 (s, 2H), 5.09-5.00 (m, 2H), 3.96 (dd, J=12.4, 4.4 Hz, 1H), 3.49 (s, 3H), 3.32 (d, J=6.0 Hz, 2H), 2.72 (app d, J=8.4 Hz, 2H), 2.32 (dq, J=3.6, 13.2 Hz, 1H), 2.14 (dq, J=13.6, 4.0 Hz, 1H), 1.85-1.49 (m, 6H), 1.56 (s, 3H), 1.41 (dd, J=12.0, 2.4 Hz, 1H), 1.24 (m, 1H), 1.23 (s, 3H), 1.07 (s, 3H), 0.97 (s, 3H); ¹³C NMR (100 MHz, CDCl₃) δ 148.5, 147.6, 137.1, 129.0, 119.8, 117.5, 115.6, 115.1, 95.6, 78.3, 68.9, 56.1, 50.6, 47.1, 39.9, 38.4, 37.5, 34.5, 34.3, 31.2, 30.8, 30.5, 25.4, 24.9, 22.4, 18.3; HRMS (FAB) calcd for C₂₆H₃₇BrO₃ [M]⁺ 476.1926. found 476.1945.

### Carboxylic Acid 57.

A suspension of OsO₄ (3.7 mg, 0.0146 mmol, 0.2 equiv) in t-BuOH (0.20 mL) and a solution of NaIO₄ (0.078 g, 0.37 mmol, 5.0 equiv) in water (1 mL) were added sequentially to a solution of 56 (0.035 g, 0.073 mmol, 1.0 equiv) and pyridine (0.018 mL, 0.22 mmol, 3.0 equiv) in THF/t-BuOH/water (2.0/0.5/0.5 mL) at 0° C. Once the addition was complete, the reaction mixture was removed from the cold bath and stirred at 25° C. for 2 h. Upon completion, the reaction contents were quenched by the addition of 5% aqueous Na₂SO₃ (10 mL), extracted with CH₂Cl₂ (3×10 mL), dried (MgSO₄), filtered, and concentrated. Filtration of the resultant residue through a silica plug (50×10 mm) using hexanes:EtOAc (1:1, 15 mL) as eluent afforded the desired intermediate aldehyde (0.031 g, 89% yield) as a light yellow foam. Next, solid NaH₂PO₄.H₂O (0.101 g, 0.65 mmol, 10 equiv) was added to a solution of the above aldehyde (0.031 g, 0.065 mmol, 1.0 equiv) in THF/t-BuOH/water (1.0/0.4/0.4 mL). The suspension was stirred vigorously for 10 min until the buffer had completely dissolved, at which time it was cooled to 0° C. 2-Methyl-2-butene (0.069 mL, 0.65 mmol, 10 equiv) was then added, followed by a solution of NaClO₂ (0.023 g, 0.26 mmol, 4.0 equiv) in water (0.2 mL). After stirring for 20 min at 0° C., the reaction mixture was quenched by the addition of solid Na₂SO₃ (0.131 g, 1.04 mmol, 16 equiv) and water (3 mL) and the resultant biphasic mixture was stirred vigorously for 5 min at 0° C. The reaction mixture was poured into 1 M HCl (3 mL) and extracted into EtOAc (3×10 mL). The combined organic layers were washed with acidic brine (10 mL), dried (MgSO₄), filtered, and concentrated. Purification of the resultant residue by flash column chromatography (silica gel, hexanes:EtOAc:AcOH 6:4:0.1) afforded carboxylic acid 57 (0.026 g, 81% yield) as a white crystalline solid. 57: R_{f}=0.52 (silica gel, hexanes:EtOAc, 1:4); IR (film) νₘₐₓ 2946, 2900 (br), 2876, 1709, 1506, 1222, 1151, 1009 cm⁻¹; ¹H NMR (400 MHz, CDCl₃) δ 6.81 (s, 1H), 6.61 (s, 1H), 5.10 (s, 2H), 3.96 (dd, J=12.4, 4.4 Hz, 1H), 3.59 (d, J=2.0 Hz, 2H), 3.46 (s, 3H), 2.72 (app d, J=7.2 Hz, 2H), 2.32 (dq, J=3.6, 13.2 Hz, 1H), 2.14 (dq, J=13.6, 4.0 Hz, 1H), 1.82-1.45 (m, 6H), 1.56 (s, 3H), 1.39 (dd, J=12.0, 2.0 Hz, 1H), 1.22 (s, 3H), 1.21 (m, 1H), 1.07 (s, 3H), 0.97 (s, 3H); ¹³C NMR (100 MHz, CDCl₃) δ 177.5, 148.7, 147.6, 122.4, 121.6, 118.6, 114.9, 95.4, 78.5, 68.8, 56.1, 50.5, 47.1, 39.9, 38.3, 37.5, 35.8, 34.5, 31.2, 30.8, 30.4, 25.5, 24.9, 22.4, 18.3; HRMS (FAB) calcd for C₂₅H₃₅BrO₅ [M]⁺ 494.1668. found 494.1684.

### Peyssonoic Acid A (51).

A solution of BCl₃ (1.0 M in CH₂Cl₂, 0.28 mL, 0.28 mmol, 6.0 equiv) was added dropwise to a solution of carboxylic acid 57 (0.023 g, 0.046 mmol, 1.0 equiv) in CH₂Cl₂ (2 mL) at −78° C. The resultant light orange solution was stirred for 1 h at −78° C. and then quenched by the addition of water (5 mL). The crude product was extracted with EtOAc (3×5 mL) and the combined organic layers were washed with acidic brine (5 mL), dried (MgSO₄), filtered, and concentrated. Purification of the resultant residue by preparatory TLC (silica gel, hexanes:EtOAc, 1:3+2% AcOH) afforded peyssonoic acid A (51, 0.015 g, 72% yield) as a light yellow amorphous solid. 51: R_{f}=0.46 (silica gel, hexanes:EtOAc:AcOH, 25:75:2); IR (film) νₘₐₓ 3258 (br), 2967, 2925, 1712, 1433, 1201, 1023, 988 cm⁻¹; ¹H NMR (400 MHz, (CD₃)₂SO) δ 8.51 (br s, 2H), 6.52 (s, 1H), 6.50 (s, 1H), 5.25 (s, 1H), 4.20 (dd, J=12.4, 3.6 Hz, 1H), 3.33 (s, 2H), 2.82 (dd, J=14.4, 6.0 Hz, 1H), 2.19-2.02 (m, 3H), 1.96-1.82 (m, 3H), 1.77 (t, J=5.6 Hz, 1H), 1.63 (dd, J=11.2, 6.0 Hz, 1H), 1.48 (s, 3H), 1.00 (s, 3H), 0.99 (m, 1H), 0.97 (s, 3H), 0.87 (s, 3H); ¹³C NMR (100 MHz, (CD₃)₂SO) δ 172.9, 147.5, 146.8, 136.4, 127.7, 119.6, 119.1, 117.2, 116.3, 70.7, 53.7, 41.8, 39.1, 36.4 (2C), 35.0, 30.8, 30.4, 29.9, 25.2, 23.7, 21.9, 17.5; HRMS (FAB) calcd for C₂₃H₃₁BrO₄ [M]⁺ 450.1406. found 450.1425. [Note: we initially found that our NMR data did not match the data given for this compound in the original isolation paper (Ref. (34a)). However, an analysis of which particular signals were out of alignment indicated that only the benzylic and aromatic signals were significantly incorrect, and of those the signals at and adjacent to the carboxylic acid were the most disparate; specifically, these peaks were too far downfield. This occurrence indicated to us that the isolated natural product was likely, in fact, the more electron-rich monoanion of peyssonoic acid A (i.e. —‘sodium peyssonoate’). Since the natural product was extracted from the organism and immediately subjected to chromatographic separation and structural characterization, it is likely that the isolated compound was never protonated from the monoanionic form that would be expected to predominate at physiological pH. This hypothesis was validated as our NMR spectra coalesced with those of the reported compound after stirring the product with excess NaHCO₃ in DMSO (see attached spectra)].

Comparison of natural and synthetic peyssonoic acid A.

| | | 51 (stirred over |
|---|---|---|
| Natural Peyssonoic Acid A | 51 | NaHCO₃) |

¹H

| | | |
|---|---|---|
| 0.86 (s, 3H) | 0.87 (s, 3H) | |
| 0.97 (s, 3H) | 0.97 (s, 3H) | |
| 0.99 (m) | 0.99 (m) | |
| 1.00 (s, 3H) | 1.00 (s, 3H) | |
| 1.49 (s, 3H) | 1.48 (s, 3H) | |
| 1.64 (m) | 1.63 (dd, J = 11.2, 6.0) | |
| 1.78 (t, J = 5.2) | 1.77 (t, J = 5.6) | |
| 1.88 (m) | 1.82-1.96 (m, 3H) | |
| 1.92 (m) | | |
| 1.93 (m) | | |
| 2.06 (m) | 2.07 (m, 1H) | |
| 2.13 (m, 2H) | 2.19-2.10 (m, 2H) | |
| 2.77 (dd, J = 14.6, 6.2) | 2.82 (dd, J = 14.4, 6.0) | 2.77 (dd, |
| | | J = 14.8, 6.0) |
| 3.09 (s, 2H) | 3.33 (s, 2H) | 3.11 (s, 2H) |
| 4.20 (dd, J = 12.5, 3.0) | 4.20 (dd, J = 12.4, 3.6) | |
| 5.23 (s) | 5.25 (s) | 5.23 (s) |
| 6.31 (s) | 6.50 (s) | 6.32 (s) |
| 6.35 (s) | 6.52 (s) | 6.36 (s) |
| 8.38 (br s, OH) | 8.51 (br s, 2 OH) | |
| 13.39 (br s, OH) | | |

¹³C

| | | |
|---|---|---|
| 17.5 | 17.5 | |
| 21.8 | 21.9 | |
| 23.6 | 23.7 | |
| 25.1 | 25.2 | |
| 29.9 | 29.9 | |
| 30.3 | 30.4 | |
| 30.8 | 30.8 | |
| 36.3 | 36.4 | |
| 36.4 | 36.4 | |
| 39.1 | 39.1 | |
| 41.8 | 41.8 | |
| 44.5 | 35.0 | 44.3 |
| 53.7 | 53.7 | |
| 70.9 | 70.7 | |
| 116.6 | 116.3 | 116.7 |
| 117.8 | 117.2 | 117.9 |
| 119.2 | 119.1 | |
| 122.6 | 119.6 | 122.6 |
| 126.6 | 127.7 | 126.8 |
| 136.7 | 136.4 | 136.7 |
| 146.1 | 146.8 | 146.3 |
| 150.1 | 147.5 | 150.0 |
| 175.3 | 172.9 | 175.7 |

### Cation-π Cyclization Precursor 58.

Prepared as in 55 from (2Z,6E)-farnesyl bromide and the aryllithium reagent derived from 34; 0.99 g (45% yield from 53) as a light yellow viscous oil. 58: R_{f}=0.45 (silica gel, hexanes:EtOAc, 9:1); IR (film) νₘₐₓ 2961, 2928, 2854, 1488, 1151, 1081, 998 cm⁻¹; ¹H NMR (400 MHz, CDCl₃) δ 7.26 (s, 1H), 6.95 (s, 1H), 5.25 (t, J=7.2 Hz, 1H), 5.15 (s, 2H), 5.12 (s, 2H), 5.18-5.08 (m, 2H), 3.52 (s, 3H), 3.47 (s, 3H), 3.28 (d, J=7.2 Hz, 2H), 2.19-1.92 (m, 8H), 1.74 (s, 3H), 1.68 (s, 3H), 1.61 (s, 3H), 1.60 (s, 3H); ¹³C NMR (100 MHz, (CDCl₃) δ 150.6, 148.7, 137.0, 135.5, 131.7, 131.5, 124.5, 124.2, 122.5, 119.3, 118.5, 110.3, 96.1, 95.2, 56.5, 56.2, 39.9, 32.2, 28.5, 26.8, 26.6, 25.8, 23.6, 17.8, 16.1; HRMS (FAB) calcd for C₂₅H₃₇BrO₄ [M]⁺ 480.1875. found 480.1885.

### Cation-π Cyclization Product 59.

A solution of BDSB (13, 0.251 g, 0.458 mmol, 1.1 equiv) in nitromethane (2 mL) pre-cooled to −25° C. was syringed quickly into a solution of 58 (0.200 g, 0.416 mmol, 1.0 equiv) in nitromethane (40 mL) at −25° C. After stirring for 5 min at −25° C., the reaction mixture was quenched by the sequential addition of 5% aqueous Na₂SO₃ (20 mL) and saturated aqueous NaHCO₃ (20 mL). This heterogeneous mixture was stirred vigorously for 1 h, then poured into brine (40 mL) and extracted into hexanes:EtOAc (1:1, 3×60 mL). The combined organic layers were washed with brine (100 mL), dried (MgSO₄), filtered, and concentrated. Purification of the resultant residue by flash column chromatography (silica gel, hexanes:EtOAc, 1:0→19:1) afforded 59 (0.112 g) contaminated with a small amount of an inseparable, unidentified diastereomer. This minor by-product could be completely removed by recrystallization from CH₂Cl₂:EtOH (1:1) with slow evaporation to afford pure 59 (0.090 g, 42% yield) as a white crystalline solid. 59: R_{f}=0.39 (silica gel, hexanes:EtOAc, 9:1); IR (film) νₘₐₓ 2947, 1483, 1390, 1151, 1088, 1011, 971 cm⁻¹; ¹H NMR (400 MHz, CDCl₃) δ 6.96 (s, 1H), 6.85 (s, 1H), 5.12 (s, 2H), 3.95 (dd, J=12.8, 4.4 Hz, 1H), 3.53 (s, 3H), 2.76-2.62 (m, 2H), 2.31 (dq, J=3.6, 13.2 Hz, 1H), 2.14 (dq, J=13.6, 4.0 Hz, 1H), 1.82-1.45 (m, 6H), 1.56 (s, 3H), 1.38 (dd, J=12.0, 2.8 Hz, 1H), 1.23 (m, 1H), 1.22 (s, 3H), 1.07 (s, 3H), 0.97 (s, 3H); ¹³C NMR (100 MHz, (CDCl₃) δ 148.7, 147.1, 121.8, 120.7, 117.3, 111.2, 96.3, 79.0, 68.7, 56.5, 50.3, 47.1, 39.9, 38.3, 37.5, 34.5, 31.1, 30.8, 30.3, 25.4, 24.9, 22.4, 28.3; HRMS (FAB) calcd for C₂₃H₃₂Br₂O₃ [M]⁺ 514.0718. found 514.0720.

### 6. Formal Total Synthesis of Aplysin-20 (64)

### Cation-π Cyclization Precursor 60.

Prepared according to the method described in our earlier communication[59] for its geranyl-derived homologue using trans,trans-farnesol as the starting material; 0.354 g (74% yield over two steps) of a light yellow viscous oil. 60: R_{f}=0.54 (silica gel, hexanes: EtOAc, 4:1); IR (film) νₘₐₓ 2966, 2919, 2855, 2249, 1448, 1383 cm⁻¹; ¹H NMR (400 MHz, CDCl₃) δ 5.16 (tq, J=6.8, 1.2 Hz, 1H), 5.11-5.05 (m, 2H), 3.04 (dd, J=6.8, 0.8 Hz, 2H), 2.15-1.93 (m, 8H), 1.68 (s, 6H), 1.60 (s, 6H); ¹³C NMR (100 MHz, CDCl₃) δ 142.4, 135.9, 131.5, 124.4, 123.4, 118.7, 111.7, 39.8, 39.3, 26.8, 26.2, 25.8, 17.8, 16.4, 16.3, 16.1; HRMS (EI) calcd for C₁₆H₂₅N [M]⁺ 231.1987. found 231.1983.

### Cation-π Cyclization Product 65.

A solution of BDSB (13, 0.060 g, 0.110 mmol, 1.1 equiv) in nitromethane (0.5 mL) was syringed quickly into a solution of 60 (0.023 g, 0.110 mmol, 1.0 equiv) in nitromethane (1.5 mL) at 25° C. After stirring for 5 min at 25° C., the reaction mixture was quenched by the sequential addition of 5% aqueous Na₂SO₃ (5 mL) and saturated aqueous NaHCO₃ (5 mL). This heterogeneous mixture was then stirred vigorously for 15 min, poured into brine (5 mL), and extracted with CH₂Cl₂ (3×5 mL). The combined organic layers were washed with brine (100 mL), dried (MgSO₄), filtered, and concentrated. Purification of the resultant residue by flash column chromatography (silica gel, hexanes:EtOAc, 9:1) afforded bicycle 65 [0.022 g of a 5.3:1.3:1.0 mixture of alkene isomers (trisubstituted:disubstituted:tetrasubstituted); 72% combined yield] as an amorphous colorless solid. Major alkene isomer of 65: R_{f}=0.54 (silica gel, hexanes:EtOAc, 4:1); IR (film) νₘₐₓ 2970, 2943, 2851, 2246, 1721, 1443, 1157, 896 cm⁻¹; ¹H NMR (400 MHz, CDCl₃) δ 5.56 (br s, 1H), 4.01 (dd, J=10.8, 4.8 Hz, 1H), 2.46 (dd, J=16.8, 4.0 Hz, 1H), 2.30-1.98 (m, 6H), 1.88 (dt, J=13.6, 3.6 Hz, 1H), 1.79 (s, 3H), 1.36 (dd, J=11.6, 4.8 Hz, 1H), 1.27 (m, 1H), 1.07 (s, 3H), 1.05 (s, 3H), 0.91 (s, 3H); ¹³C NMR (100 MHz, CDCl₃) δ 131.0, 124.8, 120.6, 68.3, 51.4, 50.5, 40.9, 39.5, 36.7, 30.8, 30.6, 25.0, 21.5, 18.2, 15.1, 14.0; HRMS (EI) calcd for C₁₆H₂₄BrN [M]⁺ 309.1092. found 309.1099. [Diagnostic ¹H NMR signals for other isomers: tetrasubstituted δ 2.94 (AB, J=57.6, 18.0 Hz, 2H), 1.79 (s, 3H), 1.08 (s, 3H), 0.98 (s, 3H); disubstituted δ 4.99 (s, 1H), 4.65 (s, 1H), 1.10 (s, 3H), 0.95 (s, 3H), 0.74 (s, 3H)].

### 7. Investigations Using IDSI

### IDSI (70).

Et₂S (0.54 mL, 5.0 mmol, 2.0 equiv) was added dropwise to a solution of I₂ (0.64 g, 2.5 mmol, 1.0 equiv) in 1,2-dichloroethane (15 mL) at 0° C., and the mixture was stirred for 5 min at 0° C. SbCl₅ (1.0 M in CH₂Cl₂, 5.0 mL, 5.0 mmol, 2.0 equiv) was then added dropwise, and the resultant solution was allowed to warm slowly to 25° C. over 30 min, then stirred for an additional 2 h at 25° C. Upon completion, and in order to collect IDSI crystals, hexanes (4 mL) was carefully pipetted onto the top of the purple solution and the layered reaction mixture was cooled to −20° C. for 24 h. The resulting orange crystals were isolated by decanting off the liquid, rinsing with hexanes (2×1 mL), and then drying under vacuum prior to use in cation-π cyclizations (1.26 g, 78% yield).

### Cation-π Cyclization of 71 Using IDSI to Generate 72.

A solution of IDSI (70, 0.097 g, 0.120 mmol, 1.2 equiv) in nitromethane (0.5 mL) was quickly added via syringe to a solution of homogeranylbenzene (71, 0.023 g, 0.100 mmol, 1.0 equiv) in nitromethane (1.5 mL) at −25° C. After stirring for 5 min at −25° C., the resulting mixture was poured into a solution of saturated aqueous NaHCO₃:5% aqueous Na₂SO₃ (1:1, 10 mL) and the resultant biphasic mixture was stirred vigorously for 15 min at 25° C. The reaction contents were then extracted with CH₂Cl₂ (3×10 mL), and the combined organic layers were dried (MgSO₄), filtered, and concentrated. The resultant residue was purified by flash column chromatography (silica gel, hexanes: CH₂Cl₂, 9:1) to afford tricycle 72 (0.034 g, 93% yield) as a colorless amorphous solid. 72: R_{f}=0.49 (silica gel, hexanes: CH₂Cl₂, 9:1); IR (film) νₘₐₓ 3058, 2965, 2945, 1488, 1475, 1377, 762, 723, 670 cm⁻¹; ¹H NMR (400 MHz, CDCl₃) δ 7.20-7.03 (m, 4H), 4.28 (dd, J=13.2, 4.0 Hz, 1H), 2.92-2.87 (m, 2H), 2.57 (dq, J=3.6, 13.6 Hz, 1H), 2.45 (dq, J=14.0, 3.6 Hz, 1H), 2.17 (dt, J=13.2, 3.6 Hz, 1H), 2.00 (m, 1H), 1.82 (m, 1H), 1.62-1.54 (m, 2H), 1.25 (s, 3H), 1.13 (s, 3H), 1.09 (s, 3H); ¹³C NMR (75 MHz, CDCl₃) δ 148.8, 134.8, 129.2, 126.0, 125.7, 124.6, 53.5, 50.0, 41.9, 39.7, 38.3, 34.5, 33.2, 31.0, 25.0, 21.8, 21.3; HRMS (EI) calcd for C₁₇H₂₃I [M]⁺ 354.0845. found 354.0840.

### Cation-π Cyclization of 71 Using Ipy₂BE₄/HBF₄ to Generate 72.

[62] A suspension of bis(pyridine)iodonium tetrafluoroborate (0.037 g, 0.100 mmol, 1.0 equiv) in CH₂Cl₂ (0.1 mL) was stirred for 5 min at 25° C. until the solid dissolved. The reaction contents were then cooled to −40° C. and HBF₄.Et₂O (0.014 mL, 0.100 mmol, 1.0 equiv) was added. After stirring for 10 min at −40° C., homogeranylbenzene (71, 0.023 g, 0.100 mmol, 1.0 equiv) was added as a solution in CH₂Cl₂ (0.8 mL). The reaction mixture was kept at −40° C. for 3 h with constant stirring. Upon completion, the reaction contents were quenched with ice-cold water (10 mL), Na₂S₂O₃ (0.100 g) was added, and the materials were extracted with CH₂Cl₂ (3×10 mL). The combined organic layers were washed with water (10 mL), dried (MgSO₄), filtered, and concentrated. The resultant brown oil was purified by flash column chromatography (silica gel, hexanes:CH₂Cl₂, 9:1) to afford tricycle 72 (0.015 g, 41% yield) as a colorless amorphous solid. [Note: since the partially-cyclized compounds are difficult to isolate, we calculated a 7% yield of partially-cyclized material and a 6% yield of proton-cyclized product based on ¹H NMR ratios of diagnostic signals. The remaining mass balance was divided between incorrect diastereomers and other unknown products. The proton-cyclized product was spectroscopically identical to previously reported material [63].

Using the above conditions with substrate 94 afforded 47% isolated yield of the tetra-substituted partially-cyclized product. The remaining mass was assigned as a mixture of di- and tri-substituted alkene isomers (partially-cyclized material), as well as trace starting material. Product 98 was not observed under these conditions.

### Cation-π Cyclization of 71 Using PPh₃ and NIS to Generate 72.

[64] A solution of homogeranylbenzene (71, 0.023 g, 0.100 mmol, 1.0 equiv) in CH₂Cl₂ (0.5 mL) was added to a solution of Ph₃P (7.9 mg, 0.0030 mmol, 30 mol %) in CH₂Cl₂ (0.5 mL) at −78° C. N-Iodosuccinimide (0.023 g, 0.100 mmol, 1.0 equiv) was added to the reaction mixture, which was kept at −78° C. for 24 h. The reaction mixture was then warmed to −40° C. and kept at −40° C. for 6 h with constant stirring. The reaction contents were then quenched with 20% aqueous Na₂S₂O₃ (5 mL) and the materials were extracted with hexanes (3×10 mL). The combined organic layers were washed with brine (10 mL), dried (MgSO₄), filtered, and concentrated. Since the partially-cyclized compounds are difficult to isolate, we calculated a 1.8:1.0 ratio of partially-cyclized material:starting material based on ¹H NMR ratios of diagnostic signals. The desired product 72 was observed in trace amounts.

Using the above conditions with substrate 94 afforded a 3.5:1.0 mixture of starting material:partially-cyclized material based on the crude ¹H NMR. The desired product 98 was not observed under these conditions.

### Preparation of Cation-π Cyclization Precursors in Table 2

76.

Prepared according to the method of Ref. 7. First, t-BuLi (1.7 M in pentane, 0.690 mL, 1.17 mmol, 3.0 equiv) was added dropwise to solution of 17 (0.100 g, 0.390 mmol, 1.0 equiv) in THF (1.5 mL) at −40° C. The reaction mixture was then stirred at −40° C. for 1 h. Next, trimethylborate (0.135 mL, 1.17 mmol, 3.0 equiv) was added in a single portion at −40° C., and the reaction mixture was then allowed to warm to 0° C. over the course of 1 h. A solution of 1 M NaOH:aqueous 30% H₂O₂ (1:1, 0.6 mL) was then carefully added at 0° C. and the reaction mixture was stirred for an additional 30 min at 0° C. The reaction contents were then quenched with saturated aqueous Na₂SO₃ (5 mL) and the resultant biphasic mixture was stirred for 10 min at 25° C. The mixture was then extracted with EtOAc (3×20 mL) and the combined organic layers were washed with water (50 mL), dried (MgSO₄), filtered, and concentrated. The resultant colorless oil was purified by flash column chromatography (silica gel, hexanes:EtOAc, 9:1) to give the desired phenol (0.096 g, 90% yield) as a colorless oil. Next, to a solution of a portion of the newly prepared phenol (0.063 g, 0.230 mmol, 1.0 equiv) and dimethyl sulfate (0.044 mL, 0.260 mmol, 2.0 equiv) in THF (1 mL) at 0° C. was slowly added NaH (60% dispersion in mineral oil, 0.040 g, 1.00 mmol, 4.3 equiv). The resulting suspension was allowed to warm to 25° C. and stirred for 3 h. Upon completion, the reaction contents were poured into water (10 mL) and extracted with Et₂O (5×15 mL). The combined organic layers were then dried (MgSO₄), filtered, and concentrated. The resulting yellow oil was purified by flash column chromatography (silica gel, hexanes:EtOAc, 20:1) to afford cyclization precursor 76 (0.063 g, 95% yield) as a light yellow oil. 76: R_{f}=0.29 (silica gel, hexanes:EtOAc, 9:1); IR (film) νₘₐₓ 2928, 2854, 1516, 1464, 1263, 1236, 1156, 1032 cm⁻¹; ¹H NMR (400 MHz, CDCl₃) δ 6.79 (dd, J=6.4, 2.0 Hz, 1H), 6.73 (dd, J=6.8, 2.0 Hz, 2H), 5.18 (tt, J=7.2, 1.2 Hz, 1H), 5.09 (tt, J=6.8, 1.2 Hz, 1H), 3.87 (s, 3H), 3.86 (s, 3H), 2.59 (t, J=7.2 Hz, 2H), 2.29 (q, J=7.6 Hz, 2H), 2.11-1.94 (m, 4H), 1.69 (s, 3H), 1.60 (s, 3H), 1.57 (s, 3H); ¹³C NMR (75 MHz, CDCl₃) δ 148.9, 147.3, 135.8, 135.3, 131.4, 124.5, 123.8, 120.4, 112.0, 111.4, 56.1, 55.9, 39.8, 35.8, 30.2, 26.9, 25.8, 17.8, 16.2; HRMS (EI) calcd for C₁₉H₂₈O₂ [M]⁺ 288.2094. found 288.2089.

84.

Prepared from nerol according to the method described by Yus and co-workers; [66] 0.546 g (85% yield) as a yellow viscous oil. 84: R_{f}=0.66 (silica gel, hexanes:EtOAc, 19:1); IR (film) νₘₐₓ 2972, 2933, 1739, 1369, 1277, 1254, 1166 cm⁻¹; ¹H NMR (400 MHz, CDCl₃) δ 5.37 (dt, J=1.2, 6.8 Hz, 1H), 5.08 (tt, J=6.8, 1.2 Hz, 1H), 4.55 (dd, J=7.2, 0.8 Hz, 2H), 2.14-2.04 (m, 4H), 1.75 (s, 3H), 1.68 (s, 3H), 1.59 (s, 3H), 1.47 (s, 9H); ¹³C NMR (100 MHz, CDCl₃) δ 153.7, 142.8, 132.3, 123.7, 119.1, 81.9, 63.6, 32.3, 27.9 (3C), 26.8, 25.8, 23.6, 17.8; HRMS (FAB) calcd for C₁₅H₂₅O₃ [M−H]⁺ 253.1804. found 253.1794.

Standard Procedure for Small-Scale Cation-π Cyclizations with IDSI (Table 2)

A solution of IDSI (70, 0.097 g, 0.120 mmol, 1.2 equiv) in nitromethane (0.5 mL) was quickly added to a solution of the substrate (0.100 mmol, 1.0 equiv) in nitromethane (1.5 mL) at the temperature indicated within Table 2. After stirring the reaction contents for the indicated time in Table 2, the mixture was poured into a solution of saturated aqueous NaHCO₃:5% aqueous Na₂SO₃ (1:1, 10 mL) and the resultant biphasic mixture was vigorously stirred for an additional 15 min at 25° C. Upon completion, the reaction contents were extracted with CH₂Cl₂ (3×10 mL) and the combined organic layers were dried (MgSO₄), filtered, and concentrated. The resultant residue was purified either by flash column chromatography or preparative TLC to yield the desired cation-cyclization products in the amount and yields indicated below.

75.

White crystalline solid, 0.035 g, 90% yield; R_{f}=0.35 (silica gel, hexanes:EtOAc, 19:1); IR (film) νₘₐₓ 2949, 2924, 2851, 1609, 1502, 1463, 1264, 1044, 870, 669 cm⁻¹; ¹H NMR (400 MHz, CDCl₃) δ 6.96 (d, J=8.4 Hz, 1H), 6.72 (d, J=2.4 Hz, 1H), 6.67 (dd, J=8.0, 2.4 Hz, 1H), 4.27 (dd, J=12.8, 4.0 Hz, 1H), 3.77 (s, 3H), 2.90-2.77 (m, 2H), 2.54 (dq, J=3.6, 13.4 Hz, 1H), 2.44 (dq, J=14.0, 4.0 Hz, 1H), 2.11 (dt, J=13.2, 3.6 Hz, 1H), 1.98 (m, 1H), 1.79 (m, 1H), 1.60 (dd, J=13.2, 4.0 Hz, 1H), 1.53 (m, 1H), 1.25 (s, 3H), 1.13 (s, 3H), 1.08 (s, 3H); ¹³C NMR (75 MHz, CDCl₃) δ 158.2, 150.4, 130.2, 127.3, 111.5, 110.6, 55.7, 53.8, 50.3, 42.2, 39.9, 38.7, 34.8, 33.5, 30.4, 25.2, 22.2, 21.6; HRMS (EI) calcd for C₁₈H₂₅IO [M]⁺ 384.0950. found 384.0952.

77.

Yellow amorphous solid, 0.030 g, 73% yield; R_{f}=0.29 (silica gel, hexanes:EtOAc, 9:1); IR (film) νₘₐₓ 2947, 2847, 1510, 1463, 1256, 1146 cm⁻¹; ¹H NMR (400 MHz, CDCl₃) δ 6.67 (s, 1H), 6.51 (s, 1H), 4.28 (dd, J=13.2, 4.4 Hz, 1H), 3.83 (s, 6H), 2.82 (dd, J=8.8, 4.4 Hz, 2H), 2.56 (dq, J=3.2, 13.2 Hz, 1H), 2.44 (dq, J=14.0, 4.0 Hz, 1H), 2.10 (dt, J=13.2, 3.6 Hz, 1H), 1.98 (m, 1H), 1.80 (m, 1H), 1.62-1.51 (m, 2H), 1.24 (s, 3H), 1.13 (s, 3H), 1.07 (s, 3H); ¹³C NMR (75 MHz, CDCl₃) δ 147.4, 147.2, 140.9, 127.0, 111.6, 111.5, 50.3 (2C), 42.2, 39.6, 38.0, 34.5, 33.2 (2C), 30.8, 25.0, 24.9, 21.9, 21.3; HRMS (EI) calcd for C₁₉H₂₇IO₂ [M]⁺ 414.1056. found 414.1068.

78.

In order to ensure completion in the final cyclization leading to tetracycle 78, methanesulfonic acid (0.100 mL, 1.50 mmol, 15 equiv) was added to the reaction mixture at −25° C. after the initial 5 min IDSI-cyclization period, and the resultant solution was stirred for 60 min at −25° C. prior to the standard reaction quench described above. 78: white amorphous solid, 0.025 g, 60% yield; R_{f}=0.47 (silica gel, hexanes: CH₂Cl₂, 9:1); IR (film) νₘₐₓ 3059, 2941, 2868, 1451, 1386, 1145, 758 cm⁻¹; ¹H NMR (400 MHz, CDCl₃) δ 7.22 (m, 1H), 7.16-7.00 (m, 3H), 4.26 (dd, J=13.2, 4.4 Hz, 1H), 2.98-2.78 (m, 2H), 2.49 (dq, J=4.0, 13.6 Hz, 1H), 2.38 (dt, J=12.4, 3.2 Hz, 1H), 2.31 (dq, J=13.6, 3.6 Hz, 1H), 1.88-1.63 (m, 5H), 1.58-1.49 (m, 2H), 1.28 (dd, J=12.0, 2.4 Hz, 1H), 1.20 (s, 3H), 1.10 (dd, J=11.6, 2.0 Hz, 1H), 1.06 (s, 3H), 1.02 (s, 3H), 0.99 (s, 3H); ¹³C NMR (75 MHz, CDCl₃) δ 149.9, 134.9, 128.9, 125.9, 125.4, 124.6, 55.5, 55.2, 55.0, 43.2, 40.8, 39.6, 38.2, 37.9, 34.0, 33.2, 30.8, 26.1, 21.8, 21.0, 18.2, 16.3; HRMS (EI) calcd for C₂₂H₃₁I [M]⁺ 422.1471. found 422.1471.

79.
Yellow crystalline solid, 0.042 g, 85% yield; R_{f}=0.64 (silica gel, hexanes: EtOAc, 4:1); IR (film) νₘₐₓ 2866, 2825, 1482, 1388, 1195, 1151, 1134, 1011, 964, 738 cm⁻¹; ¹H NMR (400 MHz, CDCl₃) δ 6.96 (s, 1H), 6.86 (s, 1H), 5.13 (s, 2H), 4.23 (dd, J=12.8, 4.0 Hz, 1H), 3.53 (s, 3H), 2.77 (dd, J=16.8, 5.6 Hz, 1H), 2.67 (dd, J=16.4, 12.8 Hz, 1H), 2.45 (dq, J=14.0, 4.0 Hz, 1H), 2.29 (dq, J=3.6, 13.6 Hz, 1H), 1.87-1.69 (m, 3H), 1.21 (s, 3H), 1.12 (s, 3H), 1.04 (s, 3H); ¹³C NMR (100 MHz, CDCl₃) δ 148.3, 147.4, 122.0, 121.4, 117.8, 111.4, 96.2, 76.3, 56.5, 49.1, 46.5, 42.4, 39.0, 34.3, 32.2, 25.8, 19.9, 19.7; HRMS (FAB) calcd for C₁₈H₂₄BrIO₃ [M]⁺ 493.9954. found 493.9931.
80.

Colorless viscous oil, 0.025 g of a 8.5:1.4:1.0 mixture of alkene isomers (trisubstituted:tetrasubstituted:disubstituted), 85% combined yield; Major alkene isomer of 80: R_{f}=0.33 (silica gel, hexanes:EtOAc, 4:1); IR (film) νₘₐₓ 2969, 2935, 2858, 2246, 1445, 1372, 1139, 844 cm⁻¹; Diagnostic ¹H NMR signals (400 MHz, CDCl₃) δ 5.32 (br s, 1H), 4.33 (dd, J=10.8, 5.6 Hz, 1H), 2.72 (dd, J=17.6, 4.8 Hz, 1H), 2.47 (dd, J=17.2, 5.6 Hz, 1H), 1.80 (s, 3H), 1.19 (s, 3H), 1.04 (s, 3H); HRMS (EI) calcd for C₁₁H₁₆IN [M]⁺ 289.0328. found 289.0315. [Diagnostic ¹H NMR signals for other isomers: tetrasubstituted δ 4.41 (dd, J=9.6, 4.4 Hz, 1H), 3.07 (AB, J=44.4, 17.6 Hz, 2H), 1.74 (s, 3H), 1.26 (s, 3H), 1.23 (s, 3H); disubstituted δ 5.06 (s, 1H), 4.78 (s, 1H), 1.22 (s, 3H), 0.88 (s, 3H)].

81.

White crystalline solid, 0.015 g, 45% yield; R_{f}=0.42 (silica gel, hexanes: EtOAc, 1:1); IR (film) νₘₐₓ 3394 (br), 2971, 2947, 1736, 1372, 1244, 1140, 1028, 913 cm⁻¹; ¹H NMR (400 MHz, CDCl₃) δ 4.45 (dd, J=12.0, 4.8 Hz, 1H), 4.31 (dd, J=12.0, 5.2 Hz, 1H), 4.16 (dd, J=12.8, 4.0 Hz, 1H), 2.53 (br s, 1H), 2.36 (dq, J=14.0, 4.0 Hz, 1H), 2.20 (dq, J=4.0, 13.6 Hz, 1H), 2.06 (s, 3H), 1.77 (t, J=5.2 Hz, 1H), 1.66 (dt, J=13.2, 3.6 Hz, 1H), 1.56 (dt, J=4.0, 13.6 Hz, 1H), 1.23 (s, 3H), 1.14 (s, 3H), 1.02 (s, 3H); ¹³C NMR (100 MHz, CDCl₃) δ 171.1, 72.0, 64.1, 54.3, 49.5, 44.9, 39.6, 34.9, 32.9, 23.8, 21.3, 20.5; HRMS (EI) calcd for C₁₂H₂₁IO₃ [M]⁺ 340.0535. found 340.0540.

83.

Light yellow crystalline solid, 0.018 g, 57% yield; R_{f}=0.26 (silica gel, hexanes: EtOAc, 3:2); IR (film) νₘₐₓ 2932, 1732, 1223, 1135, 1081 cm⁻¹; ¹H NMR (400 MHz, CDCl₃) δ 4.51 (dd, J=10.8, 5.6 Hz, 1H), 4.42 (dd, J=12.7, 10.8 Hz, 1H), 4.14 (dd, J=12.8, 4.0 Hz, 1H), 2.47 (dq, J=14.4, 4.0, 1H), 2.21 (m, 1H), 2.11 (dd, J=12.8, 5.6 Hz, 1H), 1.85 (dt, J=13.2, 3.6 Hz, 1H), 1.75 (dt, J=4.4, 0.8 Hz, 1H), 1.52 (s, 3H), 1.11 (s, 3H), 1.01 (s, 3H); ¹³C NMR (100 MHz, CDCl₃) δ 148.4, 81.0, 68.4, 45.7, 45.1, 41.2, 37.8, 33.8, 31.2, 20.9, 20.1; HRMS (FAB) calcd for C₁₁K₈IO₃ [M+H]⁺ 325.0301. found 325.0290.

85.

White amorphous solid, 0.016 g, 48% yield; R_{f}=0.26 (silica gel, hexanes: EtOAc, 3:2); IR (film) νₘₐₓ 2971, 2946, 1742, 1214, 1112 cm⁻¹; ¹H NMR (400 MHz, CDCl₃) δ 4.74 (dd, J=12.4, 6.0 Hz, 1H), 4.52 (br s, 1H), 4.47 (d, J=12.0 Hz, 1H), 2.28-2.11 (m, 2H), 2.01 (m, 1H), 1.94-1.88 (m, 2H), 1.55 (s, 3H), 1.28 (s, 3H), 1.19 (s, 3H); ¹³C NMR (75 MHz, CDCl₃) δ 149.1, 80.7, 66.4, 50.7, 40.1, 37.1, 35.5, 35.1, 28.8, 28.1, 20.1; HRMS (EI) calcd for C₁₁H₁₇IO₃ [M]⁺ 324.0222. found 324.0205.

86.

Colorless viscous oil, 0.014 g, 39% yield, contaminated with ˜15% of an inseparable, unidentified impurity; R_{f}=0.28 (silica gel, hexanes: EtOAc, 20:1); IR (film) νₘₐₓ 2980, 2934, 1784, 1370, 1232, 1023 cm⁻¹; ¹H NMR (400 MHz, CDCl₃) δ 5.47 (t, J=7.2 Hz, 1H), 4.63 (d, J=6.8 Hz, 2H), 4.16 (dd, J=11.2, 1.2 Hz, 1H), 2.40 (m, 1H), 2.28 (m, 1H), 2.15 (m, 1H), 2.08 (s, 3H), 1.92 (m, 1H), 1.88 (s, 3H), 1.75 (s, 6H); ¹³C NMR (100 MHz, CDCl₃) δ 171.2, 140.1, 120.2, 72.5, 61.3, 48.6, 39.4, 35.6, 34.2, 28.4, 21.2, 16.5; HRMS (FAB) calcd for C₁₂H₁₉ClIO₂ [M−H]⁺ 357.0118. found 357.0135.

### Alkene 87.

DBU (0.25 mL, 1.70 mmol, 20 equiv) was added to a solution of 72 (0.030 g, 0.085 mmol, 1.0 equiv) in pyridine (1 mL) at 25° C. The resultant solution was then heated with stirring at 120° C. for 12 h. Upon completion, the reaction contents were cooled to 25° C., quenched with saturated aqueous NH₄Cl (10 mL), and extracted with Et₂O (3×10 mL). The combined organic layers were then washed with water (10 mL), dried (MgSO₄), filtered, and concentrated. The resulting brown oil was purified by flash column chromatography (silica gel, hexanes:CH₂Cl₂, 9:1) to afford alkene 87 (0.017 g, 86% yield) as a white amorphous solid. 87: R_{f}=0.68 (silica gel, hexanes: CH₂Cl₂, 9:1); IR (film) νₘₐₓ 3011, 2959, 2936, 2838, 1489, 1447, 1373, 1044, 758, 729 cm⁻¹; ¹H NMR (400 MHz, CDCl₃) δ 7.29 (m, 1H), 7.18-7.02 (m, 3H), 5.62 (ddd, J=10.0, 6.0, 2.0 Hz, 1H), 5.50 (dd, J=10.0, 2.8 Hz, 1H), 2.96-2.80 (m, 2H), 2.55 (dd, J=16.8, 6.0 Hz, 1H), 2.13 (d, J=16.4 Hz, 1H), 1.87 (m, 1H), 1.76-1.65 (m, 2H), 1.27 (s, 3H), 1.06 (s, 3H), 1.00 (s, 3H); ¹³C NMR (75 MHz, CDCl₃) δ 148.1, 138.3, 135.6, 129.0, 126.2, 126.1, 125.4, 122.0, 48.3, 39.9, 37.2, 35.2, 32.0, 31.3, 25.4, 22.5, 20.1; HRMS (EI) calcd for C₁₇H₂₂ [M]⁺ 226.1722. found 226.1716.

### 8. Formal Total Synthesis of Loliolide (92), K-76 (97) and Stemodin (101)

### Cation-π Cyclization Product 93.

Following the above procedure for the cation-cyclizations with IDSI at −25° C. or 0° C. for 5 min using 88 or 89 respectively, cyclization product 93 (0.024 g, 79% yield, 19:1 inseparable diastereomers from 88 or 0.027 g, 88% yield from 89) was obtained as a white crystalline solid. 93: R_{f}=0.53 (silica gel, hexanes: EtOAc, 4:1); IR (film) νₘₐₓ 2957, 2871, 1774, 1457, 1188, 1126, 920 cm⁻¹; Major diastereomer: ¹H NMR (400 MHz, CDCl₃) δ 4.08 (dd, J=13.2, 4.8 Hz, 1H), 2.59-2.48 (m, 2H), 2.40 (dd, J=16.4, 6.8 Hz, 1H), 2.29 (m, 1H), 2.11 (dd, J=14.4, 6.8 Hz, 1H), 1.89-1.74 (m, 2H) 1.37 (s, 3H), 1.03 (s, 3H), 1.02 (s, 3H); ¹³C NMR (75 MHz, CDCl₃) δ 175.2, 84.9, 52.9, 44.9, 40.0, 38.3, 35.0, 32.1, 30.9, 20.5, 19.8; HRMS (EI) calcd for C_{H}H₁₇IO₂ [M]⁺ 308.0273. found 308.0275.

### Alkene 91.

Dry LiCl (0.125 g, 2.95 mmol, 50 equiv) was added to a solution of iodide 93 (0.018 g, 0.060 mmol, 1.0 equiv) in DMF (2 mL) at 25° C. The resulting solution was heated with stirring at 80° C. for 12 h. Upon completion, the reaction contents were quenched with saturated aqueous NH₄Cl (10 mL). Water (3 mL) was then added and the reaction mixture was extracted with Et₂O (3×5 mL). The combined organic layers were dried (MgSO₄), filtered, and concentrated. The resulting yellow oil was purified by flash column chromatography (silica gel, hexanes:EtOAc, 4:1) to afford alkene 91 (0.010 g, 97% yield) as a colorless amorphous solid. 91: R_{f}=0.56 (silica gel, hexanes: EtOAc, 4:1); IR (film) νₘₐₓ 2958, 2871, 1786, 1771, 1227, 1053, 952 cm⁻¹; ¹H NMR (400 MHz, CDCl₃) δ 5.55 (ddd, J=10.0, 5.2, 2.0 Hz, 1H), 5.50 (dd, J=10.0, 2.8 Hz, 1H), 2.53-2.27 (m, 5H), 1.33 (s, 3H), 1.07 (s, 3H), 1.04 (s, 3H); ¹³C NMR (100 MHz, CDCl₃) δ 176.2, 138.2, 121.9, 85.0, 52.3, 38.6, 35.4, 31.8, 28.8, 20.7, 20.6; HRMS (EI) calcd for C₁₁H₁₆O₂ [M]⁺ 180.1150. found 180.1146.

### Formal Total Synthesis of K-76 (97)

94
[61c]. To a solution of CuI (0.24 g, 1.3 mmol, 1.5 equiv) in Et₂O (15 mL) at 0° C. was added MeLi (1.6 M in Et₂₀, 1.6 mL, 2.6 mmol, 3.0 equiv) dropwise. After 5 min at 0° C., the reaction mixture was cooled to −78° C. and a solution of 99 (0.345 g, 0.859 mmol, 1.0 equiv) in Et₂O (2 mL) was added. The reaction mixture was allowed to slowly warm to −30° C. over the course of 2 h, then quenched with saturated aqueous NH₄Cl (10 mL). The reaction mixture was poured into water (5 mL), and extracted with hexanes:EtOAc (2:1, 3×10 mL). The combined organic layers were washed with brine (10 mL), dried (MgSO₄), filtered, and concentrated. The crude yellow viscous oil was purified by careful column flash column chromatography (silica gel, hexanes:CH₂Cl₂, 9:1→5:2) to afford 94 (0.175 g, 77% yield) as a light yellow viscous oil, spectroscopically identical to previously synthesized material.

### Cation-π Cyclization Product 98.

Following the above procedure for the cation-π cyclizations with IDSI at −25° C. for 5 min using substrate 94, cyclization product 98 (0.013 g, 77% yield) was obtained as a colorless viscous oil. 98: R_{f}=0.43 (silica gel, hexanes:EtOAc, 9:1); IR (film) νₘₐₓ 2971, 2947, 2852, 1735, 1440, 1164, 1132 cm⁻¹; ¹H NMR (400 MHz, CDCl₃) δ 5.51 (br s, 1H), 4.25 (dd, J=12.8, 4.4 Hz, 1H), 3.67 (s, 3H), 2.90 (br s, 1H), 2.42-2.25 (m, 2H), 2.20-2.02 (m, 2H), 1.60 (br s, 3H), 1.51 (q, J=4.0 Hz, 1H), 1.46 (m, 1H), 1.43 (dd, J=11.4, 5.6 Hz, 1H), 1.09 (s, 3H), 1.04 (s, 3H), 0.99 (s, 3H); ¹³C NMR (75 MHz, CDCl₃) δ 172.8, 129.1, 124.0, 61.9, 53.0, 51.3, 48.7, 43.3, 39.2, 36.5, 33.8, 33.3, 26.2, 21.3, 21.1, 15.0; HRMS (FAB) calcd for C₁₆H₂₆IO₂ [M+H]⁺ 377.0978. found 377.0977.

### Alkene 96.

DBU (0.160 mL, 1.00 mmol, 20 equiv) was added to a solution of 98 (0.020 g, 0.048 mmol, 1.0 equiv) in pyridine (1 mL) at 25° C. The resulting solution was heated with stirring at 80° C. for 12 h. Upon completion, the reaction contents were quenched with saturated aqueous NH₄Cl (10 mL) and then extracted with Et₂O (3×10 mL). The combined organic layers were washed with water (10 mL), dried (MgSO₄), filtered, and concentrated. The resulting light yellow oil was purified by flash column chromatography (silica gel, hexanes:CH₂Cl₂, 4:1) to afford alkene 96 (0.010 g, 86% yield) as a colorless viscous oil. 96: R_{f}=0.43 (silica gel, hexanes:EtOAc, 9:1); IR (film) νₘₐₓ 3011, 2955, 1722, 1431, 1281, 1211, 1021, 731 cm⁻¹; ¹H NMR (400 MHz, CDCl₃) δ 5.46 (ddd, J=10.0, 5.6, 1.6 Hz, 1H), 5.41 (dd, J=10.4, 2.8 Hz, 1H), 3.74 (s, 3H), 2.13-1.97 (m, 2H), 2.04 (d, J=16.8 Hz, 1H), 1.78 (dd, J=16.4, 5.6 Hz, 1H), 1.68 (m, 1H), 1.64 (s, 3H), 1.60-1.42 (m, 2H), 1.18 (s, 3H), 0.98 (s, 3H), 0.92 (s, 3H); ¹³C NMR (75 MHz, CDCl₃) δ 170.9, 138.1, 136.6, 133.5, 121.3, 51.2, 47.6, 37.2, 36.0, 34.9, 32.4, 31.8, 22.5, 21.3, 20.2, 19.5; HRMS (EI) calcd for C₁₆H₂₄O₂ [M]⁺ 248.1776. found 248.1761.

### Formal Total Synthesis of Stemodin (101)

99

[61c]. Methyl acetoacetate (0.30 mL, 2.8 mmol, 1.2 equiv) was added dropwise under constant flow of argon to a suspension of NaH (60% dispersion in mineral oil, 0.12 g, 3.0 mmol, 1.3 equiv) in THF (4 mL) at 0° C. The reaction mixture was stirred for 30 min at 0° C., then a solution of n-BuLi (1.6 M in hexanes, 1.7 mL, 2.8 mmol, 1.2 equiv) was added slowly and the resultant light orange solution was stirred an additional 10 min at 0° C. This dianion solution was cannulated slowly into a solution of geranyl bromide (0.50 g, 2.3 mmol, 1.0 equiv) in THF (4 mL) at 0° C. After 15 min at 0° C., diethyl chlorophosphate (0.67 mL, 4.6 mmol, 2.0 equiv) was then added dropwise at 0° C. The reaction mixture was allowed to warm slowly to 25° C. over the course of 90 min, then the reaction mixture was quenched with 0.25 M HCl (10 mL) and extracted with EtOAc (3×10 mL). The combined organic layers were washed with saturated aqueous NaHCO₃ (10 mL), dried (MgSO₄), filtered, and concentrated. The crude yellow oil was purified by flash column chromatography (silica gel, hexanes:EtOAc, 9:1→5:2) to afford 99 (0.67 g, 72% yield) as a light yellow viscous oil, spectroscopically identical to previously synthesized material.

### Cation-π Cyclization Product 102.

A solution of IDSI (70, 0.097 g, 0.120 mmol, 1.2 equiv) in nitromethane (0.5 mL) was added quickly via syringe to a solution of enol phosphate 99 (0.039 g, 0.100 mmol, 1.0 equiv) in nitromethane (3.5 mL) at 25° C. After stirring for 5 min at 25° C., the resulting mixture was poured into a solution of saturated aqueous NaHCO₃:5% aqueous Na₂SO₃ (1:1, 10 mL) and stirred for an additional 15 min at 25° C. The reaction contents were then extracted with CH₂Cl₂ (3×10 mL). The combined organic layers were concentrated and the crude partially cyclized material was then dissolved in toluene (2 mL) and cooled to 0° C. Concentrated H₂SO₄ (0.080 mL, 1.5 mmol, 15 equiv) was added dropwise to the solution and the resultant mixture was stirred for 30 min at 0° C. Upon completion, the reaction contents were slowly quenched with saturated aqueous NaHCO₃ (10 mL), and extracted with Et₂O (3×10 mL). The combined organic layers were dried (MgSO₄), filtered, and concentrated. The resulting brown oil was purified by flash column chromatography (silica gel, hexanes:EtOAc, 5:1) to afford cyclization adduct 102 (0.015 g, 40% yield) as a white crystalline solid. 102: R_{f}=0.34 (silica gel, hexanes: EtOAc, 4:1); IR (film) νₘₐₓ 2949, 1748, 1715, 1434, 1263, 1170, 1135 cm⁻¹; ¹H NMR (400 MHz, CDCl₃) δ 4.25 (dd, J=12.8, 4.4 Hz, 1H), 3.67 (s, 3H), 3.21 (s, 1H), 2.53-2.28 (m, 4H), 2.14 (m, 1H), 1.90 (dq, J=5.2, 12.8 Hz, 1H), 1.65 (dd, J=12.4, 3.2 Hz, 1H), 1.60 (dt, J=13.2, 3.6 Hz, 1H), 1.43 (dt, J=4.4, 13.2 Hz, 1H), 1.21 (s, 3H), 1.14 (s, 3H), 1.07 (s, 3H); ¹³C NMR (75 MHz, CDCl₃) δ 204.4, 168.3, 69.4, 52.1, 51.7, 51.0, 42.1, 41.9, 41.2, 39.6, 33.6, 33.5, 25.5, 21.2, 14.9; HRMS (FAB) calcd for C₁₅H₂₄IO₃ [M+H]⁺ 379.0770. found 379.0772.

### 9. Investigations Using CDSC

Chlorodiethylsulfonium hexachloroantimonate (103, CDSC).

A flask containing a stir bar and 1,2-dichloroethane (10 mL) was cooled to −30° C. Chlorine gas was then bubbled through the solvent for 1 min, yielding a transparent yellow solution. At this point the amount of Cl₂ was measured in solution (˜0.25 g, 3.5 mmol, 1.0 equiv). Et₂S (0.41 mL, 3.9 mmol, 1.1 equiv) was then added dropwise to this solution at −30° C. and the resultant mixture was stirred for 5 min at −30° C. SbCl₅ (1.0 M in CH₂Cl₂, 4.2 mL, 4.2 mmol, 1.2 equiv) was then slowly added via syringe. The resultant mixture was allowed to warm to 0° C. over the course of 30 min (precipitate dissolved). To collect CDSC, hexanes (4 mL) was carefully pipetted onto the top of the solution and the layered solution was cooled to −20° C. for 12 h. The resulting off-white powder was isolated by decanting off the liquid, rinsing with hexanes (2×1 mL), then drying under vacuum prior to use in cation-π cyclizations (1.45 g, 91% yield).

Standard Procedure for Small-Scale Cation-π Cyclizations with CDSC (Table 3)

A solution of CDSC (103, 0.050 g, 0.110 mmol, 1.1 equiv) in nitromethane (0.5 mL) was added to a solution of the substrate (0.100 mmol, 1.0 equiv) in nitromethane (1.5 mL) at the temperature indicated within Table 3. After stirring for the indicated time in Table 3, the resulting mixture was poured into a solution of saturated aqueous NaHCO₃:5% aqueous Na₂SO₃ (1:1, 10 mL) and vigorously stirred for an additional 15 min at 25° C. The reaction contents were then extracted with CH₂Cl₂ (3×10 mL). The combined organic layers were dried (MgSO₄), filtered, concentrated, and purified by flash column chromatography or preparative TLC to yield the desired cation-π cyclization products in the amount and yields shown below.

104.

Colorless viscous oil, 0.012 g of a 1.0:1.0 mixture of inseparable diastereomers, 46% combined yield; R_{f}=0.56 (silica gel, hexanes: CH₂Cl₂, 9:1); IR (film) νₘₐₓ 2945, 1452, 1262, 1027, 766, 726 cm⁻¹; HRMS (EI) calcd for C₁₇H₂₃Cl [M]⁺ 262.1488. found 262.1490; Equatorial chlorine diastereomer: ¹H NMR (400 MHz, CDCl₃) δ 7.23 (m, 1H), 7.15-7.04 (m, 3H), 3.81 (dd, J=12.0, 4.8 Hz, 1H), 3.02-2.88 (m, 2H), 2.36 (m, 1H), 2.22-2.03 (m, 2H), 1.95 (m, 1H), 1.81 (m, 1H), 1.58 (dt, J=4.2, 11.7 Hz, 1H), 1.42 (dd, J=12.0, 2.4 Hz, 1H), 1.24 (s, 3H), 1.15 (s, 3H), 1.03 (s, 3H); ¹³C NMR (75 MHz, CDCl₃) δ 148.9, 134.9, 129.1, 126.0, 125.7, 124.5, 72.8, 51.4, 40.1, 38.9, 37.8, 30.8, 30.3, 29.3, 25.0, 20.0, 16.8. Axial chlorine diastereomer: ¹H NMR (400 MHz, CDCl₃) δ 7.28 (m, 1H), 7.17-7.03 (m, 3H), 4.13 (t, J=2.8 Hz, 1H), 3.01-2.83 (m, 2H), 2.38 (m, 1H), 2.20-2.06 (m, 2H), 2.01 (m, 1H), 1.87-1.79 (m, 2H), 1.28 (dd, J=10.0, 2.4 Hz, 1H), 1.22 (s, 3H), 1.11 (s, 3H), 1.09 (s, 3H); ¹³C NMR (75 MHz, CDCl₃) δ 149.5, 135.1, 129.1, 125.9, 125.5, 124.3, 71.9, 43.1, 38.6, 37.6, 32.0, 31.0, 30.1, 27.8, 25.4, 23.0, 18.5. [Note: though the above diastereomers are inseparable, characterization was accomplished by comparing our experimental spectra to that of previously synthesized equatorial diastereomers. For instance, the equatorial diastereomer of 104 can be generated using our previously reported Hg(II)-based cation-π-cyclization of homogeranylbenzene][67].

105.

5.3 mg of a 2.2:1.0 mixture of separable diastereomers, 18% combined yield; Major diastereomer: White crystalline solid; R_{f}=0.14 (silica gel, hexanes: EtOAc, 7:3); IR (film) νₘₐₓ 3419 (br), 2974, 1736, 1369, 1245, 1030 cm⁻¹; ¹H NMR (400 MHz, CDCl₃) δ 4.42 (dd, J=12.0, 5.2 Hz, 1H), 4.33 (dd, J=12.0, 5.2 Hz, 1H), 3.78 (dd, J=12.0, 4.0 Hz, 1H), 2.48 (br s, 1H), 2.07 (s, 3H), 2.03 (m, 1H), 1.91-1.80 (m, 2H), 1.69 (t, J=5.2 Hz, 1H), 1.59 (dt, J=4.0, 13.6 Hz, 1H), 1.24 (s, 3H), 1.17 (s, 3H), 0.96 (s, 3H); ¹³C NMR (100 MHz, CDCl₃) δ 171.1, 71.9, 70.7, 63.2, 55.8, 41.8, 40.0, 30.9, 29.1, 24.0, 21.3, 16.4; HRMS (FAB) calcd for C₁₂H₂₂ClO₃ [M+H]⁺ 249.1257. found 249.1261. Minor diastereomer: White crystalline solid; R_{f}=0.21 (silica gel, hexanes: EtOAc, 7:3); IR (film) νₘₐₓ 3421 (br), 2925, 1732, 1367, 1238, 1029 cm⁻¹; ¹H NMR (400 MHz, CDCl₃) δ 4.38 (dd, J=12.0, 5.2 Hz, 1H), 4.27 (dd, J=11.6, 5.2 Hz, 1H), 4.00 (m, 1H), 2.14 (br s, 1H), 2.07 (s, 3H), 2.07-1.92 (m, 3H), 1.67 (m, 1H), 1.26 (br s, 1H), 1.24 (s, 3H), 1.18 (s, 3H), 1.07 (s, 3H); ¹³C NMR (100 MHz, CDCl₃) δ 171.1, 72.2, 70.4, 62.8, 39.5, 36.3, 29.8, 29.5, 28.6, 25.4, 23.8, 21.3; HRMS (FAB) calcd for C₁₂H₂₂ClO₃ [M+H]⁺ 249.1257. found 249.1263.

106.
White solid, 8.2 mg of a 4.0:1.0 mixture of separable diastereomers, 38% combined yield from 88, 4.4 mg of a 4.0:1.0 mixture of separable diastereomers, 20% combined yield from 89; Major diastereomer: R_{f}=0.43 (silica gel, hexanes: EtOAc, 4:1); IR (film) νₘₐₓ 2947, 1776, 922, 670 cm⁻¹; ¹H NMR (400 MHz, CDCl₃) δ 3.77 (dd, J=12.0, 4.8 Hz, 1H), 2.52 (dd, J=16.4, 14.4 Hz, 1H), 2.37 (dd, J=16.4, 6.8 Hz, 1H), 2.25 (m, 1H), 2.05 (dt, J=12.0, 3.6 Hz, 1H), 2.01-1.87 (m, 2H), 1.80 (dt, J=4.0, 12.8 Hz, 1H), 1.37 (s, 3H), 1.09 (s, 3H), 0.99 (s, 3H); ¹³C NMR (75 MHz, CDCl₃) δ 175.6, 84.7, 68.8, 54.9, 38.5, 37.6, 31.4, 29.9, 29.1, 20.6, 15.8; HRMS (FAB) calcd for C₁₁H₁₈ClO₂ [M+H]⁺ 217.0995. found 217.1007.

### 10. Chiral Cation-π Cyclization

(2R,5R)-(+)-2,5-Dimethylthiolane (precursor to 107, 108, and 109) was produced from (2S,5S)-hexanediol according to the procedure in Ref. 10a. (2S,5S)-Hexanediol was in turn produced by a yeast reduction of 2,5-hexanedione according to the procedure in Ref. 10b.

### 107 (“Chiral CDSC”).

A saturated solution of chlorine (˜1 M; ˜1 mmol, 1 equiv) was prepared by bubbling Cl₂ through CH₂Cl₂ (1 mL) at 25° C. The solution was cooled to [−78° C., and (2R,5R)-(+)-2,5-dimethylthiolane (0.116 g, 1.00 mmol, 1.0 equiv) and a solution of SbCl₅ (1.0 M in CH₂Cl₂, 1.10 mL, 1.10 mmol, 1.1 equiv) were syringed in sequentially. After 20 min at −78° C., the reaction mixture was removed from the cold bath and allowed to warm to 25° C. Stirring was ceased, and the crude reaction mixture was diluted with CH₂Cl₂ (5 mL) and layered with hexanes (5 mL). Upon standing at −25° C. for 16 h, small white needles were formed. The residual solvent was removed and the crystals were washed with a cold solution of 1:1 hexanes:CH₂Cl₂ (2×1 mL) and dried under vacuum to afford 0.244 g (50% yield) of 107.

### 108 (“Chiral BDSB”).

(2R,5R)-(+)-2,5-Dimethylthiolane (0.260 g, 2.24 mmol, 1.0 equiv) and a solution of SbCl₅ (1.0 M in CH₂Cl₂, 2.46 mL, 2.46 mmol, 1.1 equiv) were syringed sequentially into a solution of Br₂ (0.115 mL, 2.24 mmol, 1.0 equiv) in CH₂Cl₂ (2 mL) at −30° C. After 15 min at −30° C., the reaction mixture was warmed slowly in a water bath until all precipitate had dissolved (35° C.). Stirring was ceased, and the crude reaction mixture was allowed to cool slowly to 0° C. (4 h), then −25° C. (12 h). The residual solvent was removed and the small yellow needles were washed with cold CH₂Cl₂ (2×0.5 mL) and dried under vacuum to afford 0.520 g (40% yield) of 108.

### 109 (“Chiral IDSI”).

(2R,5R)-(+)-2,5-Dimethylthiolane (0.50 g, 4.0 mmol, 2.0 equiv) and a solution of SbCl₅ (1.0 M in CH₂Cl₂, 4.0 mL, 4.0 mmol, 2.0 equiv) were syringed sequentially into a solution of I_{2 (}0.51 g, 2.0 mmol, 1.0 equiv) in 1,2-dichloroethane (10 mL) at 0° C. After 15 min at 0° C., the reaction mixture was warmed slowly in a water bath until all precipitate had dissolved (35° C.). Stirring was ceased, and the crude reaction mixture was allowed to cool slowly to −20° C. (12 h). The residual solvent was removed and the small orange-yellow needles were washed with hexanes (2×0.5 mL) and dried under vacuum to afford 0.72 g (42% yield) of 109.

Asymmetric Cyclization Attempts of Homogeranylbenzene: A solution of 107 (0.053 g, 0.110 mmol, 1.1 equiv) in nitromethane (0.5 mL) was quickly added via syringe to a solution of homogeranylbenzene (71, 0.023 g, 0.100 mmol, 1.0 equiv) in nitromethane (1.5 mL) at −25° C. After stirring for 5 min at −25° C., the resulting mixture was poured into a solution of saturated aqueous NaHCO₃:5% aqueous Na₂SO₃ (1:1, mL) and the resultant biphasic mixture was stirred vigorously for 15 min at 25° C. The reaction contents were then extracted with CH₂Cl₂ (3×10 mL), and the combined organic layers were dried (MgSO₄), filtered, and concentrated. The resultant residue was purified by flash column chromatography (silica gel, hexanes: CH₂Cl₂, 9:1) to afford tricycle 104 (0.011 g contaminated with unknown impurities, <40% yield) as a colorless viscous solid. HPLC (OD column, 1.0 mL/min, 98:2 hex:IPA, 30° C., 265 nm, t_{R}=6.83 min, 9.33 min): 0% e.e. (See Ref. 9 for characterization data for 104).

The above procedure was repeated using 108 to afford 110 (0.022 g, 72% yield) as a white crystalline solid. HPLC (OD column, 1.0 mL/min, 98:2 hex:IPA, 30° C., 265 nm, t_{R}=6.92 min, 9.43 min): 0% e.e. (See Ref. 9 for characterization data for 110). The above procedure was repeated using 109 to afford 72 (0.028 g, 78% yield) as a white crystalline solid. HPLC (OD column, 1.0 mL/min, 99:1 hex:IPA, 30° C., 265 nm, t_{R}=7.54 min, 10.99 min): 0% e.e. (See Ref. [67] for characterization data for 72).

### 1,2-Dichlorotetrahydronaphthalene (112).

A solution of 1,2-dihydronaphthalene (111, 5.0 mg, 0.038 mmol, 1.0 equiv) in CH₂Cl₂ (0.2 mL) was added slowly via syringe to a solution of 107 (0.022 g, 0.046 mmol, 1.2 equiv) in CH₂Cl₂ (0.5 mL) at −78° C. The reaction mixture was stirred for 60 min at −78° C., then allowed to warm slowly to −20° C. over the course of 60 min. The reaction mixture was quenched by the addition of 5% aqueous Na₂SO₃ (3 mL) and saturated aqueous NaHCO₃ (3 mL), extracted into CH₂Cl₂ (3×5 mL), dried (MgSO₄), filtered, and concentrated. Purification by preparative TLC (silica gel, hexanes:CH₂Cl₂, 4:1) afforded 112 (4.4 mg, 57% yield) as a clear amorphous solid. 112: R_{f}=0.44 (silica gel, hexanes:CH₂Cl₂, 4:1); [α]_{D}²²+8.6° (c 0.40, CHCl₃, 14% e.e.); IR (film) νₘₐₓ 3023, 2927, 1491, 813, 737, 678, 646 cm⁻¹; ¹H NMR (400 MHz, CDCl₃) δ 7.34 (m, 1H), 7.28-7.18 (m, 2H), 7.13 (m, 1H), 5.23 (d, J=2.8 Hz, 1H), 4.66 (m, 1H), 3.16 (ddd, J=17.2, 11.2, 5.6 Hz, 1H), 2.87 (ddd, J=17.2, 6.0, 2.8 Hz, 1H), 2.67 (dddd, J=17.2, 11.2, 6.0, 2.4 Hz, 1H), 2.14 (m, 1H); ¹³C NMR (100 MHz, CDCl₃) δ 135.0, 132.5, 131.2, 129.2, 129.0, 126.8, 59.9, 59.6, 25.2, 24.0; HRMS (EI) calcd for C₁₀H₁₀Cl₂ [M]⁺ 200.0160. found 200.0151. HPLC(OD column, 1.0 mL/min, 250:1 hex:IPA, 30° C., 270 nm, t_{R} (major)=6.48 min, t_{R} (minor)=7.06 min): 14% e.e. Alternatively, a solution of 107 (0.022 g, 0.046 mmol, 1.2 equiv) in nitromethane (0.15 mL) was added via syringe to a solution of 1,2-dihydronaphthalene (111, 5.0 mg, 0.038 mmol, 1.0 equiv) in CH₂Cl₂ (0.6 mL) at −30° C. After stirring for 10 min at −30° C., the reaction mixture was quenched, extracted, and purified as above to afford 112 (3.5 mg, 45% yield) as a clear amorphous solid. HPLC (OD column, 1.0 mL/min, 250:1 hex:IPA, 30° C., 270 nm, t_{R} (major)=6.48 min, t_{R} (minor)=7.06 min): 6% e.e.

### 11. Ring-Forming Halolactonization: Synthesis of Heimol A and Hopeahainol D

### Lactone (114).

Carboxylic acid 113 (0.040 g, 0.074 mmol, 1.0 equiv) dissolved in MeCN (12 mL) at 25° C. and then a solution of IDSI (0.120 g, 0.148 mmol, 2.0 equiv) in MeCN (4 mL) added quickly via syringe. After stirring for 1 min at 25° C., the reaction contents were quenched with a mixture of 5% aqueous Na₂SO₃/saturated aqueous NaHCO₃ (1/1, 5 mL) and the resultant bi-phasic mixture was stirred vigorously for 5 min. The reaction contents were then extracted with CH₂Cl₂ (3×20 mL), and the combined organic extracts were dried (MgSO₄), filtered, and concentrated. Carrying this material forward without further purification, the newly formed lactone 9 was dissolved in CH₂Cl₂ (5 mL) at 25° C. and BBr₃ (1.9 mL, 1.0 M in CH₂Cl₂, 1.9 mmol, 25 equiv) added via syringe in a single portion. The resultant reaction mixture was then stirred at 25° C. for 24 h. Upon completion, the reaction contents quenched with water (3 mL), and the resultant bi-phasic system was stirred vigorously for 2 min and extracted with EtOAc (3×10 mL). The combined organic extracts were then washed with water (10 mL) and brine (10 mL), dried (MgSO₄), filtered, and concentrated. The resultant crude, dark red oil was purified by preparative thin-layer chromatography (silica gel, CH₂Cl₂/MeOH, 4/1) to give the desired deprotected lactone (10.5 mg, 36% yield over 2 steps) as a red oil. R_{f}=0.15 (silica gel, CH₂Cl₂/MeOH, 9/1); IR (film) μₘₐₓ 3435 (br), 2922, 2851, 1716, 1458, 1376, 1262, 1097, 1025, 802; ¹H NMR (400 MHz, acetone-d₆) δ 8.62 (s, —OH), 8.37 (s, —OH), 8.22 (s, —OH), 8.04 (s, —OH), 7.94 (s, —OH), 6.99 (d, J=8.4 Hz, 2H), 6.72 (d, J=8.8 Hz, 2H), 6.61 (d, J=2.0 Hz, 1H), 6.46 (d, J=2.4 Hz, 1H), 6.37 (d, J=2.0 Hz, 1H), 6.23 (d, J=2.0 Hz, 1H), 5.45 (d, J=2.8 Hz, 1H), 4.89 (s, 1H), 4.45 (d, J=2.8 Hz, 1H); ¹³C NMR (100 MHz, acetone-d₆) δ 172.4, 158.5, 158.4, 157.8, 156.6, 153.9, 138.5, 138.1, 134.9, 130.1, 117.2, 115.5, 115.1, 107.7, 105.2, 103.2, 84.8, 48.8, 47.9; HRMS (FAB+) calcd for C₂₂H₁₇O₇⁺[M⁺] 393.0974. found 393.0983.

12. Ring Expanding Bromoetherification: Preparation of 8- and 9-Membered Laurencia-type Bromoethers (87, 88, 89)

### I. Synthesis of Model Substrate 127

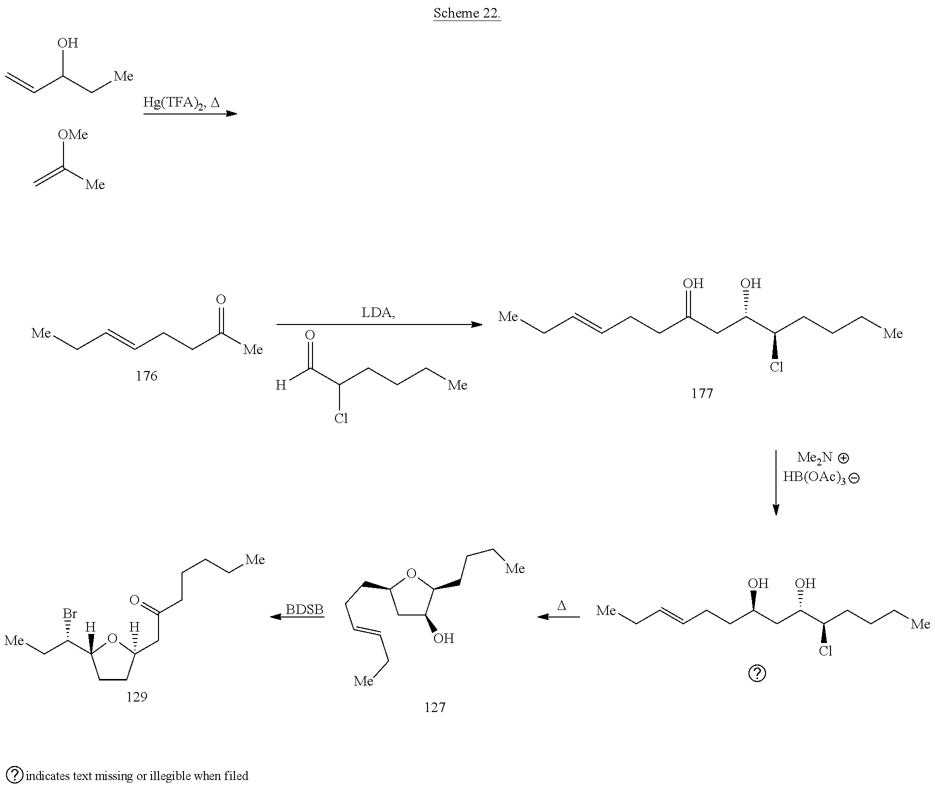

176.

1-Penten-3-ol (1.00 mL, 9.76 mmol, 1.0 equiv), 2-methoxypropene (4.69 mL, 48.8 mmol, 5.0 equiv), and Hg(TFA)₂ (0.083 g, 0.20 mmol, 0.02 equiv) were combined and sealed in a high-pressure sealed tube. The reaction mixture was heated to 125° C. and stirred at that temperature for 2 h. Upon completion, the reaction mixture was allowed to cool to 25° C., poured into water (30 mL), and extracted with Et₂O (3×20 mL). The combined organic layers were washed sequentially with water (30 mL), saturated aqueous NaHCO₃ (30 mL), and brine (30 mL), dried (MgSO₄), filtered, and carefully concentrated (˜50 mm Hg at 20° C.). The crude residue was purified by flash column chromatography (silica gel, hexanes:Et₂O, 9:1) to afford (5E)-octen-2-one (0.854 g, 72% yield) as a moderately volatile light yellow oil. 176: R_{f}=0.48 (silica gel, hexanes:EtOAc, 9:1); IR (film) νₘₐₓ 2963, 2931, 1718, 1438, 1360, 1162, 969 cm⁻¹; ¹H NMR (400 MHz, CDCl₃) δ 5.48 (m, 1H), 5.37 (m, 1H), 2.48 (t, J=7.2 Hz, 2H), 2.25 (app q, 2H), 2.13 (s, 3H), 1.98 (app quintet, 2H), 0.95 (t, J=7.6 Hz, 3H); ¹³C NMR (100 MHz, CDCl₃) δ 208.8, 133.3, 127.3, 43.8, 30.1, 27.0, 25.7, 13.9; HRMS (EI) calcd for C₈H₁₄O [M]⁺ 126.1045. found 126.1039.

177.

A solution of n-BuLi (1.5 M in hexane, 4.83 mL, 7.24 mmol, 1.1 equiv) was added dropwise to a solution of iPr₂NH (1.11 mL, 7.89 mmol, 1.2 equiv) in THF (22 mL) at −78° C. The resultant colorless solution was removed from the cold bath and allowed to warm (to ˜0° C.) over 15 min, then re-cooled to −78° C. (5E)-Octen-2-one (176, 0.830 g, 6.58 mmol, 1.0 equiv) was added dropwise to the resultant LDA solution and the colorless solution was stirred for 1 h at −78° C. A solution of 2-chlorohexanal (1.06 g, 7.89 mmol, 1.2 equiv) in THF (8 mL) was then added dropwise and the colorless solution was stirred for an additional 60 min at −78° C. Upon completion, the reaction mixture was quenched by the addition of saturated aqueous NH₄Cl (20 mL) and water (20 mL). The crude product was extracted into hexanes/EtOAc (1:1, 3×50 mL) and the combined organic layers were washed with brine (100 mL), dried (MgSO₄), filtered, and concentrated. The resultant yellow oil was purified by careful flash column chromatography (silica gel, hexanes:EtOAc, 1:07:1) to afford β-hydroxy ketone 177 (0.925 g, 54% yield) as a colorless viscous oil.

178.

A solution of tetramethylammonium triacetoxyborohydride (2.42 g, 9.20 mmol, 5.0 equiv) in MeCN (30 mL) and AcOH (18 mL) was stirred for 10 min at 25° C., and then was cooled to −40° C. A solution of β-hydroxy ketone 177 (0.480 g, 1.84 mmol, 1.0 equiv) in MeCN (6 mL) was added, and the reaction mixture was allowed to warm very slowly to 25° C. over 16 h, then quenched by the addition of 1 M sodium/potassium tartrate (30 mL) and water (70 mL). The crude product was extracted into Et₂O (3×75 mL), and the combined organic layers were washed with water (2×50 mL) then brine (50 mL), dried (MgSO₄), filtered, and concentrated (with the addition of toluene to help remove any residual AcOH by coevaporation). The resultant oil was purified by two successive recrystallizations from CH₂Cl₂:hexanes (1:4, 30 mL, then 10 mL) to afford trans-diol 178 (0.380 g, 79% yield) as a white crystalline solid. 178: R_{f}=0.57 (silica gel, hexanes:EtOAc, 7:3); IR (film) νₘₐₓ 3314 (br), 2957, 2931, 2872, 1445, 1046, 964, 738 cm⁻¹; ¹H NMR (400 MHz, CDCl₃) δ 5.52 (m, 1H), 5.42 (m, 1H), 4.50-3.93 (m, 3H), 2.13 (m, 2H), 2.00 (m, 2H), 1.88-1.75 (m, 2H), 1.73-1.52 (m, 4H), 1.43-1.27 (m, 4H), 0.97 (t, J=7.2 Hz, 3H), 0.92 (t, J=7.2 Hz, 3H); ¹³C NMR (100 MHz, CDCl₃) δ 133.1, 128.4, 71.9, 69.0, 68.1, 38.3, 37.2, 33.1, 29.1, 28.8, 25.6, 22.2, 13.9 (2C); HRMS (FAB) calcd for C₁₄H₂₈ClO₂ [M+H]⁺ 263.1778. found 263.1773.

127.

Compound 178 (0.100 g, 0.380 mmol, 1.0 equiv) was dissolved in MeOH (8 mL) and water (4 mL) in a high-pressure sealed tube. The tube was sealed and heated to 130° C. for 5 h. Upon completion, the reaction mixture was allowed to cool to 25° C., then quenched by the addition of saturated aqueous NaHCO₃ (10 mL) and water (10 mL), and extracted with EtOAc (3×20 mL). The combined organic layers were washed with brine (40 mL), dried (MgSO₄), filtered, and concentrated. The resultant oil was purified by flash column chromatography (silica gel, hexanes:EtOAc, 1:04:1) to afford the desired hydroxytetrahydrofuran 127 (0.061 g, 71% yield) as a colorless viscous oil. 127: R_{f}=0.36 (silica gel, hexanes:EtOAc, 9:1); IR (film) νₘₐₓ 3415 (br), 2959, 2933, 2858, 1458, 1088, 967 cm⁻¹; ¹H NMR (400 MHz, CDCl₃) δ 5.52-5.34 (m, 2H), 4.15 (m, 1H), 3.76 (m, 1H), 3.48 (td, J=6.8, 3.2 Hz, 1H), 2.37 (ddd, J=14.0, 8.0, 6.4 Hz, 1H), 2.16-1.94 (m, 4H), 1.76 (m, 1H), 1.71-1.48 (m, 5H), 1.46-1.30 (m, 4H), 0.95 (t, J=7.2 Hz, 3H), 0.91 (t, J=7.2 Hz, 3H); ¹³C NMR (100 MHz, CDCl₃) δ 132.4, 128.4, 83.1, 77.2, 72.9, 41.7, 36.5, 29.1, 28.5, 28.4, 25.6, 22.9, 14.0, 13.9; HRMS: No molecular ion peak could be observed.

129.

A solution of BDSB (0.0659 g, 0.120 mmol, 1.2 equiv) in MeNO₂ (0.5 mL) was added rapidly via syringe to a solution of cyclization precursor 127 (0.0226 g, 0.100 mmol, 1.0 equiv) in MeNO₂ (4.5 mL) at −25° C. After stirring for 10 min at −25° C., the reaction mixture was quenched by the addition of a combination of saturated aqueous NaHCO₃ and 5% aqueous Na₂SO₃ (1:1, 5 mL) and the resultant biphasic mixture was stirred vigorously for 20 min at 25° C. The reaction contents were added to water (10 mL) and then extracted with CH₂Cl₂ (3×10 mL). The combined organic layers were dried (MgSO₄), filtered, and concentrated. The resultant residue was purified by careful flash column chromatography (silica gel, hexanes:EtOAc, 1:0→12:1) to afford ketone 129 (0.0123 g) as a colorless viscous oil contaminated with a small amount of inseparable impurities (estimated pure yield=0.0105 g, 34%). Connectivity and stereochemistry were determined by COSY and NOESY NMR experiments (see attached spectra). A second major product was also isolated (in approximately 25% yield); its structure could not be fully elucidated, but NMR evidence suggests it is a diastereomer of 129. 11: R_{f}=0.53 (silica gel, hexanes:EtOAc, 4:1); IR (film) νₘₐₓ 2958, 2928, 2872, 1713, 1461, 1379, 1045 cm⁻¹; ¹H NMR (400 MHz, CDCl₃) δ 4.39 (m, 1H), 4.04 (ddd, J=14.0, 8.0, 6.4 Hz, 1H), 3.88 (ddd, J=10.8, 9.6, 3.2 Hz, 1H), 2.73 (dd, J=15.6, 6.8 Hz, 1H), 2.50 (dd, J=15.6, 6.0 Hz, 1H), 2.43 (t, J=7.2 Hz, 2H), 2.23-2.12 (m, 2H), 2.02 (sextet of doublets, J=7.2, 3.2 Hz, 1H), 1.88 (m, 1H), 1.72 (m, 1H), 1.61-1.48 (m, 3H), 1.35-1.22 (m, 4H), 1.05 (t, J=7.6 Hz, 3H), 0.88 (t, J=7.2 Hz, 3H); ¹³C NMR (125 MHz, CDCl₃) δ 209.5, 81.4, 76.2, 62.3, 48.7, 43.7, 32.5, 31.5, 31.1, 28.7, 23.4, 22.6, 14.1, 12.1; HRMS (FAB) calcd for C₁₄H₂₆BrO₂ [M+H]⁺ 305.1116. found 305.1108.

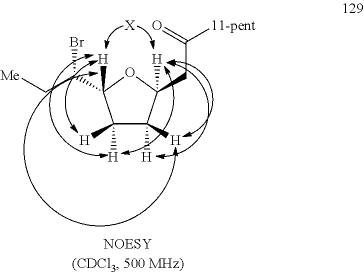

II. Synthesis of Hydroxytetrahydrofurans and their Derivatives (90, 91, 92)

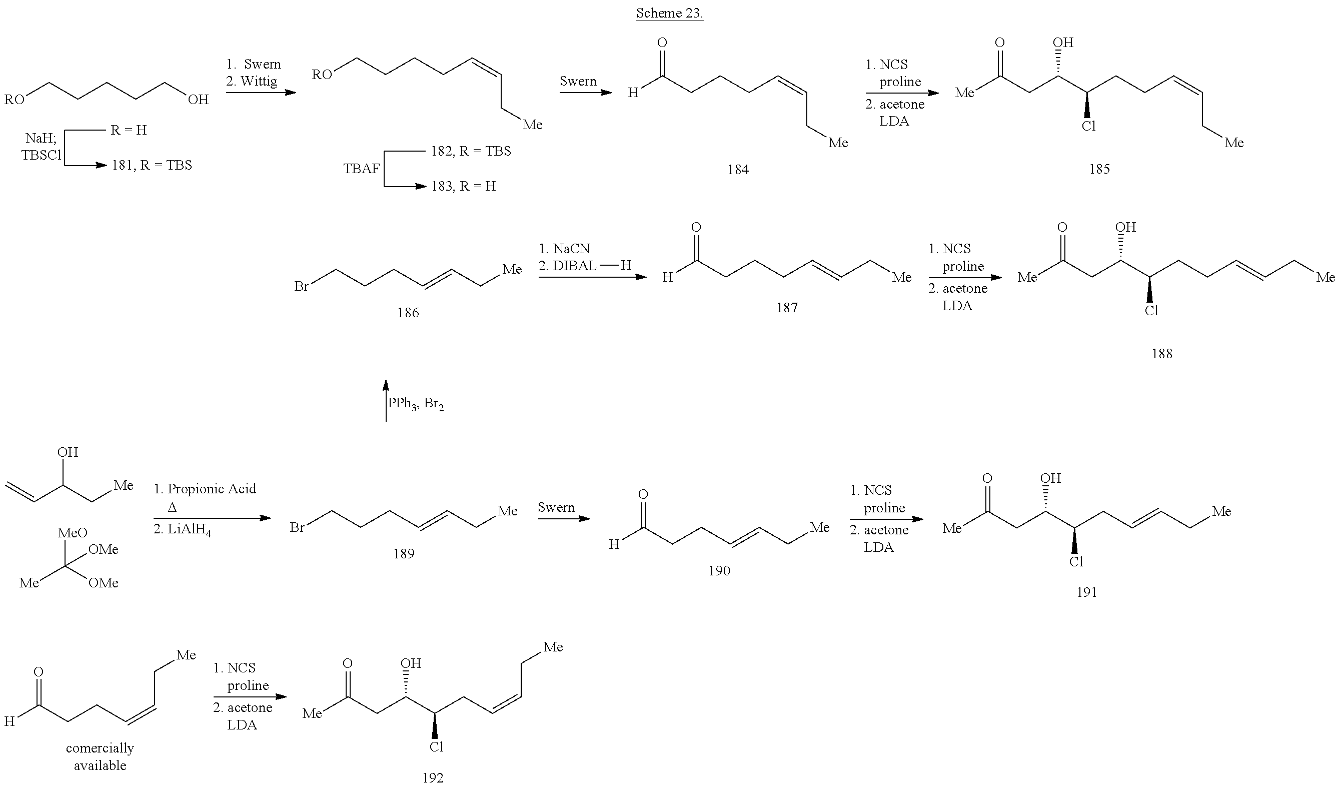

181.

1,5-Pentanediol (6.3 mL, 60. mmol, 1.0 equiv) was added dropwise to a suspension of NaH (60% dispersion in mineral oil, 2.4 g, 60. mmol, 1.0 equiv) in THF (120 mL) at 25° C. (while venting the H₂ produced). The resultant reaction mixture was stirred vigorously for 45 min at 25° C., after which TBSCl (9.0 g, 60. mmol, 1.0 equiv) was added in a single portion. The reaction mixture was then stirred for an additional 2 h at 25° C. Upon completion, the reaction contents were quenched by the careful addition of saturated aqueous NaHCO₃ (100 mL) and extracted with EtOAc (3×50 mL). The combined organic layers were then dried (MgSO₄), filtered, and concentrated. The resultant colorless oil was purified by flash column chromatography (silica gel, hexanes:EtOAc, 4:1) to afford desymmetrized alcohol 181 (10.3 g, 79% yield) as a colorless viscous oil. 181: R_{f}=0.42 (silica gel, hexanes:EtOAc, 4:1); IR (film) νₘₐₓ 3351 (br), 2933, 2859, 1470, 1388, 1254, 1101 cm⁻¹; ¹H NMR (400 MHz, CDCl₃) δ 3.65 (t, J=6.4 Hz, 2H), 3.62 (t, J=6.4 Hz, 2H), 1.63-1.51 (m, 4H), 1.46-1.37 (m, 2H), 0.89 (s, 9H), 0.05 (s, 6H); ¹³C NMR (75 MHz, CDCl₃) δ 63.5, 63.3, 32.9 (2C), 26.3 (3C), 22.4, 18.7, −4.9 (2C); HRMS (FAB) calcd for C₁₁H₂₇O₂Si [M+H]⁺ 219.1780. found 219.1779.

182.

DMSO (3.75 mL, 52.8 mmol, 2.0 equiv) was added dropwise over the course of 5 min to a solution of oxalyl chloride (2.76 mL, 31.7 mmol, 1.2 equiv) in CH₂Cl₂ (264 mL) at −78° C., and the resultant colorless solution was stirred at −78° C. for 5 min. A solution of 181 (5.77 g, 26.4 mmol, 1.0 equiv) in CH₂Cl₂ (50 mL) was then added slowly over the course of 10 min, and the resultant colorless solution was stirred for an additional 5 min at −78° C. Finally, Et₃N (14.6 mL, 106 mmol, 4.0 equiv) was added slowly via syringe, and the reaction contents were allowed to warm slowly to −40° C. over the course of 2 h. Upon completion, the reaction contents were quenched by the addition of water (200 mL) and extracted with CH₂Cl₂ (2×100 mL). The combined organic layers were then washed with 1 M HCl (100 mL), dried (MgSO₄), filtered, and concentrated to afford the desired aldehyde as a light yellow oil, which was carried forward without any additional purification. Next, KOt-Bu (1.0 M in THF, 29.0 mL, 29.0 mmol, 1.1 equiv) was added slowly to a suspension of propyltriphenylphosphonium bromide (12.2 g, 31.7 mmol, 1.2 equiv) in THF (86 mL) at 0° C. The resultant orange solution was allowed to warm to 25° C. and stirred for an additional 30 min, then re-cooled to 0° C. A solution of the aldehyde produced above (26.4 mmol assumed, 1.0 equiv) in THF (20 mL) was added slowly into the ylide solution via cannula. After stirring for 1 h at 0° C., the reaction contents were quenched by the sequential addition of saturated aqueous NH₄Cl (50 mL) and water (50 mL), and extracted with Et₂O (3×100 mL). The combined organic layers were then washed with brine (200 mL), dried (MgSO₄), filtered, and concentrated. The bulk of the triphenylphosphine oxide byproduct was removed by slowly concentrating the resultant crude product from a solution of CH₂Cl₂:hexanes (1:1, 200 mL) until approximately 50 mL solvent remained. The resultant slurry was filtered, and the precipitate was rinsed with hexanes (2×30 mL); the combined filtrate and rinses were concentrated to afford 182 as a light yellow oil, which was carried forward without any additional purification.

183.

A solution of TBAF (1.0 M in THF, 31.7 mL, 31.7 mmol, 1.2 equiv) was added to a solution of crude alkene 182 (26.4 mmol assumed, 1.0 equiv) in THF (94 mL) at 0° C., and the resultant reaction mixture was stirred at 0° C. for 1 h before being warmed to 25° C. After an additional 3 h at 25° C., the reaction contents were quenched by the sequential addition of saturated aqueous NH₄Cl (50 mL) and water (50 mL) and extracted with Et₂O (3×100 mL). The combined organic layers were washed with brine (200 mL), dried (MgSO₄), filtered, and concentrated. The resultant light brown oil was purified by flash column chromatography (silica gel, hexanes:EtOAc, 19:1-4:1) to afford 183 (2.44 g, 72% yield over 3 steps) as a colorless viscous oil. 183: R_{f}=0.44 (silica gel, hexanes:EtOAc, 4:1); IR (film) νₘₐₓ 3332 (br), 3005, 2934, 1458, 1276, 1261, 1062, 750 cm⁻¹; ¹H NMR (400 MHz, CDCl₃) δ 5.42-5.28 (m, 2H), 3.65 (t, J=6.8 Hz, 2H), 2.10-2.00 (m, 4H), 1.58 (m, 2H), 1.44 (m, 2H), 1.35 (br s, 1H), 0.96 (t, J=7.6 Hz, 3H); ¹³C NMR (100 MHz, CDCl₃) δ 132.2, 128.9, 63.1, 32.5, 26.9, 26.0, 20.7, 14.5; HRMS (EI) calcd for C₈H₁₆O [M]⁺128.1201. found 128.1199.

184.

Prepared according to the Swern procedure described above for 182. 183 (2.44 g, 19.0 mmol) was subjected to oxidation followed by purification by flash column chromatography (silica gel, hexanes:EtOAc, 1:0→19:1) to afford (5Z)-octenal (184, 2.18 g, 91% yield) as a light yellow oil. 184: R_{f}=0.36 (silica gel, hexanes:EtOAc, 19:1); IR (film) νₘₐₓ 3005, 2962, 2934, 1748, 1242 cm⁻¹; ¹H NMR (400 MHz, CDCl₃) δ 9.77 (t, J=2.0 Hz, 1H), 5.42 (m, 1H), 5.27 (m, 1H), 2.43 (td, J=7.2, 2.0 Hz, 2H), 2.12-1.97 (m, 4H), 1.70 (quintet, J=7.2 Hz, 2H), 0.96 (t, J=7.6 Hz, 3H); ¹³C NMR (100 MHz, CDCl₃) δ 202.8, 133.1, 127.8, 43.4, 26.5, 22.2, 20.7, 14.4; HRMS (FAB) calcd for C₈H₁₃O [M−H]⁺ 125.0966. found 125.0965.

186.

Bromine (1.08 mL, 21.0 mmol, 1.05 equiv) was added dropwise to a solution of Ph₃P (6.30 g, 24.0 mmol, 1.2 equiv) in CH₂Cl₂ (120 mL) at 0° C., and the resultant colorless solution was stirred for 5 min. The reaction mixture was then cooled to −20° C. and trans-4-hepten-1-ol (189 [vide infra], 2.28 g, 20.0 mmol, 1.0 equiv) was added dropwise. The resultant reaction solution was allowed to warm slowly over 2 h to 25° C. Upon completion, the reaction contents were concentrated by rotary evaporation to a volume of −20 mL, and the resultant residue was purified directly by flash column chromatography (silica gel, pentane:Et₂O, 19:1) to afford bromide 186 (2.82 g, 75% yield) as a colorless volatile oil. 186: R_{f}=0.81 (silica gel, hexanes); IR (film) νₘₐₓ 2962, 2930, 2848, 1438, 1239, 967 cm⁻¹; ¹H NMR (400 MHz, CDCl₃) δ 5.47 (m, 1H), 5.29 (m, 1H), 3.35 (t, J=6.8 Hz, 2H), 2.08 (q, J=6.8 Hz, 2H), 1.95 (m, 2H), 1.85 (quintet, J=6.8 Hz, 2H), 0.91 (t, J=7.6 Hz, 3H); ¹³C NMR (100 MHz, CDCl₃) δ 133.9, 127.1, 33.5, 32.6, 31.0, 25.7, 14.0; HRMS (FAB) calcd for C₈H₁₃O [M−H]⁺ 125.0966. found 125.0962. Next, a portion of bromide 186 (2.15 g, 12.1 mmol, 1.0 equiv) was added to a suspension of NaCN (0.892 g, 18.2 mmol, 1.5 equiv) in DMSO (18 mL) at 25° C., and the resultant reaction mixture was stirred vigorously for 3 h. Upon completion, the reaction contents were quenched with water (150 mL) and extracted with hexanes:Et₂O (1:1, 3×75 mL). The combined organic layers were then washed with water (3×50 mL), then brine (50 mL), dried (MgSO₄), filtered, and concentrated to afford the desired nitrile, which was carried forward without additional purification.

187.

DIBAL-H (1.0 M in toluene, 14.6 mL, 14.6 mmol, 1.2 equiv) was added dropwise over the course of 10 min to a solution of the crude nitrile produced above (12.1 mmol assumed, 1.0 equiv) in CH₂Cl₂ (61 mL) at −78° C. The resultant colorless solution was allowed to warm slowly to −50° C. over 90 min. Upon completion, the reaction contents were quenched by the sequential addition of acetone (120 mL), saturated aqueous NH₄Cl (10 mL), and 1 M sodium potassium tartrate (30 mL); the resultant biphasic mixture was stirred vigorously for 12 h at 25° C. The reaction contents were then poured into brine (200 mL) and extracted with Et₂O (3×100 mL). The combined organic layers were washed with brine (200 mL), dried (MgSO₄), filtered, and concentrated to afford (5E)-octenal (187, 1.26 g, 83% yield) as a light yellow viscous oil which was carried forward without additional purification. [Note: this product proved unstable to silica gel exposure, rendering column purification impractical]. 187: IR (film) νₘₐₓ 2958, 2918, 2850, 1724, 1460, 968 cm⁻¹; ¹H NMR (400 MHz, CDCl₃) δ 9.76 (t, J=2.0 Hz, 1H), 5.47 (m, 1H), 5.34 (m, 1H), 2.42 (td, J=7.2, 1.6 Hz, 2H), 2.07-1.95 (m, 4H), 1.70 (quintet, J=7.2 Hz, 2H), 0.96 (t, J=7.2 Hz, 3H); ¹³C NMR (100 MHz, CDCl₃) δ 203.0, 133.6, 127.9, 43.3, 31.9, 25.7, 22.1, 14.0; HRMS (FAB) calcd for C₈H₁₃O [M−H]⁺ 125.0966. found 125.0962.

189.

1-Penten-3-ol (10.0 g, 116 mmol, 1.0 equiv), propionic acid (0.435 mL, 5.80 mmol, 0.05 equiv), and trimethyl orthoacetate (43.6 mL, 348 mmol, 3.0 equiv) were added to a high-pressure sealed tube. The reaction vessel was then sealed and heated at 120° C. for 12 h. Upon completion, the reaction contents were cooled to 25° C., the cap was removed, and the reaction contents were reheated to 120° C. for 2 h, open to the atmosphere, to distill off the MeOH byproduct. The resultant yellow oil was then dissolved in Et₂O (20 mL) and cannulated dropwise into a suspension of LiAlH₄ (4.41 g, 116 mmol, 1.0 equiv) in Et₂O (440 mL) at 0° C. The resultant slurry was stirred at 0° C. for 60 min and then quenched by the careful dropwise addition of saturated aqueous NH₄Cl (20 mL), followed by 1 M sodium potassium tartrate solution (300 mL). The resultant biphasic mixture was stirred vigorously for 16 h at 25° C. The layers were then allowed to separate and the aqueous layer was extracted with additional Et₂O (2×200 mL). The combined organic layers were washed with brine (200 mL), dried (MgSO₄), filtered, and concentrated (→100 mm Hg at 20° C.). The resultant crude oil was purified by flash column chromatography (silica gel, hexanes:Et₂₀, 1:0→4:1) to afford (4E)-hepten-1-ol (189, 11.1 g, 84% yield) as a moderately volatile colorless oil. 189: R_{f}=0.28 (silica gel, hexanes:EtOAc, 4:1); IR (film) νₘₐₓ 3335 (br), 2962, 2934, 2874, 1454, 1058, 966 cm⁻¹; ¹H NMR (400 MHz, CDCl₃) δ 5.49 (m, 1H), 5.41 (m, 1H), 3.65 (t, J=6.4 Hz, 2H), 2.12-1.96 (m, 4H), 1.64 (quintet, J=6.8 Hz, 2H), 1.40 (br s, 1H), 0.97 (t, J=7.2 Hz, 3H); ¹³C NMR (100 MHz, CDCl₃) δ 132.9, 128.6, 62.8, 32.6, 29.0, 25.7, 14.0; HRMS (EI) calcd for C₇H₁₄O [M]⁺114.1045. found 114.1041.

190.

Prepared according to the Swern procedure described above for the oxidation of S5; (4E)-hepten-1-ol (189, 3.34 g, 29.2 mmol) was oxidized to (4E)-heptenal (190, 2.36 g, 72% yield) as a volatile light yellow oil which was carried forward without any additional purification. [Note: this product proved unstable to silica gel exposure, rendering column purification impractical]. 190: IR (film) νₘₐₓ 2963, 2719, 1726, 1441, 1243, 968 cm⁻¹; ¹H NMR (400 MHz, CDCl₃) δ 9.76 (t, J=2.0 Hz, 1H), 5.51 (m, 1H), 5.39 (m, 1

H), 2.49 (m, 2H), 2.33 (m, 2H), 1.99 (m, 2H), 0.96 (t, J=7.6 Hz, 3H); ¹³C NMR (100 MHz, CDCl₃) δ 202.6, 133.7, 126.8, 43.7, 25.6, 25.3, 13.9; HRMS: No molecular ion peak could be observed.

185.

A suspension of NCS (3.47 g, 26.0 mmol, 1.3 equiv) and L-proline (0.230 g, 2.00 mmol, 0.10 equiv) in CH₂Cl₂ (60 mL) was cooled to 0° C., and (5Z)-octenal (184, 2.52 g, 20.0 mmol, 1.0 equiv) was added. The resultant reaction mixture was allowed to warm very slowly, approaching 25° C. Once the reaction had reached ˜50% conversion as judged by NMR analysis of reaction aliquots (−3 h, 9° C.), a second portion of L-proline (0.230 g, 2.00 mmol, 0.10 equiv) was added and the reaction was stirred for an additional 3 h with continued slow warming. Upon completion (final reaction temperature: 18° C.), the reaction contents were diluted with hexanes (120 mL) and cooled to −78° C. The resultant slurry was filtered (precipitate was rinsed with 2×15 mL of cold hexanes) and the combined filtrate and rinses were concentrated (˜150 mm Hg at 20° C.) to a total volume of −20 mL. The reaction contents were then cooled to −20° C. for 16 h, during which time more precipitate formed. The filtrate was decanted and concentrated (→100 mm Hg at 20° C.). The resultant yellow oil was purified by distillation under reduced pressure (2 mm Hg at 60° C.) to afford (5Z)-2-chloro-5-octenal (2.01 g, 63% yield) as a fragrant colorless viscous oil. [Note: this unsaturated α-chloro aldehyde, as with all others produced by this procedure, was rather unstable and was used immediately]. Pressing forward, n-BuLi (1.5 M in hexane, 7.28 mL, 10.9 mmol, 1.05 equiv) was added dropwise to a solution of iPr₂NH (1.76 mL, 12.5 mmol, 1.2 equiv) in THF (52 mL) at −78° C. The resultant colorless solution was removed from the cold bath and allowed to warm (to ˜0° C.) over 15 min, then re-cooled to −78° C. Acetone (0.766 mL, 10.4 mmol, 1.0 equiv) was added dropwise to the resultant LDA solution and the reaction contents were stirred for 30 min at −78° C. The aldehyde produced above, (5Z)-2-chloro-5-octenal (2.01 g, 12.5 mmol, 1.2 equiv), was then added dropwise and the resultant colorless solution was stirred for an additional 60 min at −78° C. Upon completion, the reaction contents were quenched by the addition of saturated aqueous NH₄Cl (30 mL) and water (30 mL) and extracted with hexanes:EtOAc (1:1, 3×80 mL). The combined organic layers were then washed with brine (150 mL), dried (MgSO₄), filtered, and concentrated. The resultant crude yellow oil was purified by careful flash column chromatography (silica gel, hexanes:EtOAc, 1:07:3) to afford 185 (1.38 g, 61% yield) as a colorless viscous oil. [Note: although stable to silica gel, the aldol products tended to decompose over time and as such were used immediately].

188.

Prepared according to the procedure described above for the synthesis of 185. (5E)-Octenal (187, 1.26 g, 10.0 mmol) was α-chlorinated to yield the highly unstable (5E)-2-chloro-5-octenal (0.600 g, 37% yield after vacuum distillation: 2 mm Hg at 60° C.) as a fragrant colorless oil. The aldol addition with acetone was performed immediately to yield 188 (0.383 g, 56% yield) as a colorless viscous oil after purification by careful flash column chromatography (silica gel, hexanes:EtOAc, 1:07:3).

191.

Prepared according to the procedure described above for the synthesis of 185. (4E)-Heptenal (190, 1.82 g, 16.2 mmol) was α-chlorinated to yield (4E)-2-chloro-4-heptenal (1.88 g, 79% yield—no vacuum distillation necessary) as a fragrant colorless oil. The aldol addition with acetone was performed immediately to yield 191 (1.35 g, 62% yield) as a colorless viscous oil after purification by careful flash column chromatography (silica gel, hexanes:EtOAc, 1:07:3).

192.

Prepared according to the procedure described above for the synthesis of 185. (4Z)-Heptenal (2.24 g, 20.0 mmol) was α-chlorinated to yield (4Z)-2-chloro-4-heptenal (2.23 g, 76% yield after vacuum distillation: 2 mm Hg at 50° C.) as a fragrant colorless oil. The aldol addition with acetone was performed immediately to yield 192 (1.56 g, 60% yield) as a colorless viscous oil after purification by careful flash column chromatography (silica gel, hexanes:EtOAc, 1:07:3).

### Reduction to Diols

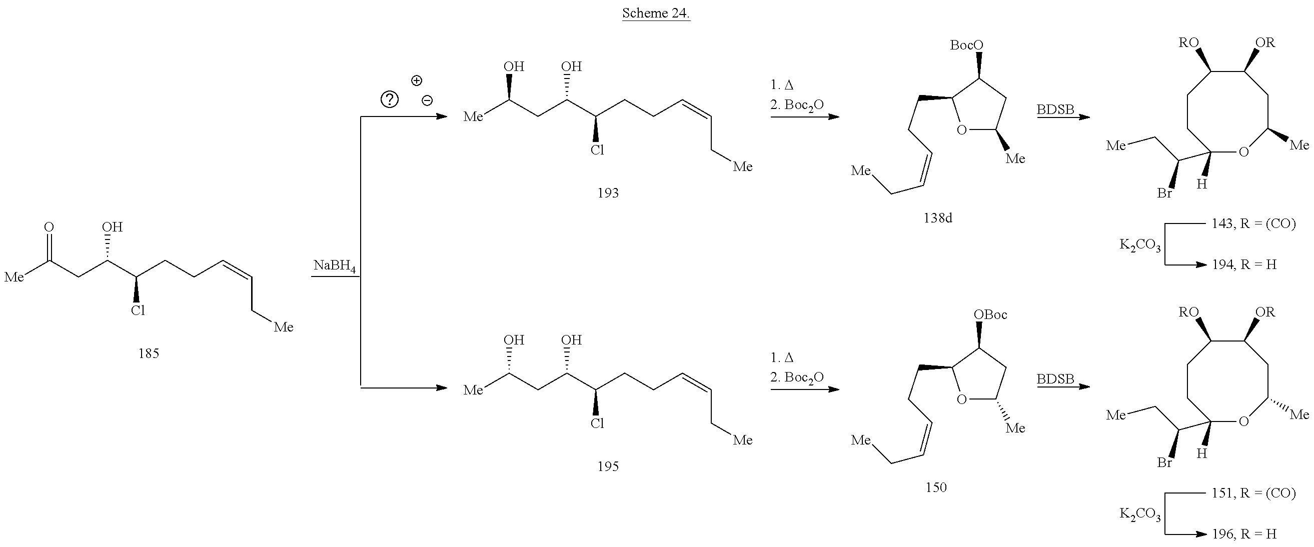

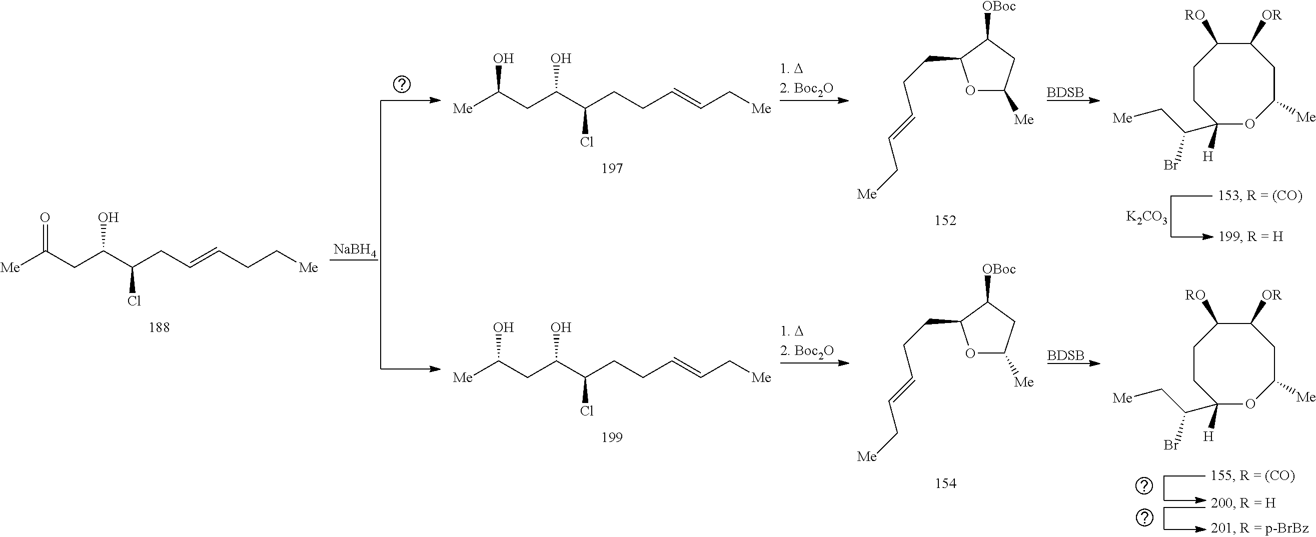

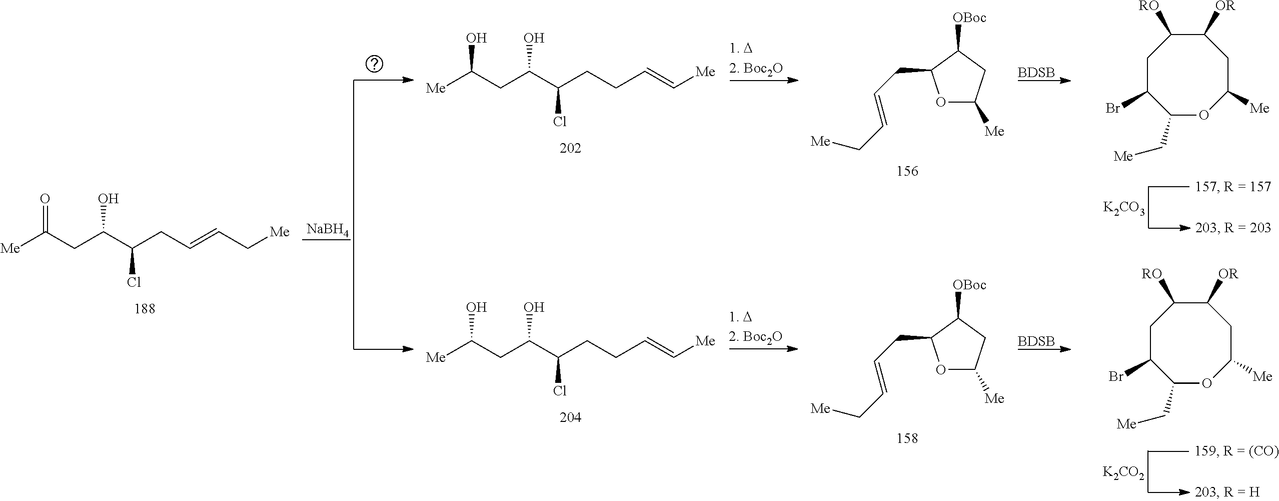

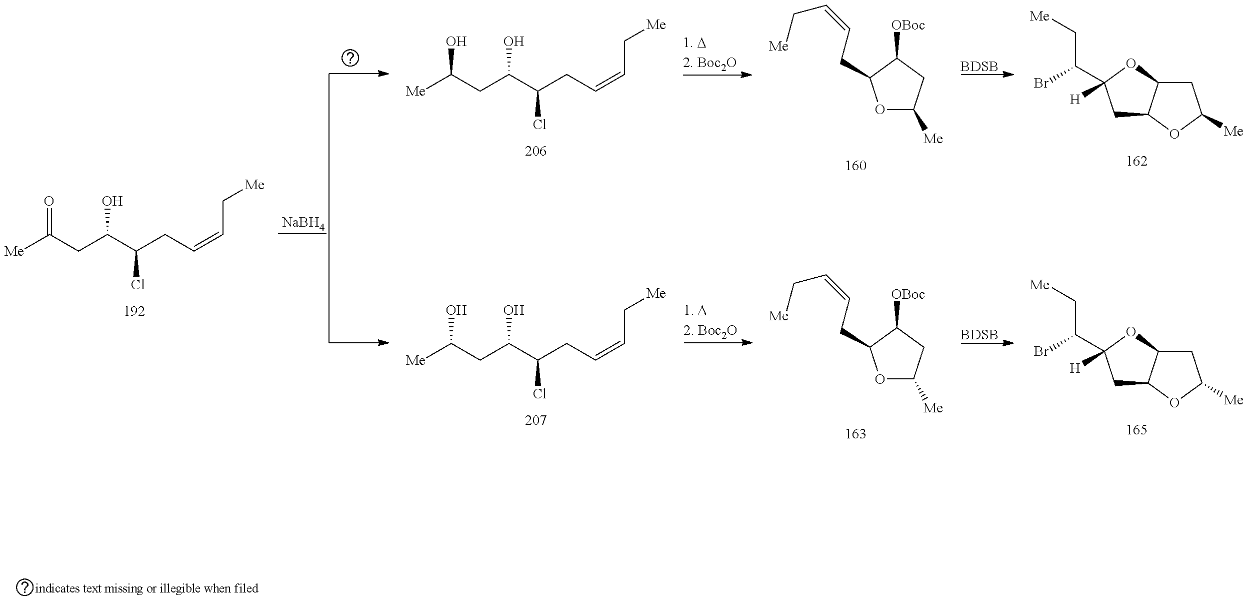

193.

A solution of tetramethylammonium triacetoxyborohydride (3.75 g, 14.3 mmol, 4.0 equiv) in MeCN (60 mL) and AcOH (35 mL) was stirred for 10 min at 25° C., then cooled to −40° C. Next, a solution of ketone 185 (0.780 g, 3.57 mmol, 1.0 equiv) in MeCN (10 mL) was added, and the reaction mixture was allowed to warm very slowly to 25° C. over the course of 12 h. Upon completion, the reaction contents were quenched by the addition of 1 M sodium potassium tartrate (60 mL) and water (150 mL), and extracted with Et₂O (3×150 mL). The combined organic layers were washed with water (2×200 mL), then brine (200 mL), dried (MgSO₄), filtered, and concentrated (with the addition of toluene to help remove any residual AcOH by coevaporation). The resultant crude oil was purified by careful flash column chromatography (silica gel, hexanes:EtOAc, 1:01:1) to afford 193 (0.622 g, 79% yield) as a colorless amorphous solid. 193: R_{f}=0.36 (silica gel, hexanes:EtOAc, 1:1); IR (Film) νₘₐₓ 3362 (br), 2965, 2933, 2874, 1455, 1069 cm⁻¹; ¹H NMR (400 MHz, CDCl₃) δ 5.44 (m, 1H), 5.28 (m, 1H), 4.19 (m, 1H), 4.06-3.95 (m, 2H), 2.82 (d, J=6.4 Hz, 1H), 2.34-2.18 (m, 2H), 2.07 (quintet, J=7.2 Hz, 2H), 1.95 (d, J=4.4 Hz, 1H), 1.93-1.70 (m, 3H), 1.63 (ddd, J=14.8, 8.8, 2.4 Hz, 1H), 1.27 (d, J=6.4 Hz, 3H), 0.97 (t, J=7.6 Hz, 3H); ¹³C NMR (100 MHz, CDCl₃) δ 133.5, 127.2, 72.0, 67.5, 65.5, 40.2, 33.4, 24.2, 24.0, 20.7, 14.5; HRMS (FAB) calcd for C₁₁H₂₂ClO₂ [M+H]⁺ 221.1308. found 221.1307.

197.

Prepared according to the procedure described above for the synthesis of 193. Reduction of 188 (0.150 g, 0.686 mmol) afforded trans-diol 197 (0.136 g, 91% yield) as a colorless amorphous solid. 197: R_{f}=0.35 (silica gel, hexanes:EtOAc, 1:1); IR (film) νₘₐₓ 3368 (br), 2964, 2931, 1451, 1376, 1065, 968 cm⁻¹; ¹H NMR (400 MHz, CDCl₃) δ 5.54 (m, 1H), 5.35 (m, 1H), 4.19 (m, 1H), 4.06-3.97 (m, 2H), 2.72 (br s, 1H), 2.29 (m, 1H), 2.11 (sextet, J=7.6 Hz, 1H), 2.00 (quintet, J=7.2 Hz, 2H), 1.93-1.72 (m, 3H), 1.62 (ddd, J=14.4, 8.4, 2.4 Hz, 1H), 1.58 (br s, 1H), 1.28 (d, J=6.4 Hz, 3H), 0.97 (t, J=7.2 Hz, 3H); ¹³C NMR (100 MHz, CDCl₃) δ 133.8, 127.3, 72.0, 67.5, 65.5, 40.2, 33.3, 29.5, 25.7, 24.0, 14.0; HRMS (FAB) calcd for C₁₁H₂₂ClO₂ [M+H]⁺ 221.1308. found 221.1306.

202.

Prepared according to the procedure described above for the synthesis of 193. Reduction of 191 (0.320 g, 1.56 mmol) afforded trans-diol 202 (0.269 g, 83% yield) as a white crystalline solid. 202: R_{f}=0.39 (silica gel, hexanes:EtOAc, 1:1); IR (film) νₘₐₓ 3363 (br), 2965, 2932, 2874, 1459, 1376, 1067, 967 cm⁻¹; ¹H NMR (400 MHz, CDCl₃) δ 5.60 (m, 1H), 5.45 (m, 1H), 4.18 (m, 1H), 4.02 (m, 1H), 3.95 (m, 1H), 2.97 (d, J=6.0 Hz, 1H), 2.56 (m, 1H), 2.42 (m, 1H), 2.14 (br s, 1H), 2.04 (quintet, J=7.2 Hz, 2H), 1.81 (ddd, J=14.4, 8.8, 2.8 Hz, 1H), 1.67 (ddd, J=14.4, 8.4, 2.8 Hz, 1H), 1.26 (d, J=6.0 Hz, 3H), 0.98 (t, J=7.6 Hz, 3H); ¹³C NMR (100 MHz, CDCl₃) δ 136.1, 124.3, 71.5, 67.0, 65.5, 40.0, 37.0, 25.7, 23.9, 13.8; HRMS (FAB) calcd for C₁₀H₂₀ClO₂ [M+H]⁺ 207.1152. found 207.1160.

206.

Prepared according to the procedure described above for the synthesis of 193. Reduction of 192 (0.328 g, 1.60 mmol) afforded trans-diol S30 (0.273 g, 85% yield) as a white crystalline solid. 206: R_{f}=0.36 (silica gel, hexanes:EtOAc, 1:1); IR (film) νₘₐₓ 3372 (br), 2966, 2934, 2876, 1458, 1376, 1069 cm⁻¹; ¹H NMR (400 MHz, CDCl₃) δ 5.51 (m, 1H), 5.39 (m, 1H), 4.18 (m, 1H), 4.05-3.89 (m, 2H), 2.74 (br s, 2H), 2.65-2.41 (m, 2H), 2.06 (quintet, J=7.6 Hz, 2H), 1.81 (ddd, J=14.4, 8.8, 2.8 Hz, 1H), 1.66 (ddd, J=14.4, 8.8, 2.4 Hz, 1H), 1.26 (d, J=6.4 Hz, 3H), 0.97 (t, J=7.6 Hz, 3H); ¹³C NMR (100 MHz, CDCl₃) δ 134.9, 124.2, 71.7, 67.0, 65.4, 40.1, 31.5, 23.9, 20.9, 14.2; HRMS (FAB) calcd for C₁₀H₂₀ClO₂ [M+H]⁺207.1152. found 207.1143.

### Production of Both Cis- and Trans-Diols (88)

195.

NaBH₄ (0.195 g, 5.16 mmol, 1.2 equiv) was added in a single portion to a solution of ketone 185 (0.940 g, 4.30 mmol, 1.0 equiv) in MeOH (43 mL) at −20° C. After 15 min at −20° C., the clear solution was quenched by the careful addition of saturated aqueous NH₄Cl (40 mL) and water (40 mL) and extracted with EtOAc (3×60 mL). The combined organic layers were washed with brine (100 mL), dried (MgSO₄), filtered, and concentrated. The resultant crude oil was purified by flash column chromatography (silica gel, hexanes:EtOAc, 1:01:1) to afford cis-diol 195 (0.604 g, 64% yield, contaminated with minor inseparable impurities) as a colorless amorphous solid, along with the separable trans-diol 195 (0.247 g, 26% yield). 195: R_{f}=0.46 (silica gel, hexanes:EtOAc, 1:1); IR (film) νₘₐₓ 3363 (br), 2965, 2933, 2874, 1455, 1136, 1073 cm⁻¹; ¹H NMR (400 MHz, CDCl₃) δ 5.43 (m, 1H), 5.28 (m, 1H), 4.06 (m, 1H), 3.97 (m, 1H), 3.90 (ddd, J=10.4, 4.8, 3.2 Hz, 1H), 3.40 (d, J=4.0 Hz, 1H), 2.87 (d, J=2.4 Hz, 1H), 2.34-2.18 (m, 2H), 2.07 (quintet of doublets, J=7.6, 0.8 Hz, 2H), 1.89-1.58 (m, 4H), 1.24 (d, J=6.4 Hz, 3H), 0.97 (t, J=7.6 Hz, 3H); ¹³C NMR (100 MHz, CDCl₃) δ 133.6, 127.2, 75.8, 68.8, 67.3, 40.5, 32.9, 24.3, 24.1, 20.7, 14.5; HRMS (FAB) calcd for C₁₁H₂₂ClO₂ [M+H]⁺ 221.1308. found 221.1306.

199.

Prepared according to the procedure described above for the synthesis of S19. Reduction of 188 (0.233 g, 1.07 mmol) afforded cis-diol 199 (0.128 g, 54% yield) as a colorless amorphous solid, along with the separable trans-diol 197 (0.062 g, 26% yield). 199: R_{f}=0.42 (silica gel, hexanes:EtOAc, 1:1); IR (film) νₘₐₓ 3367 (br), 2964, 2931, 1449, 1076, 968 cm⁻¹; ¹H NMR (400 MHz, CDCl₃) δ 5.53 (m, 1H), 5.35 (m, 1H), 4.07 (m, 1H), 4.01-3.88 (m, 2H), 2.86 (br s, 2H), 2.29 (m, 1H), 2.10 (sextet, J=7.2 Hz, 1H), 2.00 (quintet, J=7.2 Hz, 2H), 1.90-1.59 (m, 4H), 1.24 (d, J=6.0 Hz, 3H), 0.97 (t, J=7.6 Hz, 3H); ¹³C NMR (100 MHz, CDCl₃) δ 133.9, 127.3, 75.8, 68.9, 67.3, 40.5, 32.9, 29.5, 25.7, 24.3, 14.0; HRMS (FAB) calcd for C₁₁H₂₂ClO₂ [M+H]⁺ 221.1308. found 221.1300.

204.

Prepared according to the procedure described above for the synthesis of 195. Reduction of 191 (1.30 g, 6.35 mmol) afforded mostly pure cis-diol 204 (0.635 g) as a white solid along with the separable trans-diol 202 (0.397 g, 30% yield). Analytically pure 204 was obtained by recrystallization (10 mL boiling hexanes) to afford 0.460 g (35% yield) white needles. 204: R_{f}=0.43 (silica gel, hexanes:EtOAc, 1:1); IR (film) νₘₐₓ 3363 (br), 2966, 2933, 1458, 1429, 1133, 1080, 968 cm⁻¹; ¹H NMR (400 MHz, CDCl₃) δ 5.60 (m, 1H), 5.45 (m, 1H), 4.07 (m, 1H), 3.98 (m, 1H), 3.87 (m, 1H), 3.12 (br s, 2H), 2.58-2.39 (m, 2H), 2.04 (quintet, J=7.2 Hz, 2H), 1.80 (dt, J=14.4, 2.4 Hz, 1H), 1.62 (m, 1H), 1.24 (d, J=6.4 Hz, 3H), 0.98 (t, J=7.6 Hz, 3H); ¹³C NMR (100 MHz, CDCl₃) δ 136.1, 124.2, 75.2, 68.8, 67.0, 40.4, 36.6, 25.7, 24.3, 13.8; HRMS (FAB) calcd for C₁₀H₂₀ClO₂ [M+H]⁺ 207.1152. found 207.1154.

207.

Prepared according to the procedure described above for the synthesis of S19. Reduction of S16 (0.435 g) afforded cis-diol 207 (0.232 g, 53% yield) as a colorless amorphous solid, along with the separable trans-diol 206 (0.153 g, 35% yield). 207: R_{f}=0.46 (silica gel, hexanes:EtOAc, 1:1); IR (film) νₘₐₓ 3362 (br), 2967, 2933, 2875, 1457, 1134, 1074 cm⁻¹; ¹H NMR (400 MHz, CDCl₃) δ 5.55 (m, 1H), 5.41 (m, 1H), 4.09 (m, 1H), 3.99 (ddd, J=10.0, 4.8, 2.0 Hz, 1H), 3.90 (m, 1H), 2.82 (br s, 2H), 2.65-2.45 (m, 2H), 2.06 (quintet, J=7.6 Hz, 2H), 1.81 (dt, J=14.4, 2.4 Hz, 1H), 1.63 (m, 1H), 1.25 (d, J=6.0 Hz, 3H), 0.98 (t, J=7.6 Hz, 3H); ¹³C NMR (100 MHz, CDCl₃) δ 135.0, 124.1, 75.4, 68.9, 67.0, 40.5, 31.1, 24.4, 21.0, 14.2; HRMS (FAB) calcd for C₁₀H₂₀ClO₂ [M+H]⁺ 207.1152. found 207.1157.

### Cyclization/Alcohol Protection to Form Bromoetherification Substrates (89)

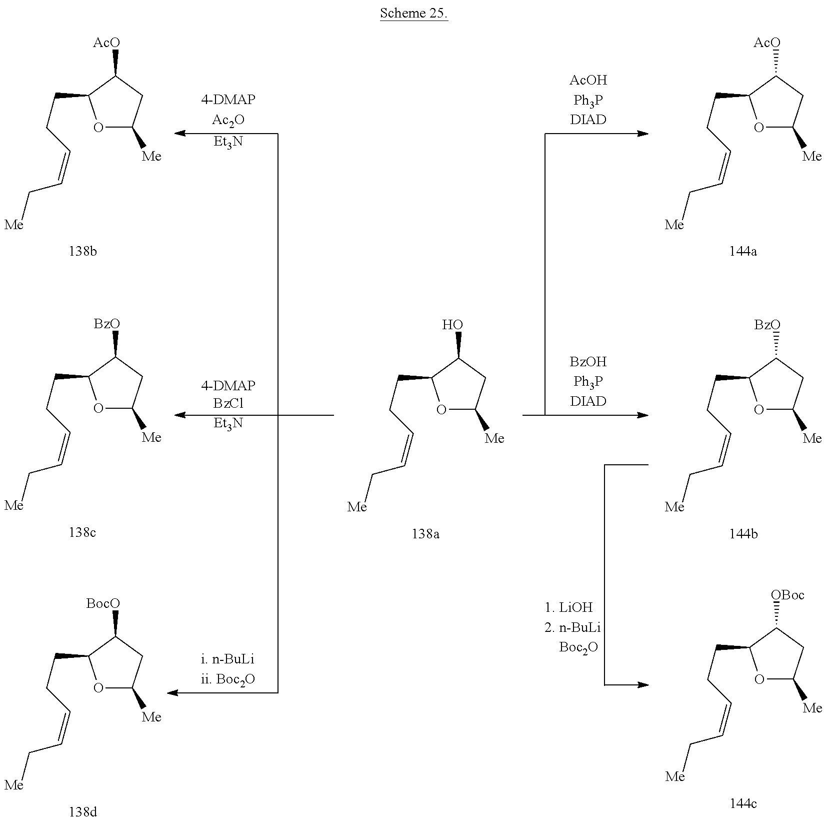

138a.

trans-Diol 193 (0.274 g, 1.24 mmol, 1.0 equiv) was dissolved in MeOH (24 mL) and water (12 mL) in a high-pressure sealed tube. The reaction vessel was then sealed and heated at 130° C. for 4 h. Upon completion, the reaction mixture was allowed to cool to 25° C., quenched by the addition of saturated aqueous NaHCO₃ (30 mL) and water (30 mL), and extracted with EtOAc (3×40 mL). The combined organic layers were washed with brine (50 mL), dried (MgSO₄), filtered, and concentrated. The resultant yellow oil was purified by flash column chromatography (silica gel, hexanes:EtOAc, 1:07:3) to afford hydroxytetrahydrofuran 138a (0.183 g, 80% yield) as a colorless viscous oil. 138a: R_{f}=0.46 (silica gel, hexanes:EtOAc, 3:2); IR (film) νₘₐₓ 3419 (br), 3004, 2965, 2933, 2870, 1453, 1072 cm⁻¹; ¹H NMR (400 MHz, CDCl₃) δ 5.45-5.31 (m, 2H), 4.16 (m, 1H), 3.90 (sextet, J=6.4 Hz, 1H), 3.52 (td, J=6.8, 3.6 Hz, 1H), 2.41 (m, 1H), 2.22-2.10 (m, 2H), 2.05 (quintet, J=7.2 Hz, 2H), 1.81-1.62 (m, 2H), 1.53 (d, J=7.6 Hz, 1H), 1.48 (ddd, J=14.0, 6.8, 2.0 Hz, 1H), 1.33 (d, J=6.4 Hz, 3H), 0.95 (t, J=7.6 Hz, 3H); ¹³C NMR (100 MHz, CDCl₃) δ 132.6, 128.5, 83.0, 73.5, 73.4, 43.7, 28.9, 24.0, 22.3, 20.7, 14.4; HRMS (FAB) calcd for C₁₁H₂₁O₂ [M+H]⁺185.1542. found 185.1549.

Acetylation with Retention (138b).

Ac₂O (0.095 mL, 1.0 mmol, 2.0 equiv) was added dropwise to a solution of hydroxytetrahydrofuran 138a (0.092 g, 0.50 mmol, 1.0 equiv), 4-DMAP (6.1 mg, 0.050 mmol, 0.1 equiv) and Et₃N (0.28 mL, 2.0 mmol, 4.0 equiv) in CH₂Cl₂ (2.5 mL) at 0° C. The resultant colorless solution was stirred for 1 h at 0° C., then quenched by the addition of MeOH (0.1 mL). The crude mixture was purified by filtration through a silica gel plug (eluted with 2:1 hexanes:EtOAc) to afford acetylated product 138b (0.111 g, 98% yield) as a colorless viscous oil [Note: as mentioned above, the Wittig reaction for the synthesis of 182 was not entirely stereoselective, as such 138b was contaminated with approximately 8% of the undesired E-alkene]. 138b: R_{f}=0.47 (silica gel, hexanes:EtOAc, 4:1); IR (film) νₘₐₓ 2967, 2934, 2871, 1739, 1374, 1242, 1080 cm⁻¹; ¹H NMR (400 MHz, CDCl₃) δ 5.41-5.25 (m, 2H), 5.21 (m, 1H), 3.93 (sextet, J=6.8 Hz, 1H), 3.67 (m, 1H), 2.49 (quintet, J=7.2 Hz, 1H), 2.18-1.97 (m, 4H), 2.06 (s, 3H), 1.72 (m, 1H), 1.60 (m, 1H), 1.50 (ddd, J=14.4, 7.2, 2.4 Hz, 1H), 1.31 (d, J=6.4 Hz, 3H), 0.95 (t, J=7.2 Hz, 3H); ¹³C NMR (100 MHz, CDCl₃) δ 170.8, 132.6, 128.3, 81.3, 75.5, 73.7, 41.2, 29.1, 24.1, 21.6, 21.2, 20.6, 14.5; HRMS (FAB) calcd for C₁₃H₂₃O₃ [M+H]⁺ 227.1647. found 227.1645.

Benzoylation with Retention (138c).

Benzoyl chloride (0.070 mL, 0.60 mmol, 2.0 equiv) was added dropwise to a solution of hydroxytetrahydrofuran 138a (0.055 g, 0.30 mmol, 1.0 equiv), 4-DMAP (3.7 mg, 0.030 mmol, 0.1 equiv), and Et₃N (0.17 mL, 1.2 mmol, 4.0 equiv) in CH₂Cl₂ (1.5 mL) at 0° C. The resultant colorless solution was allowed to warm to 25° C. and was stirred at that temperature for 5 h, then quenched by the addition of water (10 mL). The crude product was extracted into CH₂Cl₂ (3×10 mL), and the combined organic layers were washed sequentially with 1 M HCl (20 mL), saturated aqueous NaHCO₃ (20 mL), and brine (20 mL), then dried (MgSO₄), filtered, and concentrated. Purification of the resultant crude residue by flash column chromatography (silica gel, hexanes:EtOAc, 20:1+2% Et₃N) afforded 138c (0.066 g, 76% yield) as a colorless viscous oil [Note: as mentioned above, the Wittig reaction for the synthesis of 182 was not entirely stereoselective, as such 138c was contaminated with approximately 8% of the undesired E-alkene]. 138c: R_{f}=0.52 (silica gel, hexanes:EtOAc, 4:1); IR (film) νₘₐₓ 2967, 2933, 2869, 1719, 1273, 1113, 711 cm⁻¹; ¹H NMR (400 MHz, CDCl₃) δ 8.06 (d, J=7.6 Hz, 2H), 7.57 (t, J=7.6 Hz, 1H), 7.45 (t, J=7.6 Hz, 2H), 5.49 (m, 1H), 5.41-5.25 (m, 2H), 4.03 (sextet, J=6.4 Hz, 1H), 3.82 (m, 1H), 2.61 (quintet, J=7.2 Hz, 1H), 2.22-2.13 (m, 2H), 1.99 (quintet, J=7.2 Hz, 2H), 1.91-1.62 (m, 3H), 1.36 (d, J=6.4 Hz, 3H), 0.89 (t, J=7.6 Hz, 3H); ¹³C NMR (100 MHz, CDCl₃) δ 166.2, 133.2, 132.6, 130.3, 129.8 (2C), 128.6 (2C), 128.2, 81.6, 76.0, 73.8, 41.3, 29.4, 24.1, 21.9, 20.6, 14.4; HRMS (FAB) calcd for C₁₈H₂₅O₃ [M+H]⁺ 289.1804. found 289.1795.

t-Butyl Carbonate with Retention (138d).

A solution of n-BuLi (1.5 M in hexanes, 0.662 mL, 0.993 mmol, 1.0 equiv) was added to a solution of hydroxytetrahydrofuran 138a (0.183 g, 0.993 mmol, 1.0 equiv) in THF (8 mL) at 0° C. After stirring for 5 min at 0° C., a solution of Boc₂O (0.217 g, 0.993 mmol, 1.0 equiv) in THF (2 mL) was added slowly, and the resultant colorless solution was warmed to 25° C. and stirred for 1 h. Upon completion, the reaction mixture was quenched by the addition of saturated aqueous NH₄Cl (10 mL) and water (10 mL). The crude product was extracted into Et₂O (3×15 mL), and the combined organic layers were washed with brine (30 mL), dried (MgSO₄), filtered, and concentrated. Purification of the resultant yellow oil by flash column chromatography (silica gel, hexanes:EtOAc, 19:1) afforded 138d (0.232 g, 82% yield) as a colorless viscous oil [Note: as mentioned above, the Wittig reaction for the synthesis of 182 was not entirely stereoselective, as such 138d was contaminated with approximately 8% of undesired E-alkene]. 138d: R_{f}=0.43 (silica gel, hexanes:EtOAc, 9:1); IR (film) νₘₐₓ 2973, 2935, 2872, 1738, 1280, 1256, 1167 cm⁻¹; ¹H NMR (400 MHz, CDCl₃) δ 5.42-5.28 (m, 2H), 5.07 (m, 1H), 3.90 (sextet, J=6.4 Hz, 1H), 3.67 (m, 1H), 2.48 (app quintet, 1H), 2.20-2.10 (m, 2H), 2.04 (quintet, J=7.2 Hz, 2H), 1.79-1.55 (m, 3H), 1.47 (s, 9H), 1.31 (d, J=6.0 Hz, 3H), 0.94 (t, J=7.2 Hz, 3H); ¹³C NMR (100 MHz, CDCl₃) δ 153.6, 132.5, 128.4, 82.1, 81.4, 78.2, 73.6, 41.0, 29.0, 27.9 (3C), 24.1, 21.4, 20.6, 14.5; HRMS (FAB) calcd for C₁₆H₂₉O₄ [M+H]⁺ 285.2066. found 285.2065.

Acetylation with Inversion (144a).

AcOH (0.052 mL, 0.90 mmol, 3.0 equiv) was added to a solution of hydroxytetrahydrofuran 138a (0.055 g, 0.30 mmol, 1.0 equiv) and Ph₃P (0.12 g, 0.45 mmol, 1.5 equiv) in toluene (3.0 mL) at 0° C. Next, DIAD (0.071 mL, 0.36 mmol, 1.2 equiv) was added dropwise, and the resultant light yellow solution was allowed to warm to 25° C. and stirred at that temperature for 5 h, during which time significant amounts of a white precipitate formed. The reaction mixture was then quenched by the addition of saturated aqueous NaHCO₃ (10 mL), and the crude product was extracted into Et₂O (3×10 mL). The combined organic layers were washed with brine (20 mL), dried (MgSO₄), filtered, and concentrated. Purification of the resultant crude residue by flash column chromatography (silica gel, hexanes:EtOAc, 10:1) afforded 144a (0.054 g, 80% yield) as a colorless viscous oil [Note: as mentioned above, the Wittig reaction for the synthesis of S6 was not entirely stereoselective, as such 144a was contaminated with approximately 8% of the undesired E-alkene]. 144a: R_{f}=0.50 (silica gel, hexanes:EtOAc, 4:1); IR (film) νₘₐₓ 2968, 2932, 2872, 1741, 1241, 1098, 1022 cm⁻¹; ¹H NMR (400 MHz, CDCl₃) δ 5.42-5.28 (m, 2H), 4.93 (ddd, J=6.4, 2.4, 1.2 Hz, 1H), 4.12 (septet, J=5.6 Hz, 1H), 3.80 (ddd, J=8.8, 6.0, 3.2 Hz, 1H), 2.20-1.99 (m, 4H), 2.05 (s, 3H), 1.96 (ddd, J=13.6, 5.6, 1.6 Hz, 1H), 1.76-1.52 (m, 3H), 1.27 (d, J=6.0 Hz, 3H), 0.94 (t, J=7.6 Hz, 3H); ¹³C NMR (100 MHz, CDCl₃) δ 170.8, 132.5, 128.3, 83.9, 79.4, 74.4, 40.4, 34.5, 23.5, 21.3, 20.8, 20.6, 14.4; HRMS (FAB) calcd for C₁₃H₂₃O₃ [M+H]⁺227.1647. found 227.1657.

Benzoylation with Inversion (144b).

DIAD (0.236 mL, 1.20 mmol, 1.2 equiv) was added dropwise to a solution of hydroxytetrahydrofuran 138a (0.184 g, 1.00 mmol, 1.0 equiv), Ph₃P (0.393 g, 1.50 mmol, 1.5 equiv), and benzoic acid (0.366 g, 3.00 mmol, 3.0 equiv) in toluene (10 mL) at 0° C. The resultant light yellow solution was allowed to warm to 25° C. and stirred at that temperature for 2 h, then heated to 50° C. for 1 h. The reaction mixture was then allowed to cool to 25° C. and quenched by the addition of saturated aqueous NaHCO₃ (20 mL). After stirring vigorously for 1 h, the crude product was extracted into Et₂O (3×20 mL). The combined organic layers were washed with brine (30 mL), dried (MgSO₄), filtered, and concentrated. Purification of the resultant crude residue by flash column chromatography (silica gel, hexanes:CH₂Cl₂, 1:0→0:1) afforded 144b (0.234 g, 81% yield) as a colorless viscous oil [Note: as mentioned above, the Wittig reaction for the synthesis of 182 was not entirely stereoselective, as such 144b was contaminated with approximately 8% of the undesired E-alkene]. 144b: R_{f}=0.53 (silica gel, hexanes:EtOAc, 4:1); IR (film) νₘₐₓ 2967, 2931, 2872, 1719, 1273, 1112, 1098, 711 cm⁻¹; ¹H NMR (400 MHz, CDCl₃) δ 8.04 (d, J=7.2 Hz, 2H), 7.57 (t, J=7.6 Hz, 1H), 7.44 (t, J=7.6 Hz, 2H), 5.42-5.30 (m, 2H), 5.20 (m, 1H), 4.24 (septet, J=5.6 Hz, 1H), 3.99 (ddd, J=8.4, 6.0, 2.8 Hz, 1H), 2.26-2.10 (m, 3H), 2.04 (quintet, J=7.2 Hz, 2H), 1.82 (ddd, J=17.2, 10.4, 6.8 Hz, 1H), 1.77-1.61 (m, 2H), 1.32 (d, J=6.0 Hz, 3H), 0.93 (t, J=7.6 Hz, 3H); ¹³C NMR (100 MHz, CDCl₃) δ 166.2, 133.2, 132.5, 130.3, 129.7 (2C), 128.5 (2C), 128.2, 84.0, 79.9, 74.6, 40.5, 34.6, 23.5, 20.7, 20.6, 14.4; HRMS (FAB) calcd for C₁₈H₂₅O₃ [M+H]⁺289.1804. found 289.1807.

t-Butyl Carbonate with Inversion (144c).

A solution of LiOH (0.108 g, 4.5 mmol, 10 equiv) in water (2 mL) was added to a solution of benzoate 144b (0.130 g, 0.45 mmol, 1.0 equiv) in THF (12 mL) and MeOH (4 mL). The resultant transparent solution was stirred for 1 h at 25° C., then quenched by the addition of water (20 mL). The crude product was extracted into Et₂O (3×30 mL); the combined organic layers were washed with brine (50 mL), dried (MgSO₄), filtered, and concentrated. The resultant yellow oil was purified by flash column chromatography (silica gel, hexanes:EtOAc, 1:02:1) afforded the corresponding alcohol (0.081 g, 98% yield) as a colorless viscous oil. Next, a solution of n-BuLi (1.6 M in hexanes, 0.27 mL, 0.44 mmol, 1.0 equiv) was added to a solution of the alcohol produced above (0.081 g, 0.44 mmol, 1.0 equiv) in THF (4 mL) at 0° C. After stirring for 5 min at 0° C., a solution of Boc₂O (0.096 g, 0.44 mmol, 1.0 equiv) in THF (1 mL) was added slowly, and the resultant colorless solution was warmed to 25° C. and stirred for 1 h. Upon completion, the reaction mixture was quenched by the addition of saturated aqueous NH₄Cl (5 mL) and water (5 mL). The crude product was extracted into Et₂O (3×10 mL), and the combined organic layers were washed with brine (20 mL), dried (MgSO₄), filtered, and concentrated. Purification of the resultant yellow oil by flash column chromatography (silica gel, hexanes:EtOAc, 19:1) afforded 144c (0.100 g, 80% yield) as a colorless viscous oil [Note: as mentioned above, the Wittig reaction for the synthesis of 182 was not entirely stereoselective, as such 144c was contaminated with approximately 8% of the undesired E-alkene]. 144c: R_{f}=0.45 (silica gel, hexanes:EtOAc, 9:1); IR (film) νₘₐₓ 2973, 2933, 2873, 1740, 1279, 1255, 1165 cm⁻¹; ¹H NMR (400 MHz, CDCl₃) δ 5.43-5.28 (m, 2H), 4.78 (ddd, J=6.8, 2.8, 1.2 Hz, 1H), 4.14 (septet, J=5.6 Hz, 1H), 3.86 (ddd, J=7.6, 5.6, 2.8 Hz, 1H), 2.22-1.97 (m, 5H), 1.75-1.57 (m, 3H), 1.49 (s, 9H), 1.27 (d, J=6.0 Hz, 3H), 0.95 (t, J=7.2 Hz, 3H); ¹³C NMR (100 MHz, CDCl₃) δ 153.1, 132.3, 128.1, 83.6, 82.4, 81.8, 74.2, 40.3, 34.3, 27.8 (3C), 23.3, 20.5 (2C), 14.3; HRMS (FAB) calcd for C₁₆H₂₉O₄ [M+H]⁺ 285.2066. found 285.2059.

150.

Prepared according to the procedures described above for the synthesis of 138a and 138d. Cyclization of cis-diol 195 (0.590 g, 2.67 mmol) at 130° C. for 1 h afforded the desired hydroxytetrahydrofuran intermediate (0.430 g, 87% yield) as a colorless viscous oil. Carbonate formation on 0.50 mmol scale afforded 150 (0.111 g, 78% yield) as a colorless viscous oil [Note: as mentioned above, the Wittig reaction for the synthesis of S6 was not entirely stereoselective, as such 150 was contaminated with approximately 8% of undesired E-alkene 154]. 150: R_{f}=0.44 (silica gel, hexanes:EtOAc, 9:1); IR (film) νₘₐₓ 2969, 2933, 2872, 1739, 1369, 1280, 1254, 1165 cm⁻¹; ¹H NMR (400 MHz, CDCl₃) δ 5.41-5.27 (m, 2H), 5.15 (t, J=4.0 Hz, 1H), 4.31 (m, 1H), 4.00 (ddd, J=9.6, 5.6, 3.6 Hz, 1H), 2.23-1.96 (m, 5H), 1.78 (ddd, J=14.4, 9.2, 5.2 Hz, 1H), 1.74-1.50 (m, 2H), 1.48 (s, 9H), 1.23 (d, J=6.4 Hz, 3H), 0.94 (t, J=7.6 Hz, 3H); ¹³C NMR (100 MHz, CDCl₃) δ 153.5, 132.5, 128.4, 82.3, 80.2, 78.6, 73.0, 41.1, 29.3, 27.9 (3C), 24.0, 21.5, 20.6, 14.5; HRMS (FAB) calcd for C₁₆H₂₉O₄ [M+H]⁺285.2066. found 285.2055.

152.

Prepared according to the procedures described above for the synthesis of 138a and 138d. Cyclization of trans-diol 197 (0.198 g, 0.898 mmol) at 130° C. for 8 h afforded the desired hydroxytetrahydrofuran intermediate (0.118 g, 72% yield) as a colorless viscous oil. Carbonate formation on 0.50 mmol scale afforded 152 (0.105 g, 74% yield) as a colorless viscous oil. 152: R_{f}=0.39 (silica gel, hexanes:EtOAc, 9:1); IR (film) νₘₐₓ 2977, 2934, 2872, 1739, 1369, 1280, 1255, 1167 cm⁻¹; ¹H NMR (400 MHz, CDCl₃) δ 5.52-5.34 (m, 2H), 5.07 (ddd, J=7.2, 4.0, 2.8 Hz, 1H), 3.89 (m, 1H), 3.66 (ddd, J=10.0, 5.6, 3.6 Hz, 1H), 2.47 (quintet, J=7.2 Hz, 1H), 2.19-1.93 (m, 4H), 1.79-1.63 (m, 2H), 1.58 (ddd, J=14.0, 7.6, 2.8 Hz, 1H), 1.47 (s, 9H), 1.30 (d, J=6.4 Hz, 3H), 0.94 (t, J=7.2 Hz, 3H); ¹³C NMR (100 MHz, CDCl₃) δ 153.6, 132.7, 128.5, 82.1, 81.4, 78.2, 73.5, 40.9, 29.4, 28.8, 27.9 (3C), 25.7, 21.4, 14.0; HRMS (FAB) calcd for C₁₆H₂₉O₄ [M+H]⁺ 285.2066. found 285.2063.

154.

Prepared according to the procedures described above for the synthesis of 138a and 138d. Cyclization of cis-diol 199 (0.128 g, 0.580 mmol) at 130° C. for 2 h afforded the desired hydroxytetrahydrofuran intermediate (0.101 g, 94% yield) as a colorless viscous oil. Carbonate formation on 0.50 mmol scale afforded 154 (0.108 g, 76% yield) as a colorless viscous oil. 154: R_{f}=0.39 (silica gel, hexanes:EtOAc, 9:1); IR (film) νₘₐₓ 2967, 2932, 2873, 1739, 1369, 1279, 1255, 1166 cm⁻¹; ¹H NMR (400 MHz, CDCl₃) δ 5.51-5.33 (m, 2H), 5.15 (t, J=4.0 Hz, 1H), 4.29 (m, 1H), 4.00 (ddd, J=9.6, 6.0, 4.0 Hz, 1H), 2.21-1.94 (m, 5H), 1.77 (ddd, J=14.0, 9.2, 5.2 Hz, 1H), 1.71-1.51 (m, 2H), 1.47 (s, 9H), 1.22 (d, J=6.4 Hz, 3H), 0.94 (t, J=7.6 Hz, 3H); ¹³C NMR (100 MHz, CDCl₃) δ 153.4, 132.7, 128.5, 82.2, 80.2, 78.6, 73.0, 41.1, 29.3, 29.1, 27.9 (3C), 25.7, 21.5, 14.0; HRMS (FAB) calcd for C₁₆H₂₉O₄ [M+H]⁺285.2066. found 285.2073.

156.

Prepared according to the procedures described above for the synthesis of 138a and 138d. Cyclization of trans-diol 202 (0.260 g, 1.26 mmol) at 130° C. for 2 h afforded the desired hydroxytetrahydrofuran intermediate (0.122 g, 57% yield) as a colorless viscous oil. Carbonate formation on 0.50 mmol scale afforded 156 (0.104 g, 77% yield) as a colorless viscous oil. 156: R_{f}=0.44 (silica gel, hexanes:EtOAc, 9:1); IR (film) νₘₐₓ 2974, 2934, 2873, 1739, 1369, 1281, 1256, 1167 cm⁻¹; ¹H NMR (400 MHz, CDCl₃) δ 5.55 (m, 1H), 5.39 (m, 1H), 5.08 (m, 1H), 3.92 (m, 1H), 3.68 (td, J=6.8, 4.0 Hz, 1H), 2.47 (quintet, J=7.2 Hz, 1H), 2.41-2.29 (m, 2H), 2.00 (m, 2H), 1.58 (ddd, J=14.0, 7.6, 2.8 Hz, 1H), 1.47 (s, 9H), 1.31 (d, J=6.4 Hz, 3H), 0.95 (t, J=7.2 Hz, 3H); ¹³C NMR (100 MHz, CDCl₃) δ 153.5, 134.9, 124.7, 82.1, 82.0, 77.9, 73.7, 40.8, 32.4, 27.9 (3C), 25.7, 21.5, 13.8; HRMS (FAB) calcd for C₁₅H₂₇O₄ [M+H]⁺271.1909. found 271.1912.

158.

Prepared according to the procedures described above for the synthesis of 138a and 138d. Cyclization of cis-diol 204 (0.460 g, 2.23 mmol) at 130° C. for 2 h afforded the desired hydroxytetrahydrofuran intermediate (0.316 g, 83% yield) as a colorless viscous oil. Carbonate formation on 0.50 mmol scale afforded 158 (0.100 g, 74% yield) as a colorless viscous oil. 158: R_{f}=0.46 (silica gel, hexanes:EtOAc, 9:1); IR (film) νₘₐₓ 2969, 2931, 2874, 1740, 1369, 1280, 1254, 1166 cm⁻¹; ¹H NMR (400 MHz, CDCl₃) δ 5.55 (m, 1H), 5.37 (m, 1H), 5.15 (t, J=4.0 Hz, 1H), 4.32 (m, 1H), 4.01 (td, J=7.2, 3.6 Hz, 1H), 2.37-2.21 (m, 2H), 2.17 (ddd, J=14.0, 6.0, 1.2 Hz, 1H), 1.99 (m, 2H), 1.76 (ddd, J=14.0, 9.2, 5.2 Hz, 1H), 1.47 (s, 9H), 1.23 (d, J=6.0 Hz, 3H), 0.94 (t, J=7.6 Hz, 3H); ¹³C NMR (100 MHz, CDCl₃) δ 153.3, 134.9, 124.7, 82.2, 80.8, 78.3, 73.4, 41.1, 32.8, 27.9 (3C), 25.7, 21.5, 13.8; HRMS (FAB) calcd for C₁₅H₂₇O₄ [M+H]⁺271.1909. found 271.1901.

160.

Prepared according to the procedures described above for the synthesis of 138a and 138d. Cyclization of trans-diol 206 (0.263 g, 1.27 mmol) at 130° C. for 4 h afforded the desired hydroxytetrahydrofuran intermediate (0.155 g, 72% yield) as a colorless viscous oil. Carbonate formation on 0.30 mmol scale afforded 160 (0.068 g, 84% yield) as a colorless viscous oil. 160: R_{f}=0.34 (silica gel, hexanes:EtOAc, 9:1); IR (film) νₘₐₓ 2975, 2934, 2873, 1739, 1282, 1256, 1166 cm⁻¹; ¹H NMR (400 MHz, CDCl₃) δ 5.48 (m, 1H), 5.36 (m, 1H), 5.08 (m, 1H), 3.93 (m, 1H), 3.70 (td, J=7.2, 4.4 Hz, 1H), 2.48 (quintet, J=7.2 Hz, 1H), 2.41 (t, J=7.2 Hz, 2H), 2.11-2.00 (m, 2H), 1.60 (ddd, J=14.0, 7.2, 2.8 Hz, 1H), 1.47 (s, 9H), 1.31 (d, J=6.0 Hz, 3H), 0.95 (t, J=7.6 Hz, 3H); ¹³C NMR (100 MHz, CDCl₃) δ 153.5, 134.1, 124.4, 82.2, 81.7, 77.9, 73.7, 40.9, 27.9 (3C), 27.2, 21.4, 20.8, 14.3; HRMS (FAB) calcd for C₁₅H₂₇O₄ [M+H]⁺271.1909. found 271.1899.

163.

Prepared according to the procedures described above for the synthesis of 138a and 138d. Cyclization of cis-diol 207 (0.216 g, 1.04 mmol) at 130° C. for 5 h afforded the desired hydroxytetrahydrofuran intermediate (0.153 g, 86% yield) as a colorless viscous oil. Carbonate formation on 0.30 mmol scale afforded 163 (0.067 g, 83% yield) as a colorless viscous oil. 163: R_{f}=0.34 (silica gel, hexanes:EtOAc, 9:1); IR (film) νₘₐₓ 2973, 2932, 2874, 1740, 1281, 1254, 1165 cm⁻¹; ¹H NMR (400 MHz, CDCl₃) δ 5.48 (m, 1H), 5.31 (m, 1H), 5.14 (t, J=4.4 Hz, 1H), 4.32 (m, 1H), 4.03 (td, J=7.2, 4.0 Hz, 1H), 2.42-2.28 (m, 2H), 2.19 (dd, J=13.6, 5.6 Hz, 1H), 2.11-1.98 (m, 2H), 1.77 (ddd, J=14.4, 9.6, 5.2 Hz, 1H), 1.48 (s, 9H), 1.24 (d, J=6.4 Hz, 3H), 0.95 (t, J=7.6 Hz, 3H); ¹³C NMR (100 MHz, CDCl₃) δ 153.4, 134.1, 124.3, 82.3, 80.6, 78.3, 73.3, 41.1, 27.9 (3C), 27.5, 21.5, 20.8, 14.3; HRMS (FAB) calcd for C₁₅H₂₇O₄ [M+H]⁺ 271.1909. found 271.1900.

### III. BDSB Cyclizations

### General Cyclization Procedure A.

A cold (−25° C.) solution of BDSB (0.0659 g, 0.120 mmol, 1.2 equiv) in MeNO₂ (0.5 mL) was added rapidly via syringe to a solution of the cyclization precursor (0.100 mmol, 1.0 equiv) in MeNO₂ (4.5 mL) at −25° C. After stirring the resultant yellow solution for 5 min at −25° C., the flask was removed from the cold bath and stirred for an additional 5 min. Upon completion, the reaction mixture was quenched by the addition of a combination of saturated aqueous NaHCO₃ and 5% aqueous Na₂SO₃ (1:1, 5 mL), and the resultant biphasic mixture was stirred vigorously for 20 min at 25° C. The reaction contents were added to brine (10 mL) and extracted with EtOAc (3×15 mL). The combined organic layers were washed with brine (20 mL), dried (MgSO₄), filtered, and concentrated. The resultant residue was purified by flash column chromatography (silica gel, hexanes:EtOAc) to afford the desired products as detailed below.

### General Cyclization Procedure B.

Identical to cyclization Procedure A, except that 1.5 equivalents of BDSB was utilized and the reaction was stirred for 15 min at −25° C. before being removed from the cold bath and stirred for an additional 5 min prior to quench. This procedure proved ideal for the more sluggish reactions forming the 8-endo products.

### General Cyclization Procedure C.

Identical to cyclization Procedure A, except that the reaction was quenched immediately after 5 min at −25° C. without allowing any warming of the reaction mixture. This procedure was utilized for all non-carbonate substrates.

139/140.

Cyclization of 138b utilizing General Cyclization Procedure C [Note: 0.0108 mmol of starting material and 0.130 mmol BDSB were utilized, since 8% was the undesired E-alkene isomer], followed by a quick purification by flash column chromatography (silica gel, hexanes:EtOAc, 1:0→1:1), afforded a 3.6:1 mixture of 139 and 140 (0.0238 g, 74% yield) as a colorless amorphous solid. These two acetate regioisomers were only mostly separable on silica gel, a separation hindered by their facile interconversion, presumably by intramolecular transfer of the acetate group. [Note: while this transfer seemed to occur quickly on silica, it also occurred in solution or neat (even at −20° C.), albeit more slowly]. The minor isomer (140) appeared to be the more thermodynamically stable of the two, since it tended to predominate in mixtures that were allowed to interconvert over a long period of time. Analysis of COSY spectra clearly indicated the position of the acetate on each of the two isomers. Major Isomer 139: R_{f}=0.34 (silica gel, hexanes:EtOAc, 1:1); IR (film) νₘₐₓ 3450 (br), 2966, 2937, 2876, 1731, 1371, 1247, 1057, 1037 cm⁻¹; ¹H NMR (400 MHz, CDCl₃) δ 5.01 (ddd, J=11.2, 4.4, 2.0 Hz, 1H), 4.20 (sextet of doublets, J=6.4, 2.0 Hz, 1H), 4.11 (m, 1H), 3.88-3.79 (m, 2H), 3.15 (d, J=10.0 Hz, 1H), 2.07 (s, 3H), 2.05-1.75 (m, 7H), 1.72 (ddd, J=14.4, 4.4, 2.0 Hz, 1H), 1.26 (d, J=6.0 Hz, 3H), 1.06 (t, J=7.2 Hz, 3H); ¹³C NMR (100 MHz, CDCl₃) δ 171.0, 76.4, 75.4, 72.2, 67.6, 62.1, 36.3, 31.7, 27.9, 26.3, 22.1, 21.5, 12.8; HRMS (FAB) calcd for C₁₃H₂₄BrO₄ [M+H]⁺ 323.0858. found 323.0846. Minor Isomer 140: R_{f}=0.32 (silica gel, hexanes:EtOAc, 1:1); IR (film) νₘₐₓ 3452 (br), 2966, 2938, 2876, 1729, 1371, 1248, 1058, 1036 cm⁻¹; ¹H NMR (400 MHz, CDCl₃) δ 5.22 (dd, J=8.0, 3.2 Hz, 1H), 4.05-3.95 (m, 2H), 3.83-3.72 (m, 2H), 2.28 (d, J=2.4 Hz, 1H), 2.08 (s, 3H), 2.13-1.59 (m, 7H), 1.52 (dt, J=14.8, 4.0 Hz, 1H), 1.18 (d, J=6.4 Hz, 3H), 1.05 (t, J=7.2 Hz, 3H); ¹³C NMR (100 MHz, CDCl₃) δ 170.1, 77.2, 74.1, 70.5, 69.3, 62.0, 37.0, 26.5, 26.4, 25.9, 21.5, 21.1, 13.3.

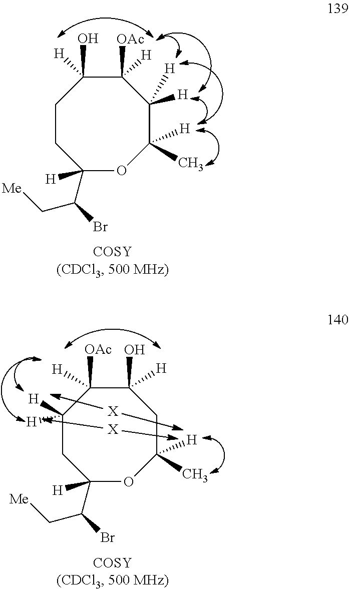

141/142.

Cyclization of 138c utilizing General Cyclization Procedure C [Note: 0.0108 mmol of starting material and 0.130 mmol BDSB were utilized, since 8% was the undesired E-alkene isomer] afforded 141 (0.0266 g, 69% yield) and 142 (2.8 mg, 7% yield) as colorless amorphous solids that were separable by preparative thin-layer chromatography (silica gel, hexanes:EtOAc, 3:2, run up two times). Unlike the acetate regioisomers described above, these two benzoate regioisomers did not appear to interconvert on silica, in solution, or neat. Analysis of the COSY spectrum clearly indicated the position of the benzoate on major isomer 141. Major Isomer 141: R_{f}=0.52 (silica gel, hexanes:EtOAc, 3:2); IR (film) νₘₐₓ 3441 (br), 2966, 2934, 1712, 1276, 1114, 1069, 713 cm⁻¹; ¹H NMR (400 MHz, CDCl₃) δ 8.04 (m, 2H), 7.57 (tt, J=7.2, 1.2 Hz, 1H), 7.44 (t, J=7.6 Hz, 2H), 5.28 (ddd, J=11.2, 4.4, 2.0 Hz, 1H), 4.30-4.20 (m, 2H), 3.90-3.81 (m, 2H), 3.21 (d, J=10.0 Hz, 1H), 2.17 (m, 1H), 2.08-1.74 (m, 7H), 1.29 (d, J=6.4 Hz, 3H), 1.08 (t, J=7.2 Hz, 3H); ¹³C NMR (100 MHz, CDCl₃) δ 166.4, 133.3, 130.3, 129.8 (2C), 128.5 (2C), 77.4, 75.9, 72.3, 67.7, 62.1, 36.4, 31.8, 27.8, 26.4, 22.1, 12.8; HRMS (FAB) calcd for C₁₈H₂₆BrO₄ [M+H]⁺385.1014. found 385.1028. Minor Isomer 142: R_{f}=0.48 (silica gel, hexanes:EtOAc, 3:2); IR (film) νₘₐₓ 3481 (br), 2965, 2876, 1711, 1273, 1116, 1058, 713 cm⁻¹; ¹H NMR (400 MHz, CDCl₃) δ 8.03 (m, 2H), 7.58 (tt, J=7.6, 1.2 Hz, 1H), 7.46 (t, J=7.6 Hz, 2H), 5.51 (dd, J=8.0, 3.2 Hz, 1H), 4.19 (m, 1H), 4.06 (sextet of doublets, J=6.4, 2.4 Hz, 1H), 3.85 (m, 1H), 3.78 (dt, J=11.2, 2.8 Hz, 1H), 2.37 (d, J=2.4 Hz, 1H), 2.22 (m, 1H), 2.12-1.79 (m, 5H), 1.75-1.60 (m, 2H), 1.22 (d, J=6.8 Hz, 3H), 1.07 (t, J=7.2 Hz, 3H); ¹³C NMR (100 MHz, CDCl₃) δ 165.8, 133.3, 130.5, 129.7 (2C), 128.6 (2C), 77.9, 74.1, 70.7, 69.4, 62.0, 37.2, 26.5 (2C), 25.9, 21.1, 13.3; HRMS (FAB) calcd for C₁₈H₂₆BrO₄ [M+H]⁺ 385.1014. found 385.1009.

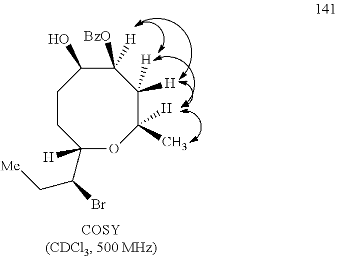

143.

Cyclization of 138d utilizing General Cyclization Procedure A [Note: 0.0108 mmol of starting material and 0.130 mmol BDSB were utilized, since 8% was the undesired E-alkene isomer], followed by purification by flash column chromatography (silica gel, hexanes:EtOAc, 4:1), afforded 143 (0.0244 g, 79% yield) as a white crystalline solid. 143: R_{f}=0.46 (silica gel, hexanes:EtOAc, 3:2); IR (film) νₘₐₓ 2969, 2937, 2877, 1800, 1192, 1041 cm⁻¹; ¹H NMR (400 MHz, CDCl₃) δ 4.85-4.72 (m, 2H), 4.00 (sextet of doublets, J=6.4, 2.8 Hz, 1H), 3.75-3.68 (app d, 2H), 2.22-2.12 (m, 2H), 2.04-1.89 (m, 4H), 1.66-1.44 (m, 2H), 1.26 (d, J=6.4 Hz, 3H), 1.05 (t, J=7.2 Hz, 3H); ¹³C NMR (100 MHz, CDCl₃) δ 154.2, 82.2, 79.0, 74.3, 70.5, 60.7, 33.9, 27.8, 26.2, 25.4, 19.1, 13.3; HRMS (FAB) calcd for C₁₂H₂₀BrO₄ [M+H]⁺307.0545. found 307.0536.

145/146.

Cyclization of 144a utilizing General Cyclization Procedure C [Note: 0.0108 mmol of starting material and 0.130 mmol BDSB were utilized, since 8% was the undesired E-alkene isomer], followed by quick purification by flash column chromatography (silica gel, hexanes:EtOAc, 1:0→1:1), afforded a 1:1.7 mixture of 145 and 146 (0.0250 g, 77% yield), inseparable by chromatography, as a colorless amorphous solid. As with diastereomers 139 and 140 described above, acetate transfer between these two regioisomers was observed on silica gel as well as in solution and with neat compounds. Analysis of a COSY spectrum of the mixture clearly indicated the position of the acetate on each of the two isomers. 145 and 146: R_{f}=0.36 (silica gel, hexanes:EtOAc, 1:1); IR (film) νₘₐₓ 3432 (br), 2966, 2934, 1725, 1373, 1251, 1124, 1070, 1031 cm⁻¹; HRMS (FAB) calcd for C₁₃H₂₂BrO₄ [M−H]⁺ 321.0701. found 321.0712. Minor Isomer (145): ¹H NMR (400 MHz, CDCl₃, identifiable peaks only) δ 4.80 (m, 1H), 4.26 (t, J=7.2 Hz, 1H), 4.15 (sextet of doublets, J=6.4, 2.4 Hz, 1H), 3.78-3.70 (m, 2H), 2.40 (br s, 1H), 2.09 (s, 3H), 1.13 (d, J=6.8 Hz, 3H), 1.04 (t, J=7.2 Hz, 3H); ¹³C NMR (100 MHz, CDCl₃) δ 171.5, 78.9, 73.9, 73.7, 67.6, 61.9, 34.4, 32.1, 27.6, 25.9, 21.4, 20.3, 13.3. Major Isomer (146): ¹H NMR (400 MHz, CDCl₃, identifiable peaks only) δ 5.16 (t, J=8.0 Hz, 1H), 4.32 (sextet of doublets, J=6.4, 2.0 Hz, 1H), 3.82 (m, 1H), 3.78-3.70 (m, 2H), 2.59 (br s, 1H), 2.07 (s, 3H), 1.14 (d, J=6.8 Hz, 3H), 1.04 (t, J=7.2 Hz, 3H); ¹³C NMR (100 MHz, CDCl₃) δ 171.8, 81.3, 73.4, 72.7, 67.3, 61.9, 36.0, 30.5, 27.0, 25.5, 21.6, 20.1, 13.3.

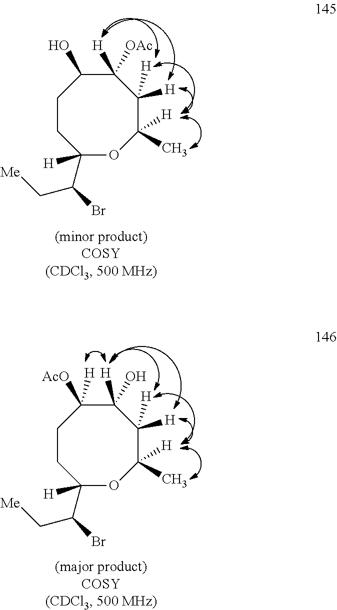

147/148.

Cyclization of 144b utilizing General Cyclization Procedure C [Note: 0.0108 mmol of starting material and 0.130 mmol BDSB were utilized, since 8% was the undesired E-alkene isomer] followed by purification by flash column chromatography (silica gel, hexanes:EtOAc, 1:0→2:1) afforded an inseparable mixture (1:1.2) of 147 and 148 (0.0280 g, 73%) as a colorless amorphous solid. As with 141 and 142, these two benzoate regioisomers did not appear to interconvert on silica, in solution, or neat. Analysis of a COSY spectrum of the mixture clearly indicates the position of the benzoate on each of the two isomers. 147/148: R_{f}=0.48 (silica gel, hexanes:EtOAc, 3:2); IR (film) νₘₐₓ 3461 (br), 2966, 2933, 2875, 1712, 1276, 1113, 1068, 713 cm⁻¹; HRMS (FAB) calcd for C₁₈H₂₅BrO₄ [M−H]⁺ 383.0858. found 383.0865. Minor Isomer (147): ¹H NMR (400 MHz, CDCl₃, identifiable peaks only) δ 5.10 (ddd, J=8.0, 4.0, 2.4 Hz, 1H), 4.47 (m, 1H), 4.29 (sextet of d, J=6.8, 2.4 Hz, 1H), 2.37 (d, J=4.0 Hz, 1H), 1.16 (d, J=6.8 Hz, 3H); ¹³C NMR (100 MHz, CDCl₃, identifiable peaks only) δ 166.7, 79.4, 73.9, 67.9, 20.5. Major Isomer (148): ¹H NMR (400 MHz, CDCl₃, identifiable peaks only) δ 5.46 (t, J=8.4 Hz, 1H), 4.41 (sextet of d, J=6.4, 2.0 Hz, 1H), 4.04 (m, 1H), 2.61 (s, 1H), 1.19 (d, J=6.4 Hz, 3H); ¹³C NMR (100 MHz, CDCl₃, identifiable peaks only) δ 167.3, 82.1, 72.9, 67.4, 20.2.

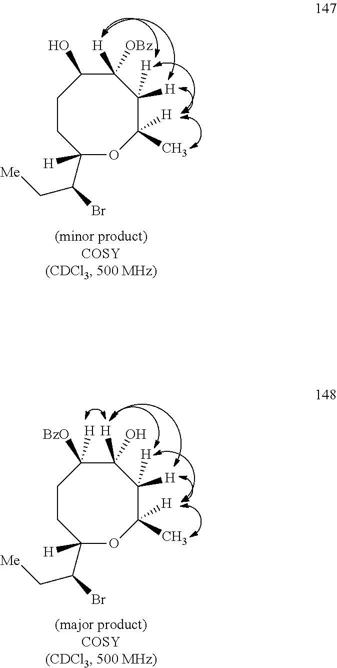

149.

Cyclization of 144c utilizing General Cyclization Procedure A [Note: 0.0108 mmol of starting material and 0.130 mmol BDSB were utilized, since 8% was the undesired E-alkene isomer], followed by purification by flash column chromatography (silica gel, hexanes:EtOAc, 4:1), afforded 149 (0.0283 g) as a colorless amorphous solid (contaminated with a small impurity, likely the product derived from the E-alkene contaminant in the starting material, ˜85% yield). 149: R_{f}=0.52 (silica gel, hexanes:EtOAc, 3:2); IR (film) νₘₐₓ 2970, 2933, 1805, 1210, 1076, 1055 cm⁻¹; ¹H NMR (400 MHz, CDCl₃) δ 4.57 (td, J=10.4, 5.6 Hz, 1H), 4.36 (m, 1H), 4.09 (septet, J=5.6 Hz, 1H), 3.86-3.78 (m, 2H), 2.45 (ddt, J=14.0, 7.6, 2.0 Hz, 1H), 2.28 (ddd, J=15.6, 10.0, 5.2 Hz, 1H), 2.12 (m, 1H), 1.99-1.72 (m, 5H), 1.37 (d, J=6.4 Hz, 3H), 1.06 (t, J=7.2 Hz, 3H); ¹³C NMR (100 MHz, CDCl₃) δ 154.0, 83.4, 81.6, 79.6, 64.3, 63.0, 39.6, 30.8, 28.6, 27.2, 22.6, 12.4; HRMS (FAB) calcd for C₁₂H₂₀BrO₄ [M+H]⁺ 307.0545. found 307.0530.

151.

Cyclization of 150 utilizing General Cyclization Procedure A [Note: 0.0108 mmol of starting material and 0.130 mmol BDSB were utilized, since 8% was the undesired E-alkene isomer] afforded 151 (0.0257 g, 84% yield) as a white crystalline solid. 151: R_{f}=0.46 (silica gel, hexanes:EtOAc, 3:2); IR (film) νₘₐₓ 2972, 2940, 2879, 1802, 1188, 1048, 1029 cm⁻¹; ¹H NMR (400 MHz, CDCl₃) δ 5.00 (ddd, J=12.4, 7.2, 3.2 Hz, 1H), 4.81 (ddd, J=10.8, 7.2, 1.2 Hz, 1H), 3.91 (quintet, J=6.4 Hz, 1H), 3.83 (m, 1H), 3.40 (ddd, J=11.6, 5.2, 2.0 Hz, 1H), 2.44 (ddd, J=18.0, 12.4, 5.6 Hz, 1H), 2.13-1.84 (m, 5H), 1.68-1.54 (m, 2H), 1.31 (d, J=6.4 Hz, 3H), 1.07 (t, J=7.2 Hz, 3H); ¹³C NMR (100 MHz, CDCl₃) δ 154.3, 82.3, 81.7, 75.7, 72.7, 62.1, 32.6, 29.7, 27.6, 27.4, 19.9, 12.5; HRMS (FAB) calcd for C₁₂H₂₀BrO₄ [M+H]⁺ 307.0545. found 307.0545.

153.

Cyclization of 152 utilizing General Cyclization Procedure A afforded 153 (0.0183 g, 60% yield) as a white crystalline solid. 153: R_{f}=0.44 (silica gel, hexanes:EtOAc, 3:2); IR (film) νₘₐₓ 2969, 2937, 2877, 1797, 1192, 1040 cm⁻¹; ¹H NMR (400 MHz, CDCl₃) δ 4.89-4.80 (m, 2H), 4.11 (sextet of doublets, J=6.8, 3.2 Hz, 1H), 3.84 (ddd, J=10.0, 4.4, 3.6 Hz, 1H), 3.43 (dt, J=10.4, 2.8 Hz, 1H), 2.22-1.94 (m, 4H), 1.89-1.68 (m, 4H), 1.27 (d, J=6.8 Hz, 3H), 1.08 (t, J=7.2 Hz, 3H); ¹³C NMR (100 MHz, CDCl₃) δ 154.2, 82.0, 78.9, 72.9, 70.5, 64.8, 34.3, 29.4, 28.3, 27.4, 19.0, 13.1; HRMS (FAB) calcd for C₁₂H₂₀BrO_{4 [M+H]}⁺ 307.0545. found 307.0545.

155.

Cyclization of 154 utilizing General Cyclization Procedure A afforded 155 (0.0254 g, 83% yield) as a colorless amorphous solid. 155: R_{f}=0.53 (silica gel, hexanes:EtOAc, 3:2); IR (film) νₘₐₓ 2973, 2938, 2878, 1804, 1188, 1050, 1034 cm⁻¹; ¹H NMR (400 MHz, CDCl₃) δ 4.98 (ddd, J=12.4, 7.6, 3.2 Hz, 1H), 4.79 (ddd, J=10.8, 7.2, 1.2 Hz, 1H), 3.91 (quintet, J=6.4 Hz, 1H), 3.72 (ddd, J=10.4, 6.4, 2.8 Hz, 1H), 3.45 (ddd, J=11.6, 6.4, 2.0 Hz, 1H), 2.45 (ddd, J=18.4, 12.4, 5.6 Hz, 1H), 2.22-1.90 (m, 5H), 1.68 (m, 1H), 1.51 (m, 1H), 1.29 (d, J=6.8 Hz, 3H), 1.07 (t, J=7.2 Hz, 3H); ¹³C NMR (100 MHz, CDCl₃) δ 154.3, 82.5, 82.3, 75.6, 73.0, 61.8, 32.6, 30.9, 27.5, 26.8, 19.8, 12.8; HRMS (FAB) calcd for C₁₂H₂₀BrO₄ [M+H]⁺ 307.0545. found 307.0556.

157.

Cyclization of 156 utilizing General Cyclization Procedure B afforded 157 (0.0198 g, 68% yield) as a colorless amorphous solid. 157: R_{f}=0.49 (silica gel, hexanes:EtOAc, 3:2); IR (film) νₘₐₓ 2971, 2939, 2881, 1805, 1192, 1041 cm⁻¹; ¹H NMR (400 MHz, CDCl₃) δ 4.86-4.77 (m, 2H), 4.02 (sextet of doublets, J=6.4, 2.8 Hz, 1H), 3.81 (m, 1H), 3.62 (td, J=10.8, 4.0 Hz, 1H), 2.60-2.47 (m, 2H), 2.19 (m, 1H), 2.06 (dt, J=14.4, 3.2 Hz, 1H), 1.91 (sextet of doublets, J=7.2, 4.0 Hz, 1H), 1.70 (sextet of doublets, J=7.2, 3.6 Hz, 1H), 1.27 (d, J=6.8 Hz, 3H), 0.89 (t, J=7.2 Hz, 3H); ¹³C NMR (100 MHz, CDCl₃) δ 153.8, 79.7, 78.8, 74.1, 70.6, 48.1, 39.3, 34.1, 27.6, 18.7, 6.9; HRMS (FAB) calcd for C₁₁H₁₈BrO₄ [M+H]⁺293.0388. found 293.0395.

159.

Cyclization of 158 utilizing General Cyclization Procedure B afforded 159 (0.0195 g, 67% yield) as a white crystalline solid. 159: R_{f}=0.54 (silica gel, hexanes:EtOAc, 3:2); IR (film) νₘₐₓ 2973, 2939, 2881, 1807, 1188, 1052, 1034 cm⁻¹; ¹H NMR (400 MHz, CDCl₃) δ 4.98 (ddd, J=12.4, 7.2, 3.6 Hz, 1H), 4.83 (ddd, J=10.8, 7.2, 1.2 Hz, 1H), 3.79 (quintet, J=6.4 Hz, 1H), 3.72 (m, 1H), 3.26 (m, 1H), 2.59 (m, 1H), 2.52-2.42 (m, 2H), 2.12 (sextet of doublets, J=7.2, 2.4 Hz, 1H), 2.02 (dd, J=14.4, 3.6 Hz, 1H), 1.44 (m, 1H), 1.29 (d, J=6.4 Hz, 3H), 0.94 (t, J=7.6 Hz, 3H); ¹³C NMR (100 MHz, CDCl₃) δ 153.9, 84.4, 80.0, 75.6, 73.4, 49.3, 39.1, 32.5, 28.0, 20.1, 9.3; HRMS (FAB) calcd for C₁₁H₁₈BrO₄ [M+H]⁺293.0388. found 293.0395.

162.

Attempted cyclization of 160 utilizing General Cyclization Procedure B afforded 44 (0.0116 g, 47% yield) as a colorless amorphous solid (as the predominant product of a complex mixture of products). Connectivity and stereochemistry were determined by COSY and NOESY NMR experiments (see attached spectra). As additional evidence for the structure of 162, cyclization of the alcohol precursor to 160 using BDSB was undertaken using Procedure C, and 162 was produced in >90% yield. 162: R_{f}=0.40 (silica gel, hexanes:EtOAc, 4:1); IR (film) νₘₐₓ 2970, 2934, 2877, 1384, 1117, 1083 cm⁻¹; ¹H NMR (400 MHz, CDCl₃) δ 4.79 (quintet, J=4.0 Hz, 1H), 4.46 (t, J=4.8 Hz, 1H), 4.22 (quintet, J=5.2 Hz, 1H), 3.92-3.84 (m, 2H), 2.35 (m, 1H), 2.17 (dd, J=13.2, 5.2 Hz, 1H), 1.98-1.73 (m, 3H), 1.55 (ddd, J=13.2, 9.6, 4.0 Hz, 1H), 1.30 (d, J=6.0 Hz, 3H), 1.09 (t, J=7.2 Hz, 3H); ¹³C NMR (75 MHz, CDCl₃) δ 85.0, 83.8, 80.4, 76.0, 60.8, 42.0, 37.5, 28.9, 20.6, 12.6; HRMS (EI) calcd for C₁₀H₁₆BrO₂ [M−H]⁺ 247.0334. found 247.0328.

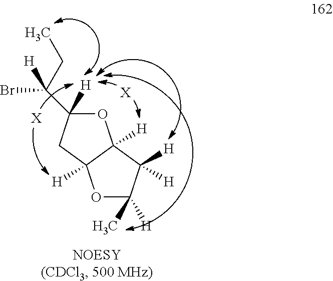

165.

Attempted cyclization of 163 utilizing General Cyclization Procedure B above afforded 47 (8.9 mg, 36% yield) as a colorless amorphous solid (as the predominant product of a complex mixture of products). Connectivity and stereochemistry were determined by COSY and NOESY NMR experiments (see attached spectra). As additional evidence for the structure of 165, cyclization of the alcohol precursor to 163 using BDSB was undertaken using Procedure C, and 165 was produced in >90% yield. 165: R_{f}=0.40 (silica gel, hexanes:EtOAc, 4:1); IR (film) νₘₐₓ 2969, 2933, 2875, 1381, 1123, 1089, 1047 cm⁻¹; ¹H NMR (400 MHz, CDCl₃) δ 4.80-4.72 (m, 2H), 4.26-4.13 (m, 2H), 3.86 (quintet, J=4.4 Hz, 1H), 2.27-2.15 (m, 2H), 1.96-1.78 (m, 3H), 1.54 (ddd, J=14.8, 10.0, 4.4 Hz, 1H), 1.24 (d, J=6.0 Hz, 3H), 1.08 (t, J=7.2 Hz, 3H); ¹³C NMR (100 MHz, CDCl₃) δ 85.4, 83.6, 82.6, 75.9, 61.7, 43.0, 39.3, 28.7, 20.8, 12.6; HRMS (EI) calcd for C₁₀H₁₆BrO₂ [M−H]⁺ 247.0334. found 247.0327.

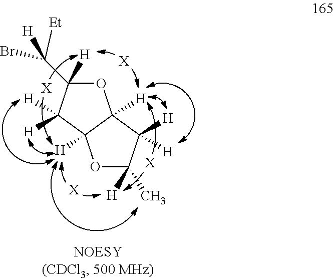

### IV. Cyclization of 20d Using Alternate Bromonium Sources

Bis(collidine)bromonium Triflate. A solution of (coll)₂BrOTf (0.0566 g, 0.120 mmol, 1.2 equiv) in MeNO₂ (0.5 mL) was syringed into a solution of 138d (0.0284 g, 0.100 mmol, 1.0 equiv) in MeNO₂ (4.5 mL) at 0° C. After 5 min at 0° C., the reaction mixture was removed from the ice bath and stirred at 25° C. for an additional 5 min. Upon completion, the reaction mixture was quenched by the addition of 5% aqueous Na₂SO₃ (5 mL). The reaction contents were added to water (5 mL) and then extracted with CH₂Cl₂ (3×10 mL). The combined organic layers were dried (MgSO₄), filtered, and concentrated. The resultant residue was purified by flash column chromatography (silica gel, hexanes:EtOAc, 1:07:3) to afford 0.0199 g of 143 contaminated by two minor, inseparable, impurities (calculated yield of pure 143 is 0.0159 g, 52% yield).

### TBCO.

TBCO (0.0492 g, 0.120 mmol, 1.2 equiv) was added in one portion to a solution of 138d (0.0284 g, 0.100 mmol, 1.0 equiv) in MeCN (5 mL) at 25° C. After 10 min at 25° C., the reaction mixture was quenched by the addition of a mixture of saturated aqueous NaHCO₃ and 5% aqueous Na₂SO₃ (1:1, 5 mL).

The reaction contents were added to water (5 mL) and then extracted with CH₂Cl₂ (3×10 mL). The combined organic layers were dried (MgSO₄), filtered, and concentrated. The resultant residue was purified by flash column chromatography (silica gel, hexanes:EtOAc, 1:0→7:3) to afford 0.0190 g (62% yield) of pure 143.

### NBS.

NBS (0.0214 g, 0.120 mmol, 1.2 equiv) was added in one portion to a solution of 138d (0.0284 g, 0.100 mmol, 1.0 equiv) in CH₂Cl₂ (0.5 mL) at 25° C. After 48 h, the solvent was removed under vacuum and the residue subjected to flash column chromatography (silica gel, hexanes:EtOAc, 1:0→1:1) to afford 3.6 mg of 143 that was approximately 75% pure by NMR (2.8 mg pure 143, 9% yield). The remaining mass balance contained approximately 50% starting material in addition to numerous unidentified byproducts. Performing the reaction at lower temperatures or higher dilution led to only recovered starting material. Utilizing excess NBS or 3 equivalents of N,N-dimethylacetamide as a nucleophilic promoter resulted in faster reactions, but with increased side product formation such that even less desired product was formed. More polar solvents such as THF and DMF resulted in an increased rate of consumption of starting material, but with no product formation at all.

### V. Post-cyclization Modification of 8-Membered Rings

### General Procedure for Acetate/Carbonate Hydrolysis.

K₂CO₃ (0.069 g, 0.50 mmol, 10 equiv) was added in a single portion to a solution of the cyclic carbonate or acetate (0.050 mmol, 1.0 equiv) in MeOH (1.8 mL) and water (0.2 mL) at 0° C. The reaction mixture was allowed to warm slowly to 25° C. and was monitored by TLC. Upon completion (˜0.5 to 2 h), the reaction mixture was quenched by the addition of saturated aqueous NH₄Cl (5 mL) and water (5 mL), and the crude product was extracted into EtOAc (3×10 mL). The combined organic layers were washed with brine (10 mL), dried (MgSO₄), filtered, and concentrated. Purification by flash column chromatography (silica gel, hexanes:EtOAc) afforded the desired diols as white crystalline solids (in all but one case—200). Recrystallization was performed by slow evaporation from a hexanes:CH₂Cl₂ mixture to afford single crystals suitable for X-ray diffraction.

194:

White crystalline solid. R_{f}=0.28 (silica gel, hexanes:EtOAc, 1:2); IR (film) νₘₐₓ 3380 (br), 2965, 2934, 2875, 1459, 1376, 1052 cm⁻¹; ¹H NMR (400 MHz, CDCl₃) δ 4.18 (m, 1H), 4.07-3.90 (m, 2H), 3.69-3.62 (app d, 2H), 2.53 (d, J=2.8 Hz, 1H), 2.21 (d, J=6.0 Hz, 1H), 2.10-1.82 (m, 4H), 1.79-1.57 (m, 4H), 1.19 (d, J=6.8 Hz, 3H), 1.05 (t, J=7.2 Hz, 3H); ¹³C NMR (100 MHz, CDCl₃) δ 74.3, 73.5, 72.1, 69.4, 62.3, 37.9, 29.6, 27.3, 26.5, 21.3, 13.2; HRMS (FAB) calcd for C₁₁H₂₂BrO₃ [M+H]⁺ 281.0752. found 281.0746; structure confirmed by single crystal X-Ray diffraction.

196:

White crystalline solid; R_{f}=0.29 (silica gel, hexanes:EtOAc, 1:2); IR (film) νₘₐₓ 3394 (br), 2969, 2936, 2876, 1462, 1113, 1062, 1039, 1009 cm⁻¹; ¹H NMR (400 MHz, CDCl₃) δ 4.23 (m, 1H), 4.14 (m, 1H), 3.91-3.82 (m, 2H), 3.50 (ddd, J=10.8, 5.2, 2.4 Hz, 1H), 2.40 (d, J=2.4 Hz, 1H), 2.18-1.99 (m, 3H), 1.92 (sextet of doublets, J=7.2, 3.6 Hz, 1H), 1.85-1.55 (m, 5H), 1.23 (d, J=6.8 Hz, 3H), 1.06 (t, J=7.6 Hz, 3H); ¹³C NMR (100 MHz, CDCl₃) δ 80.3, 73.4, 72.6, 69.4, 63.4, 37.1, 30.4, 29.3, 27.9, 20.9, 12.5; HRMS (FAB) calcd for C₁₁H₂₀BrO₂ [M-OH]⁺ 263.0647. found 263.0658; structure confirmed by single crystal X-Ray diffraction.

198:

White crystalline solid. R_{f}=0.22 (silica gel, hexanes:EtOAc, 1:2); IR (film) νₘₐₓ 3391 (br), 2965, 2935, 1459, 1376, 1144, 1059 cm⁻¹; ¹H NMR (400 MHz, CDCl₃) δ 4.21 (app d, 1H), 4.07 (sextet of doublets, J=6.4, 2.4 Hz, 1H), 3.93 (m, 1H), 3.85 (m, 1H), 3.57 (m, 1H), 2.31 (br s, 2H), 2.10-1.68 (m, 7H), 1.63 (dt, J=15.2, 4.0 Hz, 1H), 1.19 (d, J=6.4 Hz, 3H), 1.06 (t, J=7.2 Hz, 3H); ¹³C NMR (100 MHz, CDCl₃) δ 73.7, 73.4, 72.3, 69.2, 65.0, 38.3, 29.3 (2C), 28.3, 21.3, 13.0; HRMS (FAB) calcd for C₁₁H₂₂BrO₃ [M+H]⁺ 281.0752. found 281.0767; structure confirmed by single crystal X-Ray diffraction.

200:

Colorless amorphous solid. R_{f}=0.29 (silica gel, hexanes:EtOAc, 1:2); IR (film) νₘₐₓ 3373 (br), 2969, 2935, 2876, 1460, 1110, 1047, 1027, 1006 cm⁻¹; ¹H NMR (400 MHz, CDCl₃) δ 4.22 (app d, 1H), 4.13 (dt, J=10.4, 4.0 Hz, 1H), 3.85 (m, 1H), 3.74 (m, 1H), 3.53 (m, 1H), 2.24 (br s, 2H), 2.18-1.98 (m, 4H), 1.80-1.54 (m, 4H), 1.23 (d, J=6.4 Hz, 3H), 1.06 (t, J=7.6 Hz, 3H); ¹³C NMR (100 MHz, CDCl₃) δ 81.0, 73.3, 72.7, 69.4, 63.0, 36.9, 31.4, 29.3, 27.1, 20.8, 12.7; HRMS (FAB) calcd for C₁₁H₂₀BrO₂ [M-OH]⁺ 263.0647. found 263.0661.

201.

4-Bromobenzoyl chloride (0.059 g, 0.27 mmol, 4.0 equiv) was added to a solution of diol 200 (0.019 g, 0.067 mmol, 1.0 equiv), 4-DMAP (8.2 mg, 0.067 mmol, 1.0 equiv), and Et₃N (0.075 mL, 0.54 mmol, 8.0 equiv) in CH₂Cl₂ (1 mL) at 0° C. The resultant colorless solution was then allowed to warm to 25° C. and was stirred at that temperature for 3 h. Upon completion, the light orange heterogeneous reaction mixture was quenched by the addition of water (8 mL) and 1 M HCl (2 mL). The crude product was then extracted with CH₂Cl₂ (3×10 mL), and the combined organic layers were washed with saturated aqueous NaHCO₃ (30 mL) and brine (30 mL), dried (MgSO₄), filtered, and concentrated. Purification of the resultant residue by flash column chromatography (silica gel, hexanes:EtOAc, 19:1+2% Et₃N) afforded bis-bromobenzoate 201 (0.037 g, 85% yield) as a white crystalline solid. Recrystallization from boiling MeOH (8 mL) afforded single crystals suitable for X-ray diffraction. 201: R_{f}=0.44 (silica gel, hexanes:EtOAc, 9:1); IR (film) νₘₐₓ 2968, 2930, 2875, 2851, 1718, 1590, 1265, 1101, 1012, 755 cm⁻¹; ¹H NMR (400 MHz, CDCl₃) δ 7.91 (d, J=8.4 Hz, 2H), 7.76 (d, J=8.4 Hz, 2H), 7.62 (d, J=8.8 Hz, 2H), 7.50 (d, J=8.4 Hz, 2H), 5.75-5.66 (m, 2H), 3.95 (m, 1H), 3.85 (ddd, J=9.6, 6.8, 2.4 Hz, 1H), 3.72 (m, 1H), 2.41-2.22 (m, 3H), 2.11 (sextet of doublets, J=7.2, 2.8 Hz, 1H), 2.05-1.85 (m, 3H), 1.78 (m, 1H), 1.32 (d, J=6.4 Hz, 3H), 1.09 (t, J=7.2 Hz, 3H); ¹³C NMR (125 MHz, CDCl₃) δ 165.2, 165.1, 132.1 (2C), 131.8 (2C), 131.2 (4C), 129.3 (2C), 128.5, 128.2, 82.0, 76.2, 72.6 (2C), 61.8, 37.1, 30.7, 27.3, 27.1, 21.4, 12.5; HRMS: No molecular ion peak could be observed; structure confirmed by single crystal X-Ray diffraction.

203:

White crystalline solid. R_{f}=0.31 (silica gel, hexanes:EtOAc, 1:2); IR (film) νₘₐₓ 3357 (br), 2963, 2930, 2879, 1460, 1375, 1062, 1044 cm⁻¹; ¹H NMR (400 MHz, CDCl₃) δ 4.23 (td, J=7.2, 3.6 Hz, 1H), 4.01-3.90 (m, 3H), 3.63 (ddd, J=10.4, 5.6, 3.6 Hz, 1H), 2.61 (ddd, J=20.4, 12.4, 8.0 Hz, 1H), 2.44 (d, J=2.8 Hz, 1H), 2.35 (br s, 1H), 2.21 (dd, J=14.8, 4.4 Hz, 1H), 1.95-1.72 (m, 4H), 1.18 (d, J=6.8 Hz, 3H), 0.92 (t, J=7.2 Hz, 3H); ¹³C NMR (100 MHz, CDCl₃) δ 74.8 (br), 71.8 (2C), 69.4, 51.7, 42.2, 38.1, 27.8, 20.9, 8.1; HRMS (FAB) calcd for C₁₀H₂₀BrO₃ [M+H]⁺ 267.0596. found 267.0592; structure confirmed by single crystal X-Ray diffraction. [Note: Owing to the appearance of the strangely broad carbon peak, an HSQC spectrum is also attached, establishing that the peak at 74.8 ppm is real].

205:

White crystalline solid. R_{f}=0.37 (silica gel, hexanes:EtOAc, 1:2); IR (film) νₘₐₓ 3372 (br), 2969, 2933, 2877, 1101, 1064, 1031 cm⁻¹; ¹H NMR (400 MHz, CDCl₃) δ 4.27 (dd, J=7.6, 2.8 Hz, 1H), 4.13 (quintet, J=4.4 Hz, 1H), 3.90 (ddd, J=14.4, 10.4, 4.0 Hz, 1H), 3.80 (m, 1H), 3.34 (td, J=9.6, 2.4 Hz, 1H), 2.78 (ddd, J=20.4, 12.4, 10.4 Hz, 1H), 2.28 (br s, 1H), 2.20-2.08 (m, 2H), 2.02 (m, 1H), 1.83 (dt, J=14.8, 4.4 Hz, 1H), 1.58 (br s, 1H), 1.39 (m, 1H), 1.21 (d, J=6.4 Hz, 3H), 0.94 (t, J=7.2 Hz, 3H); ¹³C NMR (100 MHz, CDCl₃) δ 83.3, 73.2, 72.2, 70.0, 53.5, 41.8, 37.8, 28.4, 20.9, 9.8; HRMS (FAB) calcd for C₁₀H₂₀BrO₃ [M+H]⁺ 267.0596. found 267.0607; structure could not be fully solved by single crystal X-Ray diffraction, although the crystallographer (W. S.) is confident it matches the assigned structure.

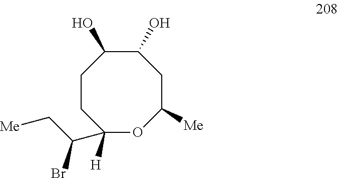 [SMILES: CC[C@H](Br)[C@@H]1CC[C@@H](O)[C@H](O)C[C@@H](C)O1]

208:

Afforded by the hydrolysis of a mixture of 145 and 146. White crystalline solid. R_{f}=0.25 (silica gel, hexanes:EtOAc, 1:3); IR (film) νₘₐₓ 3378 (br), 2966, 2933, 2876, 1121, 1074, 1033 cm⁻¹; ¹H NMR (400 MHz, CDCl₃) δ 4.28 (sextet of doublets, J=6.8, 2.0 Hz, 1H), 4.12 (t, J=8.0 Hz, 1H), 3.79-3.71 (app d, 2H), 3.60 (m, 1H), 2.55 (br s, 1H), 2.32 (br s, 1H), 2.11-1.94 (m, 3H), 1.82-1.57 (m, 5H), 1.15 (d, J=6.8 Hz, 3H), 1.05 (t, J=7.2 Hz, 3H); ¹³C NMR (100 MHz, CDCl₃) δ 76.3, 75.7, 73.4, 67.3, 62.2, 36.4, 32.4, 27.6, 25.8, 20.7, 13.3; HRMS (FAB) calcd for C₁₁H₂₂BrO₃ [M+H]⁺ 281.0752. found 281.0760; structure confirmed by single crystal X-Ray diffraction.

### Benzoate Hydrolysis.

A solution of LiOH (0.0240 g, 1.00 mmol, 20 equiv) in water (0.5 mL) was added to a solution of 141 and 142 (10:1, 0.0193 g, 0.0500 mmol, 1.0 equiv) in a mixture of THF (1.5 mL) and MeOH (0.5 mL) at 0° C. After stirring for 1 h at 0° C., the reaction mixture was quenched by the addition of saturated aqueous NH₄Cl (5 mL) and water (5 mL) and the crude product was extracted into EtOAc (3×10 mL). The combined organic layers were washed with brine (10 mL), dried (MgSO₄), filtered, and concentrated. Purification of the resultant white solid by flash column chromatography (silica gel, hexanes:EtOAc, 1:4) afforded the desired diol S18 (0.0125 g, 89% yield).

### Monobenzoylation of 194.

Benzoyl chloride (0.024 mL, 0.20 mmol, 3.0 equiv) was added dropwise to a solution of diol 194 (0.019 g, 0.068 mmol, 1.0 equiv) and Et₃N (0.19 mL, 1.4 mmol, 20 equiv) in CH₂Cl₂ (1 mL) at 0° C. The resultant colorless solution was stirred at 0° C. for 30 min and then quenched by the addition of MeOH (0.1 mL). Concentration and purification of the resultant residue by flash column chromatography (silica gel, hexanes:EtOAc, 7:3) afforded a 1:6.6 mixture of regioisomers 23 and 24 (0.0235 g, 90% yield) as a colorless amorphous solid.

### VI. Synthesis of 167 and Cyclization to 9-Exo Product 167

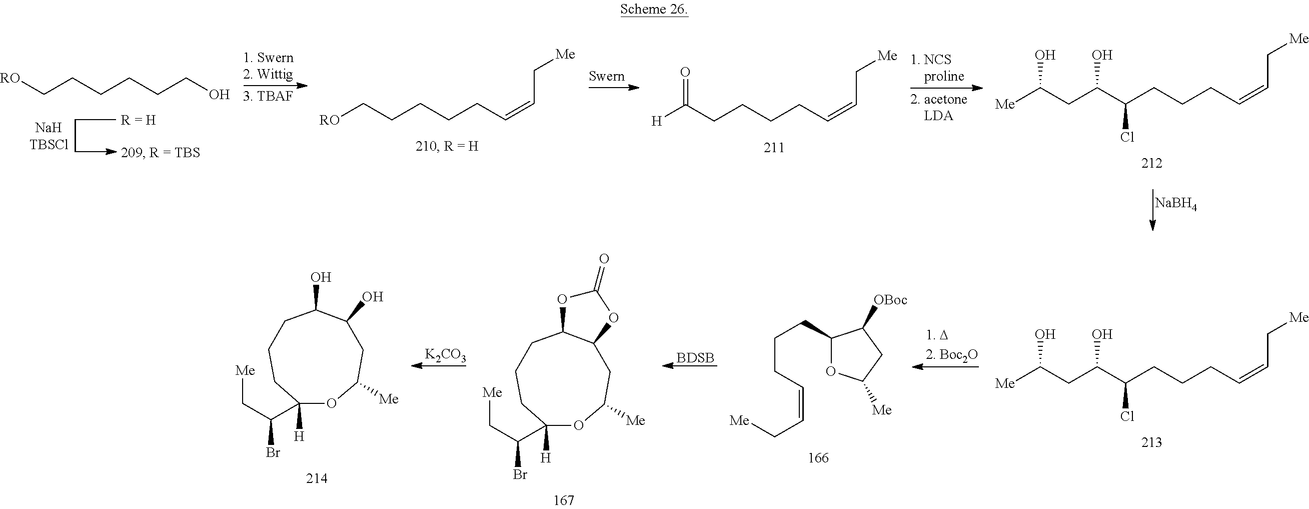

209.

TBSCl (9.0 g, 60. mmol, 1.0 equiv) and imidazole (4.9 g, 60. mmol, 1.0 equiv) were added sequentially to a solution of 1,6-hexanediol (7.1 g, 60. mmol, 1.0 equiv) in CH₂Cl₂ (300 mL) at 25° C. After 12 h at 25° C., the reaction contents were quenched by the addition of saturated aqueous NH₄Cl (100 mL) and water (50 mL). The layers were separated, and the aqueous layer was extracted with EtOAc (3×100 mL); the combined organic layers were dried (MgSO₄), filtered, and concentrated. The resultant oil was purified by flash column chromatography (silica gel, hexanes:EtOAc, 1:04:1) to afford 209 (6.8 g, 49% yield) as a colorless viscous oil. S33: R_{f}=0.31 (silica gel, hexanes:EtOAc, 4:1); IR (film) νₘₐₓ 3407 (br), 2932, 2858, 1640, 1254, 1097, 835, 775 cm⁻¹; ¹H NMR (400 MHz, CDCl₃) δ 3.64 (t, J=6.8 Hz, 2H), 3.61 (t, J=6.4 Hz, 2H), 1.62-1.48 (m, 4H), 1.42-1.32 (m, 4H), 1.22 (br s, 1H), 0.89 (s, 9H), 0.05 (s, 6H); ¹³C NMR (100 MHz, CDCl₃) δ 63.2, 63.0, 32.8 (2C), 26.0 (3C), 25.6, 25.5, 18.4, −5.3 (2C); HRMS (FAB) calcd for C₁₂H₂₉O₂Si [M+H]⁺ 233.1937. found 233.1940.

210.

209 (5.11 g, 22.0 mmol) was subject to Swern oxidation, Wittig olefination, and deprotection followed by purification by flash column chromatography (silica gel, hexanes:EtOAc, 1:04:1) to afford 210 (1.47 g, 59% yield over 3 steps) as a colorless viscous oil. S34: R_{f}=0.25 (silica gel, hexanes:EtOAc, 4:1); IR (film) νₘₐₓ 3377 (br), 3005, 2962, 2932, 2858, 1649, 1460, 1054 cm⁻¹; ¹H NMR (400 MHz, CDCl₃) δ 5.41-5.28 (m, 2H), 3.64 (t, J=6.4 Hz, 2H), 2.08-1.97 (m, 4H), 1.58 (m, 2H), 1.41-1.33 (m, 4H), 1.24 (br s, 1H), 0.95 (t, J=7.6 Hz, 3H); ¹³C NMR (100 MHz, CDCl₃) δ 131.8, 129.0, 63.0, 32.7, 29.5, 27.0, 25.4, 20.5, 14.4; HRMS (EI) calcd for C₉H₁₈O [M]⁺ 142.1358. found 142.1360.

### (6Z)-Nonenal (211).

Oxidation of 210 (1.47 g, 12.9 mmol) afforded the desired aldehyde 211 (1.35 g, 93% yield) as a light yellow oil that was used directly without purification. 211: R_{f}=0.48 (silica gel, hexanes:EtOAc, 9:1); IR (film) νₘₐₓ 3005, 2962, 2934, 2861, 2718, 1727, 1460 cm⁻¹; ¹H NMR (400 MHz, CDCl₃) δ 9.76 (t, J=2.0 Hz, 1H), 5.43-5.25 (m, 2H), 2.43 (td, J=7.2, 2.0 Hz, 2H), 2.09-1.98 (m, 4H), 1.65 (quintet, J=7.6 Hz, 2H), 1.39 (quintet, J=7.6 Hz, 2H), 0.95 (t, J=7.6 Hz, 3H); ¹³C NMR (100 MHz, CDCl₃) δ 202.7, 132.2, 128.3, 43.8, 29.2, 26.7, 21.7, 20.5, 14.3; HRMS (FAB) calcd for C₉H₁₅O [M−H]⁺ 139.1123. found 139.1118.

212.

(6Z)-Nonenal (S35, 1.35 g) was α-chlorinated to yield (6Z)-2-chloro-6-nonenal (1.99 g, 82% yield) as a colorless viscous oil that was used directly without purification. The aldol addition with acetone was performed immediately, followed by purification by careful flash column chromatography (silica gel, hexanes:EtOAc, 1:0→4:1) to afford 212 (0.963 g, 53% yield) as a colorless viscous oil. 212: R_{f}=0.28 (silica gel, hexanes:EtOAc, 4:1); IR (film) νₘₐₓ 3435 (br), 3005, 2962, 2934, 2872, 1714, 1362, 1165, 1080 cm⁻¹; ¹H NMR (400 MHz, CDCl₃) δ 5.38 (m, 1H), 5.30 (m, 1H), 4.10 (m, 1H), 3.92 (ddd, J=9.6, 6.0, 3.2 Hz, 1H), 3.24 (d, J=5.2 Hz, 1H), 2.89-2.74 (m, 2H), 2.22 (s, 3H), 2.12-1.98 (m, 4H), 1.90 (m, 1H), 1.73-1.58 (m, 2H), 1.46 (m, 1H), 0.96 (t, J=7.6 Hz, 3H); ¹³C NMR (100 MHz, CDCl₃) δ 209.3, 132.4, 128.2, 70.8, 65.8, 45.9, 33.3, 30.9, 26.4 (2C), 20.5, 14.3; HRMS (FAB) calcd for C₁₂H₂₂ClO₂ [M+H]⁺ 233.1308. found 233.1308.

213.

NaBH₄ reduction of 212 (0.421 g, 1.81 mmol) followed by purification by flash column chromatography (silica gel, hexanes:EtOAc, 4:11:1) afforded cis-diol 213 (0.282 g, 60% yield) [along with the corresponding trans-diol (0.097 g, 20% yield)] as a colorless amorphous solid. 213: R_{f}=0.29 (silica gel, hexanes:EtOAc, 7:3); IR (film) νₘₐₓ 3369 (br), 3005, 2965, 2933, 2873, 1457, 1137, 1076 cm⁻¹; ¹H NMR (400 MHz, CDCl₃) δ 5.43-5.25 (m, 2H), 4.06 (m, 1H), 3.95 (m, 1H), 3.88 (m, 1H), 3.51 (d, J=4.0 Hz, 1H), 3.00 (d, J=2.0 Hz, 1H), 2.11-1.96 (m, 4H), 1.87-1.38 (m, 6H), 1.23 (d, J=6.0 Hz, 3H), 0.95 (t, J=7.6 Hz, 3H); ¹³C NMR (100 MHz, CDCl₃) δ 132.3, 128.2, 75.6, 68.7, 67.7, 40.2, 32.4, 26.6, 26.4, 24.1, 20.5, 14.3; HRMS (FAB) calcd for C₁₂H₂₄ClO₂ [M+H]⁺ 235.1465. found 235.1466.

166.

Prepared according to the procedures described above for the synthesis of 138a and 138d. Cyclization of 213 (0.150 g, 0.64 mmol) at 130° C. for 12 h followed by flash column chromatography (silica gel, hexanes:EtOAc, 9:11:1) afforded the desired hydroxytetrahydrofuran (0.120 g, 95% yield) as a colorless viscous oil. A portion of the cyclized product (0.060 g, 0.30 mmol) was taken forward to afford carbonate 166 (0.065 g, 75% yield) after flash column chromatography (silica gel, hexanes:EtOAc, 19:1→4:1) as a colorless viscous oil. 166: R_{f}=0.35 (silica gel, hexanes:EtOAc, 9:1); IR (film) νₘₐₓ 2966, 2933, 2871, 1739, 1369, 1280, 1255, 1165 cm⁻¹; ¹H NMR (400 MHz, CDCl₃) δ 5.40-5.26 (m, 2H), 5.16 (t, J=4.0 Hz, 1H), 4.31 (m, 1H), 4.00 (m, 1H), 2.18 (ddd, J=13.6, 6.0, 1.2 Hz, 1H), 2.09-1.97 (m, 4H), 1.77 (ddd, J=14.4, 9.6, 5.2 Hz, 1H), 1.65-1.47 (m, 3H), 1.48 (s, 9H), 1.36 (m, 1H), 1.23 (d, J=6.0 Hz, 3H), 0.94 (t, J=7.6 Hz, 3H); ¹³C NMR (100 MHz, CDCl₃) δ 153.3, 131.9, 128.7, 82.1, 80.7, 78.5, 72.9, 40.9, 28.8, 27.8 (3C), 27.1, 26.4, 21.3, 20.5, 14.3; HRMS (FAB) calcd for C₁₇H₃IO₄ [M+H]⁺299.2222. found 299.2217.

167.

Prepared according to General Cyclization Procedure A described above. BDSB cyclization of 166 (0.0298 g, 0.100 mmol) followed by flash column chromatography (silica gel, hexanes:EtOAc, 1:01:1) afforded 167 (0.0110 g, 34% yield) as a colorless amorphous solid. 167: R_{f}=0.48 (silica gel, hexanes:EtOAc, 7:3); IR (film) νₘₐₓ 2969, 2939, 2876, 1799, 1748, 1379, 1243, 1199, 1121 cm⁻¹; ¹H NMR (400 MHz, CDCl₃) δ 4.97 (ddd, J=11.6, 6.4, 2.4 Hz, 1H), 4.82 (q, J=6.8 Hz, 1H), 3.94-3.83 (m, 2H), 3.53 (m, 1H), 2.60 (ddd, J=18.0, 11.6, 6.4 Hz, 1H), 2.02-1.78 (m, 5H), 1.75-1.60 (m, 4H), 1.34 (d, J=6.4 Hz, 3H), 1.06 (t, J=7.2 Hz, 3H); ¹³C NMR (100 MHz, CDCl₃) δ 154.6, 83.3, 80.9, 76.7, 73.9, 60.7, 33.9, 28.1, 27.1, 26.8, 21.7, 20.1, 12.5; HRMS (FAB) calcd for C₁₃H₂₂BrO₄ [M+H]⁺ 321.0701. found 321.0704.

214.

Prepared according to the procedure described above for general acetate/carbonate hydrolysis. Hydrolysis of 167 (0.011 g, 0.034 mmol) followed by purification by flash column chromatography (silica gel, hexanes:EtOAc, 4:1→0:1) afforded 214 (7.4 mg, 74% yield) as a white crystalline solid. Crystals suitable for X-ray diffraction were grown by slow evaporation from a CH₂Cl₂:toluene mixture. 214: R_{f}=0.20 (silica gel, hexanes:EtOAc, 1:1); IR (film) νₘₐₓ 3375 (br), 2966, 2926, 1461, 1331, 1076, 995 cm⁻¹; ¹H NMR (400 MHz, CDCl₃) δ 4.27 (m, 1H), 4.18 (m, 1H), 3.92-3.83 (m, 2H), 3.49 (ddd, J=10.4, 4.4, 2.0 Hz, 1H), 2.26 (ddd, J=14.8, 9.6, 4.4 Hz, 1H), 2.12 (br s, 1H), 2.01-1.49 (m, 10H), 1.28 (d, J=6.8 Hz, 3H), 1.06 (t, J=7.2 Hz, 3H); ¹³C NMR (100 MHz, CDCl₃) δ 81.1, 73.5, 71.9, 70.7, 62.6, 36.5, 30.9, 29.5, 270, 21.4, 20.4, 12.8; HRMS (FAB) calcd for C₁₂H₂₃BrNaO₃ [M+Na]⁺ 317.0728. found 317.0734; structure confirmed by single crystal x-ray diffraction.

### VII. Synthesis of 50 and Cyclization to 9-Endo Product 51 (93, 94)

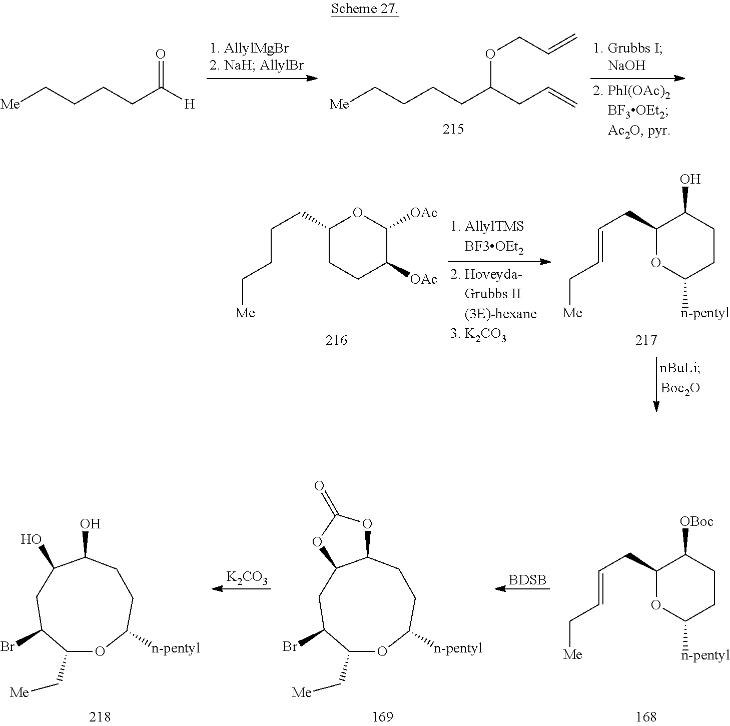

215.

A solution of allylmagnesium bromide (1.0 M in Et₂O, 12.0 mL, 12.0 mmol, 1.2 equiv) was added to a solution of hexanal (1.30 mL, 10.0 mmol, 1.0 equiv) in Et₂O (4.0 mL) at 0° C. After 30 min at 0° C., the reaction contents were quenched by the addition of saturated aqueous NH₄Cl (5 mL) and water (5 mL). The layers were separated and the aqueous layer was extracted with additional Et₂O (2×10 mL). The combined organic layers were dried (MgSO₄), filtered, and concentrated to yield a light yellow residue that was purified by flash column chromatography (silica gel, pentane:Et₂O, 49:1→9:1) to afford 1-nonen-4-ol (0.81 g, 57% yield) as a colorless oil. Next, NaH (60% dispersion in mineral oil, 0.456 g, 11.4 mmol, 2.0 equiv) was added slowly to a solution of the newly formed 1-nonen-4-ol (0.810 g, 5.69 mmol, 1.0 equiv) in THF (19 mL) at 25° C. The mixture was carefully heated to reflux for 30 min, then cooled to 25° C. Allyl bromide (0.975 mL, 11.4 mmol, 2.0 equiv) was added, and the reaction contents were again heated to reflux for 45 min. Upon completion, the colorless solution was cooled to 25° C., quenched by the addition of saturated aqueous NH₄Cl (10 mL) and water (10 mL), and extracted with Et₂O (3×10 mL). The combined organic layers were dried (MgSO₄), filtered, and concentrated to afford a light yellow residue that was purified by flash column chromatography (silica gel, hexanes:CH₂Cl₂, 9:1) to afford 215 (0.820 g, 79% yield) as a colorless viscous oil. 215: R_{f}=0.28 (silica gel, hexanes:CH₂Cl₂, 9:1); IR (film) νₘₐₓ 3078, 2957, 2931, 2859, 1642, 1460, 1084, 916 cm⁻¹; ¹H NMR (400 MHz, CDCl₃) δ 5.97-5.77 (m, 2H), 5.26 (dq, J=17.2, 1.6 Hz, 1H), 5.14 (dq, J=10.4, 1.2 Hz, 1H), 5.11-5.02 (m, 2H), 4.00 (qdt, J=12.8, 5.6, 1.2 Hz, 2H), 3.35 (quintet, J=5.6 Hz, 1H), 2.32-2.21 (m, 2H), 1.53-1.21 (m, 8H), 0.88 (t, J=6.8 Hz, 3H); ¹³C NMR (100 MHz, CDCl₃) δ 135.6, 135.3, 116.9, 116.6, 78.7, 70.1, 38.5, 33.9, 32.1, 25.2, 22.8, 14.2; HRMS (FAB) calcd for C₁₂H₂₁O [M−H]⁺ 181.1592. found 181.1599.

216.

Grubbs catalyst (1^{st} generation, 0.135 g, 0.163 mmol, 0.05 equiv) was added to a solution of 215 (0.600 g, 3.29 mmol, 1.0 equiv) in toluene (13.2 mL) at 25° C. After consumption of 215 was observed by TLC analysis (˜30 min), 2-propanol (3.3 mL) and NaOH (0.0330 g, 0.823 mmol, 0.25 equiv) were added sequentially in single portions. The resultant brown solution was heated to reflux for 12 h, then cooled to 25° C. and quenched with water (5 mL). The crude product was extracted into Et₂O (3×5 mL), and the combined organic layers were dried (MgSO₄), filtered, and concentrated. The resultant brown residue was purified by flash column chromatography (silica gel, hexanes:CH₂Cl₂, 2:1) to afford the desired enol ether product (0.442 g, 87% yield) as a colorless viscous oil. Next, PhI(OAc)₂ (1.19 g, 3.40 mmol, 1.2 equiv) and BF₃.OEt₂ (0.088 mL, 0.570 mmol, 0.20 equiv) were added sequentially in single portions to a solution of the enol ether produced above (0.442 g, 2.90 mmol, 1.0 equiv) in CH₂Cl₂ (22 mL) at −40° C. The reaction contents were stirred at −40° C. for 4 h, then pyridine (9 mL) and Ac₂O (4.50 mL, 47.6 mmol, 16.4 equiv) were then added, and the reaction contents were warmed to 25° C. and stirred at that temperature for 12 h. Upon completion, the reaction mixture was quenched by the addition of water (20 mL) and extracted with EtOAc (3×10 mL). The combined organic layers were washed with saturated aqueous NaHCO₃ (20 mL), dried (MgSO₄), filtered, and concentrated. The resultant brown residue was purified by flash column chromatography (silica gel, hexanes:EtOAc, 2:1) to afford diacetate 216 as a colorless viscous oil contaminated with small amounts of inseparable impurities (estimated pure yield=0.380 g, 49% yield). Stereochemistry was determined by COSY and NOESY NMR experiments (see attached spectra). 216: R_{f}=0.44 (silica gel, hexanes:EtOAc, 4:1); IR (film) νₘₐₓ 2932, 2861, 1747, 1371, 1241, 1060, 949 cm⁻¹; ¹H NMR (400 MHz, CDCl₃) δ 5.61 (d, J=8.4 Hz, 1H), 4.69 (m, 1H), 3.54 (m, 1H), 2.21 (m, 1H), 2.10 (s, 3

H), 2.03 (s, 3H), 1.72 (m, 1H), 1.63-1.20 (m, 10H), 0.86 (t, J=6.8 Hz, 3H); ¹³C NMR (100 MHz, CDCl₃) δ 169.9, 169.5, 94.0, 77.0, 70.1, 35.1, 31.7, 29.6, 27.9, 25.2, 22.5, 21.1, 21.0, 14.0; HRMS (FAB) calcd for C₁₄H₂₃O₅ [M−H]⁺ 271.1545. found 271.1535.

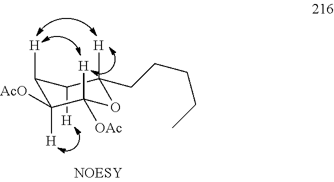 [SMILES: CCCCCC1CCC(OC(C)=O)C(OC(C)=O)O1]

217.

Allyltrimethylsilane (0.66 mL, 4.2 mmol, 3.0 equiv) was added to a solution of diacetate S40 (0.38 g, 1.4 mmol, 1.0 equiv) in CH₂Cl₂ (3.8 mL), at −78° C. Next, BF₃.OEt₂ (0.98 mL, 7.0 mmol, 5.0 equiv) was added dropwise over 5 min, and the resultant light yellow solution was allowed to slowly warm from −78° C. to 25° C. over the course of 12 h. Upon completion, the reaction mixture was quenched by the addition of saturated aqueous NaHCO₃ (10 mL), and the reaction mixture was extracted with CH₂Cl₂ (2×20 mL). The combined organic layers were dried (MgSO₄), filtered, and concentrated to yield a brown residue that was purified by flash column chromatography (silica gel, hexanes:EtOAc, 9:1) to afford the desired allylated product (0.26 g, 74% yield) as a colorless viscous oil. Next, the Hoveyda-Grubbs catalyst (2^{nd} generation, 0.0195 g, 0.31 mmol, 0.03 equiv) was added to a solution of the allylated product generated above (0.264 g, 1.04 mmol, 1.0 equiv) in trans-3-hexene (5.00 g, 59.0 mmol, 57 equiv) at 25° C. The resultant brown solution was stirred for 12 h at 25° C., then filtered through a small plug of silica gel with hexanes:EtOAc (3:1, 100 mL). The filtrate was concentrated and the resultant oil was dissolved in MeOH (50 mL) and cooled to 0° C. K₂CO₃ (1.44 g, 10.4 mmol, 10.0 equiv) was then added and the reaction mixture was stirred for 1 h at 0° C. Upon completion, the solution was quenched by the addition of saturated aqueous NH₄Cl (10 mL) and water (50 mL), and extracted with EtOAc (3×50 mL). The combined organic layers were dried (MgSO₄), filtered, and concentrated to yield an oil that was purified by flash column chromatography (silica gel, hexanes:EtOAc, 1:0→4:1) to afford 217 (0.180 g, 72% yield over two steps) as a colorless viscous oil. 217: R_{f}=0.33 (silica gel, hexanes:EtOAc, 4:1); IR (film) νₘₐₓ 3403 (br), 2933, 2860, 1460, 1376, 1080, 966 cm⁻¹; ¹H NMR (400 MHz, CDCl₃) δ 5.57 (m, 1H), 5.43 (m, 1H), 3.77-3.69 (m, 2H), 3.66 (m, 1H), 2.32 (m, 1H), 2.22 (m, 1H), 2.07-1.89 (m, 3H), 1.85-1.64 (m, 4H), 1.42-1.24 (m, 8H), 0.97 (t, J=7.6 Hz, 3H), 0.90 (t, J=6.8 Hz, 3H); ¹³C NMR (100 MHz, CDCl₃) δ 134.7, 125.2, 73.0, 71.3, 67.3, 32.5, 31.9, 31.8, 26.9, 25.9, 25.8, 25.4, 22.8, 14.2, 13.9; HRMS (FAB) calcd for C₁₅H₂₉O₂ [M+H]⁺ 241.2168. found 241.2172.

168.

Prepared according to the procedure described above for the synthesis of carbonate 138d. Carbonate formation on 0.36 mmol scale followed by flash column chromatography (silica gel, hexanes:EtOAc, 19:1→17:3) afforded 168 (0.095 g, 77% yield) as a colorless viscous oil. Stereochemistry was determined by COSY and NOESY NMR experiments (see attached spectra). 168: R_{f}=0.58 (silica gel, hexanes:EtOAc, 9:1); IR (film) νₘₐₓ 2958, 2933, 2859, 1739, 1369, 1278, 1255, 1165, 1086 cm⁻¹; ¹H NMR (400 MHz, CDCl₃) δ 5.52 (m, 1H), 5.39 (m, 1H), 4.69 (quintet, J=4.0 Hz, 1H), 3.85 (quintet, J=4.4 Hz, 1H), 3.66 (m, 1H), 2.39 (m, 1H), 2.14 (m, 1H), 2.01 (quintet, J=7.2 Hz, 2H), 1.92-1.75 (m, 3H), 1.58 (m, 1H), 1.47 (s, 9H), 1.40-1.20 (m, 8H), 0.96 (t, J=7.6 Hz, 3H), 0.88 (t, J=6.8 Hz, 3H); ¹³C NMR (100 MHz, CDCl₃) δ 153.2, 134.7, 124.8, 82.0, 72.5, 72.0, 70.0, 33.2, 32.0, 31.2, 28.0 (3C), 27.6, 27.4, 25.8, 24.0, 22.8, 14.2, 13.9; HRMS (FAB) calcd for C₂₀H₃₇O₄ [M+H]⁺ 341.2692. found 341.2678.

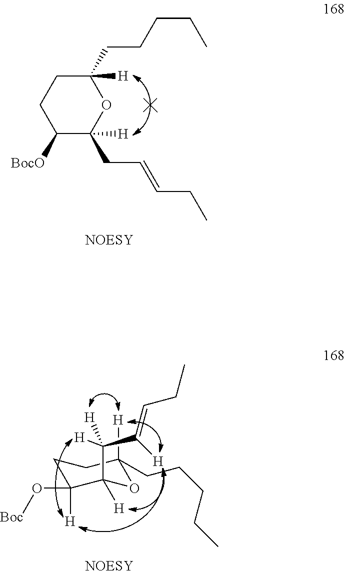

169.

Prepared according to General Cyclization Procedure A. BDSB cyclization of 168 (0.0341 g, 0.100 mmol) afforded 169 (0.0185 g, 51% yield) after flash column chromatography (silica gel, hexanes:EtOAc, 19:1→17:3) as a colorless viscous oil. 51: R_{f}=0.23 (silica gel, hexanes:EtOAc, 9:1); IR (film) νₘₐₓ 2934, 2861, 1805, 1363, 1186, 1034 cm⁻¹; ¹H NMR (400 MHz, CDCl₃) δ 4.89 (m, 1H), 4.75 (ddd, J=9.2, 7.2, 1.6 Hz, 1H), 3.90 (ddd, J=11.6, 9.2, 2.4 Hz, 1H), 3.50 (quintet, J=4.8 Hz, 1H), 3.43 (m, 1H), 2.70 (m, 1H), 2.56 (dt, J=15.2, 2.0 Hz, 1H), 2.10-1.94 (m, 4H), 1.84-1.67 (m, 2H), 1.58 (m, 1H), 1.44-1.20 (m, 7H), 0.92 (t, J=7.2 Hz, 3H), 0.89 (t, J=7.2 Hz, 3H); ¹³C NMR (100 MHz, CDCl₃) δ 154.0, 84.7, 82.8, 82.1, 81.4, 49.6, 37.4, 34.5, 31.9, 30.3, 26.4, 26.2, 24.8, 22.6, 14.0, 7.8; HRMS (FAB) calcd for C₁₆H₂₈BrO₄ [M+H]⁺ 363.1171. found 363.1163.

218.

Prepared according to the procedure described above for general acetate/carbonate hydrolysis. Hydrolysis of 169 (0.0153 g, 0.0423 mmol) followed by purification by flash column chromatography (silica gel, hexanes:EtOAc, 9:1→1:1) afforded 218 (0.0115 g, 81% yield) as a white crystalline solid. Connectivity and stereochemistry were confirmed by COSY and NOESY NMR experiments (see attached spectra). 218: R_{f}=0.20 (silica gel, hexanes:EtOAc, 7:3); IR (film) νₘₐₓ 3391 (br), 2929, 2857, 1459, 1066, 914, 746 cm⁻¹; ¹H NMR (400 MHz, CDCl₃) δ 4.27 (br s, 1H), 4.04-3.92 (m, 2H), 3.48 (dt, J=10.0, 4.0 Hz, 1H), 3.35 (m, 1H), 2.58 (ddd, J=17.2, 11.2, 6.0 Hz, 1H), 2.25 (ddd, J=15.2, 3.6, 2.0 Hz, 1H), 2.18-2.07 (m, 2H), 1.94-1.63 (m, 5H), 1.55-1.35 (m, 3H), 1.34-1.19 (m, 6H), 0.91 (t, J=7.6 Hz, 3H), 0.88 (t, J=7.2 Hz, 3H); ¹³C NMR (100 MHz, CDCl₃) δ 87.2, 85.1, 74.9, 73.3, 50.4, 42.2, 36.6, 32.0, 30.6, 29.8, 26.5, 24.7, 22.6, 14.0, 7.1; HRMS (FAB) calcd for C₁₅H₂₉BrNaO₃ [M+Na]⁺ 359.1198. found 359.1181.

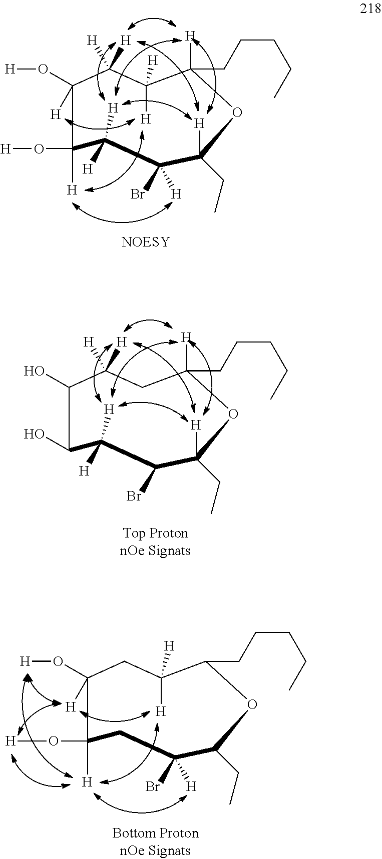

### 13. Halogenation of Aromatic Ring: Synthesis of Resveratrol Oligomers

Permethylated ampelopsin F (170, 0.560 g, 1.04 mmol, 1.0 equiv) was dissolved in CH₂Cl₂ (20 mL), cooled to −78° C., and then BDSB (0.516 g, 0.94 mmol, 0.9 equiv) was added in a single portion. The resultant solution was stirred at −78° C. for 2 h. Upon completion, the reaction contents were quenched with saturated aqueous NaHCO₃ (20 mL) and saturated aqueous Na₂SO₃ (50 mL), and extracted with EtOAc (2×50 mL). The combined organic layers were then dried (MgSO₄), filtered, and concentrated. The resultant amorphous product was purified by flash column chromatography (silica gel, hexanes:EtOAc, 10:1→4:1) to afford bromide 173 contaminated with a trace of dibromide (0.560 g total, 0.500 g 173 based on NMR integration, 78%, 85% yield based on recovered starting material) as an amorphous off-white solid and recovered permethylated ampelopsin F (170, 0.045 g, 8%). An analytical sample was obtained by running the reaction less to completion. [Note: the large scale reaction was run to test the robustness of the method; key is to note that monobromide 171 was not detected by NMR analysis]. 173: R_{f}=0.56 (silica gel, hexanes:EtOAc, 1:1); IR (film) νₘₐₓ 2935, 2835, 1606, 1583, 1510, 1462, 1434, 1336, 1320, 1248, 1209, 1178, 1140, 1080, 1036, 966, 830 cm⁻¹; ¹H NMR (400 MHz, CDCl₃) δ 7.15 (d, J=8.8 Hz, 2H), 6.84 (d, J=8.4 Hz, 2H), 6.81 (d, J=8.4 Hz, 2H), 6.66 (d, J=8.8 Hz, 2H), 6.50 (d, J=2.4 Hz, 1H), 6.27 (d, J=2.4 Hz, 1H), 6.19 (s, 1H), 4.34 (s, 1H), 4.25 (s, 1H), 3.83 (s, 3H), 3.83 (s, 3H), 3.79 (s, 3H), 3.75 (s, 3H), 3.69 (s, 1H), 3.69 (s, 4H), 3.44 (s, 3H); ¹³C NMR (100 MHz, CDCl₃) δ 159.7, 159.1, 157.9, 157.7, 156.0, 154.1, 145.6, 145.5, 138.8, 135.0, 130.0, 128.8, 128.6, 115.7, 113.7, 113.6, 113.5, 103.1, 97.2, 95.8, 57.2, 56.7, 55.8, 55.5, 55.4, 55.3 (2C), 51.0, 49.2, 43.7; HRMS (FAB) calcd for C₃₄H₃₃BrO₆⁺[M⁺] 617.5262. found 616.1465 (for ⁷⁹Br).

### 14. Determination of Anti-Viral Activity of Peyssonol A and Derivatives

A recombinant NL4-3 derived virus, termed Rep-Rluc Sac II, was constructed, in which a section of the nef gene from NL4-3 was replaced with the Renilla luciferase gene. This CXCR4-tropic virus is replication competent in cell culture and expresses the Renilla luciferase gene as a means of measuring virus growth. Virus was used to infect MT-2 cells in the presence of compounds, and after 5 days of incubation, cells are processed and quantitated for virus growth by the amount of expressed luciferase. Luciferase was quantitated using the Dual Luciferase kit from Promega (Madison, Wis.) according to manufacturer's instructions and luciferase activity was measured on a Wallac TriLux (Perkin-Elmer). Susceptibility of viruses to compounds was determined by incubation in the presence of serial dilutions of the compounds. The 50% effective concentration (EC₅₀) and 50% cytotoxicity concentration were calculated by using the exponential form of the median effect equation where (Fa)=1/[1+(ED₅₀/drug conc.)^{m}]. Compound cytotoxicity was assayed in parallel by exposing uninfected MT-2 cells to serially diluted compounds, and measuring cell viability in an XTT assay, according to the manufacturer's recommendations.

### REFERENCES

(1) For reviews, see: (a) Sutherland, J. K. in Comprehensive Organic Synthesis (Trost, B. M. ed.), Pergamon Press; Oxford, 1991, Vol. 5, pp. 341-377. (b) Yoder, R. A.; Johnston, J. N. Chem. Rev. 2005, 105, 4730-4756.
(2) (a) Stork, G.; Burgstahler, A. W. J. Am. Chem. Soc. 1955, 77, 5068-5077. (b) Eschenmoser, A.; Ruzicka, L.; Jeger, O.; Arigoni, D. Helv. Chim. Acta 1955, 38, 1890-1904. For a recent perspective on this work fifty years after its original publication, see: (c) Eschenmoser, A.; Arigoni, D. Helv. Chim. Acta 2005, 88, 3011-3050.
(3) For selected examples, see: (a) Yang, D.; Gu, S.; Yan, Y.-L.; Zhao, H.-W.; Zhu, N.-Y. Angew. Chem. Int. Ed. 2002, 41, 3014-3017. (b) Ishibashi, H.; Ishihara, K.; Yamamoto, H. J. Am. Chem. Soc. 2004, 126, 11122-11123. (c) Zhao, Y.-J.; Chng, S.-S.; Loh, T.-P. J. Am. Chem. Soc. 2007, 129, 492-493. (d) Zhao, Y.-J.; Loh, T.-P. J. Am. Chem. Soc. 2008, 130, 10024-10029. (e) Surendra, K.; Corey, E. J. J. Am. Chem. Soc. 2009, 131, 13928-13929. (f) Topczewski, J. J.; Callahan, M. P.; Neighbors, J. D.; Wiemer, D. F. J. Am. Chem. Soc. 2009, 131, 14630-14631. (g) Rendler, S.; MacMillan, D. W. C. J. Am. Chem. Soc. 2010, 132, 5027-5029. (h) Knowles, R. N.; Lin, S.; Jacobsen, E. N. J. Am. Chem. Soc. 2010, 132, 5030-5032. (i) Corey, E. J.; Lin, S. A. J. Am. Chem. Soc. 1996, 118, 8765-8776. (j) Corey, E. J.; Lee, J.; Liu, D. S. Tetrahedron Lett. 1994, 35, 9149-9152. (k) Mi, Y.; Schreiber, J. V.; Corey, E. J. J. Am. Chem. Soc. 2002, 124, 11290-11291. (1) Zhang, J.; Corey, E. J. Org. Lett. 2001, 3, 3215-3216. (m) Shenvi, R. A.; Corey, E. J. Org. Lett. 2010, 12, 3548-3551. For selected examples of classic papers in the field, see: (n) Johnson, W. S.; Gravestock, M. B.; McCarry, B. E. J. Am. Chem. Soc. 1971, 93, 4332-4334. (o) van Tamelen, E. E.; Willet, J.; Schwartz, M.; Nadeau, R. J. Am. Chem. Soc. 1966, 88, 5937-5938. (p) van Tamelen, E. E.; Hwu, J. R. J. Am. Chem. Soc. 1983, 105, 2490-2491.
(4) (a) Hoye, T. R.; Kurth, M. J. J. Org. Chem. 1979, 44, 3461-3467. (b) Hoye, T. R.; Caruso, A. J.; Kurth, M. J. J. Org. Chem. 1981, 46, 3550-3552. (c) Nishizawa, M.; Takenaka, H.; Nishide, H.; Hayashi, Y. Tetrahedron Lett. 1983, 24, 2581-2584. (d) Nishizawa, M.; Takenaka, H.; Hayashi, Y. J. Org. Chem. 1986, 51, 806-813. (e) Corey, E. J.; Reid, J. G.; Myers, A. G.; Hahl, R. W. J. Am. Chem. Soc. 1987, 109, 918-919. (f) Gopalan, A. S.; Prieto, R.; Mueller, B.; Peters, D. Tetrahedron Lett. 1992, 33, 1679-1682. (g) Nishizawa, M.; Morikuni, E.; Asoh, K.; Kan, Y.; Uenoyama, K.; Imagawa, H. Synlett 1995, 169-170. (h) Nishizawa, M.; Yadav, A.; Iwamoto, Y.; Imagawa, H. Tetrahedron 2004, 60, 9223-9234. (i) Takao, H.; Wakabayashi, A.; Takahashi, K.; Imagawa, H.; Sugihara, T.; Nishizawa, M. Tetrahedron Lett. 2004, 45, 1079-1082. (j) Kucera, D. J.; O'Connor, S. J.; Overman, L. E. J. Org. Chem. 1993, 58, 5304-5306. (k) Overman, L. E.; Abelman, M. M.; Kucera, D. J.; Tran, V. D.; Ricca, D. J. Pure App. Chem. 1992, 64, 1813-1819. (1) Koh, J. H.; Gagné, M. R. Angew. Chem. Int. Ed. 2004, 43, 3459-3461. (m) Mullen, C. A.; Campbell, A. N.; Gagné, M. R. Angew. Chem. Int. Ed. 2008, 47, 6011-6014. (n) Feducia, J. A.; Gagné, M. R. J. Am. Chem. Soc. 2008, 130, 592-599. (O) Fürstner, A.; Davies, P. W. Angew. Chem. Int. Ed. 2007, 46, 3410-3449. (p) Fürstner, A.; Morency, L. Angew. Chem. Int. Ed. 2008, 47, 5030-5033. (q) Sethofer, S. G.; Mayer, T.; Toste, F. D. J. Am. Chem. Soc. 2010, 132, 8276-8277.
(5) Other approaches to such frameworks have been developed as well. For a recent example featuring a carbonyl-ene reaction, see: Zhao, Y.-J.; Li, B.; Tan, L.-J. S.; Shen, Z.-L.; Loh, T.-P. J. Am. Chem. Soc. 2010, 132, 10242-10244.
(6) For reviews, see: (a) Vailancourt, F. A.; Yeh, E.; Vosburg, D. A.; Garneau-Tsodikova, S.; Walsh, C. T. Chem. Rev. 2006, 106, 3364-3378. (b) Butler, A.; Walker, J. V. Chem. Rev. 1993, 93, 1937-1944. For work illustrating that the napyradiomycins are prepared via vanadium-based haloperoxidases, see: (c) Winter, J. M.; Moffitt, M. C.; Zazopoulos, E.; McAlpine, J. B.; Dorrestein, P. C.; Moore, B. S. J. Biol. Chem. 2007, 282, 16362-16368.
(7) For the isolation of these natural products, see: (a) Shiomi, K.; Nakamura, H.; Iinuma, H.; Naganawa, H.; Takeuchi, T.; Umezawa, H.; Iitaka, Y. J. Antibiotics 1987, 40, 1213-1219 and references therein. (b) Cho, J. Y.; Kwon, H. C.; Williams, P. G.; Jensen, P. R.; Fenical, W. Org. Lett. 2006, 8, 2471-2474. (c) Talpir, R.; Rudi, A.; Kashman, Y.; Loya, Y.; Hizi, A. Tetrahedron 1994, 50, 4179-4184. (d) Loya, S.; Bakhanaskvili, M.; Kashman, Y.; Hizi, A. Archives of Biochem. Biophys. 1995, 316, 789-796. (e) Howard, B. M.; Fenical, W. Tetrahedron Lett. 1976, 17, 41-44. (f) Wall, M. E.; Wani, M. C.; Manikumar, G.; Taylor, H.; Hughes, T. J.; Gaetano, K.; Gerwick, W. H.; McPhail, A. T.; McPhail, D. R. J. Nat. Prod. 1989, 52, 1092-1099.
(8) Gribble, G. N. Acc. Chem. Res. 1998, 31, 141-152.
(9) Carter-Franklin, J. N.; Butler, A. J. Am. Chem. Soc. 2004, 126, 15060-15066.
(10) Only radical approaches have allowed access to relevant frameworks, typically through halogenation of every alkene within the substrate: (a) Yang, D.; Yan, Y.-L.; Zheng, B.-F.; Gao, Q.; Zhu, N.-Y. Org. Lett. 2006, 8, 5757-5760. (b) Helliwell, M.; Fengas, D.; Knight, C. K.; Parker, J.; Quayle, P.; Raftery, J.; Richards, S, N. Tetrahedron Lett. 2005, 46, 7129-7134.
(11) For representative examples, see: (a) van Tamelen, E. E.; Hessler, J. Chem. Comm. 1966, 411-413. (b) Kato, T.; Ichinose, I.; Kumazawa, S.; Kitahara, Y. Bioorg. Chem. 1975, 4, 188-193. (c) Kato, T.; Ichinose, I.; Kamoshida, A.; Kitahara, Y. J. Chem. Soc., Chem. Commun. 1976, 518-519. (d) Wolinsky, L. E.; Faulkner, D. J. J. Org. Chem. 1976, 41, 597-600. (e) González, A. G.; Martín, J. D.; Pérez, C.; Ramirez, M. A. Tetrahedron Lett. 1976, 17, 137-138. (f) Hoye, T. R.; Kurth, M. J. J. Org. Chem. 1978, 43, 3693-3697. (g) Kato, T.; Ichinose, I. J. Chem. Soc., Perkin Trans. 11980, 1051-1056. (h) Shieh, H.-M.; Prestwich, G. D. Tetrahedron Lett. 1982, 23, 4643-4646. (i) Kato, T.; Mochizuki, M.; Hirano, T.; Fujiwara, S.; Uyehara, T. J. Chem. Soc., Chem. Commun. 1984, 1077-1078. (j) Yamaguchi, Y.; Uyehara, T.; Kato, T. Tetrahedron Lett. 1985, 26, 343-346. (k) Fujiwara, S.; Takeda, K.; Uyehara, T.; Kato, T. Chem. Lett. 1986, 1763-1766. (1) Tanaka, A.; Sato, M.; Yamashita, K. Agric. Biol. Chem. 1990, 54, 121-123. (m) Tanaka, A.; Oritani, T. Biosci. Biotech. Biochem. 1995, 59, 516-517.
(12) (a) Sakakura, A.; Ukai, A.; Ishihara, K. Nature 2007, 445, 900-903. (b) Barluenga, J.; Trincado, M.; Rubio, E.; González, J. M. J. Am. Chem. Soc. 2004, 126, 3416-3417. (c) Barluenga, J.; Alvarez-Pérez, M.; Rodríguez, F.; Fañanás, F. J.; Cuesta, J. A.; García-Granda, S. J. Org. Chem. 2003, 68, 6583-6586.
(13) For selected examples of other halonium-induced cyclization reactions not using terpene-based materials, as well as approaches that form related structures through nucleophilic attack of halogen onto carbon electrophiles, see: (a) Inoue, T.; Kitagawa, O.; Ochiai, O.; Shiro, M.; Taguchi, T. Tetrahedron Lett. 1995, 36, 9333-9336. (b) Cui, X.-L.; Brown, R. S. J. Org. Chem. 2000, 65, 5653-5658. (c) Appelbe, R.; Casey, M.; Dunne, A.; Pascarella, E. Tetrahedron Lett. 2003, 44, 7641-7644. (d) Hajra, S.; Maji, B.; Karmakar, A. Tetrahedron Lett. 2005, 46, 8599-8603. (e) Hanessian, S.; Tremblay, M.; Marzi, M.; Del Valle, J. R. J. Org. Chem. 2005, 70, 5070-5085. (f) Barluenga, J.; Trincado, M.; Rubio, E.; González, J. Angew. Chem. Int. Ed. 2003, 42, 2406-2409. (g) Barluenga, J.; Vázquez-Villa, H.; Ballesteros, A.; González, J. M. J. Am. Chem. Soc. 2003, 125, 9028-9029. (h) Barluenga, J.; González, J. M.; Campos, P. C.; Asensio, G. Angew. Chem. Int. Ed. 1988, 27, 1546-1547. (i) Kang, S. H.; Lee, S. B.; Park, C. M. J. Am. Chem. Soc. 2003, 125, 15748-15749. (j) Haas, J.; Piguel, S.; Wirth, T. Org. Lett. 2002, 4, 297-300. (k) Grossman, R. B.; Trupp, R. J. Can. J. Chem. 1998, 76, 1233-1237. (1) Zhang, W.; Zheng, S.; Liu, N.; Werness, J. B.; Guzei, I. A.; Tang, W. J. Am. Chem. Soc. 2010, 132, 3664-3665.
(14) Part of the challenge may lie in the rapid transfer of bromonium to unreacted alkene, thereby eroding enatioselectivity: (a) Brown, R. S. Acc. Chem. Res. 1997, 30, 131-137; For similar challenges in achieving the enantioselective addition of chalcogens such as sulfur onto alkenes, see: (b) Denmark, S. E.; Collins, W. R.; Cullen, M. D. J. Am. Chem. Soc. 2009, 131, 3490-3492. The same problem may not exist for iodine, and has recently been indicated not to be as profound an issue for chlorine: (c) Denmark, S. E.; Burk, M. T.; Hoover, A. J. J. Am. Chem. Soc. 2010, 132, 1232-1233.
(15) (a) Couladouros, E. A.; Vidali, V. P. Chem. Eur. 12004, 10, 3822-3835. (b) Murai, A.; Abiko, A.; Masamune, T. Tetrahedron Lett. 1984, 25, 4955-4958.
(16) Replacement with iodine is often not completely stereoselective due to competing electrophilic and radical substitution pathways. (a) Jensen, F. R.; Gale, L. H. J. Am. Chem. Soc. 1960, 82, 148-151. (b) DePuy, C. H.; McGirk, R. H. J. Am. Chem. Soc. 1974, 96, 1121-1132.
(17) Snyder, S. A.; Treitler, D. S.; Schall, A. Tetrahedron 2010, 66, 4796-4804.
(18) (a) Su, W.-C.; Fang, J.-M.; Cheng, Y.-S. Tetrahedron Lett. 1995, 36, 5367-5370. (b) Pulicani, J.-P.; Bouchard, H.; Bourzat, J.-V.; Commerçon, A. Tetrahedron Lett. 1994, 35, 9709-9712.
(19) Conceptually, BDSB is an example where a Lewis base has activated a Lewis acid, see: Denmark, S. E.; Beutner, G. L. Angew. Chem. Int. Ed. 2008, 47, 1560-1638.
(20) (a) Bohme, H.; Boll, E. Zeit. Anorg. Alleg. Chem. 1957, 290, 17-23.) Goetz-Grandmont, G. J.; Leroy, M. J. F. J. Chem. Res. (S) 1982, 160-161. (c) Minkwitz, R.; Gerhard, V.; Werner, A. Zeit. Anorg. Alleg. Chem. 1989, 575, 137-144. (d) Askew, H. F.; Gates, P. N.; Muir, A. S. J. Raman Spectroscopy 1991, 22, 265-274. (e) Minkwitz, R.; Baeck, B. Zeit. Naturforschung B: Chem. Sci. 1993, 48, 694-696. (f) Regelmann, B.; Klinkhammer, K. W.; Schmidt, A. Zeit. Anorg. Alleg. Chem. 1997, 623, 1633-1638.
(21) (a) Arsenate anions were not investigated due to their toxicity. (b) The di-t-butyl variant decomposed rapidly, likely due to the weakness of the tertiary carbon-sulfur bond, while both the methyl and isopropyl variants were solid, crystalline materials. In terms of stability, solubility, and cost, however, 13 was superior.
(22) Snyder, S. A.; Treitler, D. S. Organic Syntheses 2010, in press.
(23) 13 is fully soluble at ambient temperature in MeNO₂, EtNO₂, MeCN, DMSO, DMF, and EtOAc, moderately to slightly soluble in CH₂Cl₂, 1,2-dichloroethane, chloroform, and toluene, and insoluble in benzene, hexanes, and pentane. We have observed that 13 is soluble in acetone, methanol, ethanol, and THF, but reacts with these solvents.
(24) Allegra, G.; Wilson, G. E.; Benedetti, E.; Pedone, C.; Albert, R. J. Am. Chem. Soc. 1970, 92, 4002-4007.
(25) Snyder, S. A.; Treitler, D. S. Angew. Chem. Int. Ed. 2009, 48, 7899-7903.
(26) These assessments were made with various terpene-like polyenes possessing different substitution patterns in the terminal alkene position (the typical site of initiation for a cation-π cyclization).
(27) Generally formed in higher amounts on large scale, this side-product could be suppressed by using dilute reaction concentrations (0.01 M) and adding a nitromethane solution of BDSB rapidly to the substrate.
(28) Examples of polyene cyclizations involving Z-alkene geometries to prepare cis-fused decalin systems are in fact quite rare. For the seminal example, see: (a) Smit, W. A.; Semenovzky, A. V.; Kucherov, V. P. Tetrahedron Lett. 1964, 5, 2299-2306. For a more recent example, see: (b) Snowden, R. L.; Eichenberger, J.-C.; Linder, S. M.; Sonnay, C. V.; Schulte-Elte, K. H. J. Org. Chem. 1992, 57, 955-960. In general, such systems are prepared by other methods, including the cyclization of partially cyclized materials, formation of the ring junction using a Friedel-Crafts approach, or post-cyclization modification of a trans-fused system: (c) Saito, A.; Matsushita, H.; Kaneko, H. Chem. Lett. 1984, 591-594. (d) Ishihara, K.; Ishibashi, H.; Yamamoto, H. J. Am. Chem. Soc. 2002, 124, 3647-3655. (e) Bhar, S. S.; Ramana, M. M. V. Tetrahedron Lett. 2006, 47, 7805-7807. (f) Von Schlatter, H.-R.; Lüthy, C.; Graf, W. Helv. Chim. Acta 1974, 57, 1044-1055. (g) Raeppel, F.; Heissler, D. Tetrahedron Lett. 2003, 44, 3487-3488.
(29) (a) Kato, T.; Suzuki, M.; Toyohiko, K.; Moore, B. P. J. Org. Chem. 1980, 45, 1126-1130. (b) Yu, J. S.; Kleckley, T. S.; Wiemer, D. F. Org. Lett. 2005, 7, 4803-4806.
(30) The rate determining transition state for the formation of products 32 and 44 is associated with the first C—C bond formation. The activation barrier leading to 32 was estimated to be approximately 4.3 kcal/mol higher in energy than that leading to 44. Geometries and energetics were obtained from semi-emperical (PM3) calculations using the GAMESS suite of programs. Schmidt, M. W.; Baldridge, K. K.; Boatz, J. A.; Elbert, S. T.; Gordon, M. S.; Jensen, J. H.; Koseki, S.; Matsunaga, N.; Nguyen, K. A.; Su, S. J.; Windus, T. L.; Dupuis, M.; Montgomery, J. A. J. Comput. Chem. 1993, 14, 1347-1363.
(31) We note that the use of a bulkier base such as i-Pr₂NEt afforded inferior regioselectivity in this dehydration step.
(32) The Materials and Methids section contains a complete comparison table. Compound 50 possesses ¹H NMR data that are in excellent agreement with the natural isolate; their ¹³C spectra possess small discrepancies, most of which are 0.5 ppm or less. Efforts based on altering water content as well as adding base or acid never afforded spectra that were perfectly identical in terms of reported values, though such factors can affect the ¹³C NMR spectra of compounds of this type. None of the other isomers synthesized (3, 40, and 45) had ¹H or ¹³C NMR data that even remotely resembled those of the natural product. Prof. Kashman (Tel Aviv University) was contacted in hopes of obtaining either a natural sample or copies of the original physical spectra of peyssonol A, but unfortunately neither could be located, making direct comparison impossible.
(33) The Supporting Information section contains the complete synthetic route employed to access the revised structure of peyssonol A (50), for which further route improvement allowed for the shortening of the sequence described in Scheme 3 by one step to ultimately achieve an overall yield of 14% from (2Z,6E)-farnesol.
(34) (a) Lane, A. L.; Mular, L.; Drenkard, E. J.; Shearer, T. L.; Engel, S.; Fredericq, S.; Fairchild, C. R.; Prudhomme, J.; Le Roch, K.; Hay, M. E.; Aalbersberg, W.; Kubanek, J. Tetrahedron 2010, 66, 455-461. For a review on misassigned natural product structures, see: (b) Nicolaou, K. C.; Snyder, S. A. Angew. Chem. Int. Ed. 2005, 44, 1012-1044. For recent examples of reassignment involving a major architectural change within two fused 6-membered carbon rings, see: (c) Maugel, N.; Mann, F. M.; Hillwig, M. L.; Peters, R. J.; Snider, B. B. Org. Lett. 2010, 12, 2626-2629. (d) Spangler, J. E.; Carson, C. A.; Sorensen, E. J. Chem. Sci. 2010, 1, 202-205.
(35) For an example of an epoxide-induced cyclization leading to such a framework, see: van Tamelen, E. E.; Coates, R. M. Bioorganic Chem. 1982, 11, 171-196.
(36) Specifically, these products should arise if the second ring forms as a boat.
(37) The reaction temperature was essential to preventing lactone formation between the pendant carboxylic acid and the adjacent phenol.
(38) The synthetic sequence produced the fully protonated version of peyssonoic acid A. Initial NMR spectroscopic analysis of synthetic 51, however, did not match that reported for the natural isolate (Ref. 34), primarily around the carboxylic acid residue, aromatic ring, and adjoining methylene group. Subsequent exposure of our material to NaHCO₃ provided the sodium salt of the carboxylate form; spectra obtained from this material fully matched the reported data. See Supporting Information for all relevant spectra and NMR data tables.
(39) (a) Matsuda, H.; Tomiie, Y.; Yamamura, S.; Hirata, Y. J. Chem. Soc., Chem. Commun. 1967, 898-899. (b) Yamamura, S.; Hirata, Y. Bull. Chem. Soc. Jpn. 1971, 44, 2560-2562.
(40) Kato, T.; Kumazawa, S.; Kabuto, C.; Honda, T.; Kitahara, Y. Tetrahedron Lett. 1975, 16, 2319-2322. A total synthesis of aplysin-20 was also achieved via a TBCO-mediated cation-π cyclization of a related starting material, albeit in low yield (cf. Ref. 11i).
(41) IDSI (70) is fully soluble at 25° C. in MeNO₂, EtNO₂, MeCN, CH₂Cl₂, DMSO, DMF, acetone, and EtOAc. It is moderately to slightly soluble in dioxane and CHCl₃, and insoluble in benzene, toluene, and hexanes.
(42) The lengths of the I—Cl bonds within this material are 2.814 Å and 2.714 Å. These values compare favorably to related compounds such as a chlorine-linked NIS-dimer which has very similar bond lengths (2.845 Å and 2.910 Å) and is a source of ICI as well: Ghassenzadeh, M.; Dehnicke, K.; Goesmann, H.; Fenske, D. Zeit. Naturforschung B: Chem. Sci. 1994, 49, 602-608. We note that the average I—Cl bond length is 2.553 Åaccording to the Cambridge Structural Database, version 5.31, 2009. The dimethysulfonium variant of IDSI was reported as a monomeric species: Minkwitz, R.; Prenzel, H. Zeit. Anorg. Alleg. Chem. 1987, 548, 97-102.
(43) See Materials and Methods section for complete details.
(44) IDSI should be viewed as having complementary reactivity to Barluenga's reagent. For instance, electron-rich aromatic rings undergo electrophilic aromatic substitution when pendant monosubstituted double bonds are activated with Ipy₂BF₄; IDSI will not cleanly perform such reactions.
(45) For selected examples of the elimination of alkyl iodides to form alkenes, see: (a) Furber, M.; Kraft-Klaunzer, P.; Mander, L. N.; Pour, M.; Yamauchi, T.; Murofushi, N.; Yamane, H.; Schraudolf, H. Aust. J. Chem. 1995, 48, 427-444. (b) Jin, L.; Nemoto, T.; Nakamura, H.; Hamada, Y. Tetrahedron: Asymmetry 2008, 19, 1106-1113. (c) Eidman, K. F.; MacDougall, B. S. J. Org. Chem. 2006, 71, 9513-9516. (d) Soorukram, D.; Qu, T.; Barrett, A. G. M. Org. Lett. 2008, 10, 3833-3835.
(46) (a) Rouessac, F.; Zamarlik, H.; Gnonlonfoun, N. Tetrahedron Lett. 1983, 24, 2247-2250. For other papers in the series, see: (b) Rouessac, A.; Rouessac, F.; Zamarlik, H. Bull. Chim. Soc. Fr. 1981, 199-203. (c) Zamarlik, H.; Gnonlonfoun, N.; Rouessac, F. Can. J. Chem. 1984, 62, 2326-2329.
(47) (a) White, E. P. New Zealand J. Agric. Res. 1958, 1, 859-865. (b) Hodges, R.; Porte, A. L. Tetrahedron 1964, 20, 1463-1467.
(48) (a) McMurry, J. E.; Erion, M. D. J. Am. Chem. Soc. 1985, 107, 2712-2720. (b) Erion, M. D.; McMurry, J. E. Tetrahedron Lett. 1985, 26, 559-562.
(49) Kaise, H.; Shinohara, M.; Miyazaki, W.; Izawa, T.; Nakano, Y.; Sugawara, M.; Sugawara, K. J. Chem. Soc., Chem. Commun. 1979, 726-727.
(50) For an alternate total synthesis of K-76, see: Mori, K.; Komatsu, M. Liebigs Ann. Chem. 1988, 107-119. This route did not utilize a cation-cyclization, but used the same key intermediate, compound 96, to achieve their synthesis. Compound 96 was accessed in 9 steps and 2.5% overall yield from commercial materials.
(51) Corey, E. J.; Tius, M. A.; Das, J. J. Am. Chem. Soc. 1980, 102, 7612-7613.
(52) Manchand, P. S.; White, J. D.; Wright, H.; Clardy, J. J. Am. Chem. Soc. 1973, 95, 2705-2706.
(53) The Corey group also used a similar route to prepare the natural product K-76: Corey, E. J.; Das, J. J. Am. Chem. Soc. 1982, 104, 5551-5553.
(54) Meerwein, H.; Zenner, K.-F. Gipp, R. Justus Liebigs Ann. Chem. 1965, 67-77.
(55) Several experimental studies (pioneered by the Olah group) have postulated that some halonium ions, especially chloronium ions, exist not as traditional 3-membered rings, but as “open” carbocations, or perhaps as an equilibrium between the two species. This appears especially true when one or more of the carbons of the potential chloronium ion is tertiary: (a) Olah, G. A.; Westerman, P. W.; Melby, E. G.; Mo, Y. K. J. Am. Chem. Soc. 1974, 96, 3565-3573. (b) Olah, G. A.; Bollinger, J. M.; Mo, Y. K.; Brinich, J. M. J. Am. Chem. Soc. 1972, 94, 1164-1168. (c) Olah, G. A.; Bollinger, J. M. J. Am. Chem. Soc. 1968, 90, 947-953. (d) Berman, D. W.; Anicich, V.; Beauchamp, J. L. J. Am. Chem. Soc. 1979, 101, 1239-1248. A more recent NMR analysis of tetrasubstituted chloronium ions indicates that open structures predominate: (e) Ohta, B. K.; Hough, R. E.; Schubert, J. W. Org. Lett. 2007, 9, 2317-2320. Theoretical studies also corroborate that the open carbocation is favored in the specific case of the trisubstituted chloronium ion: (f) Yamabe, S.; Tsuji, T.; Hirao, K. Chem. Phys. Lett. 1988, 146, 236-242. As noted by a referee, a plausible alternative explanation for the lack of stereocontrol in chloronium ion cyclizations is reaction through both chair/chair and boat/chair conformations, perhaps owing to the higher reactivity and electrophilicity of the chloronium ion electrophile compared to the bromonium and iodonium counterparts.
(56) C₂-symmetric ligands were chosen due to our previous success in utilizing them for asymmetric Hg(II)-induced cyclizations (cf. Ref. 17).
(57) (a) Julienne, K.; Metzner, P. J. Org. Chem. 1998, 63, 4532-4534. (b) Braun, W.; Calmuschi, B.; Haberland, J.; Hummel, W.; Liese, A.; Nickel, T.; Steizer, O, Salzer, A. Eur. J. Inorg. Chem. 2004, 11, 2235-2243.
(58) For a recent total synthesis featuring an enantioselective chlorination of an isolated alkene, see: Snyder, S. A.; Tang, Z.-Y.; Gupta, R. J. Am. Chem. Soc. 2009, 131, 5744-5745.
(59) Weber, J. F. F.; Wahab, I. A.; Marzuki, A.; Thomas, N. A.; Kadir, A. A.; Hadi A. H. A.; Awang, K.; Latiff, A. A.; Richomme, P.; Delaunay, J. Tetrahedron Lett. 2001, 42, 4895-4897.
(60) Aznam, S.; Arianingrum, R. Takaya, Y.; Masalak, N. Journal of Physical Science 2008, 19, 7-21.
(61) Gribble, G. N. Acc. Chem. Res. 1998, 31, 141.
(62) Irie, T.; Suzuki, M.; Masamune, T. Tetrahedron Lett. 1965, 1091.
(63) For total and formal syntheses of 1, see: (a) Masamune, T.; Murase, H.; Matsue, H.; Murai, A. Bull. Chem. Soc. Jpn. 1979, 52, 135. (b) Tsushima, K.; Murai, A. Tetrahedron Lett. 1992, 33, 4345. (c) Bratz, M.; Bullock, W. H.; Overman, L. E.; Takemoto, T. J. Am. Chem. Soc. 1995, 117, 5958. (d) Mujica, M. T.; Afonso, M. M.; Galindo, A.; Palenzuela, J. A. Synlett 1996, 983. (e) Burton, J. W.; Clark, J. S.; Derrer, S.; Stork, T. C.; Bendall, J. G.; Holmes, A. B. J. Am. Chem. Soc. 1997, 119, 7483. (f) Krüger, J.; Hoffmann, R. W. J. Am. Chem. Soc. 1997, 119, 7499. (g) Crimmins, M. T.; Choy, A. L. J. Am. Chem. Soc. 1999, 121, 5653. (h) Crimmins, M. T.; Emmitte, K. A. Org. Lett. 1999, 1, 2029. (i) Kadota, I.; Uyehara, H.; Yamamoto, Y. Tetrahedron 2004, 60, 7361. (j) Baek, S.; Jo, H.; Kim, H.; Kim, H.; Kim, S.; Kim, D. Org. Lett. 2005, 7, 75. (k) Fujiwara, K.; Yoshimoto, S.; Takizawa, A.; Souma, S.-I.; Mishima, H.; Murai, A.; Kawai, H.; Suzuki, T. Tetrahedron Lett. 2005, 46, 6819. (1) Adsool, V. A.; Pansare, S. V. Org. Biomol. Chem. 2008, 6, 2011. (m) Ortega, N.; Martín, V. S.; Martín, T. J. Org. Chem. 2010, 75, 6660.
(64) (a) Moore, R. E. “Algal Nonisoprenoids” in Marine Natural Products, Chemical and Biological Perspectives (Vol. 1), Scheuer, P. J. (Ed.), Academic Press: New York, 1978, pp. 43-124. (b) Erickson, K. L. “Constituents of Laurencia” in Marine Natural Products, Chemical and Biological Perspectives (Vol. 5), Scheuer, P. J. (Ed.), Academic Press: New York, 1983, pp. 131-257. (c) Blunt, J. W.; Copp, B. R.; Munro, M. H. G.; Northcote, P. T.; Prinsep, M. R. Nat. Prod. Rep. 2011, 28, 196 and earlier reviews in this series.
(65) (a) Murai, A. “Biosynthesis of Cyclic Bromoethers from Red Algae” in Comprehensive Natural Products Chemistry (Vol. 1), Sankawa, U. (Ed), Elsevier: New York, 1999, pp. 303-324. (b) Fukuzawa, A.; Aye, M.; Takasugi, Y.; Nakamura, M.; Tamura, M.; Murai, A. Chem. Lett. 1994, 2307.
(66) For comments on the highly unfavorable entropic and enthalpic factors involved in medium ring formation, see: Illuminati, G.; Mandolini, L. Acc. Chem. Res. 1981, 14, 95.
(67) The epoxide derivative of 7 is, in fact, a natural product: Fukuzawa, A.; Aye, M.; Takaya, Y.; Fukui, H.; Masamune, T.; Murai, A. Tetrahedron Lett. 1989, 30, 3665.
(68) Other examples of ring-expanding halocyclization are known, although none convert a tetrahydrofuran to an oxocane: (a) Fujioka, H.; Kitagawa, H.; Nagatomi, Y.; Kita, Y. J. Org. Chem. 1996, 61, 7309. (b) Lakshmipathi, P.; Grée, D.; Grée, R. Org. Lett. 2002, 4, 451. (c) Bravo, F.; McDonald, F. E.; Neiwert, W. A.; Hardcastle, K. I. Org. Lett. 2004, 6, 4487. (d) Tanuwidjaja, J.; Ng, S.-S.; Jamison, T. F. J. Am. Chem. Soc. 2009, 131, 12084.
(69) Examples of tetrahydrofuran ring-expansions to oxocanes and oxocenes are known, but none have been initiated by halonium electrophiles, see: (a) Kamada, T.; Ge-Qing; Abe, M.; Oku, A. J. Chem. Soc., Perkin Trans. 1 1996, 413. (b) Mukai, C.; Yamashita, H.; Ichiryu, T.; Hanaoka, M. Tetrahedron 2000, 56, 2203. (c) Sakamoto, Y.; Tamegai, K.; Nakata, T. Org. Lett. 2002, 4, 675. (d) Li, J.; Suh, J. M.; Chin, E. Org. Lett. 2010, 12, 4712.
(70) (a) Braddock, D. C. Org. Lett. 2006, 8, 6055. (b) Braddock, D. C.; Millan, D. S.; Perez-Fuertes, Y.; Pouwer, R. H.; Sheppard, R. N.; Solanki, S.; White, A. J. P. J. Org. Chem. 2009, 74, 1835.
(71) The marilzabicycloallenes were isolated soon after Braddock's work was published: Gutiérrez-Cepeda, A.; Fernández, J. J.; Norte, M.; Souto, M. L. Org. Lett. 2011, 13, 2690.
(72) Kim, B.; Lee, M.; Kim, M. J.; Lee, H.; Kim, S.; Kim, D.; Koh, M.; Park, S. B.; Shin, K. J. J. Am. Chem. Soc. 2008, 130, 16807.
(73) For an example of ring-opening of a cyclic oxonium towards a different cyclic ether natural product, one which precedes the work of both Braddock and Kim (Refs. 10 and 12), see: Clark, J. S.; Wong, Y.-S. Chem. Commun. 2000, 1079.
(74) This comment excludes enzymatic syntheses (see Ref. 5). For a review, see: (a) Fujiwara, K. “Total Synthesis of Medium-Ring Ethers from Laurencia Red Algae” in Topics in Heterocyclic Chemistry (Vol. 5), Kiyota, H. (Ed.), Springer-Verlag: Berlin, 2006, pp. 97-148. Additional syntheses: (b) Overman, L. E.; Thompson, A. S. J. Am. Chem. Soc. 1988, 110, 2248. (c) Crimmins, M. T.; Tabet, E. A. J. Am. Chem. Soc. 2000, 122, 5473. (d) Fujiwaraa, K.; Souma, S.-I.; Mishima, H.; Murai, A. Synlett 2002, 1493. (e) Boeckman Jr., R. K.; Zhang, J.; Reeder, M. R. Org. Lett. 2002, 4, 3891. (f) Saitoh, T.; Suzuki, T.; Sugimoto, M.; Hagiwara, H.; Hoshi, T. Tetrahedron Lett. 2003, 44, 3175. (g) Kim, H.; Choi, W. J.; Jung, J.; Kim, S.; Kim, D. J. Am. Chem. Soc. 2003, 125, 10238. (h) Kim, B.; Cheon, G.; Park, J.; Lee, H.; Kim, H.; Kim, S.; Kim, D. Heterocycles 2007, 74, 171. (i) Sasaki, M.; Hashimoto, A.; Tanaka, K.; Kawahata, M.; Yamaguchi, K.; Takeda, K. Org. Lett. 2008, 10, 1803. (j) Sasaki, M.; Oyamada, K.; Takeda, K. J. Org. Chem. 2010, 75, 3941. (k) Kim, B.; Sohn, T.-I.; Kim, S.; Kim, D.; Lee, J. Heterocycles 2011, 82, 1113.
(75) 8-membered bromoetherifications are rare, with low or no yield unless rigidifying elements are present; even then, the cyclizations are often non-diastereoselective: (a) Brunel, Y.; Rousseau, G. J. Org. Chem. 1996, 61, 5793. (b) Rousseau, G.; Homsi, F. Chem. Soc. Rev. 1997, 26, 453. (c) Mendes, C.; Renard, S.; Rofoo, M.; Roux, M.-C.; Rousseau, G. Eur. J. Org. Chem. 2003, 463.
(76) (a) Snyder, S. A.; Treitler, D. S. Angew. Chem. Int. Ed. 2009, 48, 7899. (b) Snyder, S. A.; Treitler, D. S.; Brucks, A. P. J. Am. Chem. Soc. 2010, 132, 14303. (c) Snyder, S. A.; Treitler, D. S. Org. Synth. 2011, 88, 54.
(77) This mechanism has been postulated for similar rearrangements at least twice before: (a) Sugimoto, M.; Suzuki, T.; Hagiwara, H.; Hoshi, T. Tetrahedron Lett. 2007, 48, 1109. (b) See Ref. 12.
(78) Such neighboring-group participation was observed in analogous transannular systems by Braddock (Ref. 10b) and Clark (Ref. 13). For a recent example of an intermolecular opening of a cyclic oxonium ion, see: Mascal, M.; Hafezi, N.; Toney, M. D. J. Am. Chem. Soc. 2010, 132, 10662.
(79) Carling, R. W.; Holmes, A. B. J. Chem. Soc., Chem. Commun. 1986, 565.
(80) The Woodward-Brutcher modification of the Prévost reaction provides a mechanism for this neighboring group participation: Wiberg, K. B.; Saegebarth, K. A. J. Am. Chem. Soc. 1957, 79, 6256.
(81) (a) Kang, B.; Mowat, J.; Pinter, T.; Britton, R. Org. Lett. 2009, 11, 1717. (b) Kang, B.; Chang, S.; Decker, S.; Britton, R. Org. Lett. 2010, 12, 1716.
(82) Bromonium ion transfer has been extensively studied: (a) Bennet, A. J.; Brown, R. S.; McClung, R. E. D.; Klobukowski, M.; Aarts, G. H. M.; Santarsiero, B. D.; Bellucci, G.; Bianchini, R. J. Am. Chem. Soc. 1991, 113, 8532. (b) Rodebaugh, R.; Fraser-Reid, B. Tetrahedron 1996, 52, 7663. (c) Denmark, S. E.; Burk, M. T.; Hoover, A. J. J. Am. Chem. Soc. 2010, 132, 1232.
(83) Intramolecular benzoate transfers are known to be less facile: Haines, A. H. Adv. Carbohydr. Chem. Biochem. 1976, 33, 11.
(84) Ahmad, S. M.; Braddock, D. C.; Cansell, G.; Hermitage, S. A. Tetrahedron Lett. 2007, 48, 915.
(85) Bedford, S. B.; Bell, K. E.; Fenton, G.; Hayes, C. J.; Knight, D. W.; Shaw, D. Tetrahedron Lett. 1992, 33, 6511.
(86) Snyder, S. A.; Treitler, D. S. Angew. Chem. Int. Ed. 2009, 48, 7899-7903.
(87) Reddy, D. S. Org. Lett. 2004, 6, 3345-3347.
(88) Kang, B.; Mowat, J.; Pinter, T.; Britton, R. Org. Lett. 2009, 11, 1717-1720.
(89) Kang, B.; Chang, S.; Decker, S.; Britton, R. Org. Lett. 2010, 12, 1716-1719.
(90) McDougal, P. G.; Rico, J. G.; Oh, Y.-I.; Condon, B. D. J. Org. Chem. 1986, 51, 3388-3390.
(91) Shakhmaev, R. N.; Ishbaeva, A. U.; Shayakhmetova, I. S. Russ. J. Gen. Chem. 2009, 79, 1171-1174.
(92) Uyanik, M.; Ishihara, K.; Yamamoto, H. Org. Lett. 2006, 8, 5649-5652.
(93) Schmidt, B. J. Org. Chem. 2004, 69, 7672-7687.
(94) Wang, P.; Zhu, J.; Yuan, Y.; Danishefsky, S. J. J. Am. Chem. Soc. 2009, 131, 16669-16671.

1-122. (canceled)

123. A compound having the structure:

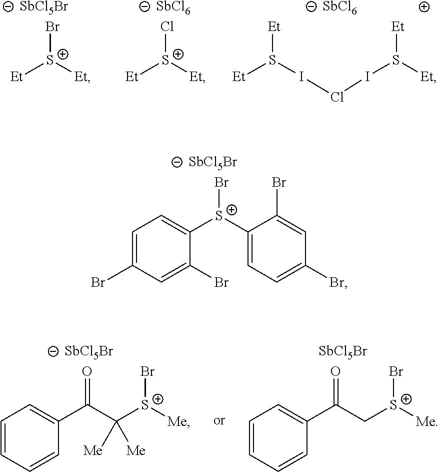

124. A process for cyclizing an alkene or halogenating an aromatic ring comprising contacting the alkene or aromatic ring with the compound of claim 123 under conditions permitting cyclization of the alkene or halogenation of the aromatic ring.

125. The process of claim 124, wherein the alkene is a polyene, alkenoic acid or alkenyl alkyl ether.

126. The process of claim 124, wherein the cyclization is a ring-forming halolactonization or ring-expanding bromoetherification.

127. The process of claim 124, wherein the halogenation is a mono-halogenation and the aromatic ring is a substituted phenyl.

128. A process for producing the compound of claim 123 having the structure: 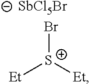
comprising contacting Br, with excess Et₂S and SbCl₅ in a suitable solvent at a suitable temperature so as to thereby produce the compound; or
having the structure: 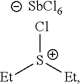
comprising contacting Cl₂ with Et₂S and SbCl₅ in a suitable solvent at a first suitable temperature, and subsequently contacting the resulting product with hexanes prior to cooling to a second suitable temperature so as to thereby produce the compound; or
having the structure: 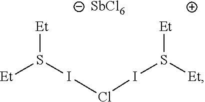
comprising contacting I₂ with excess Et₂S and SbCl₅ in a suitable solvent at a first suitable temperature, warming the resulting product, and subsequently contacting the resulting product with hexanes at a second suitable temperature so as to thereby produce the compound.

129. A compound having the structure: 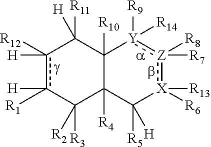
wherein
Y and X are, independently, a C atom or an O atom,
wherein when X is O, R₆ and R₁₃ are absent and when Y is O, R₉ and R₁, are absent;
Z is a carbon atom;
α, β, and γ are, independently, present or absent, and when present each is a bond;
R₁ is OH or a halogen, or is absent if bond γ is present;
R₂, R₃, R₅, R₁₀, R₁₁ and R₁₂ are, independently, H, OH or a C₁₋₄ alkyl;
R₄ is H, OH or a C_{I}, alkyl;
R₆ is H, OH or a C₄ alkyl, or R₈ with R₇ forms a substituted aryl;
R₇ is H, OH, a C₁₋₄ alkyl, or R₇ with R₈ forms a ═CH₂, or R₇ with R₈ forms a ═O, or R₇ is absent when (a) R₈ is joined to R₉ to form a substituted aryl or unsubstituted aryl, or (b) bond α is present;
R₈ is H, OH or a C₁₋₄ alkyl, or R₈ with R₉ forms a substituted oxane, or R₆ with R₉ forms a substituted dioxane, or R₈ with R₉ forms a substituted aryl or an unsubstituted aryl, or R₈ with R₉ forms the structure: 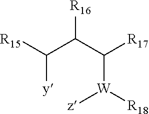
wherein W is a C atom or an O atom, and when W is an O atom, R₁₈ is absent;
wherein end y′ is bonded to atom Y and end z′ is bonded to atom Z and
wherein when W is a C atom, R₁₅ and R₁₆ are each, independently, H or OH, and R₁₇ and R₁₈ are each, independently, H or OH, or R₁₇ and R₁₈ together form a substituted or unsubstituted aryl,
and wherein when W is an O atom R₁₅ is H or OH, and wherein R₁₆ and R₁₇ are, independently, H or OH, or R₁₆ and R₁₇ together form a substituted aryl or unsubstituted aryl;
R₉ is H, —CHO, —CH₂OAc, —C(═O)(OEt) or —C(═O)(OMe),
wherein R₁₉ is a substituted aryl or an unsubstituted aryl;
R₁₂ is H, OH or a C₁₋₄ alkyl, or is absent if bond γ is present;
R₁₃ is H or, is absent when (a) R₆ is joined to R₇ to form a substituted aryl or (b) bond β is present;
R₁₄ is H or, is absent when (a) R₉ is joined to R₈ to form a substituted aryl or unsubstituted aryl, or (b) Y is an O atom, or (c) bond α is present;
wherein bond α is only present if bond γ is present and R₉ is —C(═O)(OEt) or —C(═O)(OMe), and Y and Z are each a C atom;
wherein bond β is only present if bonds α and γ are absent and R₉ is —C(═O)(OEt) or —C(═O)(OMe), and Z and X are carbon atoms, and R₇ together with R₈ is other than ═O;
wherein when Y and X are C atoms, R₁ is Br, R₂, R₃ and R₁₀ are CH₃, R₄, R₈, R₆, R₁₁, R₁₂, R₁₃ and R₁₄ are H, R₇ and R₈ form a ═CH₂, and R₉ is —CH₂—R₁₉ with R₁₉ having the structure: 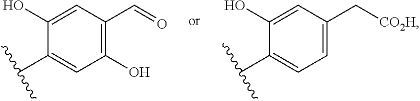 [SMILES: *c1cc(O)c(C=O)cc1O ; "or" ; *c1ccc(CC(=O)O)cc1O]
then R₉ and R₁₀ have the following stereochemistry: 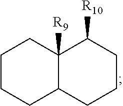
wherein when Y and X are C atoms, R₁ is Br, R₂, R₃, R₈ and R₁₀ are CH₃, R₄, R₅, R₆, R₁₁, R₁₂, R₁₃ and R₁₄ are H, and R₇ is OH, then R₉ is other than —C₂H₄C(CH₃)(CHCH₂OH);
wherein when R₉ is —C(═O)(OMe) and bonds α, β, and γ are absent, and R₇ and R₈ together from ═O, then R₁ is other than I, or a pharmaceutically acceptable salt thereof; or
a composition comprising a compound having the structure: 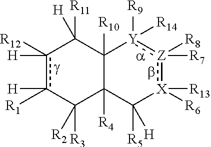
wherein
Y and X are, independently, a C atom or an O atom,
wherein when X is O, R₆ and R₁₃ are absent and when Y is O, R₉ and R₁₄ are absent;
Z is a carbon atom;
α, β, and γ are, independently, present or absent, and when present each is a bond;
R₁ is OH, CH₃ or a halogen, or is absent if bond γ is present;
R₂, R₂, R₅, R₁₀, R₁₁ and R₁₂ are, independently, H, OH or a C₁₋₄ alkyl;
R₄ is H, OH or a C₁₋₄ alkyl;
R₆ is H, OH or a C₁₋₄ alkyl, or R₆ with R₇ forms a substituted aryl;
R₇ is H, OH, a C₁₋₄ alkyl, or R₇ with R₈ forms a ═CH, or R₇ with R₈ forms a ═O, or R₇ is absent when (a) Rₐ is joined to R₉ to form a substituted aryl or unsubstituted aryl or (b) bond α is present;
R₈ is H, OH or a C₁₋₄ alkyl, or R₈ with R₉ forms a substituted oxane, or R₈ with R₉ forms a substituted dioxane, or R₆ with R₉ forms a substituted aryl or an unsubstituted aryl, or R₈ with R₉ forms the structure: 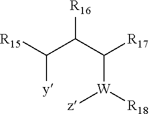
wherein W is a C atom or an O atom, and when W is an O atom, R₁₈ is absent;
wherein end y′ is bonded to atom Y and end z′ is bonded to atom Z and
wherein when W is a C atom, R₁₅ and R¹⁶ are each, independently, H, or OH, and R₁₇ and R₁₆ are each, independently, H, or OH, or R₁₇ and R₁₈ together form a substituted or unsubstituted aryl,
and wherein when W is an O atom R₁₅ is H, or OH, and wherein R₁₆ and R₁₇ are, independently, H, or OH, or R₁₆ and R₁₇ together form a substituted aryl or unsubstituted aryl;
R₉ is H, —CHO, —CH₂OAc, —C(═O)(OEt) or —C(═O)(OMe),
wherein R₁₉ is a substituted aryl or an unsubstituted aryl;
R₁₂ is H, OH or a C₁₋₄ alkyl, or is absent if bond γ is present;
R₁₃ is H or, is absent when (a) R₆ is joined to R₇ to form a substituted aryl or (b) bond β is present;
R₁₄ is H or, is absent when (a) R₉ is joined to R₈ to form a substituted aryl or unsubstituted aryl, or (b) Y is an O atom, or (c) bond α is present;
wherein bond α is only present if bond γ is present and R₉ is —C(═O)(OEt) or —C(═O)(OMe), and Y and Z are carbon atoms;
wherein bond β is only present if bonds α and γ are absent and R₉ is —C(═O)(OEt) or —C(═O)(OMe), and Z and X are carbon atoms, and R₇ together with R₈ is other than ═O;
wherein when Y and X are C atoms, R₁ is Br, R₂, R₃, R₈ and R₁₀ are CH₃, R₄, R₅, R₆, R₁₁, R₁₂, R₁₃ and R₁₄ are H, R₇ is OH then R₉ is other than —C₂H₄C(CH₃)(CHCH₂OH), or a pharmaceutically acceptable salt thereof,
wherein the composition is free of plant extract.

130. The compound of claim 129 having the structure:

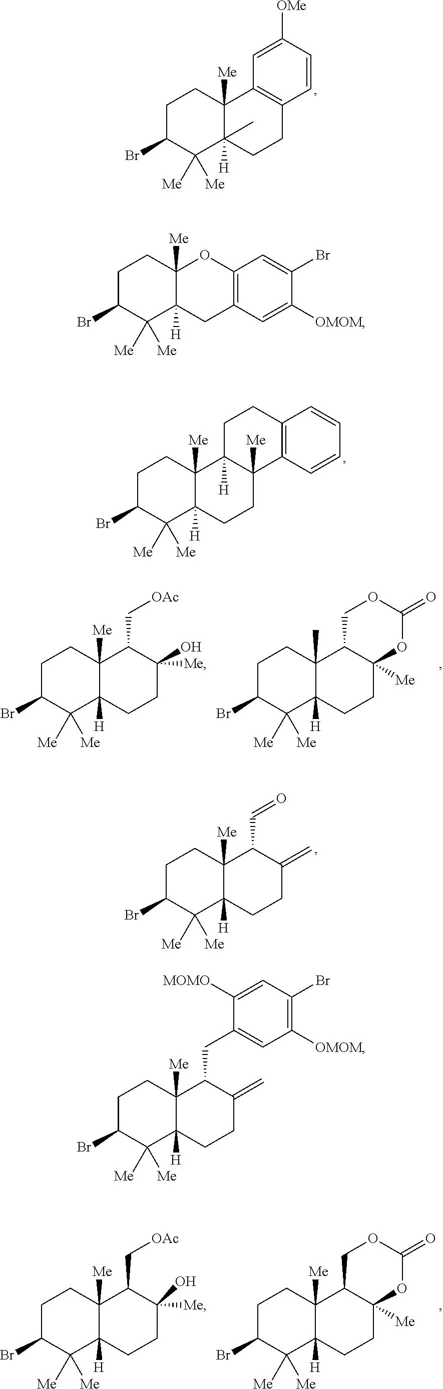

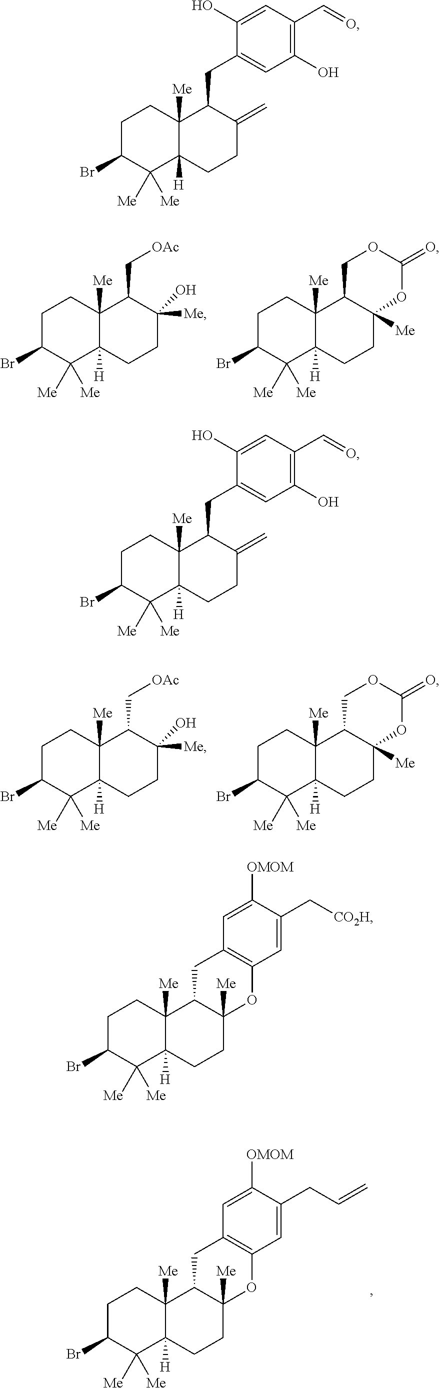

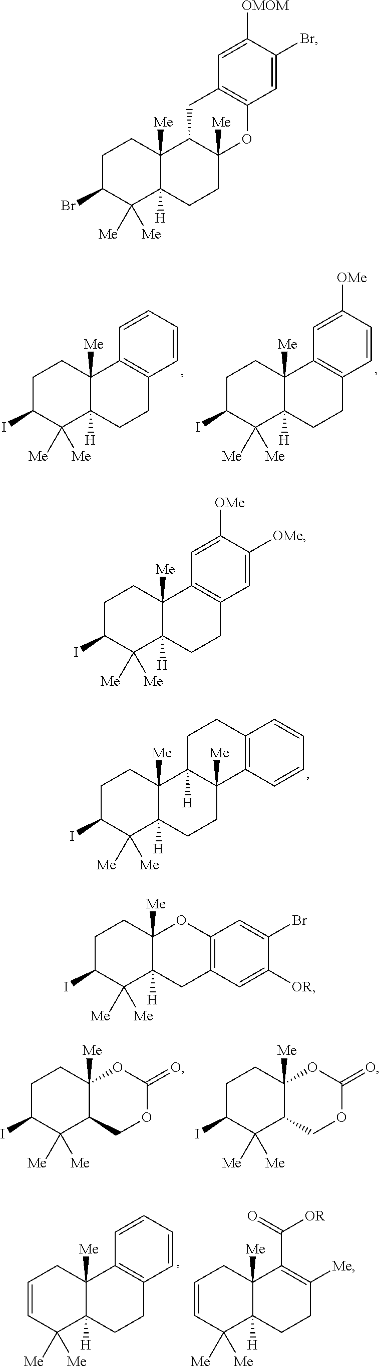

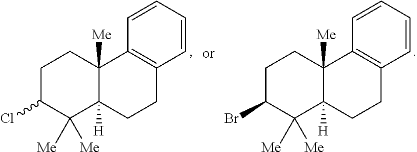 [SMILES: CC1(C)C(Cl)CC[C@]2(C)c3ccccc3CC[C@@H]12 ; "," ; CC1(C)[C@@H](Br)CC[C@]2(C)c3ccccc3CC[C@@H]12]

131. The composition of claim 129 comprising the compound having the structure:

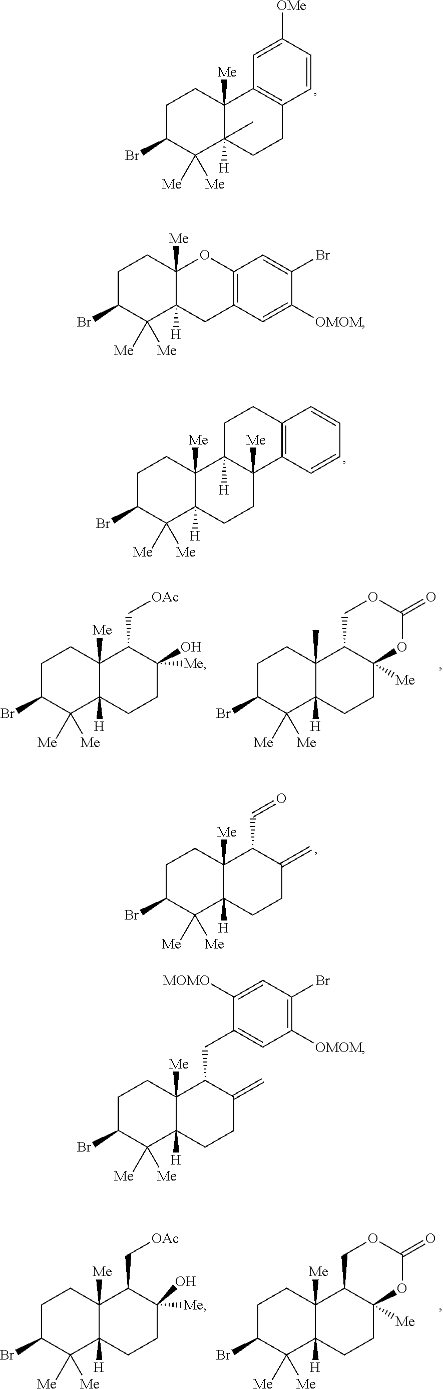

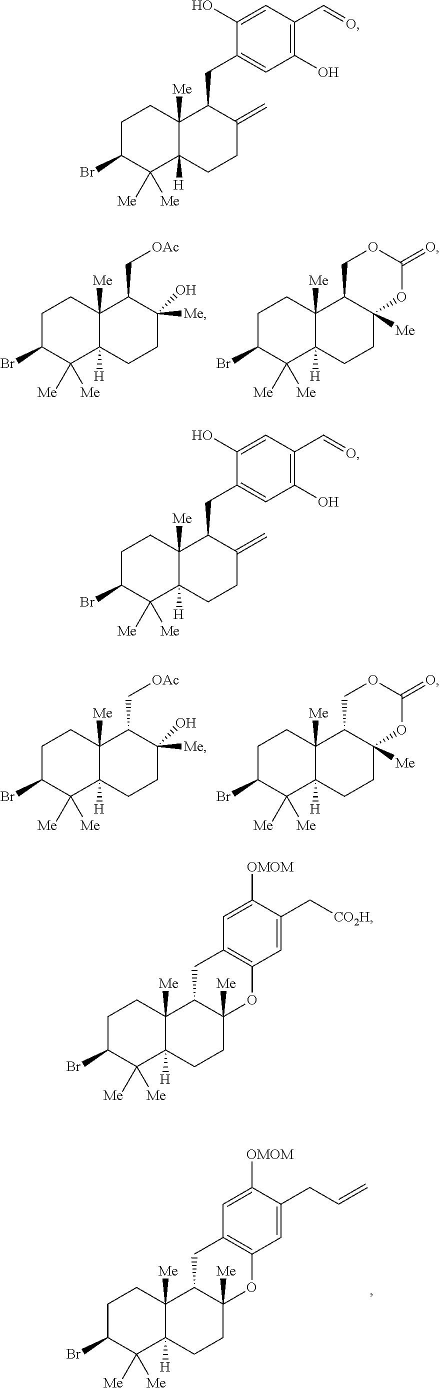

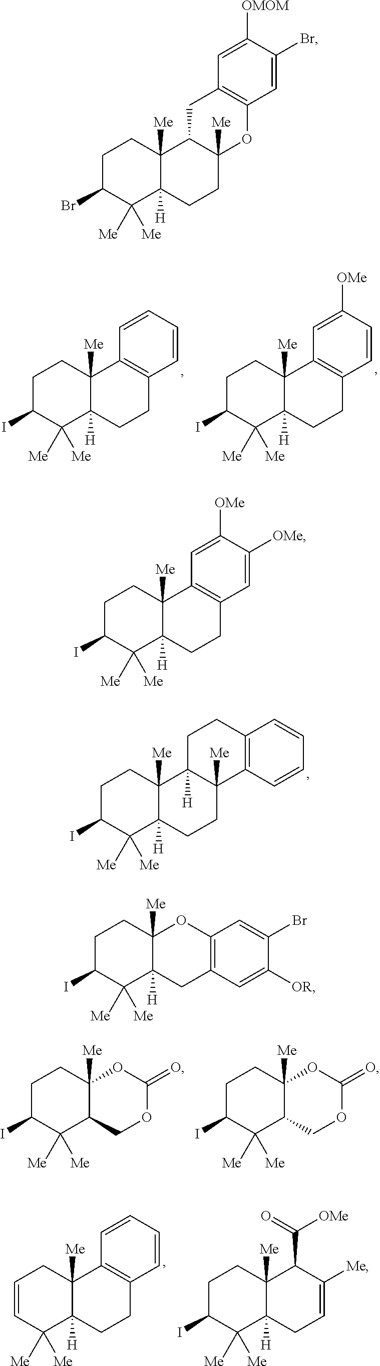

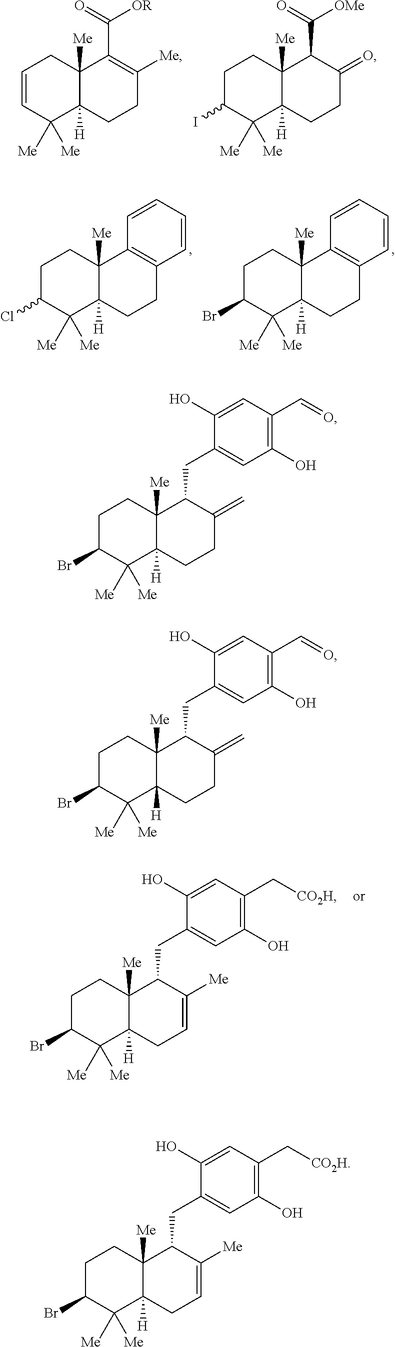

132. A process for producing the compound of claim 129 comprising reacting a polyene having the structure: 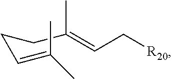
wherein R₂₀ is —CN, an ether, an ester, an acetate, OH, C₁₋₆ alkyl, C₂₋₆ alkenyl, a ketone, an ester, cycloalkyl, cycloalkenyl, a substituted or unsubstituted aryl, a substituted or unsubstituted heteroaryl, a substituted or unsubstituted heterocyclyl,
wherein each occurrence of alkyl, alkenyl, cycloalkyl, and cycloalkenyl is substituted or unsubstituted,
with a second compound having the structure: 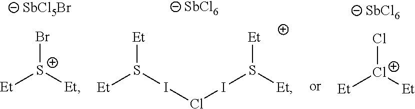
in a suitable solvent at a suitable temperature so as to thereby produce the compound,
wherein R₂₀ is 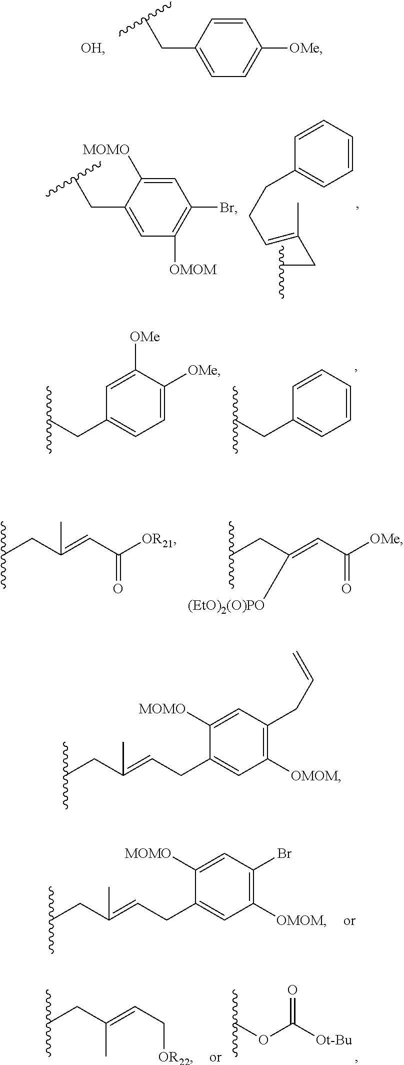
wherein R₂₁ is CH₃ or C₂H₃, and R₂₂ is H, Ac or Boc.

133. A process for producing the composition of claim 129 comprising:
a) reacting a polyene having the structure: 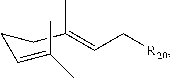
wherein R₂₀ is —CN, an ether, an ester, an acetate, OH, C₁₋₆ alkyl, C₂₋₆ alkenyl, a ketone, an ester, cycloalkyl, cycloalkenyl, a substituted or unsubstituted aryl, a substituted or unsubstituted heteroaryl, a substituted or unsubstituted heterocyclyl,
wherein each occurrence of alkyl, alkenyl, cycloalkyl, and cycloalkenyl is substituted or unsubstituted,
with a second compound having the structure: 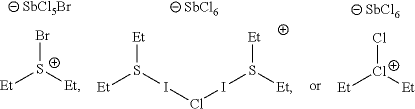
in a suitable solvent at a suitable temperature so as to thereby produce the compound of the composition; and
b) admixing the product of step a) with a carrier so as to thereby produce the composition,
wherein R₂₀ is: 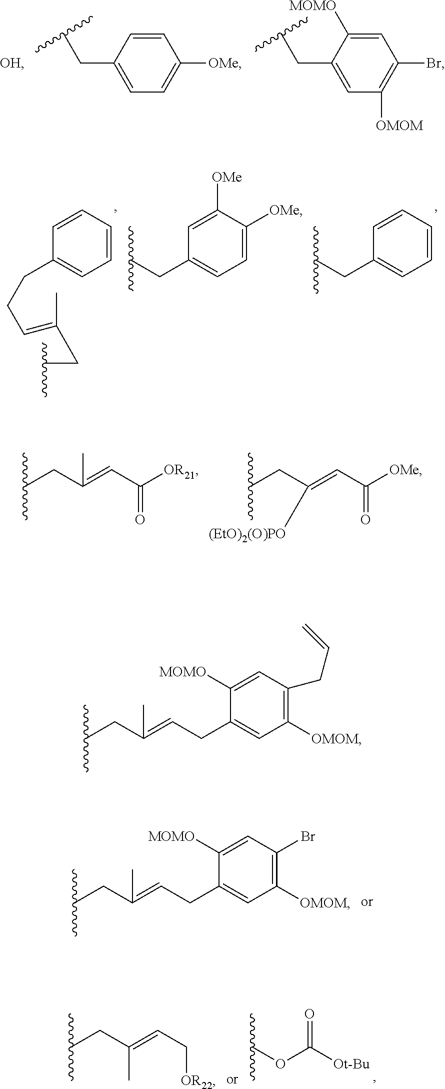
wherein R₂₁ is CH₃ or C₂H₃ and R₂₂ is H, Ac or Boc.

134. A compound having the structure: 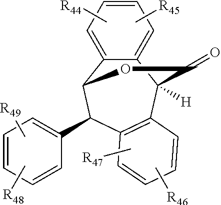
wherein
R₄₄, R₄₅, R₄₆, R₄₇, R₄₈, and R₄₉ are independently H, CN, acetate, OH, OR₅₀, a substituted or unsubstituted C₁₋₆ alkyl, a substituted or unsubstituted C₂₋₆ alkenyl, a ketone, an ester, a substituted or unsubstituted cycloalkyl, a substituted or unsubstituted cycloalkenyl, a substituted or unsubstituted aryl, a substituted or unsubstituted heteroaryl, a substituted or unsubstituted heterocyclyl,
wherein each occurrence of R₅₀ is independently H, methyl, substituted or unsubstituted alkyl, substituted or unsubstituted heteroalkyl substituted or unsubstituted aryl, substituted or unsubstituted heteroaryl, phosphate, sulfate, sulfonic ester, or ester,
or a pharmaceutically acceptable salt thereof; or
a composition comprising a compound having the structure: 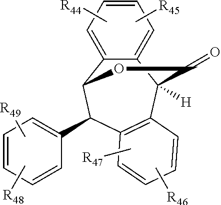
wherein
R₄₄, R₄₅, R₄₆, R₄₇, R₄₈, and R₄₉ are independently H, CN, acetate, OH, OR₅₀, a substituted or unsubstituted C₁₋₆ alkyl, a substituted or unsubstituted C₂₋₆ alkenyl, a ketone, an ester, a substituted or unsubstituted cycloalkyl, a substituted or unsubstituted cycloalkenyl, a substituted or unsubstituted aryl, a substituted or unsubstituted heteroaryl, a substituted or unsubstituted heterocyclyl,
wherein each occurrence of R₅₀ is independently H, methyl, substituted or unsubstituted alkyl, substituted or unsubstituted heteroalkyl substituted or unsubstituted aryl, substituted or unsubstituted heteroaryl, phosphate, sulfate, sulfonic ester, or ester,
or a pharmaceutically acceptable salt thereof,
wherein the composition is free of plant extract.

135. The compound or composition of claim 134, wherein the compound has the structure:

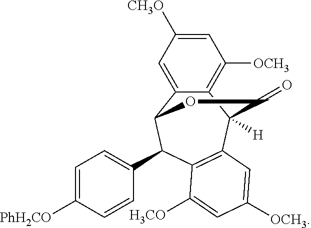

136. A process for producing the compound or composition of claim 134 comprising reacting an alkenoic acid having the structure: 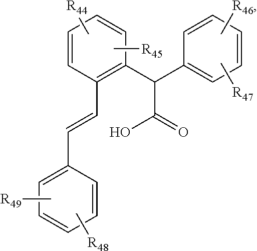
wherein
R₄₄, R₄₅, R₄₆, R₄₇, R₄₈, and R₄₉ are independently H, CN, acetate, OH, OR, a substituted or unsubstituted C₁₋₆ alkyl, a substituted or unsubstituted C₂₋₆ alkenyl, a ketone, an ester, a substituted or unsubstituted cycloalkyl, a substituted or unsubstituted cycloalkenyl, a substituted or unsubstituted aryl, a substituted or unsubstituted heteroaryl, a substituted or unsubstituted heterocyclyl,
wherein each occurrence of R₅₀ is independently H, methyl, substituted or unsubstituted alkyl, substituted or unsubstituted heteroalkyl substituted or unsubstituted aryl, substituted or unsubstituted heteroaryl, phosphate, sulfate, sulfonic ester, or ester,
with a second compound having the structure: 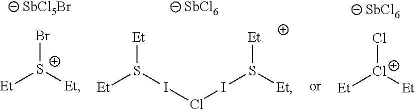
in a suitable solvent at a suitable temperature so as to thereby produce the compound; or
a) reacting an alkenoic acid having the structure: 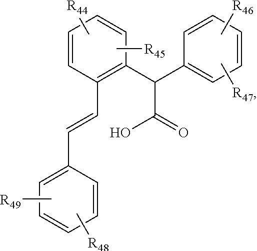
wherein
R₄₄, R₄₅, R₄₆, R₄₇, R₄₈, and R₄₉ are independently H, CN, acetate, OH, OR₅₀, a substituted or unsubstituted alkyl, a substituted or unsubstituted C₂₋₆ alkenyl, a ketone, an ester, a substituted or unsubstituted cycloalkyl, a substituted or unsubstituted cycloalkenyl, a substituted or unsubstituted aryl, a substituted or unsubstituted heteroaryl, a substituted or unsubstituted heterocyclyl,
wherein each occurrence of R₅₀ is independently H, methyl, substituted or unsubstituted alkyl, substituted or unsubstituted heteroalkyl substituted or unsubstituted aryl, substituted or unsubstituted heteroaryl, phosphate, sulfate, sulfonic ester, or ester,
with a second compound having the structure: 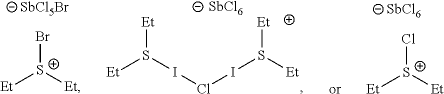
in a suitable solvent at a suitable temperature so as to thereby produce the compound of the composition; and
b) admixing the product of the step a) with a carrier so as to thereby produce the composition.

137. A compound having the structure: 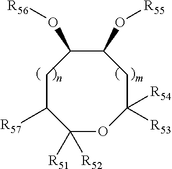
wherein
n=1 or 2;
m=1 or 2;
R₅₁, R₅₂, R₅₃ and R₅₄ are independently H, alkyl, or a haloalkyl;
R₅₅ and R₅₆ are both H or combine to form a carbonate; and
R₅₇ is H, Br, I or Cl,
or a pharmaceutically acceptable salt, diastereomer, or enantiomer thereof; or
a composition comprising a compound having the structure: 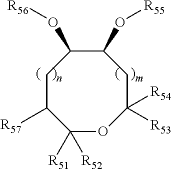
wherein
n=1 or 2;
m=1 or 2;
R₅₁, R₅₂, R₅₃ and R₅₄ are independently H, alkyl, or a haloalkyl;
R₅₅ and R₅₆ are both H or combine to form a carbonate; and
R₅₇ is H, Br, I or Cl,
or a pharmaceutically acceptable salt, diastereomer, or enantiomer thereof,
wherein the composition is free of plant extract.

138. The compound or composition of claim 137, wherein the compound has the structure:

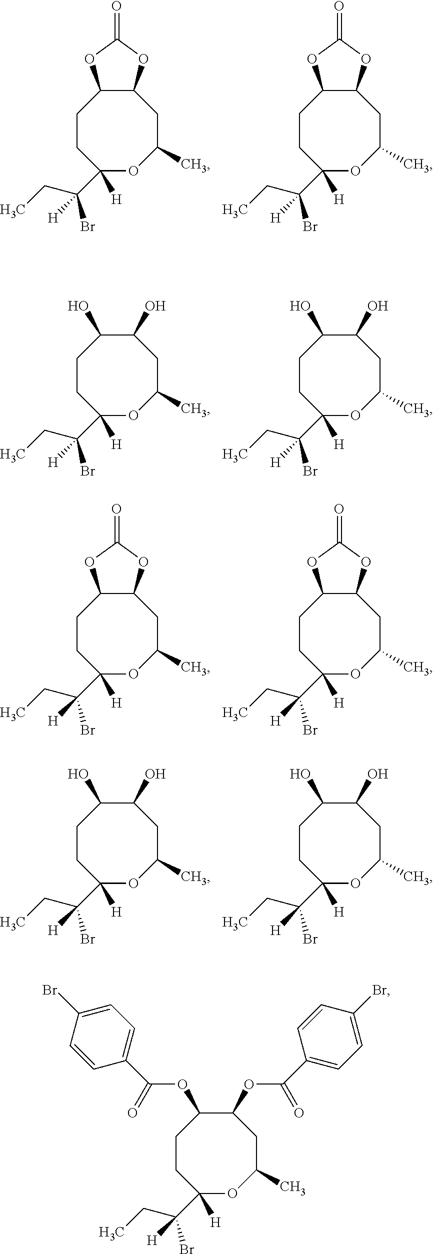

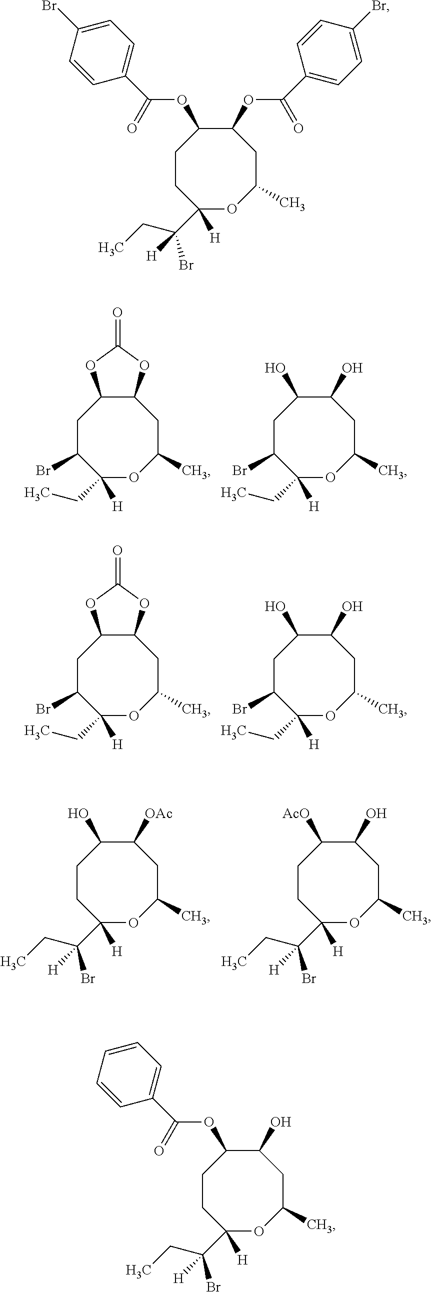

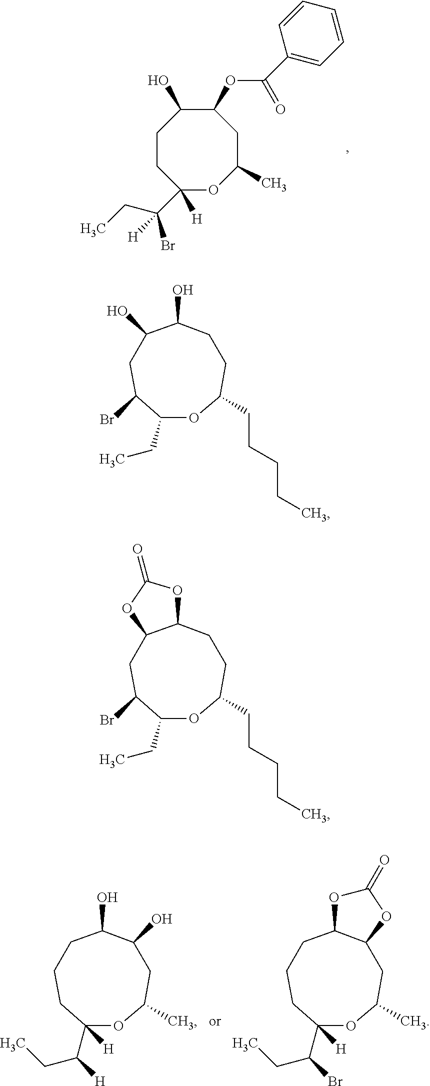

139. A process for producing the compound or composition of claim 137 comprising reacting the alkenyl alkyl ether having the structure: 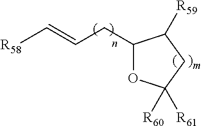
wherein
n=1, 2 or 3;
m=1 or 2;
R₅₈ is alkyl;
R₅₉ is OAc, OBoc, or OBz; and
R₆₀ and R₆₁ are independently H or alkyl;
with a second compound having the structure: 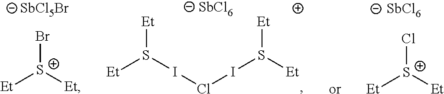
in a suitable solvent at a suitable temperature so as to thereby produce the compound; or
comprising
a) reacting the alkenyl alkyl ether having the structure: 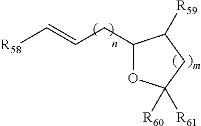
wherein
n=1, 2 or 3;
m=1 or 2;
R₅₈ is alkyl;
R₅₉ is OAc, OBoc, or OBz; and
R₆₀ and R₆₁ are independently H or alkyl;
with a second compound having the structure: 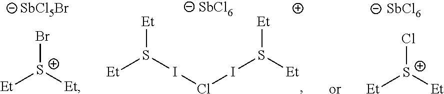
in a suitable solvent at a suitable temperature so as to thereby produce the compound of the composition; and
b) admixing the product of the step a) with a carrier so as to thereby produce the composition.

140. A compound having the structure having the structure: 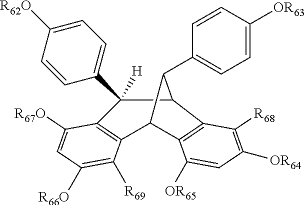
wherein
R₆₂, R₆₃, R₆₄, R₆₅, R₆₆, and R₆₇ are independently H, methyl, substituted or unsubstituted alkyl, substituted or unsubstituted heteroalkyl, substituted or unsubstituted aryl, substituted or unsubstituted heteroaryl, phosphate, sulfate, sulfonic ester, or ester; and
R₆₈ and R₆₉ are independently H, Cl, Br or I,
or a pharmaceutically acceptable salt thereof; or
a composition comprising a compound having the structure: 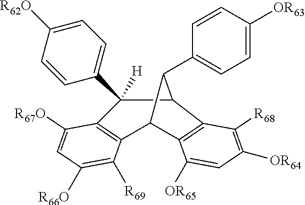
wherein
R₆₂, R₆₃, R₆₄, R₆₅, R₆₆, and R₆₇ are independently H, methyl, substituted or unsubstituted alkyl, substituted or unsubstituted heteroalkyl, substituted or unsubstituted aryl, substituted or unsubstituted heteroaryl, phosphate, sulfate, sulfonic ester, or ester; and
R₆₈ and R₆₉ are independently H, Cl, Br or I,
or a pharmaceutically acceptable salt thereof,
wherein the composition is free of plant extract.

141. The compound or composition of claim 140, wherein the structure is

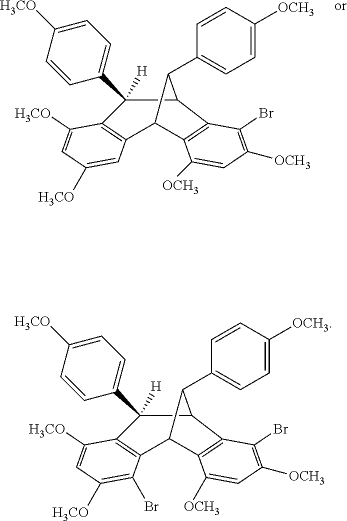 [SMILES: COc1ccc([C@H]2c3c(OC)cc(OC)cc3C3c4c(OC)cc(OC)c(Br)c4[C@@H]2[C@@H]3c2ccc(OC)cc2)cc1 ; "or" ; COc1ccc([C@H]2c3c(OC)cc(OC)c(Br)c3C3c4c(OC)cc(OC)c(Br)c4[C@@H]2[C@@H]3c2ccc(OC)cc2)cc1]

142. A process for producing the compound or composition of claim 140 comprising reacting an aromatic ring-containing compound having the structure: 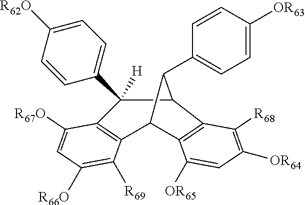
wherein
R₆₂, R₆₃, R₆₄, R₆₅, R₆₆, and R₆₇ are independently H, methyl, substituted or unsubstituted alkyl, substituted or unsubstituted heteroalkyl, substituted or unsubstituted aryl, substituted or unsubstituted heteroaryl, phosphate, sulfate, sulfonic ester, or ester;
R₆₈ is H, Cl, Br or I; and
R₆₉ is H,
with a second compound having the structure: 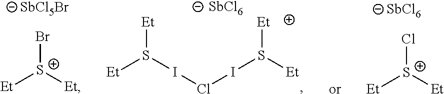
in a suitable solvent at a suitable temperature so as to thereby produce the compound; or
a) reacting a aromatic ring-containing compound having the structure: 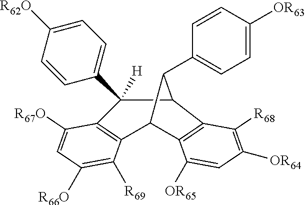
wherein
R₆₂, R₆₃, R₆₄, R₆₅, R₆₆, and R₆₇ are independently H, methyl, substituted or unsubstituted alkyl, substituted or unsubstituted heteroalkyl, substituted or unsubstituted aryl, substituted or unsubstituted heteroaryl, phosphate, sulfate, sulfonic ester, or ester;
R₆₈ is H, Cl, Br or I; and
R₆₉ is H,
with a second compound having the structure 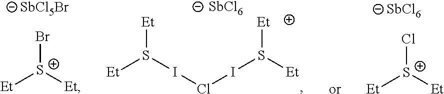
in a suitable solvent at a suitable temperature so as to thereby produce the compound of the composition; and
b) admixing the product of the step a) with a carrier so as to thereby produce the composition.
